(19) United States
(12) Patent Application Publication
(10) Pub. No.: US 2024/0200114 A1
(43) Pub. Date: Jun. 20, 2024

(54) BIOSYNTHESIS OF MOGROSIDES

(71) Applicant: GINKGO BIOWORKS, INC., Boston, MA (US)

(72) Inventors: Guillaume Beaudoin, Cambridge, MA (US); Alexandra Exner, Boston, MA (US); Annapurna Kamineni, Watertown, MA (US); Matthew McMahon, Melrose, MA (US); Joshua Trueheart, Waltham, MA (US)

(73) Assignee: GINKGO BIOWORKS, INC., Boston, MA (US)

(21) Appl. No.: 18/285,018

(22) PCT Filed: Apr. 1, 2022
(86) PCT No.: PCT/US2022/023173

§ 371 Date: Sep. 29, 2023

Related U.S. Application Data
(60) Provisional application No. 63/170,324, filed on Apr. 2, 2021.

(51) Int. Cl. C12P 19/56; C12N 9/02; C12N 9/10; C12N 9/14; C12N 9/90; C12P 19/60

(52) U.S. Cl. CPC C12P 19/56; C12N 9/0071; C12N 9/1051; C12N 9/14; C12N 9/90; C12P 19/60; C12Y 114/19; C12Y 204/01017; C12Y 303/02009; C12Y 504/99007

(57) ABSTRACT

Described in this application are proteins and host cells involved in methods of producing mogrol precursors, mogrol, and/or mogrosides.

### CROSS-REFERENCE TO RELATED APPLICATIONS

This application claims the benefit under 35 U.S.C. § 119(e) of U.S. Provisional Application No. 63/170,324, filed Apr. 2, 2021, entitled “BIOSYNTHESIS OF MOGROSIDES,” the entire disclosure of which is hereby incorporated by reference in its entirety.

### REFERENCE TO A SEQUENCE LISTING SUBMITTED AS A TEXT FILE VIA EFS-WEB

The instant application contains a Sequence Listing which has been submitted in ASCII format via EFS-Web and is hereby incorporated by reference in its entirety. The ASCII file, created on Apr. 1, 2022, is named G091970077WO00-SEQ-FL.TXT and is 886,131 bytes in size.

### FIELD OF THE INVENTION

The present disclosure relates to the production of mogrol precursors, mogrol and mogrosides in recombinant cells.

### BACKGROUND

Mogrosides are glycosides of cucurbitane derivatives. Highly sought after as sweeteners and sugar alternatives, mogrosides are naturally synthesized in the fruits of plants, including Siraitia grosvenorii (S. grosvenorii). Although anti-cancer, anti-oxidative, and anti-inflammatory properties have been ascribed to mogrosides, characterization of the exact proteins involved in mogroside biosynthesis is limited. Furthermore, mogroside extraction from fruit is labor-intensive and the structural complexity of mogrosides often hinders de novo chemical synthesis.

### SUMMARY

Aspects of the disclosure relate to host cells for producing mogrol, one or more mogrol precursors, and/or one or more mogrosides. In some embodiments, the host cell comprises a heterologous polynucleotide encoding a lanosterol synthase with reduced activity as compared to a wild-type lanosterol synthase, wherein the host cell is capable of producing:
(a) one or more mogrol precursors selected from the group consisting of: squalene, 2-3-oxidosqualene, 2,3,22,23-dioxidosqualene, cucurbitadienol, 24, 25-expoxycucurbitadienol, 11-hydroxycucurbitadienol, 11-hydroxy-24,25-epoxycucurbitadienol, 11-hydroxycucurbitadienol, 11-oxo-cucurbitadienol, and 24,25-dihydroxycucurbitadienol;
(b) mogrol; and/or
(c) one or more mogrosides.

In some embodiments, the host cell comprises a heterologous polynucleotide encoding a lanosterol synthase, wherein the lanosterol synthase comprises an amino acid substitution or deletion relative to SEQ ID NO: 1 at one or more residues corresponding to position 14, 33, 47, 50, 66, 80, 83, 85, 92, 94, 107, 122, 132, 145, 158, 170, 172, 184, 193, 197, 198, 212, 213, 227, 228, 231, 235, 248, 249, 260, 282, 286, 287, 289, 295, 296, 309, 314, 316, 329, 344, 360, 370, 371, 372, 398, 407, 414, 417, 423, 432, 437, 442, 444, 452, 474, 479, 491, 498, 515, 526, 529, 536, 544, 552, 559, 560, 564, 578, 586, 608, 610, 617, 619, 620, 631, 638, 650, 655, 660, 679, 686, 702, 710, 726, 736, 738, and/or 742 in SEQ ID NO: 1.

In some embodiments, the lanosterol synthase comprises 2, 3, 4, 5, 6, 7, 8, 9, 10, 11, or 12 amino acid substitutions and/or deletions relative to SEQ ID NO: 1. In some embodiments, the lanosterol synthase comprises: the amino acid Y at the residue corresponding to position 14 in SEQ ID NO:1; the amino acid Q at the residue corresponding to position 33 in SEQ ID NO:1; the amino acid E at the residue corresponding to position 47 in SEQ ID NO:1; the amino acid G at the residue corresponding to position 50 in SEQ ID NO:1; the amino acid R at the residue corresponding to position 66 in SEQ ID NO: 1; the amino acid G at the residue corresponding to position 80 in SEQ ID NO: 1; the amino acid L at the residue corresponding to position 83 in SEQ ID NO: 1; the amino acid N at the residue corresponding to position 85 in SEQ ID NO:1; the amino acid I at the residue corresponding to position 92 in SEQ ID NO: 1; the amino acid S at the residue corresponding to position 94 in SEQ ID NO:1; the amino acid D at the residue corresponding to position 107 in SEQ ID NO:1; the amino acid C at the residue corresponding to position 122 in SEQ ID NO:1; the amino acid S at the residue corresponding to position 132 in SEQ ID NO:1; the amino acid C at the residue corresponding to position 145 in SEQ ID NO:1; the amino acid S at the residue corresponding to position 158 in SEQ ID NO:1; the amino acid A at the residue corresponding to position 170 in SEQ ID NO: 1; the amino acid N at the residue corresponding to position 172 in SEQ ID NO:1; the amino acid W at the residue corresponding to position 184 in SEQ ID NO:1; the amino acid C or H at the residue corresponding to position 193 in SEQ ID NO:1; the amino acid V at the residue corresponding to position 197 in SEQ ID NO:1; the amino acid I at the residue corresponding to position 198 in SEQ ID NO: 1; the amino acid I at the residue corresponding to position 212 in SEQ ID NO: 1; the amino acid L at the residue corresponding to position 213 in SEQ ID NO:1; the amino acid L at the residue corresponding to position 227 in SEQ ID NO:1; the amino acid T at the residue corresponding to position 228 in SEQ ID NO: 1; the amino acid V at the residue corresponding to position 231 in SEQ ID NO: 1; the amino acid M at the residue corresponding to position 235 in SEQ ID NO:1; the amino acid F at the residue corresponding to position 248 in SEQ ID NO:1; the amino acid L at the residue corresponding to position 249 in SEQ ID NO:1; the amino acid R at the residue corresponding to position 260 in SEQ ID NO:1; the amino acid I at the residue corresponding to position 282 in SEQ ID NO:1; the amino acid F at the residue corresponding to position 286 in SEQ ID NO: 1; the amino acid G at the residue corresponding to position 287 in SEQ ID NO:1; the amino acid G at the residue corresponding to position 289 in SEQ ID NO: 1; the amino acid I at the residue corresponding to position 295 in SEQ ID NO: 1; the amino acid T at the residue corresponding to position 296 in SEQ ID NO: 1; the amino acid F at the residue corresponding to position 309 in SEQ ID NO: 1; the amino acid S at the residue corresponding to position 314 in SEQ ID NO:1; the amino acid R at the residue corresponding to position 316 in SEQ ID NO:1; the amino acid N at the residue corresponding to position 329 in SEQ ID NO: 1; the amino acid A at the residue corresponding to position 344 in SEQ ID NO: 1; the amino acid S at the residue corresponding to position 360 in SEQ ID NO:1; the amino acid L at the residue corresponding to position 370 in SEQ ID NO:1; the amino acid V at the residue corresponding to position 371 in SEQ ID NO:1; the amino acid P at the residue corresponding to position 372 in SEQ ID NO:1; the amino acid I at the residue corresponding to position 398 in SEQ ID NO: 1; the amino acid V at the residue corresponding to position 407 in SEQ ID NO:1; the amino acid S at the residue corresponding to position 414 in SEQ ID NO:1; the amino acid S at the residue corresponding to position 417 in SEQ ID NO:1; the amino acid L at the residue corresponding to position 423 in SEQ ID NO:1; the amino acid I or S at the residue corresponding to position 432 in SEQ ID NO:1; the amino acid L at the residue corresponding to position 437 in SEQ ID NO:1; the amino acid V at the residue corresponding to position 442 in SEQ ID NO:1; the amino acid M or S at the residue corresponding to position 444 in SEQ ID NO:1; the amino acid G at the residue corresponding to position 452 in SEQ ID NO:1; the amino acid V at the residue corresponding to position 474 in SEQ ID NO:1; the amino acid S at the residue corresponding to position 479 in SEQ ID NO:1; the amino acid Q at the residue corresponding to position 491 in SEQ ID NO:1; the amino acid N at the residue corresponding to position 498 in SEQ ID NO: 1; the amino acid L at the residue corresponding to position 515 in SEQ ID NO:1; the amino acid T at the residue corresponding to position 526 in SEQ ID NO: 1; the amino acid T at the residue corresponding to position 529 in SEQ ID NO: 1; the amino acid F at the residue corresponding to position 536 in SEQ ID NO:1; the amino acid Y at the residue corresponding to position 544 in SEQ ID NO:1; the amino acid E at the residue corresponding to position 552 in SEQ ID NO:1; the amino acid A at the residue corresponding to position 559 in SEQ ID NO:1; the amino acid M at the residue corresponding to position 560 in SEQ ID NO: 1; the amino acid C or N at the residue corresponding to position 564 in SEQ ID NO:1; the amino acid P at the residue corresponding to position 578 in SEQ ID NO:1; the amino acid F at the residue corresponding to position 586 in SEQ ID NO:1; the amino acid T at the residue corresponding to position 608 in SEQ ID NO:1; the amino acid I at the residue corresponding to position 610 in SEQ ID NO: 1; the amino acid V at the residue corresponding to position 617 in SEQ ID NO:1; the amino acid L at the residue corresponding to position 619 in SEQ ID NO:1; the amino acid S at the residue corresponding to position 620 in SEQ ID NO:1; the amino acid E or R at the residue corresponding to position 631 in SEQ ID NO:1; the amino acid D at the residue corresponding to position 638 in SEQ ID NO:1; the amino acid L at the residue corresponding to position 650 in SEQ ID NO:1; the amino acid A at the residue corresponding to position 655 in SEQ ID NO:1; the amino acid H at the residue corresponding to position 660 in SEQ ID NO:1; the amino acid S at the residue corresponding to position 679 in SEQ ID NO:1; the amino acid E at the residue corresponding to position 686 in SEQ ID NO: 1; the amino acid D at the residue corresponding to position 702 in SEQ ID NO: 1; the amino acid Q at the residue corresponding to position 710 in SEQ ID NO:1; the amino acid L or V at the residue corresponding to position 726 in SEQ ID NO: 1; the amino acid F at the residue corresponding to position 736 in SEQ ID NO:1; the amino acid M at the residue corresponding to position 738 in SEQ ID NO:1; and/or a truncation that results in deletion of the residue corresponding to position 742 in SEQ ID NO: 1.

In some embodiments, the lanosterol synthase comprises the amino acid substitution E617V, G107D, and/or K631E relative to SEQ ID NO: 1. In some embodiments, relative to SEQ ID NO: 1, the lanosterol synthase comprises: R33Q, R193C, D289G, N295I, S296T, N620S, and Y736F; R184W, L235M, L260R, and E710Q; K47E, L92I, T360S, S372P, T444M, and R578P; D50G, K66R, N94S, G417S, E617V, and F726L; N14Y, N132S, Y145C, R193H, I286F, L316R, F432I, E442V, T444S, I479S, K631R, and T655A; F432S, D452G, and I536F; E287G, K329N, E617V, and F726V; E231V, A407V, Q423L, A529T, and Y564C; V248F, D371V, and G702D; L197V, K282I, N314S, P370L, A608T, G638D, and F650L; L491Q, Y586F, and R660H; G122C, H249L, and K738M; P227L, E474V, V559A, and Y564N; K85N, G158S, S515L, P526T, Q619L, and a truncation resulting in a deletion of the residue corresponding to Q742 in SEQ ID NO: 1; G107D and K631E; T212I, W213L, N544Y, and V552E; I172N, C414S, L560M, and G679S; R193C, D289G, N295I, S296T, N620S, and Y736F; K85N and G158S; L197V, K282I, N314S, and P370L; I172N, C414S, and L560M; D371V, M610I, and G702D; D371V, K498N, M610I, and G702D; D80G, P83L, T170A, T198I, and A228T; T360S, S372P, T444M, and R578P; D50G, K66R, N94S, G417S, and E617V; or L309F, V344A, T398I, and K686E.

In some embodiments, relative to SEQ ID NO: 1, the lanosterol synthase comprises the following amino acid substitutions: R193C, D289G, N295I, S296T, N620S, and Y736F; F432S, D452G, and I536F; K85N and G158S; L197V, K282I, N314S, and P370L; I172N, C414S, L560M, and G679S; I172N, C414S, and L560M; D371V, M610I, and G702D; D371V, K498N, M610I, and G702D; D80G, P83L, T170A, T198I, and A228T; D50G, K66R, N94S, G417S, E617V, and F726L; T360S, S372P, T444M, and R578P; D50G, K66R, N94S, G417S, and E617V; and L309F, V344A, T398I, and K686E.

In some embodiments, relative to SEQ ID NO: 1, the lanosterol synthase comprises the following amino acid substitutions: D50G, K66R, N94S, G417S, E617V, and F726L; K85N and G158S; K47E, L92I, T360S, S372P, T444M, and R578P; F432S, D452G, and I536F; T360S, S372P, T444M, and R578P; L491Q, Y586F, and R660H; K85N, G158S, S515L, P526T, Q619L, and a truncation that results in deletion of the residue corresponding to position 742 in SEQ ID NO: 1; or I172N, C414S, L560M, and G679S.

In some embodiments, the lanosterol synthase comprises an amino acid substitution or deletion relative to SEQ ID NO: 1 at one or more residues corresponding to position 14, 33, 47, 50, 66, 85, 92, 94, 122, 132, 145, 158, 193, 231, 248, 249, 286, 287, 289, 295, 296, 316, 329, 360, 371, 372, 407, 417, 423, 432, 442, 444, 479, 515, 526, 529, 564, 578, 617, 619, 620, 631, 655, 702, 726, 736, 738, and/or 742 in SEQ ID NO: 1. In some embodiments, the lanosterol synthase comprises relative to SEQ ID NO: 1: R33Q, R193C, D289G, N295I, S296T. N620S, and Y736F; K47E, L92I, T360S, S372P, T444M, and R578P; D50G, K66R, N94S, G417S, E617V, and F726L; N14Y, N132S, Y145C, R193H, I286F, L316R, F432I, E442V, T444S, I479S, K631R, and T655A; E287G, K329N, E617V, and F726V; E231V, A407V, Q423L, A529T, and Y564C; V248F, D371V, and G702D; G122C, H249L, and K738M; or K85N, G158S, S515L, P526T, and Q619L, and a truncation resulting in a deletion of the residue corresponding to Q742 in SEQ ID NO: 1.

In some embodiments, the lanosterol synthase comprises a sequence that is at least 90% identical to SEQ ID NO: 3, 83-87, 89-92, 94-95, 99, 118-120, 316-319, 321-326, 329, or 331. In some embodiments, the lanosterol synthase comprises SEQ ID NO: 3, 83-87, 89-92, 94-95, 99, 118-120, 316-319, 321-326, 329, or 331. In some embodiments, the heterologous polynucleotide comprises a sequence that is at least 90% identical to SEQ ID NO: 4, 62-66, 68-71, 73-74, 78, 103-109, 111-117, 328, or 330. In some embodiments, the heterologous polynucleotide comprises the sequence of SEQ ID NO: 4, 62-66, 68-71, 73-74, 78, 103-109, 111-117, 328, or 330.

Further aspects of the disclosure relate to host cells that comprise a heterologous polynucleotide encoding a lanosterol synthase, wherein the lanosterol synthase comprises a sequence that is at least 90% identical to SEQ ID NO: 3, 83-87, 89-92, 94-95, 99, 100-102, 118-120, 316-319, 321-326, 329, or 331. In some embodiments, the lanosterol synthase comprises the sequence of SEQ ID NO: 3, 83-87, 89-92, 94-95, 99, 100-102, 118-120, 316-319, 321-326, 329, or 331.

Further aspects of the disclosure relate to host cells that comprise a heterologous polynucleotide encoding a lanosterol synthase, wherein the lanosterol synthase comprises relative to SEQ ID NO: 1: R33Q, R193C, D289G, N295I, S296T, N620S, and Y736F; K47E, L92I, T360S, S372P, T444M, and R578P; D50G, K66R, N94S, G417S, E617V, and F726L; N14Y, N132S, Y145C, R193H, I286F, L316R, F432I, E442V, T444S, I479S, K631R, and T655A; E287G, K329N, E617V, and F726V; E231V, A407V, Q423L, A529T, and Y564C; V248F, D371V, and G702D; G122C, H249L, and K738M; or K85N, G158S, S515L, P526T, and Q619L, and a truncation resulting in a deletion of the residue corresponding to Q742 in SEQ ID NO: 1.

Further aspects of the disclosure relate to host cells that comprise a heterologous polynucleotide encoding a lanosterol synthase, wherein the heterologous polynucleotide comprises a sequence that is at least 90% identical to SEQ ID NO: 4, 62-66, 68-71, 73-74, 78, 80-82, 103-109, 111-117, 328, or 330. In some embodiments, the heterologous polynucleotide comprises SEQ ID NO: 4, 62-66, 68-71, 73-74, 78, 80-82, 103-109, 111-117, 328, or 330.

In some embodiments, the host cell comprises a heterologous polynucleotide encoding a lanosterol synthase, wherein the lanosterol synthase comprises an amino acid substitution or deletion relative to SEQ ID NO: 313 at one or more residues corresponding to position 64, 120, 121, 136, 226, 268, 275, 281, 300, 322, 333, 438, 502, 604, 619, 628, 656, 693, 726, 727, 728, 729, 730, and/or 731.

In some embodiments, the lanosterol synthase comprises: the amino acid G at the residue corresponding to position 64 in SEQ ID NO: 313; the amino acid V at the residue corresponding to position 120 in SEQ ID NO: 313; the amino acid S at the residue corresponding to position 121 in SEQ ID NO: 313; the amino acid V at the residue corresponding to position 136 in SEQ ID NO: 313; the amino acid I at the residue corresponding to position 226 in SEQ ID NO: 313; the amino acid S at the residue corresponding to position 268 in SEQ ID NO: 313; the amino acid I at the residue corresponding to position 275 in SEQ ID NO: 313; the amino acid A at the residue corresponding to position 281 in SEQ ID NO: 313; the amino acid G at the residue corresponding to position 300 in SEQ ID NO: 313; the amino acid G at the residue corresponding to position 322 in SEQ ID NO: 313; the amino acid A at the residue corresponding to position 333 in SEQ ID NO: 313; the amino acid E at the residue corresponding to position 438 in SEQ ID NO: 313; the amino acid L at the residue corresponding to position 502 in SEQ ID NO: 313; the amino acid N at the residue corresponding to position 604 in SEQ ID NO: 313; the amino acid S at the residue corresponding to position 619 in SEQ ID NO: 313; the amino acid E at the residue corresponding to position 628 in SEQ ID NO: 313; the amino acid T at the residue corresponding to position 656 in SEQ ID NO: 313; the amino acid G at the residue corresponding to position 693 in SEQ ID NO: 313; and/or deletion of residues corresponding to positions 726-731 in SEQ ID NO: 313.

In some embodiments, the lanosterol synthase comprises relative to SEQ ID NO: 313: P121S, A136V, S300G, V322G, K438E, F502L, K628E, and deletion of residues corresponding to positions 726-731 in SEQ ID NO: 313; K268S, T281A, F502L, T604N, A656T, and E693G; or C619S, F275I, I120V, M226I, R64G, and T333A.

In some embodiments, the lanosterol synthase comprises a sequence that is at least 90% identical to any one of SEQ ID NOs: 100-102.

In some embodiments, the lanosterol synthase comprises a sequence selected from SEQ ID NOs: 100-102.

In some embodiments, the heterologous polynucleotide encoding the lanosterol synthase comprises a sequence that is at least 90% identical to a sequence selected from SEQ ID NOs: 80-82.

In some embodiments, the heterologous polynucleotide encoding the lanosterol synthase comprises a sequence selected from SEQ ID NOs: 80-82.

In some embodiments, the host cell is capable of producing mevalonate. In some embodiments, the host cell is capable of producing at least 0.2 g/L mevalonate. In some embodiments, the host cell is capable of producing at least 0.7 g/L mevalonate. In some embodiments, the host cell is capable of producing at least 9 mg/L cucurbitadienol. In some embodiments, the host cell is capable of producing at least 1.1 fold more cucurbitadienol than a control host cell comprising SEQ ID NO: 1 and/or a control host cell comprising SEQ ID NO: 313. In some embodiments, the host cell is capable of producing at least 3 fold more cucurbitadienol than a control host cell comprising SEQ ID NO: 1 and/or a control host cell comprising SEQ ID NO: 313. In some embodiments, the host cell is capable of producing at most 200 mg/L lanosterol. In some embodiments, the host cell is capable of producing at least 5 mg/L oxidosqualene.

In some embodiments, the host cell is capable of producing more mevalonate than a control host cell that does not comprise the heterologous polynucleotide.

In some embodiments, the host cell further comprises one or more heterologous polynucleotides encoding one or more of: a UDP-glycosyltransferases (UGT) enzyme, a cucurbitadienol synthase (CDS) enzyme, a C11 hydroxylase, an epoxide hydrolase (EPH), and squalene epoxidase (SQE). In some embodiments, the UGT enzyme comprises a sequence that is at least 90% identical to SEQ ID NO: 121. In some embodiments, the CDS enzyme comprises a sequence that is at least 90% identical to any one of SEQ ID NOs: 226, SEQ ID NO: 235, SEQ ID NO: 232, and SEQ ID NO: 256. In some embodiments, the C11 hydroxylase comprises a sequence that is at least 90% identical to any one of SEQ ID NOS: 280-281, 305, and 315. In some embodiments, the EPH comprises a sequence that is at least 90% identical to any one of SEQ ID NO: 284-292 and 309-310. In some embodiments, the SQE comprises a sequence that is at least 90% identical to any one of SEQ ID NOs: 293-295 and 312. In some embodiments, the host cell further comprises a heterologous polynucleotide encoding a cytochrome P450 reductase. In some embodiments, the cytochrome P450 reductase comprises a sequence that is at least 90% identical to any one of SEQ ID NOs: 282-283 and 306-307. In some embodiments, the host cell further comprises a heterologous polynucleotide encoding a cytochrome P450 reductase with reduced activity as compared to a control cytochrome P450 reductase or a heterologous polynucleotide that reduces cytochrome P450 activity. In some embodiments, the control cytochrome P450 reductase is a wild-type P450 reductase.

In some embodiments, the host cell is a yeast cell, a plant cell, or a bacterial cell. In some embodiments, the host cell is a yeast cell. In some embodiments, the yeast cell is a Saccharomyces cerevisiae cell. In some embodiments, the yeast cell is a Yarrowia lipolytica cell. In some embodiments, the host cell is a bacterial cell. In some embodiments, the bacterial cell is an E. coli cell.

In some embodiments, the host cell further comprises a heterologous polynucleotide encoding an acetoacetyl CoA synthase. In some embodiments, the acetoacetyl CoA synthase comprises a sequence that is at least 90% identical to SEQ ID NO: 6. In some embodiments, the heterologous polynucleotide encoding the acetoacetyl CoA synthase comprises a sequence that is at least 90% identical to SEQ ID NO: 7.

In some embodiments, the one or more mogrosides is selected from mogroside I-A1 (MIA1), mogroside IE (MIE), mogroside II-A1 (MIIA1), mogroside II-A2 (MIIA2), mogroside III-A1 (MIIIA1), mogroside II-E (MIIE), mogroside III (MIII), siamenoside I, mogroside IV (MIV), mogroside IVa (MIVA), isomogroside IV, mogroside III-E (MIIIE), mogroside V (MV), mogroside VIA (MVIA), mogroside VIB (MVIB), isomogroside V, mogroside VIa1 (MVIa1), and/or mogroside VI (MVI).

Further aspects of the disclosure relate to methods of producing a mogroside comprising culturing any of the host cells associated with the disclosure.

Further aspects of the disclosure relate to methods of producing mogrol comprising culturing any of the host cells associated with the disclosure.

In some embodiments, the mogroside is selected from mogroside I-A1 (MIA1), mogroside IE (MIE), mogroside II-A1 (MIIA1), mogroside II-A2 (MIIA2), mogroside III-A1 (MIIIA1), mogroside II-E (MIIE), mogroside III (MIII), siamenoside I, mogroside IV (MIV), mogroside IVa (MIVA), isomogroside IV, mogroside III-E (MIIIE), mogroside V (MV), mogroside VIA (MVIA), mogroside VIB (MVIB), isomogroside V, mogroside VIa1 (MVIa1), and/or mogroside VI (MVI).

Further aspects of the disclosure relate to methods of producing mogrol, one or more mogrol precursors, and/or one or more mogrosides comprising culturing a host cell that comprises a heterologous polynucleotide encoding a lanosterol synthase, wherein the lanosterol synthase comprises an amino acid substitution or deletion relative to SEQ ID NO: 1 at one or more residues corresponding to position 14, 33, 47, 50, 66, 80, 83, 85, 92, 94, 107, 122, 132, 145, 158, 170, 172, 184, 193, 197, 198, 212, 213, 227, 228, 231, 235, 248, 249, 260, 282, 286, 287, 289, 295, 296, 309, 314, 316, 329, 344, 360, 370, 371, 372, 398, 407, 414, 417, 423, 432, 437, 442, 444, 452, 474, 479, 491, 498, 515, 526, 529, 536, 544, 552, 559, 560, 564, 578, 586, 608, 610, 617, 619, 620, 631, 638, 650, 655, 660, 679, 686, 702, 710, 726, 736, 738, and/or 742 in SEQ ID NO: 1 and wherein the host cell is capable of producing:
(a) one or more mogrol precursors selected from the group consisting of: squalene, 2-3-oxidosqualene, 2,3,22,23-dioxidosqualene, cucurbitadienol, 24, 25-expoxycucurbitadienol, 11-hydroxycucurbitadienol, 11-hydroxy-24,25-epoxycucurbitadienol, 11-hydroxycucurbitadienol, 11-oxo-cucurbitadienol, and 24,25-dihydroxycucurbitadienol;
(b) mogrol; and/or
(c) one or more mogrosides.

In some embodiments, the lanosterol synthase comprises 2, 3, 4, 5, 6, 7, 8, 9, 10, 11, or 12 amino acid substitutions and/or deletions relative to SEQ ID NO: 1. In some embodiments, the lanosterol synthase comprises: the amino acid Y at the residue corresponding to position 14 in SEQ ID NO:1; the amino acid Q at the residue corresponding to position 33 in SEQ ID NO:1; the amino acid E at the residue corresponding to position 47 in SEQ ID NO:1; the amino acid G at the residue corresponding to position 50 in SEQ ID NO:1; the amino acid R at the residue corresponding to position 66 in SEQ ID NO:1; the amino acid G at the residue corresponding to position 80 in SEQ ID NO: 1; the amino acid L at the residue corresponding to position 83 in SEQ ID NO: 1; the amino acid N at the residue corresponding to position 85 in SEQ ID NO:1; the amino acid I at the residue corresponding to position 92 in SEQ ID NO:1; the amino acid S at the residue corresponding to position 94 in SEQ ID NO:1; the amino acid D at the residue corresponding to position 107 in SEQ ID NO:1; the amino acid C at the residue corresponding to position 122 in SEQ ID NO:1; the amino acid S at the residue corresponding to position 132 in SEQ ID NO:1; the amino acid C at the residue corresponding to position 145 in SEQ ID NO: 1; the amino acid S at the residue corresponding to position 158 in SEQ ID NO:1; the amino acid A at the residue corresponding to position 170 in SEQ ID NO: 1; the amino acid N at the residue corresponding to position 172 in SEQ ID NO:1; the amino acid W at the residue corresponding to position 184 in SEQ ID NO:1; the amino acid C or H at the residue corresponding to position 193 in SEQ ID NO:1; the amino acid V at the residue corresponding to position 197 in SEQ ID NO:1; the amino acid I at the residue corresponding to position 198 in SEQ ID NO: 1; the amino acid I at the residue corresponding to position 212 in SEQ ID NO:1; the amino acid L at the residue corresponding to position 213 in SEQ ID NO:1; the amino acid L at the residue corresponding to position 227 in SEQ ID NO:1; the amino acid T at the residue corresponding to position 228 in SEQ ID NO: 1; the amino acid V at the residue corresponding to position 231 in SEQ ID NO:1; the amino acid M at the residue corresponding to position 235 in SEQ ID NO:1; the amino acid F at the residue corresponding to position 248 in SEQ ID NO:1; the amino acid L at the residue corresponding to position 249 in SEQ ID NO:1; the amino acid R at the residue corresponding to position 260 in SEQ ID NO:1; the amino acid I at the residue corresponding to position 282 in SEQ ID NO:1; the amino acid F at the residue corresponding to position 286 in SEQ ID NO: 1; the amino acid G at the residue corresponding to position 287 in SEQ ID NO:1; the amino acid G at the residue corresponding to position 289 in SEQ ID NO: 1; the amino acid I at the residue corresponding to position 295 in SEQ ID NO: 1; the amino acid T at the residue corresponding to position 296 in SEQ ID NO: 1; the amino acid F at the residue corresponding to position 309 in SEQ ID NO: 1; the amino acid S at the residue corresponding to position 314 in SEQ ID NO: 1; the amino acid R at the residue corresponding to position 316 in SEQ ID NO:1; the amino acid N at the residue corresponding to position 329 in SEQ ID NO:1; the amino acid A at the residue corresponding to position 344 in SEQ ID NO: 1; the amino acid S at the residue corresponding to position 360 in SEQ ID NO:1; the amino acid L at the residue corresponding to position 370 in SEQ ID NO:1; the amino acid V at the residue corresponding to position 371 in SEQ ID NO:1; the amino acid P at the residue corresponding to position 372 in SEQ ID NO:1; the amino acid I at the residue corresponding to position 398 in SEQ ID NO: 1; the amino acid V at the residue corresponding to position 407 in SEQ ID NO:1; the amino acid S at the residue corresponding to position 414 in SEQ ID NO:1; the amino acid S at the residue corresponding to position 417 in SEQ ID NO:1; the amino acid L at the residue corresponding to position 423 in SEQ ID NO: 1; the amino acid I or S at the residue corresponding to position 432 in SEQ ID NO: 1; the amino acid L at the residue corresponding to position 437 in SEQ ID NO:1; the amino acid V at the residue corresponding to position 442 in SEQ ID NO:1; the amino acid M or S at the residue corresponding to position 444 in SEQ ID NO:1; the amino acid G at the residue corresponding to position 452 in SEQ ID NO:1; the amino acid V at the residue corresponding to position 474 in SEQ ID NO:1; the amino acid S at the residue corresponding to position 479 in SEQ ID NO:1; the amino acid Q at the residue corresponding to position 491 in SEQ ID NO:1; the amino acid N at the residue corresponding to position 498 in SEQ ID NO: 1; the amino acid L at the residue corresponding to position 515 in SEQ ID NO:1; the amino acid T at the residue corresponding to position 526 in SEQ ID NO:1; the amino acid T at the residue corresponding to position 529 in SEQ ID NO:1; the amino acid F at the residue corresponding to position 536 in SEQ ID NO:1; the amino acid Y at the residue corresponding to position 544 in SEQ ID NO:1; the amino acid E at the residue corresponding to position 552 in SEQ ID NO:1; the amino acid A at the residue corresponding to position 559 in SEQ ID NO: 1; the amino acid M at the residue corresponding to position 560 in SEQ ID NO:1; the amino acid C or N at the residue corresponding to position 564 in SEQ ID NO:1; the amino acid P at the residue corresponding to position 578 in SEQ ID NO:1; the amino acid F at the residue corresponding to position 586 in SEQ ID NO:1; the amino acid T at the residue corresponding to position 608 in SEQ ID NO:1; the amino acid I at the residue corresponding to position 610 in SEQ ID NO: 1; the amino acid V at the residue corresponding to position 617 in SEQ ID NO: 1; the amino acid L at the residue corresponding to position 619 in SEQ ID NO:1; the amino acid S at the residue corresponding to position 620 in SEQ ID NO:1; the amino acid E or R at the residue corresponding to position 631 in SEQ ID NO: 1; the amino acid D at the residue corresponding to position 638 in SEQ ID NO: 1; the amino acid L at the residue corresponding to position 650 in SEQ ID NO: 1; the amino acid A at the residue corresponding to position 655 in SEQ ID NO:1; the amino acid H at the residue corresponding to position 660 in SEQ ID NO:1; the amino acid S at the residue corresponding to position 679 in SEQ ID NO:1; the amino acid E at the residue corresponding to position 686 in SEQ ID NO: 1; the amino acid D at the residue corresponding to position 702 in SEQ ID NO:1; the amino acid Q at the residue corresponding to position 710 in SEQ ID NO:1; the amino acid L or V at the residue corresponding to position 726 in SEQ ID NO:1; the amino acid F at the residue corresponding to position 736 in SEQ ID NO:1; the amino acid M at the residue corresponding to position 738 in SEQ ID NO:1; and/or a truncation that results in deletion of the residue corresponding to position 742 in SEQ ID NO: 1.

In some embodiments, the lanosterol synthase comprises the amino acid substitution E617V, G107D, and/or K631E relative to SEQ ID NO: 1. In some embodiments, relative to SEQ ID NO: 1, the lanosterol synthase comprises: R33Q, R193C, D289G, N295I, S296T, N620S, and Y736F; R184W, L235M, L260R, and E710Q; K47E, L92I, T360S, S372P, T444M, and R578P; D50G, K66R, N94S, G417S, E617V, and F726L; N14Y, N132S, Y145C, R193H, I286F, L316R, F432I, E442V, T444S, I479S, K631R, and T655A; F432S, D452G, and I536F; E287G, K329N, E617V, and F726V; E231V, A407V, Q423L, A529T, and Y564C; V248F, D371V, and G702D; L197V, K282I, N314S, P370L, A608T, G638D, and F650L; L491Q, Y586F, and R660H; G122C, H249L, and K738M; P227L, E474V, V559A, and Y564N; K85N, G158S, S515L, P526T, Q619L, and a truncation resulting in a deletion of the residue corresponding to Q742 in SEQ ID NO: 1; G107D and K631E; T212I, W213L, N544Y, and V552E; I172N, C414S, L560M, and G679S; R193C, D289G, N295I, S296T, N620S, and Y736F; K85N and G158S; L197V, K282I, N314S, and P370L; I172N, C414S, and L560M; D371V, M610I, and G702D; D371V, K498N, M610I, and G702D; D80G, P83L, T170A, T198I, and A228T; T360S, S372P, T444M, and R578P; D50G, K66R, N94S, G417S, and E617V; or L309F, V344A, T398I, and K686E.

In some embodiments, relative to SEQ ID NO: 1, the lanosterol synthase comprises the following amino acid substitutions: R193C, D289G, N295I, S296T, N620S, and Y736F; F432S, D452G, and I536F; K85N and G158S; L197V, K282I, N314S, and P370L; I172N, C414S, L560M, and G679S; I172N, C414S, and L560M; D371V, M610I, and G702D; D371V, K498N, M610I, and G702D; D80G, P83L, T170A, T198I, and A228T; D50G, K66R. N94S, G417S, E617V, and F726L; T360S, S372P, T444M, and R578P; D50G, K66R. N94S, G417S, and E617V; and L309F, V344A, T398I, and K686E.

In some embodiments, relative to SEQ ID NO: 1, the lanosterol synthase comprises the following amino acid substitutions: D50G, K66R, N94S, G417S, E617V, and F726L; K85N and G158S; K47E, L92I, T360S, S372P, T444M, and R578P; F432S, D452G, and I536F; T360S, S372P, T444M, and R578P; L491Q, Y586F, and R660H; K85N, G158S, S515L, P526T, Q619L, and a truncation that results in deletion of the residue corresponding to position 742 in SEQ ID NO: 1; or I172N, C414S, L560M, and G679S.

In some embodiments, the lanosterol synthase comprises an amino acid substitution or deletion relative to SEQ ID NO: 1 at one or more residues corresponding to position 14, 33, 47, 50, 66, 85, 92, 94, 122, 132, 145, 158, 193, 231, 248, 249, 286, 287, 289, 295, 296, 316, 329, 360, 371, 372, 407, 417, 423, 432, 442, 444, 479, 515, 526, 529, 564, 578, 617, 619, 620, 631, 655, 702, 726, 736, 738, and/or 742 in SEQ ID NO: 1. In some embodiments, the lanosterol synthase comprises relative to SEQ ID NO: 1: R33Q, R193C, D289G, N295I, S296T. N620S, and Y736F; K47E, L92I, T360S, S372P, T444M, and R578P; D50G, K66R, N94S, G417S, E617V, and F726L; N14Y, N132S, Y145C, R193H, I286F, L316R, F432I, E442V, T444S, I479S, K631R, and T655A; E287G, K329N, E617V, and F726V; E231V, A407V, Q423L, A529T, and Y564C; V248F, D371V, and G702D; G122C, H249L, and K738M; or K85N, G158S, S515L, P526T, and Q619L, and a truncation resulting in a deletion of the residue corresponding to Q742 in SEQ ID NO: 1.

In some embodiments, the lanosterol synthase comprises a sequence that is at least 90% identical to SEQ ID NO: 3, 83-87, 89-92, 94-95, 99, 118-120, 316-319, 321-326, 329, or 331. In some embodiments, the lanosterol synthase comprises SEQ ID NO: 3, 83-87, 89-92, 94-95, 99, 118-120, 316-319, 321-326, 329, or 331. In some embodiments, the heterologous polynucleotide comprises a sequence that is at least 90% identical to SEQ ID NO: 4, 62-66, 68-71, 73-74, 78, 103-109, 111-117, 328, or 330. In some embodiments, the heterologous polynucleotide comprises the sequence of SEQ ID NO: 4, 62-66, 68-71, 73-74, 78, 103-109, 111-117, 328, or 330.

Further aspects of the disclosure relate to methods of producing mogrol, one or more mogrol precursors, and/or one or more mogrosides comprising culturing a host cell that comprises a heterologous polynucleotide encoding a lanosterol synthase, wherein the lanosterol synthase comprises an amino acid substitution or deletion relative to SEQ ID NO: 313 at one or more residues corresponding to position 64, 120, 121, 136, 226, 268, 275, 281, 300, 322, 333, 438, 502, 604, 619, 628, 656, 693, 726, 727, 728, 729, 730, and/or 731.

In some embodiments, the lanosterol synthase comprises: the amino acid G at the residue corresponding to position 64 in SEQ ID NO: 313; the amino acid V at the residue corresponding to position 120 in SEQ ID NO: 313; the amino acid S at the residue corresponding to position 121 in SEQ ID NO: 313; the amino acid V at the residue corresponding to position 136 in SEQ ID NO: 313; the amino acid I at the residue corresponding to position 226 in SEQ ID NO: 313; the amino acid S at the residue corresponding to position 268 in SEQ ID NO: 313; the amino acid I at the residue corresponding to position 275 in SEQ ID NO: 313; the amino acid A at the residue corresponding to position 281 in SEQ ID NO: 313; the amino acid G at the residue corresponding to position 300 in SEQ ID NO: 313; the amino acid G at the residue corresponding to position 322 in SEQ ID NO: 313; the amino acid A at the residue corresponding to position 333 in SEQ ID NO: 313; the amino acid E at the residue corresponding to position 438 in SEQ ID NO: 313; the amino acid L at the residue corresponding to position 502 in SEQ ID NO: 313; the amino acid N at the residue corresponding to position 604 in SEQ ID NO: 313; the amino acid S at the residue corresponding to position 619 in SEQ ID NO: 313; the amino acid E at the residue corresponding to position 628 in SEQ ID NO: 313; the amino acid T at the residue corresponding to position 656 in SEQ ID NO: 313; the amino acid G at the residue corresponding to position 693 in SEQ ID NO: 313; and/or deletion of residues corresponding to positions 726-731 in SEQ ID NO: 313.

In some embodiments, the lanosterol synthase comprises relative to SEQ ID NO: 313: P121S, A136V, S300G, V322G, K438E, F502L, K628E, and deletion of residues corresponding to positions 726-731 in SEQ ID NO: 313; K268S, T281A, F502L, T604N, A656T, and E693G; or C619S, F275I, I120V, M226I, R64G, and T333A.

In some embodiments, the lanosterol synthase comprises a sequence that is at least 90% identical to any one of SEQ ID NOs: 100-102.

In some embodiments, the lanosterol synthase comprises a sequence selected from SEQ ID NOs: 100-102.

In some embodiments, the heterologous polynucleotide encoding the lanosterol synthase comprises a sequence that is at least 90% identical to a sequence selected from SEQ ID NOs: 80-82.

In some embodiments, the heterologous polynucleotide encoding the lanosterol synthase comprises a sequence selected from SEQ ID NOs: 80-82.

In some embodiments, the host cell is capable of producing mevalonate. In some embodiments, the host cell is capable of producing at least 0.2 g/L mevalonate. In some embodiments, the host cell is capable of producing at least 0.7 g/L mevalonate. In some embodiments, the host cell is capable of producing at least 9 mg/L cucurbitadienol. In some embodiments, the host cell is capable of producing at least 1.1 fold more cucurbitadienol than a control host cell comprising SEQ ID NO: 1 and/or a control host cell comprising SEQ ID NO: 313. In some embodiments, the host cell is capable of producing at least 3 fold more cucurbitadienol than a control host cell comprising SEQ ID NO: 1 and/or a control host cell comprising SEQ ID NO: 313. In some embodiments, the host cell is capable of producing at most 200 mg/L lanosterol. In some embodiments, the host cell is capable of producing at least 5 mg/L oxidosqualene.

In some embodiments, the host cell is capable of producing more mevalonate than a control host cell that does not comprise the heterologous polynucleotide.

In some embodiments, the host cell further comprises one or more heterologous polynucleotides encoding one or more of: a UDP-glycosyltransferases (UGT) enzyme, a cucurbitadienol synthase (CDS) enzyme, a C11 hydroxylase, an epoxide hydrolase (EPH), and squalene epoxidase (SQE). In some embodiments, the UGT enzyme comprises a sequence that is at least 90% identical to SEQ ID NO: 121. In some embodiments, the CDS enzyme comprises a sequence that is at least 90% identical to any one of SEQ ID NOs: 226, SEQ ID NO: 235, SEQ ID NO: 232, and SEQ ID NO: 256. In some embodiments, the C11 hydroxylase comprises a sequence that is at least 90% identical to any one of SEQ ID NOS: 280-281, 305, and 315. In some embodiments, the EPH comprises a sequence that is at least 90% identical to any one of SEQ ID NO: 284-292 and 309-310. In some embodiments, the SQE comprises a sequence that is at least 90% identical to any one of SEQ ID NOs: 293-295 and 312. In some embodiments, the host cell further comprises a heterologous polynucleotide encoding a cytochrome P450 reductase. In some embodiments, the cytochrome P450 reductase comprises a sequence that is at least 90% identical to any one of SEQ ID NOs: 282-283 and 306-307. In some embodiments, the host cell further comprises a heterologous polynucleotide encoding a cytochrome P450 reductase with reduced activity as compared to a control cytochrome P450 reductase or a heterologous polynucleotide that reduces cytochrome P450 activity. In some embodiments, the control cytochrome P450 reductase is a wild-type P450 reductase.

In some embodiments, the host cell is a yeast cell, a plant cell, or a bacterial cell. In some embodiments, the host cell is a yeast cell. In some embodiments, the yeast cell is a Saccharomyces cerevisiae cell. In some embodiments, the yeast cell is a Yarrowia lipolytica cell. In some embodiments, the host cell is a bacterial cell. In some embodiments, the bacterial cell is an E. coli cell.

In some embodiments, the host cell further comprises a heterologous polynucleotide encoding an acetoacetyl CoA synthase. In some embodiments, the acetoacetyl CoA synthase comprises a sequence that is at least 90% identical to SEQ ID NO: 6. In some embodiments, the heterologous polynucleotide encoding the acetoacetyl CoA synthase comprises a sequence that is at least 90% identical to SEQ ID NO: 7.

In some embodiments, the mogroside is selected from mogroside I-A1 (MIA1), mogroside IE (MIE), mogroside II-A1 (MIIA1), mogroside II-A2 (MIIA2), mogroside III-A1 (MIIIA1), mogroside II-E (MIIE), mogroside III (MIII), siamenoside I, mogroside IV (MIV), mogroside IVa (MIVA), isomogroside IV, mogroside III-E (MIIIE), mogroside V (MV), mogroside VIA (MVIA), mogroside VIB (MVIB), isomogroside V, mogroside VIa1 (MVIa1), and/or mogroside VI (MVI).

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways.

### BRIEF DESCRIPTION OF DRAWINGS

The accompanying drawings are not intended to be drawn to scale. The drawings are illustrative only and are not required for enablement of the disclosure. For purposes of clarity, not every component may be labeled in every drawing. In the drawings:

FIGS. 1A-1F include schematic overviews of the mevalonate pathway and putative mogrol biosynthesis pathways. SQS indicates squalene synthase, EPD indicates epoxidase, P450 indicates C11 hydroxylase, EPH indicates epoxide hydrolase, and CDS indicates cucurbitadienol synthase. FIGS. 1A-1B provide a non-limiting example of how the mevalonate pathway provides precursors for mogrol biosynthesis. FIG. 1B shows a sterol biosynthesis pathway and is a continuation of FIG. 1A. FIG. 1C and FIG. 1D show how the mevalonate pathway products feed into putative mogrol biosynthesis pathways. FIG. 1E shows non-limiting examples of primary UGT activity. FIG. 1F shows non-limiting examples of secondary UGT activity.

FIG. 2 is a graph depicting mevalonate production by Yarrowia strains comprising a lanosterol synthase.

FIG. 3 is a graph depicting cucurbitadienol production by strains comprising a lanosterol synthase (erg7 allele). Strain 870688 comprising SEQ ID NO: 1 was used as a control.

FIG. 4 is a graph depicting cucurbitadienol, ergosterol, lanosterol, and mevalonate production by strains comprising a lanosterol synthase (erg7 allele). Strain 887779 comprising SEQ ID NO: 1 was used as a control.

FIG. 5 is a graph depicting oxidosqualene production in lanosterol synthase temperature sensitive mutant (erg7 mutant) strains at 30° C. and 35° C. Three lanosterol synthase mutant strains 756247, 756248 and 756249 comprising SEQ ID NOs: 100-102, respectively, were tested and the parent BY4742 Saccharomyces cerevisiae strain was included as the negative control.

FIG. 6 is a graph depicting production of ergosterol, ethanol, and mevalonate and consumption of glucose in lanosterol synthase temperature sensitive mutant (erg7 mutant) strains at 30° C. Three lanosterol synthase mutant strains 756247, 756248 and 756249 comprising SEQ ID NOs: 100-102, respectively, were tested and the parent BY4742 Saccharomyces cerevisiae strain was included as the negative control.

FIG. 7 is a graph depicting production of ergosterol, ethanol, and mevalonate and consumption of glucose in lanosterol synthase temperature sensitive mutant (erg7 mutant) strains at 35° C. Three lanosterol synthase mutant strains 756247, 756248 and 756249 comprising SEQ ID NOs: 100-102, respectively, were tested and the parent BY4742 Saccharomyces cerevisiae strain was included as the negative control.

### DETAILED DESCRIPTION

Mogrosides are widely used as natural sweeteners, for example, in beverages. However, de novo synthesis and mogroside extraction from natural sources often involve high production costs and low yield. This disclosure provides host cells that are engineered to efficiently produce mogrol (or 11, 24, 25-trihydroxy cucurbitadienol), mogrosides, and precursors thereof. Methods include use of host cells which feature a variant of lanosterol synthase enzyme (e.g., a mutant with decreased but not abolished enzymatic activity). Examples 1 and 3-4 describe the identification and functional characterization of lanosterol synthases that can be used to increase production of mogrol precursors, mogrol, and mogrosides. In some embodiments, the host cell also features the heterologous expression of (e.g., the increased expression, level and/or activity of) any of various enzymes involved in synthesis of mogrol, mogrol precursors, mogroside precursors, and mogrosides, including but not limited to: cucurbitadienol synthase (CDS) enzymes, UDP-glycosyltransferase (UGT) enzymes, C11 hydroxylase enzymes, epoxide hydrolase (EPH) enzymes, squalene epoxidase (SQE) enzymes, or combinations thereof. In some embodiments, wherein 11-oxo mogrol is not a desired product, the level, expression and/or activity of a cytochrome P450 reductase, which is involved in synthesis of 11-oxo mogrol, is decreased in the host cell.

In some embodiments, the host cell further comprises a heterologous polynucleotide encoding an acetoacetyl CoA synthase (e.g., an acetoacetyl CoA synthase comprising the amino acid sequence provided in SEQ ID NO: 6).

### Synthesis of Mogrol and Mogrosides

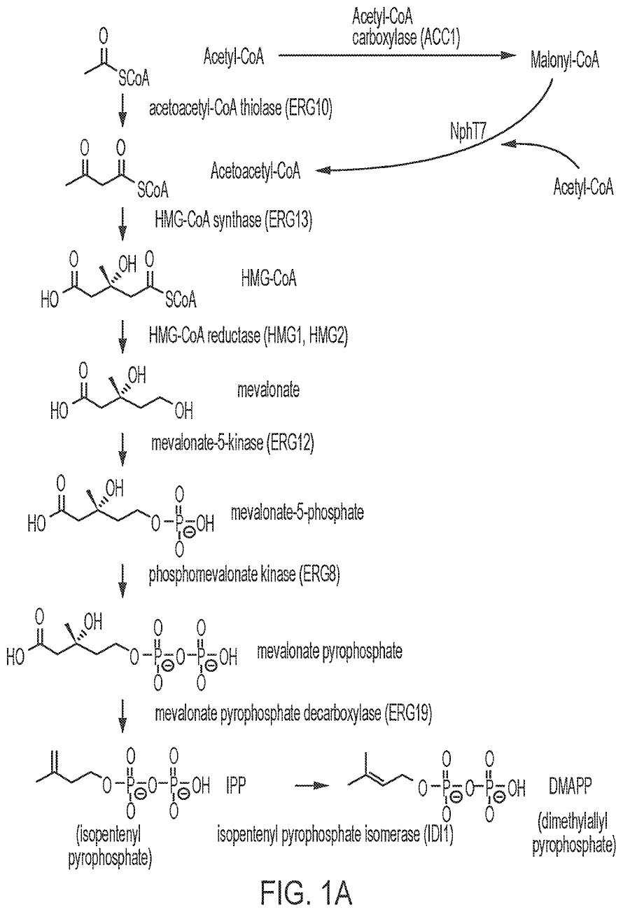

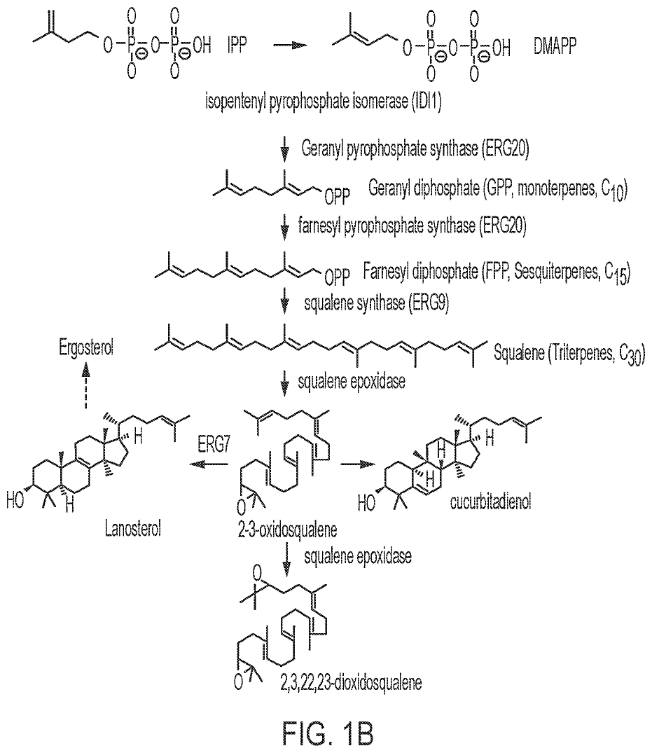

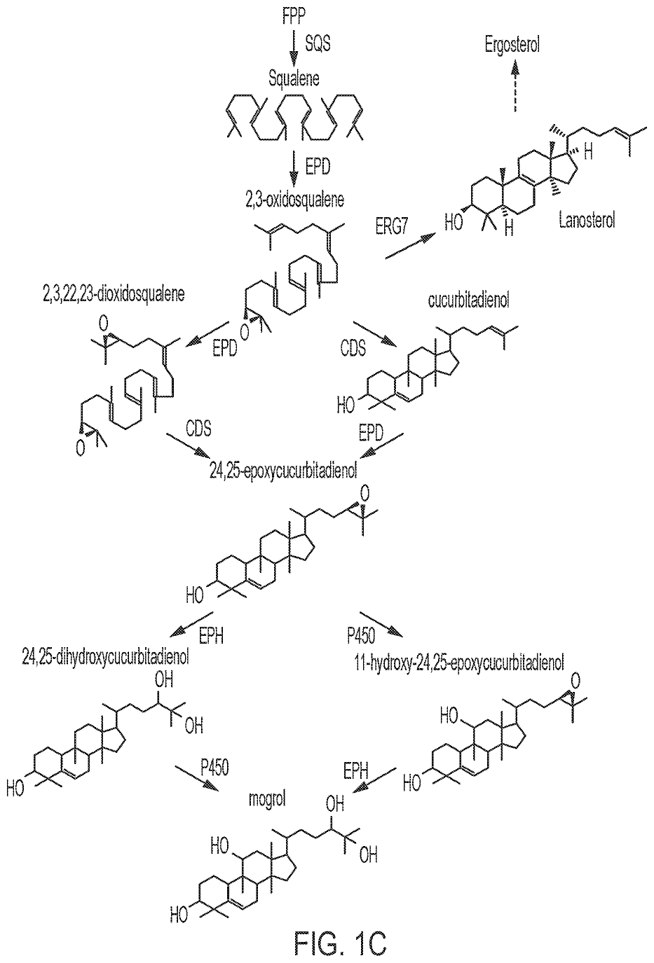

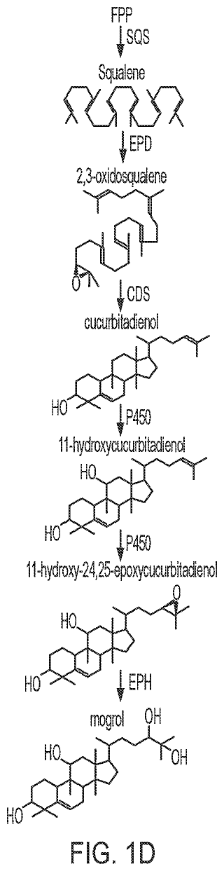

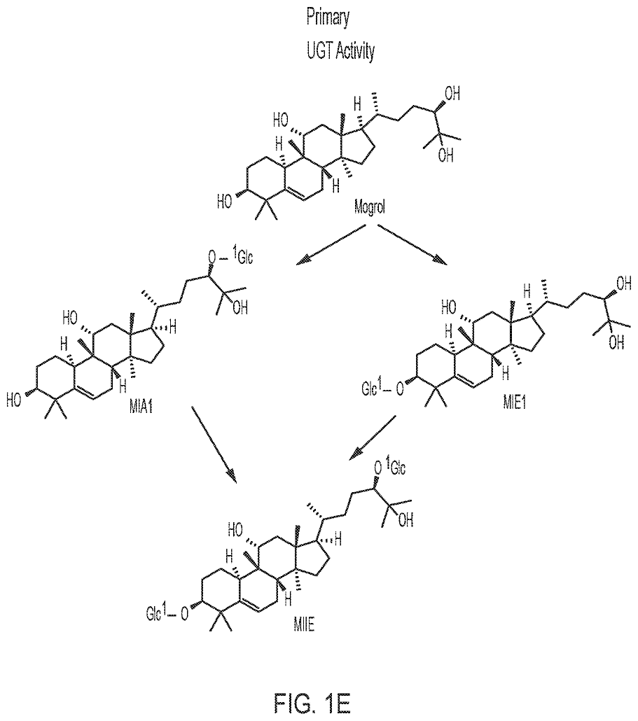

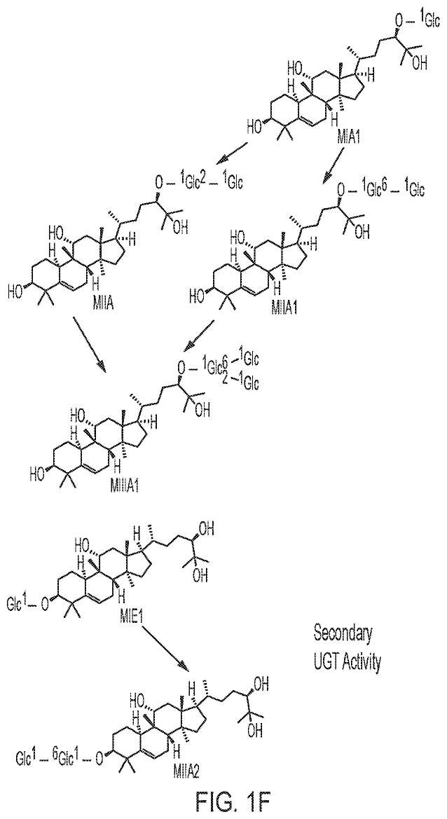

FIGS. 1A-1B show how the mevalonate pathway provides precursors for mogrol synthesis. First, two acetyl-CoA molecules are condensed to form acetoacetyl-CoA, which is then condensed to form 3-hydroxy-3-methyl-glutaryl-CoA (HMG-COA). Then, HMG-COA is reduced to form mevalonate. Mevalonate is subsequently, via multiple enzymatic steps, converted into isopentenyl pyrophosphate (IPP) and dimethylallyl pyrophosphate (DMAPP). FIGS. 1C-1D show putative mogrol synthesis pathways. An early step involves conversion of squalene to 2,3-oxidosqualene. As shown in FIG. 1C, 2,3-oxidosqualene can be first cyclized to cucurbitadienol followed by epoxidation to form 24,25-epoxycucurbitadienol, or 2,3-oxidosqualene can be epoxidized to 2,3,22,23-dioxidosqualene and then cyclized to 24,25-epoxycucurbitadienol. Next, the 24,25-epoxycucurbitadienol can be converted to mogrol (an aglycone of mogrosides) following epoxide hydrolysis and then oxidation, or oxidation and then epoxide hydrolysis. As shown in FIG. 1D, 2,3-oxidosqualene can be first cyclized to cucurbitadienol, which is then converted to 11-hydroxycucurbitadienol by a cytochrome P450 C11 hydroxylase. Then, a cytochrome P450 C11 hydroxylase may convert 11-hydroxycucurbitadienol to 11-hydroxy-24,25-epoxycucurbitadienol. 11-hydroxy-24,25-epoxycucurbitadienol may be converted to mogrol by epoxide hydrolase. C11 hydroxylases act in conjunction with cytochrome P450 reductases (not shown in FIGS. 1C-1D).

Mogrol can be distinguished from other cucurbitane triterpenoids by oxygenations at C3, C11, C24, and C25. Glycosylation of mogrol, for example at C3 and/or C24, leads to the formation of mogrosides.

Mogrol precursors include but are not limited to acetyl-CoA, acetoacetyl-CoA, HMG-CoA, mevalonate, mevalonate-5-phosphate, mevalonate pyrophosphate, isopentenyl pyrophosphate (IPP), dimethylallyl pyrophosphate (DMAPP), geranyl pyrophosphate (GPP), farnesyl diphosphate (FPP), squalene, 2-3-oxidosqualene, 2,3,22,23-dioxidosqualene, cucurbitadienol, 24, 25-expoxycucurbitadienol, 11-hydroxycucurbitadienol, 11-hydroxy-24,25-epoxycucurbitadienol, 11-hydroxy-cucurbitadienol, 11-oxo-cucurbitadienol, and 24,25-dihydroxycucurbitadienol. The term “dioxidosqualene” may be used to refer to 2,3,22,23-diepoxy squalene or 2,3,22,23-dioxido squalene. The term “2,3-epoxysqualene” may be used interchangeably with the term “2-3-oxidosqualene.” As used in this application, mogroside precursors include mogrol precursors, mogrol and mogrosides.

Examples of mogrosides include, but are not limited to, mogroside I-A1 (MIA1), mogroside IE (MIE or MIE), mogroside II-A1 (MIIA1 or M2A1), mogroside II-A2 (MIIA2 or M2A2), mogroside III-A1 (MIIIA1 or M3A1), mogroside II-E (MIIE or M2E), mogroside III (MIII or M3), siamenoside I, mogroside IV (MIV or M4), mogroside IVa (MIVA or M4A), isomogroside IV, mogroside III-E (MIIIE or M3E), mogroside V (MV or M5), mogroside VIA (MVIA), mogroside VIB (MVIB), isomogroside V, mogroside VIa1 (MVIa1), and mogroside VI (MVI or M6). In some embodiments, the mogroside produced is siamenoside I, which may be referred to as Siam. In some embodiments, the mogroside produced is MIIIE. Unless otherwise noted, when used in the plural, the terms “M1s”, “MIs”, “M2s”, “MIIs”, “M3s”, “MIIIs”, “M4s”, “MIVs”, “MVs”, “M5s”, “M6s”, and “MVIs” each refer to a class of mogrosides. As a non-limiting example, M2s or MIIs may include MIIA1, MIIA, MIIA2, and/or MIIE.

In other embodiments, a mogroside is a compound of Formula 1:

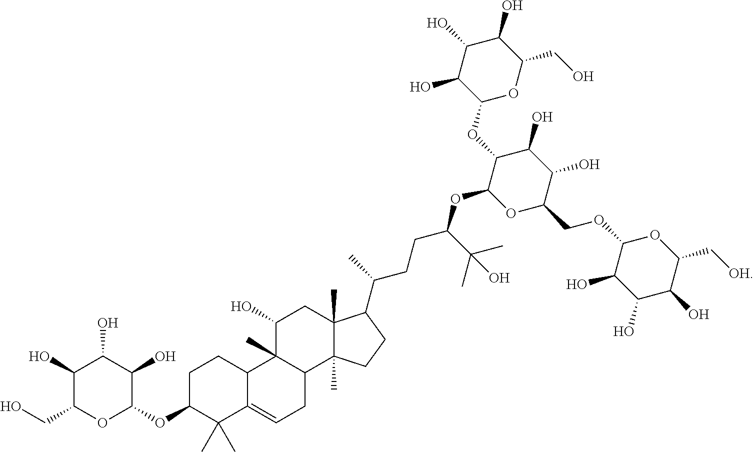 [SMILES: C[C@H](CC[C@@H](O[C@@H]1O[C@H](CO[C@@H]2O[C@H](CO)[C@@H](O)[C@H](O)[C@H]2O)[C@@H](O)[C@H](O)[C@H]1O[C@H]1O[C@@H](CO)[C@H](O)[C@@H](O)[C@@H]1O)C(C)(C)O)C1CC[C@@]2(C)C3CC=C4C(CC[C@H](O[C@@H]5O[C@H](CO)[C@@H](O)[C@H](O)[C@H]5O)C4(C)C)[C@]3(C)[C@H](O)C[C@]12C]

In some embodiments, the methods described in this application may be used to produce any of the compounds described in and incorporated by reference from US 2019/0071705 (which granted as U.S. Pat. No. 11,060,124), including compounds 1-20 as disclosed in US 2019/0071705. In some embodiments, the methods described in this application may be used to produce variants of any of the compounds described in and incorporated by reference from US 2019/0071705, including variants of compounds 1-20 as disclosed in US 2019/0071705. For example, a variant of a compound described in US 2019/0071705 can comprise a substitution of one or more alpha-glucosyl linkages in a compound described in US 2019/0071705 with one or more beta-glucosyl linkages. In some embodiments, a variant of a compound described in US 2019/0071705 comprises a substitution of one or more beta-glucosyl linkages in a compound described in US 2019/0071705 with one or more alpha-glucosyl linkages. In some embodiments, a variant of a compound described in US 2019/0071705 is a compound of Formula 1 shown above.

In some embodiments, a host cell comprising one or more proteins described herein (e.g., a lanosterol synthase, an acetoacetyl CoA synthase, a cytochrome b5 (CB5), a UDP-glycosyltransferase (UGT) enzyme, a cucurbitadienol synthase (CDS) enzyme, a C11 hydroxylase enzyme, a cytochrome P450 reductase enzyme, an epoxide hydrolase enzyme (EPH), a squalene epoxidase enzyme (SQE) and/or any proteins associated with the disclosure) is capable of producing at least 0.005 mg/L, at least 0.01 mg/L, at least 0.02 mg/L, at least 0.03 mg/L, at least 0.04 mg/L, at least 0.05 mg/L, at least 0.06 mg/L, at least 0.07 mg/L, at least 0.08 mg/L, at least 0.09 mg/L, at least 0.1 mg/L, at least 0.2 mg/L, at least 0.3 mg/L, at least 0.4 mg/L, at least 0.5 mg/L, at least 0.6 mg/L, at least 0.7 mg/L, at least 0.8 mg/L, at least 0.9 mg/L, at least 1 mg/L, at least 2 mg/L, at least 3 mg/L, at least 4 mg/L, at least 5 mg/L, at least 6 mg/L, at least 7 mg/L, at least 8 mg/L, at least 9 mg/L, at least 10 mg/L, at least 11 mg/L, at least 12 mg/L, at least 13 mg/L, at least 14 mg/L, at least 15 mg/L, at least 16 mg/L, at least 17 mg/L, at least 18 mg/L, at least 19 mg/L, at least 20 mg/L, at least 21 mg/L, at least 22 mg/L, at least 23 mg/L, at least 24 mg/L, at least 25 mg/L, at least 26 mg/L, at least 27 mg/L, at least 28 mg/L, at least 29 mg/L, at least 30 mg/L, at least 31 mg/L, at least 32 mg/L, at least 33 mg/L, at least 34 mg/L, at least 35 mg/L, at least 36 mg/L, at least 37 mg/L, at least 38 mg/L, at least 39 mg/L, at least 40 mg/L, at least 41 mg/L, at least 42 mg/L, at least 43 mg/L, at least 44 mg/L, at least 45 mg/L, at least 46 mg/L, at least 47 mg/L, at least 48 mg/L, at least 49 mg/L, at least 50 mg/L, at least 51 mg/L, at least 52 mg/L, at least 53 mg/L, at least 54 mg/L, at least 55 mg/L, at least 56 mg/L, at least 57 mg/L, at least 58 mg/L, at least 59 mg/L, at least 60 mg/L, at least 61 mg/L, at least 62 mg/L, at least 63 mg/L, at least 64 mg/L, at least 65 mg/L, at least 66 mg/L, at least 67 mg/L, at least 68 mg/L, at least 69 mg/L, at least 70 mg/L, at least 75 mg/L, at least 80 mg/L, at least 85 mg/L, at least 90 mg/L, at least 95 mg/L, at least 100 mg/L, at least 125 mg/L, at least 150 mg/L, at least 175 mg/L, at least 200 mg/L, at least 225 mg/L, at least 250 mg/L, at least 275 mg/L, at least 300 mg/L, at least 325 mg/L, at least 350 mg/L, at least 375 mg/L, at least 400 mg/L, at least 425 mg/L, at least 450 mg/L, at least 475 mg/L, at least 500 mg/L, at least 1,000 mg/L, at least 2,000 mg/L, at least 3,000 mg/L, at least 4,000 mg/L, at least 5,000 mg/L, at least 6,000 mg/L, at least 7,000 mg/L, at least 8,000 mg/L, at least 9,000 mg/L, at least 10,000 mg/L, at least 11 g/L, at least 12 g/L, at least 13 g/L, at least 14 g/L, at least 15 g/L, at least 16 g/L, at least 17 g/L, at least 18 g/L, at least 19 g/L, at least 20 g/L, at least 21 g/L, at least 22 g/L, at least 23 g/L, at least 24 g/L, at least 25 g/L, at least 26 g/L, at least 27 g/L, at least 28 g/L, at least 29 g/L, at least 30 g/L, at least 31 g/L, at least 32 g/L, at least 33 g/L, at least 34 g/L, at least 35 g/L, at least 36 g/L, at least 37 g/L, at least 38 g/L, at least 39 g/L, at least 40 g/L, at least 41 g/L, at least 42 g/L, at least 43 g/L, at least 44 g/L, at least 45 g/L, at least 46 g/L, at least 47 g/L, at least 48 g/L, at least 49 g/L, at least 50 g/L, at least 51 g/L, at least 52 g/L, at least 53 g/L, at least 54 g/L, at least 55 g/L, at least 56 g/L, at least 57 g/L, at least 58 g/L, at least 59 g/L, at least 60 g/L, at least 61 g/L, at least 62 g/L, at least 63 g/L, at least 64 g/L, at least 65 g/L, at least 66 g/L, at least 67 g/L, at least 68 g/L, at least 69 g/L, at least 70 g/L, at least 75 g/L, at least 80 g/L, at least 85 g/L, at least 90 g/L, at least 95 g/L, at least 100 g/L, at least 125 g/L, at least 150 g/L, at least 175 g/L, at least 200 g/L, at least 225 g/L, at least 250 g/L, at least 275 g/L, at least 300 g/L, at least 325 g/L, at least 350 g/L, at least 375 g/L, at least 400 g/L, at least 425 g/L, at least 450 g/L, at least 475 g/L, at least 500 g/L, at least 1,000 g/L, at least 2,000 g/L, at least 3,000 g/L, at least 4,000 g/L, at least 5,000 g/L, at least 6,000 g/L, at least 7,000 g/L, at least 8,000 g/L, at least 9,000 g/L, or at least 10,000 g/L of one or more mogrosides and/or mogroside precursors. In some embodiments, the mogroside is mogroside I-A1 (MIA1), mogroside IE (MIE or MIE), mogroside II-A1 (MIIA1 or M2A1), mogroside II-A2 (MIIA2 or M2A2), mogroside III-A1 (MIIIA1 or M3A1), mogroside II-E (MIIE or M2E), mogroside III (MIII or M3), siamenoside I, mogroside IV (MIV or M4), mogroside IVa (MIVA or M4A), isomogroside IV, mogroside III-E (MIIIE or M3E), mogroside V (MV or M5), mogroside VIA (MVIA), mogroside VIB (MVIB), isomogroside V, mogroside VIa1 (MVIa1), or mogroside VI (MVI or M6). In some embodiments, the mogroside precursor is oxidosqualene. In some embodiments, the mogroside precursor is cucurbitadienol. In some embodiments, the mogrol or mogroside precursor is mevalonate.

### Lanosterol Synthases

Aspects of the present disclosure provide lanosterol synthases, which may be useful, for production of various compounds, including for example, mogrol precursors, mogrol, and/or mogrosides. As used in this disclosure, a lanosterol synthase is an enzyme that is capable of catalyzing cyclization of 2-3-oxidosqualene to produce lanosterol. In some embodiments, a lanosterol synthase disclosed herein is a hypomorph of lanosterol synthase, e.g., a variant of lanosterol synthase that has reduced (e.g., decreased but not abolished) lanosterol synthase expression, level and/or activity. Without being bound by any particular theory, the present disclosure suggests that complete inactivation of lanosterol synthase is lethal in yeast, as lanosterol synthase may be needed to produce a hydrophobic component of the cell membrane important for maintaining the integrity of the cell. In some embodiments, a lanosterol synthase disclosed herein is useful for mogrol and/or mogroside production, and/or production of their precursors, as reduction in lanosterol synthase activity increases flux through the mevalonate pathway and/or reduces competition for oxidosqualene. Structurally, a lanosterol synthase may comprise the catalytic motif DCTAE (SEQ ID NO: 5). See e.g., Corey et al. PNAS 1994 Mar. 15; 91(6):2211-5 and Shi et al. 1994 Jul. 19; 91(15):7370-4. In some embodiments, in a host cell in which lanosterol synthase expression, level or activity is decreased, the cell retains enough functional lanosterol synthase to maintain the integrity of its cell and remain viable, but a decreased proportion of 2-3-oxidosqualene is converted to lanosterol (e.g., as compared to a similar cell comprising a wild-type ERG7). In some aspects, the present disclosure pertains to a host cell which comprises a mevalonate pathway (or a portion thereof, wherein a portion of a mevalonate pathway comprises at least one enzyme of a mevalonate pathway, including but not limited to: acetoacetyl COA synthase, ERG10, ERG13, HMG, ERG12, ERG8, ERG19, IDI, ERG20, ERG9, a UDP-glycosyltransferases (UGT) enzyme (e.g., a primary or secondary UGT), a cucurbitadienol synthase (CDS) enzyme, a C11 hydroxylase, an epoxide hydrolase (EPH), and squalene epoxidase (SQE), further comprising a variant of lanosterol synthase described herein.

As a non-limiting example, a lanosterol synthase may be ERG7 and may comprise the amino acid sequence:

(SEQ ID NO: 1)
MGIHESVSKQFAKNGHSKYRSDRYGLPKTDLRRWTFHASDLGAQWWKYDD
TTPLEELEKRATDYVKYSLELPGYAPVTLDSKPVKNAYEAALKNWHLFAS
LQDPDSGAWQSEYDGPQFMSIGYVTACYFGGNEIPTPVKTEMIRYIVNTA
HPVDGGWGLHKEDKSTCFGTSINYVVLRLLGLSRDHPVCVKARKTLLTKF
GGAINNPHWGKTWLSILNLYKWEGVNPAPGELWLLPYFVPVHPGRWWVHT
RWIYLAMGYLEAAEAQCELTPLLEELRDEIYKKPYSEIDFSKHCNSISGV
DLYYPHTGLLKFGNALLRRYRKFRPQWIKEKVKEEIYNLCLREVSNTRHL
CLAPVNNAMTSIVMYLHEGPDSANYKKIAARWPEFLSLNPSGMFMNGTNG
LQVWDTAFAVQYACVCGFAELPQYQKTIRAAFDFLDRSQINEPTEENSYR
DDRVGGWPFSTKTQGYPVSDCTAEALKAIIMVQNTPGYEDLKKQVSDKRK
HTAIDLLLGMQNVGSFEPGSFASYEPIRASSMLEKINPAEVFGNIMVEYP
YVECTDSVVLGLSYFRKYHDYRNEDVDRAISAAIGYIIREQQPDGGFFGS
WGVCYCYAHMFAMEALETQNLNYNNCSTVQKACDFLAGYQEADGGWAEDF
KSCETQMYVRGPHSLVVPTAMALLSLMSGRYPQEDKIHAAARFLMSKQMS
NGEWLKEEMEGVFNHTCAIEYPNYRFYFVMKALGLYFKGYCQ.

SEQ ID NO: 1 may be encoded by the nucleotide sequence:

(SEQ ID NO: 2)
ATGGGAATCCACGAAAGTGTGTCGAAACAGTTTGCGAAAAACGGACATTC
CAAGTACCGCAGCGACCGATACGGCTTACCTAAGACGGATCTGCGACGAT
GGACGTTCCACGCGTCCGATCTGGGGGCGCAATGGTGGAAGTATGACGAT
ACCACACCGCTGGAAGAGCTGGAAAAGAGGGCTACCGACTACGTCAAATA
CTCGCTGGAGCTGCCGGGATACGCGCCCGTGACTCTGGACTCCAAGCCCG
TGAAAAATGCCTACGAAGCGGCTCTCAAAAACTGGCATCTGTTTGCGTCG
CTGCAAGACCCCGACTCCGGCGCATGGCAGTCGGAATACGACGGACCGCA
GTTCATGTCGATCGGTTATGTGACGGCGTGCTACTTTGGCGGCAACGAGA
TCCCCACGCCGGTCAAAACCGAAATGATCAGATACATTGTCAACACAGCC
CACCCAGTTGACGGAGGCTGGGGCCTTCACAAAGAAGACAAGAGCACCTG
TTTCGGTACCAGCATCAACTACGTGGTCCTGCGACTACTGGGCCTGTCAC
GGGATCATCCGGTCTGCGTCAAGGCGCGCAAAACGCTGCTCACCAAGTTT
GGCGGCGCCATCAACAACCCCCATTGGGGCAAGACCTGGCTGTCGATTCT
CAATCTCTACAAATGGGAGGGTGTGAATCCGGCCCCTGGCGAGCTCTGGC
TGTTGCCCTACTTTGTTCCTGTTCATCCGGGCCGATGGTGGGTCCATACC
CGGTGGATCTACCTTGCCATGGGCTATCTGGAGGCTGCGGAGGCCCAATG
CGAACTCACTCCGTTGCTGGAGGAGCTCCGAGACGAAATCTACAAAAAGC
CCTACTCGGAGATTGATTTCTCCAAACATTGCAACTCCATCTCCGGAGTC
GACCTCTACTATCCCCACACCGGCCTTTTGAAGTTTGGCAACGCGCTTCT
CCGACGATACCGCAAGTTCAGACCGCAGTGGATCAAAGAAAAGGTCAAGG
AGGAAATTTACAACTTGTGCCTTCGAGAGGTTTCCAACACACGACACTTG
TGTCTCGCTCCCGTCAACAATGCCATGACCTCCATTGTCATGTATCTCCA
TGAGGGGCCCGATTCGGCGAATTACAAAAAGATTGCGGCCCGATGGCCCG
AATTTCTGTCTCTGAATCCGTCGGGAATGTTTATGAACGGCACCAACGGT
CTGCAGGTCTGGGATACTGCGTTTGCCGTGCAATACGCGTGTGTTTGTGG
CTTTGCCGAACTTCCCCAGTACCAGAAGACGATCCGAGCGGCGTTTGATT
TTCTCGATCGGTCCCAGATCAACGAGCCGACGGAGGAAAATTCCTATCGA
GACGACCGCGTCGGAGGATGGCCCTTTAGTACCAAGACCCAGGGGTATCC
AGTCTCCGACTGTACTGCCGAGGCTCTCAAGGCCATCATCATGGTCCAGA
ATACGCCTGGATACGAGGATCTGAAGAAACAAGTGTCTGACAAGCGGAAA
CACACTGCCATCGATCTACTTTTGGGAATGCAGAACGTGGGCTCGTTTGA
ACCGGGCTCTTTCGCCTCCTATGAGCCTATCCGGGCGTCGTCCATGCTGG
AGAAGATCAATCCGGCCGAGGTGTTTGGAAACATCATGGTGGAGTATCCG
TACGTGGAATGCACTGATTCTGTTGTTCTGGGTCTGTCCTACTTTCGAAA
GTACCACGATTACCGCAACGAAGACGTGGACCGAGCCATCTCTGCTGCCA
TTGGATACATTATTCGAGAGCAGCAGCCTGACGGCGGCTTCTTTGGCTCC
TGGGGCGTGTGCTACTGCTACGCTCACATGTTTGCCATGGAGGCTCTGGA
GACGCAGAATCTCAACTATAACAACTGTTCCACGGTTCAAAAGGCGTGCG
ACTTTCTGGCGGGCTACCAGGAAGCAGATGGAGGCTGGGCCGAGGACTTT
AAGTCGTGCGAGACTCAGATGTACGTGCGCGGACCCCATTCGCTGGTCGT
GCCTACTGCCATGGCCCTGTTGAGTTTGATGAGTGGTCGGTATCCCCAGG
AGGACAAGATTCATGCTGCGGCCCGGTTTCTCATGAGCAAGCAGATGAGC
AACGGTGAGTGGCTCAAGGAGGAGATGGAGGGGGTGTTTAACCATACTTG
TGCCATTGAGTATCCCAACTACCGGTTTTATTTTGTCATGAAGGCTTTGG
GGTTGTATTTCAAGGGATATTGCCAGTGA.

In some embodiments, a lanosterol synthase comprises the amino acid sequence:

(SEQ ID NO: 3)
MGIHESVSKQFAKNGHSKYRSDRYGLPKTDLRRWTFHASDLGAQWWKYDG
TTPLEELEKRATDYVRYSLELPGYAPVTLDSKPVKNAYEAALKSWHLFAS
LQDPDSGAWQSEYDGPQFMSIGYVTACYFGGNEIPTPVKTEMIRYIVNTA
HPVDGGWGLHKEDKSTCFGTSINYVVLRLLGLSRDHPVCVKARKTLLTKF
GGAINNPHWGKTWLSILNLYKWEGVNPAPGELWLLPYFVPVHPGRWWVHT
RWIYLAMGYLEAAEAQCELTPLLEELRDEIYKKPYSEIDFSKHCNSISGV
DLYYPHTGLLKFGNALLRRYRKFRPQWIKEKVKEEIYNLCLREVSNTRHL
CLAPVNNAMTSIVMYLHEGPDSANYKKIAARWPEFLSLNPSGMFMNGTNG
LQVWDTAFAVQYACVCSFAELPQYQKTIRAAFDFLDRSQINEPTEENSYR
DDRVGGWPFSTKTQGYPVSDCTAEALKAIIMVQNTPGYEDLKKQVSDKRK
HTAIDLLLGMQNVGSFEPGSFASYEPIRASSMLEKINPAEVFGNIMVEYP
YVECTDSVVLGLSYFRKYHDYRNEDVDRAISAAIGYIIREQQPDGGFFGS
WGVCYCYAHMFAMEALVTQNLNYNNCSTVQKACDFLAGYQEADGGWAEDF
KSCETQMYVRGPHSLVVPTAMALLSLMSGRYPQEDKIHAAARFLMSKQMS
NGEWLKEEMEGVFNHTCAIEYPNYRLYFVMKALGLYFKGYCQ.

In some embodiments, a lanosterol synthase comprising SEQ ID NO: 3 is encoded by the nucleotide sequence:

(SEQ ID NO: 4)
ATGGGAATCCACGAAAGTGTGTCGAAACAGTTTGCGAAAAACGGACATTC
CAAGTACCGCAGCGACCGATACGGCTTACCTAAGACGGATCTGCGACGAT
GGACGTTCCACGCGTCCGATCTGGGGGCGCAATGGTGGAAGTATGACGGT
ACCACACCGCTGGAAGAGCTGGAAAAGAGGGCTACCGACTACGTCAGATA
CTCGCTGGAGCTGCCGGGATACGCGCCCGTGACTCTGGACTCCAAGCCCG
TGAAAAATGCCTACGAAGCGGCTCTCAAAAGCTGGCATCTGTTTGCGTCG
CTGCAAGACCCCGACTCCGGCGCATGGCAGTCGGAATACGACGGACCGCA
GTTCATGTCGATCGGTTATGTGACGGCGTGCTACTTTGGCGGCAACGAGA
TCCCCACGCCGGTCAAAACCGAAATGATCAGATACATTGTCAACACAGCC
CACCCAGTTGACGGAGGCTGGGGCCTTCACAAAGAAGACAAGAGCACCTG
TTTCGGTACCAGCATCAACTACGTGGTCCTGCGACTACTGGGCCTGTCAC
GGGATCATCCGGTCTGCGTCAAGGCGCGCAAAACGCTGCTCACCAAGTTT
GGCGGCGCCATCAACAACCCCCATTGGGGCAAGACCTGGCTGTCGATTCT
CAATCTCTACAAATGGGAGGGTGTGAATCCGGCCCCTGGCGAGCTCTGGC
TGTTGCCCTACTTTGTTCCTGTTCATCCGGGCCGATGGTGGGTCCATACC
CGGTGGATCTACCTTGCCATGGGCTATCTGGAGGCTGCGGAGGCCCAATG
CGAACTCACTCCGTTGCTGGAGGAGCTCCGAGACGAAATCTACAAAAAGC
CCTACTCGGAGATTGATTTCTCCAAACATTGCAACTCCATCTCCGGAGTC
GACCTCTACTATCCCCACACCGGCCTTTTGAAGTTTGGCAACGCGCTTCT
CCGACGATACCGCAAGTTCAGACCGCAGTGGATCAAAGAAAAGGTCAAGG
AGGAAATTTACAACTTGTGCCTTCGAGAGGTTTCCAACACACGACACTTG
TGTCTCGCTCCCGTCAACAATGCCATGACCTCCATTGTCATGTATCTCCA
TGAGGGGCCCGATTCGGCGAATTACAAAAAGATTGCGGCCCGATGGCCCG
AATTTCTGTCTCTGAATCCGTCGGGAATGTTTATGAACGGCACCAACGGT
CTGCAGGTCTGGGATACTGCGTTTGCCGTGCAATACGCGTGTGTTTGTAG
CTTTGCCGAACTTCCCCAGTACCAGAAGACGATCCGAGCGGCGTTTGATT
TTCTCGATCGGTCCCAGATCAACGAGCCGACGGAGGAAAATTCCTATCGA
GACGACCGCGTCGGAGGATGGCCCTTTAGTACCAAGACCCAGGGGTATCC
AGTCTCCGACTGTACTGCCGAGGCTCTCAAGGCCATCATCATGGTCCAGA
ATACGCCTGGATACGAGGATCTGAAGAAACAAGTGTCTGACAAGCGGAAA
CACACTGCCATCGATCTACTTTTGGGAATGCAGAACGTGGGCTCGTTTGA
ACCGGGCTCTTTCGCCTCCTATGAGCCTATCCGGGCGTCGTCCATGCTGG
AGAAGATCAATCCGGCCGAGGTGTTTGGAAACATCATGGTGGAGTATCCG
TACGTGGAATGCACTGATTCTGTTGTTCTGGGTCTGTCCTACTTTCGAAA
GTACCACGATTACCGCAACGAAGACGTGGACCGAGCCATCTCTGCTGCCA
TCGGATACATTATTCGAGAGCAGCAGCCTGACGGTGGCTTCTTTGGCTCC
TGGGGCGTGTGCTACTGCTACGCTCACATGTTTGCCATGGAGGCTCTGGT
GACGCAGAATCTCAACTATAACAACTGTTCCACGGTTCAAAAGGCGTGCG
ACTTTCTGGCGGGCTACCAGGAAGCAGATGGAGGCTGGGCCGAGGACTTT
AAGTCGTGCGAGACTCAGATGTACGTGCGCGGACCCCATTCGCTGGTCGT
GCCTACTGCCATGGCCCTGTTGAGTTTGATGAGTGGTCGGTATCCCCAGG
AGGACAAGATTCATGCTGCGGCCCGGTTTCTCATGAGCAAGCAGATGAGC
AACGGTGAGTGGCTCAAGGAGGAGATGGAGGGGGTGTTTAACCATACTTG
TGCCATTGAGTATCCCAACTACCGGTTATATTTTGTCATGAAGGCTTTGG
GGTTGTATTTCAAGGGATATTGCCAGTGA.

In some embodiments, a lanosterol synthase of the present disclosure comprises a sequence (e.g., nucleic acid or amino acid sequence) that is at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical, including all values in between, to any one of SEQ ID NOs: 1-4, 61-66, 68-71, 73-74, 78-87, 89-92, 94-95, 99-109, 111-120, 304, 313, 316-319, 321-326, and 328-331, any lanosterol synthase in Tables 11 and 14, or any lanosterol synthase sequence disclosed in this application or known in the art.

In some embodiments, a lanosterol synthase comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, or at least 100 amino acid changes relative to SEQ ID NO: 1 or 313.

In some embodiments, a lanosterol synthase comprises at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 21, at most 22, at most 23, at most 24, at most 25, at most 26, at most 27, at most 28, at most 29, at most 30, at most 31, at most 32, at most 33, at most 34, at most 35, at most 36, at most 37, at most 38, at most 39, at most 40, at most 41, at most 42, at most 43, at most 44, at most 45, at most 46, at most 47, at most 48, at most 49, at most 50, at most 51, at most 52, at most 53, at most 54, at most 55, at most 56, at most 57, at most 58, at most 59, at most 60, at most 61, at most 62, at most 63, at most 64, at most 65, at most 66, at most 67, at most 68, at most 69, at most 70, at most 71, at most 72, at most 73, at most 74, at most 75, at most 76, at most 77, at most 78, at most 79, at most 80, at most 81, at most 82, at most 83, at most 84, at most 85, at most 86, at most 87, at most 88, at most 89, at most 90, at most 91, at most 92, at most 93, at most 94, at most 95, at most 96, at most 97, at most 98, at most 99, or at most 100 amino acid changes relative to SEQ ID NO: 1 or 313.

In some embodiments, a lanosterol synthase comprises between 1-5, between 1-10, between 1-15, between 1-20, between 1-25, between 1-30, between 1-35, between 1-40, between 1-45, between 1-50, between 5-10, between 5-20, between 5-30, between 5-40, between 5-50, between 5-60, between 5-70, between 5-80, between 5-90, between 5-100, between 10-20, between 10-30, between 10-40, between 10-50, between 10-60, between 10-70, between 10-80, between 10-90, or between 10-100 amino acid changes, including all values in between, relative to SEQ ID NO: 1 or 313.

In some embodiments, a lanosterol synthase comprises an amino acid change at one or more positions selected from position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, or 742 of SEQ ID NO: 1.

In some embodiments, a lanosterol synthase comprises an amino acid change at one or more positions selected from position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, or 731 of SEQ ID NO: 313.

In some embodiments, the amino acid change is a substitution, insertion, or a deletion. In some embodiments, the amino acid change results in a truncation or lengthening of a lanosterol synthase relative to a control. In some embodiments, a control is a wild-type lanosterol synthase. In some embodiments, a control is a different lanosterol synthase. As a non-limiting example, a lanosterol synthase may comprise one or more changes indicated in Tables 3, 5, 6A-6B, 7, 8, 9, 10, and 11 relative to SEQ ID NO: 1 or 313.

In some embodiments, a lanosterol synthase comprises an amino acid substitution or deletion relative to SEQ ID NO: 1 at one or more residues corresponding to position 14, 33, 47, 50, 66, 80, 83, 85, 92, 94, 107, 122, 132, 145, 158, 170, 172, 184, 193, 197, 198, 212, 213, 227, 228, 231, 235, 248, 249, 260, 282, 286, 287, 289, 295, 296, 309, 314, 316, 329, 344, 360, 370, 371, 372, 398, 407, 414, 417, 423, 432, 437, 442, 444, 452, 474, 479, 491, 498, 515, 526, 529, 536, 544, 552, 559, 560, 564, 578, 586, 608, 610, 617, 619, 620, 631, 638, 650, 655, 660, 679, 686, 702, 710, 726, 736, 738, and/or 742 in SEQ ID NO: 1. In some embodiments, a lanosterol synthase comprises: the amino acid Y at the residue corresponding to position 14 in SEQ ID NO:1; the amino acid Q at the residue corresponding to position 33 in SEQ ID NO:1; the amino acid E at the residue corresponding to position 47 in SEQ ID NO:1; the amino acid G at the residue corresponding to position 50 in SEQ ID NO:1; the amino acid R at the residue corresponding to position 66 in SEQ ID NO: 1; the amino acid G at the residue corresponding to position 80 in SEQ ID NO: 1; the amino acid L at the residue corresponding to position 83 in SEQ ID NO: 1; the amino acid N at the residue corresponding to position 85 in SEQ ID NO:1; the amino acid I at the residue corresponding to position 92 in SEQ ID NO:1; the amino acid S at the residue corresponding to position 94 in SEQ ID NO:1; the amino acid D at the residue corresponding to position 107 in SEQ ID NO:1; the amino acid C at the residue corresponding to position 122 in SEQ ID NO:1; the amino acid S at the residue corresponding to position 132 in SEQ ID NO: 1; the amino acid C at the residue corresponding to position 145 in SEQ ID NO:1; the amino acid S at the residue corresponding to position 158 in SEQ ID NO:1; the amino acid A at the residue corresponding to position 170 in SEQ ID NO: 1; the amino acid N at the residue corresponding to position 172 in SEQ ID NO:1; the amino acid W at the residue corresponding to position 184 in SEQ ID NO:1; the amino acid C or H at the residue corresponding to position 193 in SEQ ID NO:1; the amino acid V at the residue corresponding to position 197 in SEQ ID NO:1; the amino acid I at the residue corresponding to position 198 in SEQ ID NO: 1; the amino acid I at the residue corresponding to position 212 in SEQ ID NO:1; the amino acid L at the residue corresponding to position 213 in SEQ ID NO:1; the amino acid L at the residue corresponding to position 227 in SEQ ID NO:1; the amino acid T at the residue corresponding to position 228 in SEQ ID NO: 1; the amino acid V at the residue corresponding to position 231 in SEQ ID NO: 1; the amino acid M at the residue corresponding to position 235 in SEQ ID NO:1; the amino acid F at the residue corresponding to position 248 in SEQ ID NO:1; the amino acid L at the residue corresponding to position 249 in SEQ ID NO:1; the amino acid R at the residue corresponding to position 260 in SEQ ID NO:1; the amino acid I at the residue corresponding to position 282 in SEQ ID NO:1; the amino acid F at the residue corresponding to position 286 in SEQ ID NO: 1; the amino acid G at the residue corresponding to position 287 in SEQ ID NO:1; the amino acid G at the residue corresponding to position 289 in SEQ ID NO: 1; the amino acid I at the residue corresponding to position 295 in SEQ ID NO: 1; the amino acid T at the residue corresponding to position 296 in SEQ ID NO: 1; the amino acid F at the residue corresponding to position 309 in SEQ ID NO: 1; the amino acid S at the residue corresponding to position 314 in SEQ ID NO:1; the amino acid R at the residue corresponding to position 316 in SEQ ID NO: 1; the amino acid N at the residue corresponding to position 329 in SEQ ID NO:1; the amino acid A at the residue corresponding to position 344 in SEQ ID NO: 1; the amino acid S at the residue corresponding to position 360 in SEQ ID NO:1; the amino acid L at the residue corresponding to position 370 in SEQ ID NO:1; the amino acid V at the residue corresponding to position 371 in SEQ ID NO:1; the amino acid P at the residue corresponding to position 372 in SEQ ID NO:1; the amino acid I at the residue corresponding to position 398 in SEQ ID NO: 1; the amino acid V at the residue corresponding to position 407 in SEQ ID NO:1; the amino acid S at the residue corresponding to position 414 in SEQ ID NO:1; the amino acid S at the residue corresponding to position 417 in SEQ ID NO:1; the amino acid L at the residue corresponding to position 423 in SEQ ID NO:1; the amino acid I or S at the residue corresponding to position 432 in SEQ ID NO: 1; the amino acid L at the residue corresponding to position 437 in SEQ ID NO:1; the amino acid V at the residue corresponding to position 442 in SEQ ID NO:1; the amino acid M or S at the residue corresponding to position 444 in SEQ ID NO:1; the amino acid G at the residue corresponding to position 452 in SEQ ID NO: 1; the amino acid V at the residue corresponding to position 474 in SEQ ID NO:1; the amino acid S at the residue corresponding to position 479 in SEQ ID NO:1; the amino acid Q at the residue corresponding to position 491 in SEQ ID NO:1; the amino acid N at the residue corresponding to position 498 in SEQ ID NO: 1; the amino acid L at the residue corresponding to position 515 in SEQ ID NO:1; the amino acid T at the residue corresponding to position 526 in SEQ ID NO:1; the amino acid T at the residue corresponding to position 529 in SEQ ID NO:1; the amino acid F at the residue corresponding to position 536 in SEQ ID NO:1; the amino acid Y at the residue corresponding to position 544 in SEQ ID NO:1; the amino acid E at the residue corresponding to position 552 in SEQ ID NO:1; the amino acid A at the residue corresponding to position 559 in SEQ ID NO:1; the amino acid M at the residue corresponding to position 560 in SEQ ID NO: 1; the amino acid C or N at the residue corresponding to position 564 in SEQ ID NO:1; the amino acid P at the residue corresponding to position 578 in SEQ ID NO:1; the amino acid F at the residue corresponding to position 586 in SEQ ID NO:1; the amino acid T at the residue corresponding to position 608 in SEQ ID NO:1; the amino acid I at the residue corresponding to position 610 in SEQ ID NO: 1; the amino acid V at the residue corresponding to position 617 in SEQ ID NO:1; the amino acid L at the residue corresponding to position 619 in SEQ ID NO:1; the amino acid S at the residue corresponding to position 620 in SEQ ID NO:1; the amino acid E or R at the residue corresponding to position 631 in SEQ ID NO:1; the amino acid D at the residue corresponding to position 638 in SEQ ID NO: 1; the amino acid L at the residue corresponding to position 650 in SEQ ID NO:1; the amino acid A at the residue corresponding to position 655 in SEQ ID NO:1; the amino acid H at the residue corresponding to position 660 in SEQ ID NO:1; the amino acid S at the residue corresponding to position 679 in SEQ ID NO:1; the amino acid E at the residue corresponding to position 686 in SEQ ID NO: 1; the amino acid D at the residue corresponding to position 702 in SEQ ID NO:1; the amino acid Q at the residue corresponding to position 710 in SEQ ID NO:1; the amino acid L or V at the residue corresponding to position 726 in SEQ ID NO:1; the amino acid F at the residue corresponding to position 736 in SEQ ID NO:1; the amino acid M at the residue corresponding to position 738 in SEQ ID NO:1; and/or a truncation that results in deletion of the residue corresponding to position 742 in SEQ ID NO: 1. In some embodiments, a lanosterol synthase comprises the amino acid substitution E617V, G107D, and/or K631E relative to SEQ ID NO: 1.

In some embodiments, relative to SEQ ID NO: 1, a lanosterol synthase comprises: R33Q, R193C, D289G, N295I, S296T, N620S, and Y736F; R184W, L235M, L260R, and E710Q; K47E, L92I, T360S, S372P, T444M, and R578P; D50G, K66R, N94S, G417S, E617V, and F726L; N14Y, N132S, Y145C, R193H, I286F, L316R, F432I, E442V, T444S, I479S, K631R, and T655A; F432S, D452G, and I536F; E287G, K329N, E617V, and F726V; E231V, A407V, Q423L, A529T, and Y564C; V248F, D371V, and G702D; L197V, K282I, N314S, P370L, A608T, G638D, and F650L; L491Q, Y586F, and R660H; G122C, H249L, and K738M; P227L, E474V, V559A, and Y564N; K85N, G158S, S515L, P526T, Q619L, and a truncation resulting in a deletion of the residue corresponding to Q742 in SEQ ID NO: 1; G107D and K631E; T212I, W213L, N544Y, and V552E; I172N, C414S, L560M, and G679S; R193C, D289G, N295I, S296T, N620S, and Y736F; K85N and G158S; L197V, K282I, N314S, and P370L; I172N, C414S, and L560M; D371V, M610I, and G702D; D371V, K498N, M610I, and G702D; D80G, P83L, T170A, T198I, and A228T; T360S, S372P, T444M, and R578P; D50G, K66R, N94S, G417S, and E617V; or L309F, V344A, T398I, and K686E.

In some embodiments, relative to SEQ ID NO: 1, the lanosterol synthase comprises the following amino acid substitutions: R193C, D289G, N295I, S296T, N620S, and Y736F; F432S, D452G, and I536F; K85N and G158S; L197V, K282I, N314S, and P370L; I172N, C414S, L560M, and G679S; I172N, C414S, and L560M; D371V, M610I, and G702D; D371V, K498N, M610I, and G702D; D80G, P83L, T170A, T198I, and A228T; D50G, K66R, N94S, G417S, E617V, and F726L; T360S, S372P, T444M, and R578P; D50G, K66R, N94S, G417S, and E617V; and L309F, V344A, T398I, and K686E.

In some embodiments, relative to SEQ ID NO: 1, the lanosterol synthase comprises the following amino acid substitutions: D50G, K66R, N94S, G417S, E617V, and F726L; K85N and G158S; K47E, L92I, T360S, S372P, T444M, and R578P; F432S, D452G, and I536F; T360S, S372P, T444M, and R578P; L491Q, Y586F, and R660H; K85N, G158S, S515L, P526T, Q619L, and a truncation that results in deletion of the residue corresponding to position 742 in SEQ ID NO: 1; or I172N, C414S, L560M, and G679S.

In some embodiments, a lanosterol comprises an amino acid substitution or deletion relative to SEQ ID NO: 1 at one or more residues corresponding to position 14, 33, 47, 50, 66, 85, 92, 94, 122, 132, 145, 158, 193, 231, 248, 249, 286, 287, 289, 295, 296, 316, 329, 360, 371, 372, 407, 417, 423, 432, 442, 444, 479, 515, 526, 529, 564, 578, 617, 619, 620, 631, 655, 702, 726, 736, 738, and/or 742 in SEQ ID NO: 1. In some embodiments, relative to SEQ ID NO: 1, a lanosterol synthase comprises: R33Q, R193C, D289G, N295I, S296T, N620S, and Y736F; K47E, L92I, T360S, S372P, T444M, and R578P; D50G, K66R, N94S, G417S, E617V, and F726L; N14Y, N132S, Y145C, R193H, I286F, L316R, F432I, E442V, T444S, I479S, K631R, and T655A; E287G, K329N, E617V, and F726V; E231V. A407V, Q423L, A529T, and Y564C; V248F, D371V, and G702D; G122C, H249L, and K738M; or K85N, G158S, S515L, P526T, and Q619L, and a truncation resulting in a deletion of the residue corresponding to Q742 in SEQ ID NO: 1.

In some embodiments, the host cell comprises a heterologous polynucleotide encoding a lanosterol synthase, wherein the lanosterol synthase comprises an amino acid substitution or deletion relative to SEQ ID NO: 313 at one or more residues corresponding to position 64, 120, 121, 136, 226, 268, 275, 281, 300, 322, 333, 438, 502, 604, 619, 628, 656, 693, 726, 727, 728, 729, 730, and/or 731.

In some embodiments, the lanosterol synthase comprises: the amino acid G at the residue corresponding to position 64 in SEQ ID NO: 313; the amino acid V at the residue corresponding to position 120 in SEQ ID NO: 313; the amino acid S at the residue corresponding to position 121 in SEQ ID NO: 313; the amino acid V at the residue corresponding to position 136 in SEQ ID NO: 313; the amino acid I at the residue corresponding to position 226 in SEQ ID NO: 313; the amino acid S at the residue corresponding to position 268 in SEQ ID NO: 313; the amino acid I at the residue corresponding to position 275 in SEQ ID NO: 313; the amino acid A at the residue corresponding to position 281 in SEQ ID NO: 313; the amino acid G at the residue corresponding to position 300 in SEQ ID NO: 313; the amino acid G at the residue corresponding to position 322 in SEQ ID NO: 313; the amino acid A at the residue corresponding to position 333 in SEQ ID NO: 313; the amino acid E at the residue corresponding to position 438 in SEQ ID NO: 313; the amino acid L at the residue corresponding to position 502 in SEQ ID NO: 313; the amino acid N at the residue corresponding to position 604 in SEQ ID NO: 313; the amino acid S at the residue corresponding to position 619 in SEQ ID NO: 313; the amino acid E at the residue corresponding to position 628 in SEQ ID NO: 313; the amino acid T at the residue corresponding to position 656 in SEQ ID NO: 313; the amino acid G at the residue corresponding to position 693 in SEQ ID NO: 313; and/or deletion of residues corresponding to positions 726-731 in SEQ ID NO: 313.

In some embodiments, the lanosterol synthase comprises relative to SEQ ID NO: 313: P121S, A136V, S300G, V322G, K438E, F502L, K628E, and deletion of residues corresponding to positions 726-731 in SEQ ID NO: 313; K268S, T281A, F502L, T604N, A656T, and E693G; or C619S, F275I, I120V, M226I, R64G, and T333A.

It should be appreciated that activity, such as specific activity, of a lanosterol synthase can be measured by any means known to one of ordinary skill in the art. In some embodiments, production of mogrol, one or more mogrol precursors, and/or one or more mogrosides can be used to determine lanosterol activity. As a non-limiting example, mevalonate production may be used as a readout of lanosterol synthase activity. For example, a lanosterol synthase with reduced activity may increase mevalonate production in a host cell relative to a control. In some embodiments, a control is a host cell with a different lanosterol synthase. In some embodiments, a control is a host cell with a wild-type lanosterol synthase.

The activity of a lanosterol synthase may be altered using any suitable method known in the art. In some embodiments, one or more amino acid changes reduces the activity of a lanosterol synthase as compared to a control lanosterol synthase. In some embodiments, a control lanosterol synthase is a wild-type lanosterol synthase. In some embodiments, the expression of a lanosterol synthase is altered to affect lanosterol synthase activity. In some embodiments, a host cell comprises a heterologous polynucleotide that is capable of reducing lanosterol synthase activity. In some embodiments, a reduction in lanosterol synthase expression in a host cell reduces lanosterol synthase activity. In some embodiments, the activity of a lanosterol synthase is reduced using: a weak promoter to drive expression of the lanosterol synthase, one or more codons that are not optimized for a particular host cell, use of an antisense nucleic acid, a genetic modification that alters gene expression and/or introduces one or more alterations, alteration of a promoter driving expression of a lanosterol synthase and/or altering the coding sequence of a lanosterol synthase.

In some embodiments, a lanosterol synthase is capable of increasing production of a mogrol precursor, mogrol, and/or a mogroside by a host cell by at least 0.01%, at least 0.05%, at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 150%, at least 200%, at least 250%, at least 300%, at least 350%, at least 400%, at least 450%, at least 500%, at least 550%, at least 600%, at least 650%, at least 700%, at least 750%, at least 800%, at least 850%, at least 900%, at least 950%, or at least 1000%, including all values in between as compared to production of the mogrol precursor, mogrol, and/or the mogroside by a host cell that does not comprise the lanosterol synthase. In some embodiments, a lanosterol synthase is capable of increasing production of a mogrol precursor, mogrol, and/or a mogroside by a host cell at most 5%, at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90%, at most 95%, at most 100%, at most 150%, at most 200%, at most 250%, at most 300%, at most 350%, at most 400%, at most 450%, at most 500%, at most 550%, at most 600%, at most 650%, at most 700%, at most 750%, at most 800%, at most 850%, at most 900%, at most 950%, or at most 1000%, including all values in between as compared to production of the mogrol precursor, mogrol, and/or the mogroside by a host cell that does not comprise the lanosterol synthase. In some embodiments, a lanosterol synthase is capable of increasing production of a mogrol precursor, mogrol, and/or a mogroside by a host cell between 0.01% and 1%, between 1% and 10%, between 10% and 20%, between 10% and 50%, between 50% and 100%, between 100% and 200%, between 200% and 300%, between 300% and 400%, between 400% and 500%, between 500% and 600%, between 600% and 700%, between 700% and 800%, between 800% and 900%, between 900% and 1000%, between 1% and 50%, between 1% and 100%, between 1% and 500%, or between 1% and 1,000%, including all values in between as compared to production of the mogrol precursor, mogrol, and/or the mogroside by a host cell that does not comprise the lanosterol synthase. In some embodiments, a lanosterol synthase is capable of increasing production of a mogrol precursor, mogrol, and/or a mogroside by a host cell at least 1.1 fold, at least 1.2 fold, at least 1.3 fold, at least 1.4 fold, at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3 fold, at least 3.1 fold, at least 3.2 fold, at least 3.3 fold, at least 3.4 fold, at least 3.5 fold, at least 3.6 fold, at least 3.7 fold, at least 3.8 fold, at least 3.9 fold, at least 4 fold, at least 4.1 fold, at least 4.2 fold, at least 4.3 fold, at least 4.4 fold, at least 4.5 fold, at least 4.6 fold, at least 4.7 fold, at least 4.8 fold, at least 4.9 fold, at least 5 fold, at least 5.1 fold, at least 5.2 fold, at least 5.3 fold, at least 5.4 fold, at least 5.5 fold, at least 5.6 fold, at least 5.7 fold, at least 5.8 fold, at least 5.9 fold, at least 6 fold, at least 6.1 fold, at least 6.2 fold, at least 6.3 fold, at least 6.4 fold, at least 6.5 fold, at least 6.6 fold, at least 6.7 fold, at least 6.8 fold, at least 6.9 fold, at least 7 fold, at least 7.1 fold, at least 7.2 fold, at least 7.3 fold, at least 7.4 fold, at least 7.5 fold, at least 7.6 fold, at least 7.7 fold, at least 7.8 fold, at least 7.9 fold, at least 8 fold, at least 8.1 fold, at least 8.2 fold, at least 8.3 fold, at least 8.4 fold, at least 8.5 fold, at least 8.6 fold, at least 8.7 fold, at least 8.8 fold, at least 8.9 fold, at least 9 fold, at least 9.1 fold, at least 9.2 fold, at least 9.3 fold, at least 9.4 fold, at least 9.5 fold, at least 9.6 fold, at least 9.7 fold, at least 9.8 fold, at least 9.9 fold, at least 10 fold, at least 11 fold, at least 12 fold, at least 13 fold, at least 14 fold, at least 15 fold, at least 16 fold, at least 17 fold, at least 18 fold, at least 19 fold, at least 20 fold, at least 21 fold, at least 22 fold, at least 23 fold, at least 24 fold, at least 25 fold, at least 26 fold, at least 27 fold, at least 28 fold, at least 29 fold, at least 30 fold, at least 31 fold, at least 32 fold, at least 33 fold, at least 34 fold, at least 35 fold, at least 36 fold, at least 37 fold, at least 38 fold, at least 39 fold, at least 40 fold, at least 41 fold, at least 42 fold, at least 43 fold, at least 44 fold, at least 45 fold, at least 46 fold, at least 47 fold, at least 48 fold, at least 49 fold, at least 50 fold, at least 51 fold, at least 52 fold, at least 53 fold, at least 54 fold, at least 55 fold, at least 56 fold, at least 57 fold, at least 58 fold, at least 59 fold, at least 60 fold, at least 61 fold, at least 62 fold, at least 63 fold, at least 64 fold, at least 65 fold, at least 66 fold, at least 67 fold, at least 68 fold, at least 69 fold, at least 70 fold, at least 71 fold, at least 72 fold, at least 73 fold, at least 74 fold, at least 75 fold, at least 76 fold, at least 77 fold, at least 78 fold, at least 79 fold, at least 80 fold, at least 81 fold, at least 82 fold, at least 83 fold, at least 84 fold, at least 85 fold, at least 86 fold, at least 87 fold, at least 88 fold, at least 89 fold, at least 90 fold, at least 91 fold, at least 92 fold, at least 93 fold, at least 94 fold, at least 95 fold, at least 96 fold, at least 97 fold, at least 98 fold, at least 99 fold, at least 100 fold, at least 200 fold, at least 300 fold, at least 400 fold, at least 500 fold, at least 600 fold, at least 700 fold, at least 800 fold, at least 900 fold, or at least 1000 fold, including all values in between as compared to production of the mogrol precursor, mogrol, and/or the mogroside by a host cell that does not comprise the lanosterol synthase. In some embodiments, the mogrol precursor is mevalonate. In some embodiments, the mogrol precursor is 2-3-oxidosqualene. In some embodiments, the mogrol precursor is cucurbitadienol.

In some embodiments, a host cell comprising a lanosterol synthase is capable of producing at least 0.01 mg/L, at least 0.05 mg/L, at least 1 mg/L, at least 5 mg/L, at least 10 mg/L, at least 15 mg/L, at least 20 mg/L, at least 25 mg/L, at least 30 mg/L, at least 35 mg/L, at least 40 mg/L, at least 45 mg/L, at least 50 mg/L, at least 55 mg/L, at least 60 mg/L, at least 65 mg/L, at least 70 mg/L, at least 75 mg/L, at least 80 mg/L, at least 85 mg/L, at least 90 mg/L, at least 95 mg/L, at least 100 mg/L, at least 150 mg/L, at least 200 mg/L, at least 250 mg/L, at least 300 mg/L, at least 350 mg/L, at least 400 mg/L, at least 450 mg/L, at least 500 mg/L, at least 550 mg/L, at least 600 mg/L, at least 650 mg/L, at least 700 mg/L, at least 750 mg/L, at least 800 mg/L, at least 850 mg/L, at least 900 mg/L, at least 950 mg/L, at least 1 g/L, at least 1.1 g/L, at least 1.2 g/L, at least 1.3 g/L, at least 1.4 g/L, at least 1.5 g/L, at least 1.6 g/L, at least 1.7 g/L, at least 1.8 g/L, at least 1.9 g/L, at least 2 g/L, at least 2.1 g/L, at least 2.2 g/L, at least 2.3 g/L, at least 2.4 g/L, at least 2.5 g/L, at least 2.6 g/L, at least 2.7 g/L, at least 2.8 g/L, at least 2.9 g/L, at least 3 g/L, at least 3.1 g/L, at least 3.2 g/L, at least 3.3 g/L, at least 3.4 g/L, at least 3.5 g/L, at least 3.6 g/L, at least 3.7 g/L, at least 3.8 g/L, at least 3.9 g/L, at least 4 g/L, at least 4.1 g/L, at least 4.2 g/L, at least 4.3 g/L, at least 4.4 g/L, at least 4.5 g/L, at least 4.6 g/L, at least 4.7 g/L, at least 4.8 g/L, at least 4.9 g/L, at least 5 g/L, at least 5.1 g/L, at least 5.2 g/L, at least 5.3 g/L, at least 5.4 g/L, at least 5.5 g/L, at least 5.6 g/L, at least 5.7 g/L, at least 5.8 g/L, at least 5.9 g/L, at least 6 g/L, at least 6.1 g/L, at least 6.2 g/L, at least 6.3 g/L, at least 6.4 g/L, at least 6.5 g/L, at least 6.6 g/L, at least 6.7 g/L, at least 6.8 g/L, at least 6.9 g/L, at least 7 g/L, at least 7.1 g/L, at least 7.2 g/L, at least 7.3 g/L, at least 7.4 g/L, at least 7.5 g/L, at least 7.6 g/L, at least 7.7 g/L, at least 7.8 g/L, at least 7.9 g/L, at least 8 g/L, at least 8.1 g/L, at least 8.2 g/L, at least 8.3 g/L, at least 8.4 g/L, at least 8.5 g/L, at least 8.6 g/L, at least 8.7 g/L, at least 8.8 g/L, at least 8.9 g/L, at least 9 g/L, at least 9.1 g/L, at least 9.2 g/L, at least 9.3 g/L, at least 9.4 g/L, at least 9.5 g/L, at least 9.6 g/L, at least 9.7 g/L, at least 9.8 g/L, at least 9.9 g/L, at least 10 g/L, at least 20 g/L, at least 30 g/L, at least 40 g/L, at least 50 g/L, at least 60 g/L, at least 70 g/L, at least 80 g/L, at least 90 g/L, at least 100 g/L, at least 200 g/L, at least 300 g/L, at least 400 g/L, at least 500 g/L, at least 600 g/L, at least 700 g/L, at least 800 g/L, at least 900 g/L, or at least 1000 g/L including all values in between of a mogrol precursor, mogrol, and/or a mogroside. In some embodiments, a host cell comprising a lanosterol synthase is capable of producing at most 5 mg/L, at most 10 mg/L, at most 15 mg/L, at most 20 mg/L, at most 25 mg/L, at most 30 mg/L, at most 35 mg/L, at most 40 mg/L, at most 45 mg/L, at most 50 mg/L, at most 55 mg/L, at most 60 mg/L, at most 65 mg/L, at most 70 mg/L, at most 75 mg/L, at most 80 mg/L, at most 85 mg/L, at most 90 mg/L, at most 95 mg/L, at most 100 mg/L, at most 150 mg/L, at most 200 mg/L, at most 250 mg/L, at most 300 mg/L, at most 350 mg/L, at most 400 mg/L, at most 450 mg/L, at most 500 mg/L, at most 550 mg/L, at most 600 mg/L, at most 650 mg/L, at most 700 mg/L, at most 750 mg/L, at most 800 mg/L, at most 850 mg/L, at most 900 mg/L, at most 950 mg/L, at most 1 g/L, at most 1.1 g/L, at most 1.2 g/L, at most 1.3 g/L, at most 1.4 g/L, at most 1.5 g/L, at most 1.6 g/L, at most 1.7 g/L, at most 1.8 g/L, at most 1.9 g/L, at most 2 g/L, at most 2.1 g/L, at most 2.2 g/L, at most 2.3 g/L, at most 2.4 g/L, at most 2.5 g/L, at most 2.6 g/L, at most 2.7 g/L, at most 2.8 g/L, at most 2.9 g/L, at most 3 g/L, at most 3.1 g/L, at most 3.2 g/L, at most 3.3 g/L, at most 3.4 g/L, at most 3.5 g/L, at most 3.6 g/L, at most 3.7 g/L, at most 3.8 g/L, at most 3.9 g/L, at most 4 g/L, at most 4.1 g/L, at most 4.2 g/L, at most 4.3 g/L, at most 4.4 g/L, at most 4.5 g/L, at most 4.6 g/L, at most 4.7 g/L, at most 4.8 g/L, at most 4.9 g/L, at most 5 g/L, at most 5.1 g/L, at most 5.2 g/L, at most 5.3 g/L, at most 5.4 g/L, at most 5.5 g/L, at most 5.6 g/L, at most 5.7 g/L, at most 5.8 g/L, at most 5.9 g/L, at most 6 g/L, at most 6.1 g/L, at most 6.2 g/L, at most 6.3 g/L, at most 6.4 g/L, at most 6.5 g/L, at most 6.6 g/L, at most 6.7 g/L, at most 6.8 g/L, at most 6.9 g/L, at most 7 g/L, at most 7.1 g/L, at most 7.2 g/L, at most 7.3 g/L, at most 7.4 g/L, at most 7.5 g/L, at most 7.6 g/L, at most 7.7 g/L, at most 7.8 g/L, at most 7.9 g/L, at most 8 g/L, at most 8.1 g/L, at most 8.2 g/L, at most 8.3 g/L, at most 8.4 g/L, at most 8.5 g/L, at most 8.6 g/L, at most 8.7 g/L, at most 8.8 g/L, at most 8.9 g/L, at most 9 g/L, at most 9.1 g/L, at most 9.2 g/L, at most 9.3 g/L, at most 9.4 g/L, at most 9.5 g/L, at most 9.6 g/L, at most 9.7 g/L, at most 9.8 g/L, at most 9.9 g/L, at most 10 g/L, at most 20 g/L, at most 30 g/L, at most 40 g/L, at most 50 g/L, at most 60 g/L, at most 70 g/L, at most 80 g/L, at most 90 g/L, at most 100 g/L, at most 200 g/L, at most 300 g/L, at most 400 g/L, at most 500 g/L, at most 600 g/L, at most 700 g/L, at most 800 g/L, at most 900 g/L, or at most 1000 g/L of a mogrol precursor, mogrol, and/or mogroside. In some embodiments, a host cell comprising a lanosterol synthase is capable of producing between 0.01 mg/L and 1 mg/L, between 1 mg/L and 10 mg/L, between 10 mg/L and 20 mg/L, between 10 mg/L and 50 mg/L, between 50 mg/L and 100 mg/L, between 100 mg/L and 200 mg/L, between 200 mg/L and 300 mg/L, between 300 mg/L and 400 mg/L, between 400 mg/L and 500 mg/L, between 500 mg/L and 600 mg/L, between 600 mg/L and 700 mg/L, between 700 mg/L and 800 mg/L, between 800 mg/L and 900 mg/L, between 900 mg/L and 1000 mg/L, between 1 mg/L and 50 mg/L, between 1 mg/L and 100 mg/L, between 1 mg/L and 500 mg/L, between 1 mg/L and 1,000 mg/L, between 1 g/L and 10 g/L, between 10 g/L and 20 g/L, between 10 g/L and 50 g/L, between 50 g/L and 100 g/L, between 100 g/L and 200 g/L, between 200 g/L and 300 g/L, between 300 g/L and 400 g/L, between 400 g/L and 500 g/L, between 500 g/L and 600 g/L, between 600 g/L and 700 g/L, between 700 g/L and 800 g/L, between 800 g/L and 900 g/L, between 900 g/L and 1000 g/L, between 1 g/L and 50 g/L, between 1 g/L and 100 g/L, between 1 g/L and 500 g/L, or between 1 g/L and 1,000 g/L, including all values in between of a mogrol precursor, mogrol, and/or the mogroside. In some embodiments, the mogrol precursor is mevalonate. In some embodiments, the mogrol precursor is 2-3-oxidosqualene. In some embodiments, the mogrol precursor is cucurbitadienol.

In some embodiments, lanosterol is used as a readout of lanosterol synthase activity. For example, a lanosterol synthase with reduced activity may produce less lanosterol from 2-3-oxidosqualene relative to a control. In some embodiments, a control is a different lanosterol synthase. In some embodiments, a control is a wild-type lanosterol synthase. Lanosterol synthase activity may be determined using a cell lysate, a purified enzyme, or in a host cell.

In some embodiments, a lanosterol synthase is capable of decreasing production of lanosterol by a host cell by at least 0.01%, at least 0.05%, at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 150%, at least 200%, at least 250%, at least 300%, at least 350%, at least 400%, at least 450%, at least 500%, at least 550%, at least 600%, at least 650%, at least 700%, at least 750%, at least 800%, at least 850%, at least 900%, at least 950%, or at least 1000%, including all values in between as compared to production of lanosterol by a host cell that does not comprise the lanosterol synthase. In some embodiments, a lanosterol synthase is capable of decreasing production of lanosterol by a host cell at most 5%, at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90%, at most 95%, at most 100%, at most 150%, at most 200%, at most 250%, at most 300%, at most 350%, at most 400%, at most 450%, at most 500%, at most 550%, at most 600%, at most 650%, at most 700%, at most 750%, at most 800%, at most 850%, at most 900%, at most 950%, or at most 1000%, including all values in between as compared to production of lanosterol by a host cell that does not comprise the lanosterol synthase. In some embodiments, a lanosterol synthase is capable of decreasing production of lanosterol by a host cell between 0.01% and 1%, between 1% and 10%, between 10% and 20%, between 10% and 50%, between 50% and 100%, between 100% and 200%, between 200% and 300%, between 300% and 400%, between 400% and 500%, between 500% and 600%, between 600% and 700%, between 700% and 800%, between 800% and 900%, between 900% and 1000%, between 1% and 50%, between 1% and 100%, between 1% and 500%, or between 1% and 1,000%, including all values in between as compared to production of lanosterol by a host cell that does not comprise the lanosterol synthase.

In some embodiments, lanosterol synthase activity in a host cell is determined by the level of ergosterol produced by a cell. Ergosterol is a fungal cell membrane sterol that is produced from lanosterol. Sec, e.g., Klug and Daum, FEMS Yeast Res. 2014 May; 14(3):369-88. In some embodiments, a host cell comprising a lanosterol synthase is capable of producing at most 5 mg/L, at most 10 mg/L, at most 15 mg/L, at most 20 mg/L, at most 25 mg/L, at most 30 mg/L, at most 35 mg/L, at most 40 mg/L, at most 45 mg/L, at most 50 mg/L, at most 55 mg/L, at most 60 mg/L, at most 65 mg/L, at most 70 mg/L, at most 75 mg/L, at most 80 mg/L, at most 85 mg/L, at most 90 mg/L, at most 95 mg/L, at most 100 mg/L, at most 150 mg/L, at most 200 mg/L, at most 250 mg/L, at most 300 mg/L, at most 350 mg/L, at most 400 mg/L, at most 450 mg/L, at most 500 mg/L, at most 550 mg/L, at most 600 mg/L, at most 650 mg/L, at most 700 mg/L, at most 750 mg/L, at most 800 mg/L, at most 850 mg/L, at most 900 mg/L, at most 950 mg/L, at most 1 g/L, at most 1.1 g/L, at most 1.2 g/L, at most 1.3 g/L, at most 1.4 g/L, at most 1.5 g/L, at most 1.6 g/L, at most 1.7 g/L, at most 1.8 g/L, at most 1.9 g/L, at most 2 g/L, at most 2.1 g/L, at most 2.2 g/L, at most 2.3 g/L, at most 2.4 g/L, at most 2.5 g/L, at most 2.6 g/L, at most 2.7 g/L, at most 2.8 g/L, at most 2.9 g/L, at most 3 g/L, at most 3.1 g/L, at most 3.2 g/L, at most 3.3 g/L, at most 3.4 g/L, at most 3.5 g/L, at most 3.6 g/L, at most 3.7 g/L, at most 3.8 g/L, at most 3.9 g/L, at most 4 g/L, at most 4.1 g/L, at most 4.2 g/L, at most 4.3 g/L, at most 4.4 g/L, at most 4.5 g/L, at most 4.6 g/L, at most 4.7 g/L, at most 4.8 g/L, at most 4.9 g/L, at most 5 g/L, at most 5.1 g/L, at most 5.2 g/L, at most 5.3 g/L, at most 5.4 g/L, at most 5.5 g/L, at most 5.6 g/L, at most 5.7 g/L, at most 5.8 g/L, at most 5.9 g/L, at most 6 g/L, at most 6.1 g/L, at most 6.2 g/L, at most 6.3 g/L, at most 6.4 g/L, at most 6.5 g/L, at most 6.6 g/L, at most 6.7 g/L, at most 6.8 g/L, at most 6.9 g/L, at most 7 g/L, at most 7.1 g/L, at most 7.2 g/L, at most 7.3 g/L, at most 7.4 g/L, at most 7.5 g/L, at most 7.6 g/L, at most 7.7 g/L, at most 7.8 g/L, at most 7.9 g/L, at most 8 g/L, at most 8.1 g/L, at most 8.2 g/L, at most 8.3 g/L, at most 8.4 g/L, at most 8.5 g/L, at most 8.6 g/L, at most 8.7 g/L, at most 8.8 g/L, at most 8.9 g/L, at most 9 g/L, at most 9.1 g/L, at most 9.2 g/L, at most 9.3 g/L, at most 9.4 g/L, at most 9.5 g/L, at most 9.6 g/L, at most 9.7 g/L, at most 9.8 g/L, at most 9.9 g/L, at most 10 g/L, at most 20 g/L, at most 30 g/L, at most 40 g/L, at most 50 g/L, at most 60 g/L, at most 70 g/L, at most 80 g/L, at most 90 g/L, at most 100 g/L, at most 200 g/L, at most 300 g/L, at most 400 g/L, at most 500 g/L, at most 600 g/L, at most 700 g/L, at most 800 g/L, at most 900 g/L, or at most 1000 g/L of ergosterol. In some embodiments, a lanosterol synthase is capable of producing between 0.01 mg/L and 1 mg/L, between 1 mg/L and 10 mg/L, between 10 mg/L and 20 mg/L, between 10 mg/L and 50 mg/L, between 50 mg/L and 100 mg/L, between 100 mg/L and 200 mg/L, between 200 mg/L and 300 mg/L, between 300 mg/L and 400 mg/L, between 400 mg/L and 500 mg/L, between 500 mg/L and 600 mg/L, between 600 mg/L and 700 mg/L, between 700 mg/L and 800 mg/L, between 800 mg/L and 900 mg/L, between 900 mg/L and 1000 mg/L, between 1 mg/L and 50 mg/L, between 1 mg/L and 100 mg/L, between 1 mg/L and 500 mg/L, between 1 mg/L and 1,000 mg/L, between 1 g/L and 10 g/L, between 10 g/L and 20 g/L, between 10 g/L and 50 g/L, between 50 g/L and 100 g/L, between 100 g/L and 200 g/L, between 200 g/L and 300 g/L, between 300 g/L and 400 g/L, between 400 g/L and 500 g/L, between 500 g/L and 600 g/L, between 600 g/L and 700 g/L, between 700 g/L and 800 g/L, between 800 g/L and 900 g/L, between 900 g/L and 1000 g/L, between 1 g/L and 50 g/L, between 1 g/L and 100 g/L, between 1 g/L and 500 g/L, or between 1 g/L and 1,000 g/L, including all values in between of ergosterol.

In some embodiments, a lanosterol synthase is capable of producing at most 5 mg/L, at most 10 mg/L, at most 15 mg/L, at most 20 mg/L, at most 25 mg/L, at most 30 mg/L, at most 35 mg/L, at most 40 mg/L, at most 45 mg/L, at most 50 mg/L, at most 55 mg/L, at most 60 mg/L, at most 65 mg/L, at most 70 mg/L, at most 75 mg/L, at most 80 mg/L, at most 85 mg/L, at most 90 mg/L, at most 95 mg/L, at most 100 mg/L, at most 150 mg/L, at most 200 mg/L, at most 250 mg/L, at most 300 mg/L, at most 350 mg/L, at most 400 mg/L, at most 450 mg/L, at most 500 mg/L, at most 550 mg/L, at most 600 mg/L, at most 650 mg/L, at most 700 mg/L, at most 750 mg/L, at most 800 mg/L, at most 850 mg/L, at most 900 mg/L, at most 950 mg/L, at most 1 g/L, at most 1.1 g/L, at most 1.2 g/L, at most 1.3 g/L, at most 1.4 g/L, at most 1.5 g/L, at most 1.6 g/L, at most 1.7 g/L, at most 1.8 g/L, at most 1.9 g/L, at most 2 g/L, at most 2.1 g/L, at most 2.2 g/L, at most 2.3 g/L, at most 2.4 g/L, at most 2.5 g/L, at most 2.6 g/L, at most 2.7 g/L, at most 2.8 g/L, at most 2.9 g/L, at most 3 g/L, at most 3.1 g/L, at most 3.2 g/L, at most 3.3 g/L, at most 3.4 g/L, at most 3.5 g/L, at most 3.6 g/L, at most 3.7 g/L, at most 3.8 g/L, at most 3.9 g/L, at most 4 g/L, at most 4.1 g/L, at most 4.2 g/L, at most 4.3 g/L, at most 4.4 g/L, at most 4.5 g/L, at most 4.6 g/L, at most 4.7 g/L, at most 4.8 g/L, at most 4.9 g/L, at most 5 g/L, at most 5.1 g/L, at most 5.2 g/L, at most 5.3 g/L, at most 5.4 g/L, at most 5.5 g/L, at most 5.6 g/L, at most 5.7 g/L, at most 5.8 g/L, at most 5.9 g/L, at most 6 g/L, at most 6.1 g/L, at most 6.2 g/L, at most 6.3 g/L, at most 6.4 g/L, at most 6.5 g/L, at most 6.6 g/L, at most 6.7 g/L, at most 6.8 g/L, at most 6.9 g/L, at most 7 g/L, at most 7.1 g/L, at most 7.2 g/L, at most 7.3 g/L, at most 7.4 g/L, at most 7.5 g/L, at most 7.6 g/L, at most 7.7 g/L, at most 7.8 g/L, at most 7.9 g/L, at most 8 g/L, at most 8.1 g/L, at most 8.2 g/L, at most 8.3 g/L, at most 8.4 g/L, at most 8.5 g/L, at most 8.6 g/L, at most 8.7 g/L, at most 8.8 g/L, at most 8.9 g/L, at most 9 g/L, at most 9.1 g/L, at most 9.2 g/L, at most 9.3 g/L, at most 9.4 g/L, at most 9.5 g/L, at most 9.6 g/L, at most 9.7 g/L, at most 9.8 g/L, at most 9.9 g/L, at most 10 g/L, at most 20 g/L, at most 30 g/L, at most 40 g/L, at most 50 g/L, at most 60 g/L, at most 70 g/L, at most 80 g/L, at most 90 g/L, at most 100 g/L, at most 200 g/L, at most 300 g/L, at most 400 g/L, at most 500 g/L, at most 600 g/L, at most 700 g/L, at most 800 g/L, at most 900 g/L, or at most 1000 g/L of ergosterol.

In some embodiments, a lanosterol synthase is capable of producing between 0.01 mg/L and 1 mg/L, between 1 mg/L and 10 mg/L, between 10 mg/L and 20 mg/L, between 10 mg/L and 50 mg/L, between 50 mg/L and 100 mg/L, between 100 mg/L and 200 mg/L, between 200 mg/L and 300 mg/L, between 300 mg/L and 400 mg/L, between 400 mg/L and 500 mg/L, between 500 mg/L and 600 mg/L, between 600 mg/L and 700 mg/L, between 700 mg/L and 800 mg/L, between 800 mg/L and 900 mg/L, between 900 mg/L and 1000 mg/L, between 1 mg/L and 50 mg/L, between 1 mg/L and 100 mg/L, between 1 mg/L and 500 mg/L, between 1 mg/L and 1,000 mg/L, between 1 g/L and 10 g/L, between 10 g/L and 20 g/L, between 10 g/L and 50 g/L, between 50 g/L and 100 g/L, between 100 g/L and 200 g/L, between 200 g/L and 300 g/L, between 300 g/L and 400 g/L, between 400 g/L and 500 g/L, between 500 g/L and 600 g/L, between 600 g/L and 700 g/L, between 700 g/L and 800 g/L, between 800 g/L and 900 g/L, between 900 g/L and 1000 g/L, between 1 g/L and 50 g/L, between 1 g/L and 100 g/L, between 1 g/L and 500 g/L, or between 1 g/L and 1,000 g/L, including all values in between of ergosterol.

### Acetoacetyl COA Synthases

Aspects of the present invention provide acetoacetyl COA synthases, which catalyze the condensation of acetyl-CoA and malonyl-CoA to form acetoacetyl-CoA and CoA, but do not accept malonyl-[acyl-carrier-protein] as a substrate. Acetoacetyl CoA synthases can also convert malonyl-CoA into acetyl-CoA via decarboxylation of malonyl-CoA. Aspects of the present invention provide an acetoacetyl COA synthase which increases levels of acetoacetyl-CoA, which is a precursor in a pathway to produce 2,3-oxidosqualene.

In some embodiments, the acetoacetyl COA synthase is encoded by a NphT7 gene. NphT7 catalyzes an alternative path to acetoacetyl-CoA and is present in the mevalonate (MEV) pathway from Saccharomyces cerevisiae. See, e.g., FIG. 1A. In some embodiments, the acetoacetyl COA synthase comprises the amino acid sequence:

(SEQ ID NO: 6)
MTDVRFRIIGTGAYVPERIVSNDEVGAPAGVDDDWITRKTGIRQRRWAAD
DQATSDLATAAGRAALKAAGITPEQLTVIAVATSTPDRPQPPTAAYVQHH
LGATGTAAFDVNAVCSGTVFALSSVAGTLVYRGGYALVIGADLYSRILNP
ADRKTVVLFGDGAGAMVLGPTSTGTGPIVRRVALHTFGGLTDLIRVPAGG
SRQPLDTDGLDAGLQYFAMDGREVRRFVTEHLPQLIKGFLHEAGVDAADI
SHFVPHQANGVMLDEVFGELHLPRATMHRTVETYGNTGAASIPITMDAAV
RAGSFRPGELVLLAGFGGGMAASFALIEW.

In some embodiments, an acetoacetyl COA synthase comprising SEQ ID NO: 6 is encoded by a polynucleotide having a sequence of:

(SEQ ID NO: 7)
ATGACCGACGTCCGATTCCGAATTATCGGTACTGGTGCCTACGTTCCCGA
ACGAATCGTTTCCAACGATGAAGTCGGTGCTCCTGCCGGTGTTGACGACG
ACTGGATCACCCGAAAGACCGGTATTCGACAGCGACGATGGGCTGCCGAT
GACCAGGCCACCTCTGATCTGGCCACTGCTGCCGGTCGAGCTGCCCTGAA
GGCCGCTGGTATCACTCCCGAGCAGCTGACCGTTATTGCTGTTGCCACCT
CCACTCCCGATCGACCCCAGCCTCCCACTGCTGCCTATGTTCAGCACCAC
CTCGGAGCCACCGGTACTGCTGCCTTCGACGTCAACGCTGTCTGCTCCGG
TACCGTTTTCGCCCTGTCCTCTGTTGCTGGCACCCTCGTTTACCGAGGTG
GTTACGCTCTGGTCATTGGCGCTGACCTGTACTCTCGAATCCTCAACCCT
GCCGACCGAAAGACCGTCGTTCTGTTCGGTGATGGTGCCGGTGCCATGGT
TCTCGGTCCTACCTCCACCGGTACTGGTCCCATTGTTCGACGAGTTGCCC
TGCACACCTTCGGTGGTCTGACCGACCTGATTCGAGTCCCCGCTGGTGGT
TCTCGACAGCCCCTGGACACTGATGGCCTCGATGCTGGACTGCAGTACTT
CGCTATGGACGGTCGTGAGGTCCGACGATTCGTCACTGAGCACCTCCCCC
AGCTGATCAAGGGTTTCCTGCACGAGGCCGGTGTCGACGCTGCCGACATC
TCTCACTTCGTCCCTCATCAGGCCAACGGTGTCATGCTCGACGAGGTCTT
CGGCGAGCTGCATCTGCCTCGAGCTACCATGCACCGAACTGTCGAGACTT
ACGGCAACACCGGAGCTGCCTCCATTCCCATCACCATGGACGCTGCCGTT
CGAGCCGGTTCCTTCCGACCTGGTGAGCTGGTCCTGCTGGCCGGTTTCGG
TGGCGGTATGGCCGCTTCCTTCGCCCTGATCGAGTGGTAG.

Acetoacetyl COA synthases of the present disclosure may comprise a sequence that is at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100% identical, including all values in between, with the acetoacetyl CoA synthase sequence set forth as SEQ ID NO: 6 or 7, or to any acetoacetyl CoA synthase sequence disclosed in this application or known in the art. The present disclosure also pertains to a host cell comprising such an acetoacetyl COA synthase, polynucleotides encoding such an acetoacetyl COA synthase, and/or methods of use of such a host cell.

In some embodiments, an acetoacetyl CoA synthase of the present disclosure is capable of promoting formation of acetoacetyl-CoA.

Activity, such as specific activity, of a recombinant acetoacetyl CoA synthase may be measured as the concentration of acetoacetyl-CoA produced per unit of enzyme per unit of time. In some embodiments, an acetoacetyl CoA synthase of the present disclosure has an activity, such as specific activity, of at least 0.0000001 μmol/min/mg (e.g., at least 0.000001 μmol/min/mg, at least 0.00001 μmol/min/mg, at least 0.0001 μmol/min/mg, at least 0.001 μmol/min/mg, at least 0.01 μmol/min/mg, at least 0.1 μmol/min/mg, at least 1 μmol/min/mg, at least 10 μmol/min/mg, or at least 100 μmol/min/mg, including all values in between).

In some embodiments, the activity, such as specific activity, of an acetoacetyl CoA synthase is at least 1.1 fold (e.g., at least 1.3 fold, at least 1.5 fold, at least 1.7 fold, at least 1.9 fold, at least 2 fold, at least 2.5 fold, at least 3 fold, at least 4 fold, at least 5 fold, at least 10 fold, at least 20 fold, at least 30 fold, at least 40 fold, at least 50 fold, or at least 100 fold, including all values in between) greater than that of a control acetoacetyl CoA synthase.

In various aspects, the present disclosure pertains to: an acetoacetyl COA synthase as provided in SEQ ID NO: 6; a polynucleotide encoding an acetoacetyl CoA synthase as provided in SEQ ID NO: 7; a host cell comprising an acetoacetyl COA synthase as provided in SEQ ID NO: 6; or a host cell comprising a polynucleotide encoding an acetoacetyl CoA synthase as provided in SEQ ID NO: 7. In some aspects, the present disclosure pertains to: a method of making a compound of interest, wherein the compound of interest is a mogrol, a mogrol precursor, a mogroside, or a mogroside precursor, wherein the method comprises the step of: producing the compound of interest in a host cell comprising an acetoacetyl CoA synthase as provided in SEQ ID NO: 6, and/or a polynucleotide encoding an acetoacetyl CoA synthase as provided in SEQ ID NO: 7.

### UDP-Glycosyltransferases (UGT) Enzymes

Aspects of the present disclosure provide UDP-glycosyltransferase enzymes (UGTs), which may be useful, for example, in the production of a mogroside (e.g., mogroside I-A1 (MIA1), mogroside I-E (MIE), mogroside II-A1 (MIIA1), mogroside II-A2 (MIIA2), mogroside III-A1 (MIIIA1), mogroside II-E (MIIE), mogroside III (MIII), siamenoside I, mogroside III-E (MIIIE), mogroside IV, mogroside IVa, isomogroside IV, mogroside V, mogroside VIA (MVIA), mogroside VIB (MVIB), isomogroside V, mogroside VIa1 (MVIa1), or mogroside VI).

As used in this disclosure, a “UGT” refers to an enzyme that is capable of catalyzing the addition of the glycosyl group from a UTP-sugar to a compound (e.g., mogroside or mogrol). A UGT may be a primary and/or a secondary UGT.

A “primary” UGT, or a UGT that has “primary glycosylation activity,” refers to a UGT that is capable of catalyzing the addition of a glycosyl group to a position on a compound that does not comprise a glycosyl group. For example, a primary UGT may be capable of adding a glycosyl group to the C3 and/or C24 position of an isoprenoid substrate (e.g., mogrol). Sec, e.g., FIG. 1E.

A “secondary” UGT, or a UGT that has “secondary glycosylation activity,” refers to a UGT that is capable of catalyzing the addition of a glycosyl group to a position on a compound that already comprises a glycosyl group. See, e.g., FIG. 1F. As a non-limiting example, a secondary UGT may add a glycosyl group to a mogroside I-A1 (MIA1), mogroside I-E (MIE), mogroside II-A1 (MIIA1), mogroside II-A2 (MIIA2), mogroside III-A1 (MIIIA1), mogroside II-E (MIIE), mogroside III (MIII), siamenoside I, mogroside III-E (MIIIE), mogroside IV, mogroside IVa, isomogroside IV, mogroside V, mogroside VIA (MVIA), mogroside VIB (MVIB), isomogroside V, mogroside VIa1 (MVIa1), and/or mogroside VI.

In some embodiments, a UGT (e.g., primary or secondary UGT) of the present disclosure comprises a sequence (e.g., nucleic acid or amino acid sequence) that is at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical, including all values in between, to any UGT sequence disclosed in this application or known in the art.

The UGTs of the present disclosure may be capable of glycosylating mogrol or a mogroside at any of the oxygenated sites (e.g., at C3, C11, C24, and C25). In some embodiments, the UGT is capable of branching glycosylation (e.g., branching glycosylation of a mogroside at C3 or C24).

Non-limiting examples of suitable substrates for the UGTs of the present disclosure include mogrol and mogrosides (e.g., mogroside IA1 (MIA1), mogroside IE (MIE), mogroside II-A1 (MIIA1), mogroside III-A1 (MIIIA1), mogroside II-E (MIIE), mogroside III (MIII), or mogroside III-E (MIIIE), siamenoside I).

In some embodiments, the UGTs of the present disclosure are capable of producing mogroside IA1 (MIA1), mogroside IE (MIE), mogroside II-A1 (MIIA1), mogroside II-A2 (MIIA2), mogroside III-A1 (MIIIA1), mogroside II-E (MIIE), mogroside III (MIII), siamenoside I, mogroside III-E (MIIIE), mogroside IV, mogroside IVa, isomogroside IV, mogroside VIA (MVIA), mogroside VIB (MVIB), isomogroside V, mogroside VIa1 (MVIa1), and/or mogroside V.

In some embodiments, the UGT is capable of catalyzing the conversion of mogrol to MIA1; mogrol to MIE1; MIA1 to MIIA1; MIE1 to MIIE; MIIA1 to MIIIA1; MIA1 to MIIE; MIIA1 to MIII; MIIIA1 to siamenoside I; MIIE to MIII; MIII to siamenoside I; MIIE to MIIE; and/or MIIIE to siamenoside I.

It should be appreciated that activity, such as specific activity, of a UGT can be measured by any means known to one of ordinary skill in the art. In some embodiments, the activity, such as specific activity, of a UGT may be determined by measuring the amount of glycosylated mogroside produced per unit enzyme per unit time. For example, the activity, such as specific activity, may be measured in mmol glycosylated mogroside target produced per gram of enzyme per hour. In some embodiments, a UGT of the present disclosure may have an activity, such as specific activity, of at least 0.1 mmol (e.g., at least 1 mmol, at least 1.5 mmol, at least 2 mmol, at least 2.5 mmol, at least 3, at least 3.5 mmol, at least 4 mmol, at least 4.5 mmol, at least 5 mmol, at least 10 mmol, including all values in between) glycosylated mogroside target produced per gram of enzyme per hour.

In some embodiments, the activity, such as specific activity, of a UGT of the present disclosure is at least 1.1 fold (e.g., at least 1.3 fold, at least 1.5 fold, at least 1.7 fold, at least 1.9 fold, at least 2 fold, at least 2.5 fold, at least 3 fold, at least 4 fold, at least 5 fold, at least 10 fold, at least 20 fold, at least 30 fold, at least 40 fold, at least 50 fold, or at least 100 fold, including all values in between) greater than that of a control UGT. In some embodiments, the control UGT is a primary UGT. In some embodiments, the control UGT is a secondary UGT. In some embodiments, the control UGT is UGT94-289-1 (a wildtype UGT sequence from the monk fruit Siraitia grosvenorii provided by SEQ ID NO: 121). In some embodiments, for a UGT that has an amino acid substitution, a control UGT is the same UGT but without the amino acid substitution.

It should be appreciated that one of ordinary skill in the art would be able to characterize a protein as a UGT enzyme based on structural and/or functional information associated with the protein. For example, a protein can be characterized as a UGT enzyme based on its function, such as the ability to produce one or more mogrosides in the presence of a mogroside precursor, such as mogrol.

A UGT enzyme can be further characterized as a primary UGT based on its function of catalyzing the addition of a glycosyl group to a position on a compound that does not comprise a glycosyl group. A UGT enzyme can be characterized as a secondary UGT based on its function of catalyzing the addition of a glycosyl group to a position on a compound that already comprises a glycosyl group. In some embodiments, a UGT enzyme can be characterized as a both primary and a secondary UGT enzyme.

In other embodiments, a protein can be characterized as a UGT enzyme based on the percent identity between the protein and a known UGT enzyme. For example, the protein may be at least 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100% identical, including all values in between, to any of the UGT sequences described in this application or the sequence of any other UGT enzyme.

In other embodiments, a protein can be characterized as a UGT enzyme based on the presence of one or more domains in the protein that are associated with UGT enzymes. For example, in certain embodiments, a protein is characterized as a UGT enzyme based on the presence of a sugar binding domain and/or a catalytic domain, characteristic of UGT enzymes known in the art. In certain embodiments, the catalytic domain binds the substrate to be glycosylated.

In other embodiments, a protein can be characterized as a UGT enzyme based on a comparison of the three-dimensional structure of the protein compared to the three-dimensional structure of a known UGT enzyme. For example, a protein could be characterized as a UGT based on the number or position of alpha helical domains, beta-sheet domains, etc. It should be appreciated that a UGT enzyme can be a synthetic protein.

Structurally, UGTs often comprise a UDPGT (Prosite: PS00375) domain and a catalytic dyad. As a non-limiting example, one of ordinary skill in the art may identify a catalytic dyad in a UGT by aligning the UGT sequence to UGT94-289-1 and identifying the two residues in the UGT that correspond to histidine 21 (H21) and aspartate 122 (D122) of UGT94-289-1.

The amino acid sequence for UGT94-289-1 is:

(SEQ ID NO: 121)
MDAQRGHTTTILMFPWLGYGHLSAFLELAKSLSRRNFHIYFCSTSVNLDA
IKPKLPSSSSSDSIQLVELCLPSSPDQLPPHLHTTNALPPHLMPTLHQAF
SMAAQHFAAILHTLAPHLLIYDSFQPWAPQLASSLNIPAINFNTTGASVL
TRMLHATHYPSSKFPISEFVLHDYWKAMYSAAGGAVTKKDHKIGETLANC
LHASCSVILINSFRELEEKYMDYLSVLLNKKVVPVGPLVYEPNQDGEDEG
YSSIKNWLDKKEPSSTVFVSFGSEYFPSKEEMEEIAHGLEASEVHFIWVV
RFPQGDNTSAIEDALPKGFLERVGERGMVVKGWAPQAKILKHWSTGGFVS
HCGWNSVMESMMFGVPIIGVPMHLDQPFNAGLAEEAGVGVEAKRDPDGKI
QRDEVAKLIKEVVVEKTREDVRKKAREMSEILRSKGEEKMDEMVAAISLF
LKI.

A non-limiting example of a nucleic acid sequence encoding UGT94-289-1 is:

(SEQ ID NO: 60)
atggacgcgcaacgcggacatacgactaccatcctgatgtttccgtggtt
ggggtacggccaccttagtgcattcctcgaattagccaagagcttgtcgc
gtaggaactttcatatttatttctgttccacatctgtcaatttagatgct
ataaaacccaaactaccatcatcttcaagttccgattctattcagcttgt
agagttatgcttgccttcctcgccagaccaactacccccacacctgcata
caactaatgctctacctccacatctaatgcctaccctgcaccaggccttt
tcaatggcagctcaacattttgcagctatattacatactttagcaccgca
cttgttaatctatgattcgttccagccttgggcgccacaattggccagct
ctcttaacattcctgctattaattttaataccacgggtgccagtgtgcta
acaagaatgttacacgcgactcattacccatcttcaaagttcccaatctc
cgaatttgttttacatgattattggaaagcaatgtattcagcagctggtg
gtgctgttacaaaaaaggaccataaaataggagaaaccttggcaaactgt
ttacacgcttcttgctcggtaattctgatcaattcattcagagagttgga
agaaaaatacatggattacttgtctgtcttactaaacaagaaagttgtgc
ccgtgggtccgcttgtttatgagccaaaccaagatggcgaagacgaaggt
tatagttcgataaagaattggctcgataaaaaggagccctcctcaactgt
ctttgtttccttcgggtccgaatattttccgtccaaagaagaaatggaag
aaattgcccatggcttggaggctagcgaggtacactttatttgggtcgtt
agattcccacaaggagacaatacttctgcaattgaagatgcccttcctaa
gggttttcttgagcgagtgggcgaacgtggaatggtggttaagggttggg
ctcctcaggccaaaattttgaaacattggagcacaggcggtttcgtaagt
cattgtggatggaatagtgttatggagagcatgatgtttggtgtacccat
aataggtgttccgatgcatttagatcaaccatttaatgcagggctcgcgg
aagaagcaggagtaggggtagaggctaaaagggaccctgatggtaagata
cagagagatgaagtcgctaaactgatcaaagaagtggttgtcgaaaaaac
gcgcgaagatgtcagaaagaaggctagggaaatgtctgaaattttacgtt
cgaaaggtgaggaaaagatggacgagatggttgcagccattagtctcttc
ttgaagatataa.

One of ordinary skill in the art would readily recognize how to determine for any UGT enzyme what amino acid residue corresponds to a specific amino acid residue in a reference UGT such as UGT94-289-1 (SEQ ID NO: 121) by, for example, aligning sequences and/or by comparing secondary or tertiary structures.

In certain embodiments, a UGT of the present disclosure comprises one or more structural motifs corresponding to a structural motif in wild-type UGT94-289-1 (e.g., corresponding to a structural motif that is shown in Table 1). In some embodiments, a UGT comprises structural motifs corresponding to all structural motifs in Table 1. In some embodiments, a UGT comprises a structural motif that corresponds to some but not all structural motifs shown in Table 1. In some embodiments, some structural motifs may diverge by having different lengths or different helicity. For example, a UGT of the present disclosure may comprise extended versions of loops 11, 16, 20, or a combination thereof. A UGT of the present disclosure may comprise loops that have greater helicity than their counterpart in UGT94-289-1 (e.g., loops 11, 16, 20, or a combination thereof in UGT94-289-1).

TABLE 1
Non-limiting Examples of Structural Motifs in
Reference Sequence UGT94-289-1 (SEQ ID NO: 121)

| | | | SEQ |
|---|---|---|---|
| Structural Motif | Borders | Sequence | ID NO |
| Loop 1 | Met1-Thr9 | MDAQRGHTT | 122 |
| Beta Sheet 1 | Thr10-Phe14 | TILMF | 123 |
| Loop 2 | Pro15-Gly18 | PWLG | 124 |
| Alpha Helix 1 | Tyr19-Arg34 | YGHLSAFLELAKSLSR | 125 |
| Loop3 | Arg35-Phe37 | RNF | 126 |
| Beta Sheet 2 | His38-Phe41 | HIYF | 127 |
| Loop 4 | Cys42-Thr44 | CST | 128 |
| Alpha Helix 2 | Ser45-Ala50 | SVNLDA | 129 |
| Loop 5 | Ile51-Ser61 | IKPKLPSSSSS | 130 |
| Beta Sheet 3 | Asp62-Gln65 | DSIQ | 131 |
| Loop 6 | Leu66-Leu88 | LVELCLPSSPDQLPPHLHTTNAL | 132 |
| Alpha Helix 3 | Pro89-Ala109 | PPHLMPTLHQAFSMAAQHFAA | 133 |
| Loop 7 | Ile110-His117 | ILHTLAPH | 134 |
| Beta Sheet 4 | Leu118-Asp122 | LLIYD | 135 |
| Loop 8 | Ser123-Pro126 | SFQP | 136 |
| Alpha Helix 4 | Trp127-Leu134 | WAPQLASSL | 137 |
| Loop 9 | Asn135-Pro137 | NIP | 138 |
| Beta Sheet 5 | Ala138-Asn143 | AINFN | 139 |
| Loop 10 | Thr144-Gly146 | TTG | 140 |
| Alpha Helix 5 | Ala147-His158 | ASVLTRMLHATH | 141 |
| Loop 11 | Tyr159-Tyr179 | YPSSKFPISEFVLHDYWKAMY | 142 |
| Alpha Helix 6 | Ser180-Gly183 | SAAG | 143 |
| Loop 12 | Gly184-Lys189 | GAVTKK | 144 |
| Alpha Helix 7 | Asp190-Ser204 | DHKIGETLANCLHAS | 145 |
| Loop 13 | Cys205-Ser206 | CS | 146 |
| Beta Sheet 6 | Val207-Ile210 | VILI | 147 |
| Loop 14 | Asn211-Glu217 | NSFRELE | 148 |
| Alpha Helix 8 | Glu218-Leu227 | EKYMDYLSVL | 149 |
| Loop 15 | Leu228-Asn229 | LN | 150 |
| Beta Sheet 7 | Lys230-Val232 | KKV | 151 |
| Loop 16 | Val233-Ser252 | VPVGPLVYEPNQDGEDEGYS | 152 |
| Alpha Helix 9 | Ser253-Lys261 | SIKNWLDKK | 153 |
| Loop 17 | Glu262-Ser265 | EPSS | 154 |
| Beta Sheet 8 | Thr266-Ser270 | TVFVS | 155 |
| Loop 18 | Phe271-Ser278 | FGSEYFPS | 156 |
| Alpha Helix 10 | Lys279-Ser292 | KEEMEEIAHGLEAS | 157 |
| Loop 19 | Glu293-His295 | EVH | 158 |
| Beta Sheet 9 | Phe296-Val300 | FIWVV | 159 |
| Alpha Helix 11 | Arg301-Asn307 | RFPQGDN | 160 |
| Loop 20 | Thr308-Gly318 | TSAIEDALPKG | 161 |
| Alpha Helix 12 | Phe319-Val323 | FLERV | 162 |
| Loop 21 | Gly324-Gly327 | GERG | 163 |
| Beta Sheet 10 | Met328-Lys331 | MVVK | 164 |
| Loop 22 | Gly332-Pro335 | GWAP | 165 |
| Alpha Helix 13 | Gln336-Lys341 | QAKILK | 166 |
| Loop 23 | His342-Gly346 | HWSTG | 167 |
| Beta Sheet 11 | Gly347-Ser350 | GFVS | 168 |
| Loop 24 | His351-Gly353 | HCG | 169 |
| Alpha Helix 14 | Trp354-Phe363 | WNSVMESMMF | 170 |
| Loop 25 | Gly364-Pro366 | GVP | 171 |
| Beta Sheet 12 | Ile367-Val370 | IIGV | 172 |
| Loop 26 | Pro371-Leu374 | PMHL | 173 |
| Alpha Helix 15 | Asp375-Ala386 | DQPFNAGLAEEA | 174 |
| Loop 27 | Gly387-Val388 | GV | 175 |
| Beta Sheet 13 | Gly389-Glu391 | GVE | 176 |
| Loop 28 | Ala392-Gln401 | AKRDPDGKIQ | 177 |
| Alpha Helix 16 | Arg402-Val414 | RDEVAKLIKEVVV | 178 |
| Loop 29 | Glu415 | E | 179 |
| Alpha Helix 17 | Lys416-Gly436 | KTREDVRKKAREMSEILRSKG | 180 |
| Loop 30 | Glu437-Met440 | EEKM | 181 |
| Alpha Helix 18 | Asp441-Leu451 | DEMVAAISLFL | 182 |
| Loop 31 | Lys452-Ile453 | KI | 183 |

In some embodiments, a UGT is a circularly permutated version of a reference UGT. In some embodiments, a UGT comprises a sequence that includes at least two motifs from Table 1 in a different order than a reference UGT. For example, if a reference UGT comprises a first motif that is located C-terminal to a second motif, the first motif may be located N-terminal to the second motif in a circularly permutated UGT.

A UGT may comprise one or more motifs selected from Loop 1, Beta Sheet 1, Loop 2, Alpha Helix 1, Loop 3, Beta Sheet 2, Loop 4, Alpha Helix 2, Loop 5, Beta Sheet 3, Loop 6, Alpha Helix 3, Loop 7, Beta Sheet 4, Loop 8, Alpha Helix 4, Loop 9, Beta Sheet 5, Loop 10, Alpha Helix 5, Loop 11, Alpha Helix 6, Loop 12, Alpha Helix 7, Loop 13, Beta Sheet 6, Loop 14, Alpha Helix 8, and Loop 15 from Table 1 located C-terminal to one or more motifs corresponding to one or more motifs selected from Beta Sheet 7, Loop 16, Alpha Helix 9, Loop 17, Beta Sheet 8, Loop 18, Alpha Helix 10, Loop 19, Beta Sheet 9, Alpha Helix 11, Loop 20, Alpha Helix 12, Loop 21, Beta Sheet 10, Loop 22, Alpha Helix 13, Loop 23, Beta Sheet 11, Loop 24, Alpha Helix 14, Loop 25, Beta Sheet 12, Loop 26, Alpha Helix 15, Loop 27, Beta Sheet 13, Loop 28, Alpha Helix 16, Loop 29, Alpha Helix 17, Loop 30, Alpha Helix 18, and Loop 31 in Table 1.

In some embodiments, the N-terminal portion of a UGT comprises a catalytic site, including a catalytic dyad, and/or a substrate-binding site. In some embodiments, the C-terminal portion of a UGT comprises a cofactor-binding site. Aspects of the disclosure include UGTs that have been circularly permutated. In some embodiments, in a circularly permutated version of a UGT, the N-terminal portion and the C-terminal portions may be reversed in whole or in part. For example, the C-terminal portion of a circularly permutated UGT may comprise a catalytic site, including a catalytic dyad, and/or a substrate-binding site, while the N-terminal portion may comprise a cofactor-binding site. In some embodiments, a circularly permutated version of a UGT comprises a heterologous polynucleotide encoding a UGT, wherein the UGT comprises: a catalytic dyad and a cofactor binding site, wherein the catalytic dyad is located C-terminal to the cofactor-binding site.

A circularly permutated UGT encompassed by the disclosure may exhibit different properties from the same UGT that has not undergone circular permutation. In some embodiments, a host cell expressing such a circularly permutated version of a UGT produces in the presence of at least one mogroside precursor at least 10%, 20%, 30%, 40%, 50%, 60%, 70%, 80%, 90%, or 100% more of one or more mogrosides relative to a host cell that comprises a heterologous polynucleotide encoding a reference UGT that is not circularly permutated, such as wild-type UGT94-289-1 (SEQ ID NO: 121). In some embodiments, a host cell expressing such a circularly permutated version of a UGT produces in the presence of at least one mogroside precursor at least 10%, 20%, 30%, 40%, 50%, 60%, 70%, 80%, 90%, or 100% less of one or more mogrosides relative to a host cell that comprises a heterologous polynucleotide encoding a reference UGT that is not circularly permutated, such as wild-type UGT94-289-1 (SEQ ID NO: 121).

### Cucurbitadienol Synthase (CDS) Enzymes

Aspects of the present disclosure provide cucurbitadienol synthase (CDS) enzymes, which may be useful, for example, in the production of a cucurbitadienol compound, such as 24-25 epoxy-cucurbitadienol or cucurbitadienol. CDSs are capable of catalyzing the formation of cucurbitadienol compounds, such as 24-25 epoxy-cucurbitadienol or cucurbitadienol from oxidosqualene (e.g., 2-3-oxidosqualene or 2,3; 22,23-diepoxysqualene).

In some embodiments, CDSs have a leucine at a residue corresponding to position 123 of SEQ ID NO: 256 that distinguishes them from other oxidosqualene cyclases, as discussed in Takase et al. Org. Biomol. Chem., 2015, 13, 7331-7336, which is incorporated by reference in its entirety.

CDSs of the present disclosure may comprise a sequence that is at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical, including all values in between, to a nucleic acid or amino acid sequence in Table 12, to a sequence selected from SEQ ID NO: 184-263, 299, 308, 327, or 332, or to any other CDS sequence disclosed in this application or known in the art.

In some embodiments a CDS enzyme corresponds to AquAgaCDS16 (SEQ ID NO: 226), CSPI06G07180.1 (SEQ ID NO: 235), or A0A1S3CBF6 (SEQ ID NO: 232). In some embodiments a CDS enzyme corresponds to SgCDS1 (SEQ ID NO: 256).

In some embodiments, a nucleic acid sequence encoding a CDS enzyme may be codon optimized for expression in a particular host cell, including S. cerevisiae. In some embodiments, a codon-optimized nucleic acid sequence encoding a CDS enzyme corresponds to SEQ ID NO: 186, 195, 192, or 327. In some embodiments, a codon-optimized nucleic acid sequence encoding a CDS enzyme corresponds to SEQ ID NO: 332.

In some embodiments, a CDS of the present disclosure is capable of using oxidosqualene (e.g., 2,3-oxidosqualene or 2,3; 22,23-diepoxysqualene) as a substrate. In some embodiments, a CDS of the present disclosure is capable of producing cucurbitadienol compounds (e.g., 24-25 epoxy-cucurbitadienol or cucurbitadienol). In some embodiments, a CDS of the present disclosure catalyzes the formation of cucurbitadienol compounds (e.g., 24-25 epoxy-cucurbitadienol or cucurbitadienol) from oxidosqualene (e.g., 2-3-oxidosqualene or 2,3; 22,23-diepoxysqualene).

It should be appreciated that activity of a CDS can be measured by any means known to one of ordinary skill in the art. In some embodiments, the activity of a CDS may be measured as the normalized peak area of cucurbitadienol produced. In some embodiments, this activity is measured in arbitrary units. In some embodiments, the activity, such as specific activity, of a CDS of the present disclosure is at least 1.1 fold (e.g., at least 1.3 fold, at least 1.5 fold, at least 1.7 fold, at least 1.9 fold, at least 2 fold, at least 2.5 fold, at least 3 fold, at least 4 fold, at least 5 fold, at least 10 fold, at least 20 fold, at least 30 fold, at least 40 fold, at least 50 fold, or at least 100 fold, including all values in between) greater than that of a control CDS.

It should be appreciated that one of ordinary skill in the art would be able to characterize a protein as a CDS enzyme based on structural and/or functional information associated with the protein. For example, in some embodiments, a protein can be characterized as a CDS enzyme based on its function, such as the ability to produce cucurbitadienol compounds (e.g., 24-25 epoxy-cucurbitadienol or cucurbitadienol) using oxidosqualene (e.g., 2,3-oxidosqualene or 2,3; 22,23-diepoxysqualene) as a substrate. In some embodiments, a protein can be characterized, at least in part, as a CDS enzyme based on the presence of a leucine residue at a position corresponding to position 123 of SEQ ID NO: 256.

In some embodiments, a host cell that comprises a heterologous polynucleotide encoding a CDS enzyme produces at least 10%, 20%, 30%, 40%, 50%, 60%, 70%, 80%, 90%, or 100% more cucurbitadienol compound compared to the same host cell that does not express the heterologous gene.

In other embodiments, a protein can be characterized as a CDS enzyme based on the percent identity between the protein and a known CDS enzyme. For example, the protein may be at least 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100% identical, including all values in between, to any of the CDS sequences described in this application or the sequence of any other CDS enzyme. In other embodiments, a protein can be characterized as a CDS enzyme based on the presence of one or more domains in the protein that are associated with CDS enzymes. For example, in certain embodiments, a protein is characterized as a CDS enzyme based on the presence of a substrate channel and/or an active-site cavity characteristic of CDS enzymes known in the art. In some embodiments, the active site cavity comprises a residue that acts a gate to this channel, helping to exclude water from the cavity. In some embodiments, the active-site comprises a residue that acts a proton donor to open the epoxide of the substrate and catalyze the cyclization process.

In other embodiments, a protein can be characterized as a CDS enzyme based on a comparison of the three-dimensional structure of the protein compared to the three-dimensional structure of a known CDS enzyme. It should be appreciated that a CDS enzyme can be a synthetic protein.

### C11 Hydroxylase Enzymes

Aspects of the present disclosure provide C11 hydroxylase enzymes, which may be useful, for example, in the production of mogrol.

A C11 hydroxylase of the present disclosure may comprise a sequence that is at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100% identical, including all values in between, with a C11 hydroxylase sequence (e.g., nucleic acid or amino acid sequence) in Tables 13 and 14, with a sequence set forth as SEQ ID NO: 264-265, 280-281, 296, 305, or 314-315 or to any C11 hydroxylase sequence disclosed in this application or known in the art.

In some embodiments, a C11 hydroxylase of the present disclosure is capable of oxidizing mogrol precursors (e.g., cucurbitadienol, 11-hydroxycucurbitadienol, 24,25-dihydroxy-cucurbitadienol, and/or 24,25-epoxy-cucurbitadienol). In some embodiments, a C11 hydroxylase of the present disclosure catalyzes the formation of mogrol.

It should be appreciated that activity, such as specific activity, of a C11 hydroxylase can be determined by any means known to one of ordinary skill in the art. In some embodiments, activity (e.g., specific activity) of a C11 hydroxylase may be measured as the concentration of a mogrol precursor produced or mogrol produced per unit of enzyme per unit time. In some embodiments, a C11 hydroxylase of the present disclosure has an activity (e.g., specific activity) of at least 0.0001-0.001 μmol/min/mg, at least 0.001-0.01 μmol/min/mg, at least 0.01-0.1 μmol/min/mg, or at least 0.1-1 μmol/min/mg, including all values in between.

In some embodiments, the activity, such as specific activity, of a C11 hydroxylase is at least 1.1 fold (e.g., at least 1.3 fold, at least 1.5 fold, at least 1.7 fold, at least 1.9 fold, at least 2 fold, at least 2.5 fold, at least 3 fold, at least 4 fold, at least 5 fold, at least 10 fold, at least 20 fold, at least 30 fold, at least 40 fold, at least 50 fold, at least 100 fold, at least 1000 fold or at least 10000 fold, including all values in between) greater than that of a control C11 hydroxylase.

### Cytochrome P450 Reductase Enzymes

Aspects of the present disclosure provide cytochrome P450 reductase enzymes, which may be useful, for example, in the production of mogrol. Cytochrome P450 reductase is also referred to as NADPH: ferrihemoprotein oxidoreductase, NADPH:hemoprotein oxidoreductase, NADPH:P450 oxidoreductase, P450 reductase, POR, CPR, and CYPOR. These reductases can promote C11 hydroxylase activity by catalyzing electron transfer from NADPH to a C11 hydroxylase.

Cytochrome P450 reductases of the present disclosure may comprise a sequence that is at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100% identical, including all values in between, with a cytochrome P450 reductase sequence (e.g., nucleic acid or amino acid sequence) in Tables 13 and 14, with a sequence set forth as SEQ ID NO: 266-267, 282-283, 297-298, or 306-307, or to any cytochrome p450 reductase disclosed in this application or known in the art.

In some embodiments, a cytochrome P450 reductase of the present disclosure is capable of promoting oxidation of a mogrol precursor (e.g., cucurbitadienol, 11-hydroxycucurbitadienol, 24,25-dihydroxy-cucurbitadienol, and/or 24,25-epoxy-cucurbitadienol). In some embodiments, a P450 reductase of the present disclosure catalyzes the formation of a mogrol precursor or mogrol.

It should be appreciated that activity (e.g., specific activity) of a cytochrome P450 reductase can be measured by any means known to one of ordinary skill in the art. In some embodiments, activity (e.g., specific activity) of a recombinant cytochrome P450 reductase may be measured as the concentration of a mogrol precursor produced or mogrol produced per unit enzyme per unit time in the presence of a C11 hydroxylase. In some embodiments, a cytochrome P450 reductase of the present disclosure has a activity (e.g., specific activity) of at least 0.0001-0.001 μmol/min/mg, at least 0.001-0.01 μmol/min/mg, at least 0.01-0.1 μmol/min/mg, or at least 0.1-1 μmol/min/mg, including all values in between.

In some embodiments, the activity (e.g., specific activity) of a cytochrome P450 reductase is at least 1.1 fold (e.g., at least 1.3 fold, at least 1.5 fold, at least 1.7 fold, at least 1.9 fold, at least 2 fold, at least 2.5 fold, at least 3 fold, at least 4 fold, at least 5 fold, at least 10 fold, at least 20 fold, at least 30 fold, at least 40 fold, at least 50 fold, at least 100 fold, at least 1000 fold or at least 10000 fold, including all values in between) greater than that of a control cytochrome P450 reductase.

In some embodiments, wherein 11-oxo mogrol is not a desired product, the level, expression and/or activity of a cytochrome P450 reductase, which is involved in synthesis of 11-oxo mogrol, is decreased in the host cell relative to a control host cell. In some embodiments, relative to a control host cell, the activity of a cytochrome P450 reductase is reduced in a host cell that comprises a heterologous polynucleotide that encodes a cytochrome P450 with reduced activity as compared to a control cytochrome P450 or in a host cell that comprises a heterologous polynucleotide that reduces cytochrome P450 activity. In some embodiments, the control host cell does not comprise a heterologous polynucleotide that encodes a cytochrome P450 with reduced activity as compared to a control cytochrome P450 or is a host cell that does not comprise a heterologous polynucleotide that reduces cytochrome P450 activity.

In some embodiments, the activity (e.g., specific activity) of a cytochrome P450 reductase is reduced at least 1.1 fold (e.g., at least 1.3 fold, at least 1.5 fold, at least 1.7 fold, at least 1.9 fold, at least 2 fold, at least 2.5 fold, at least 3 fold, at least 4 fold, at least 5 fold, at least 10 fold, at least 20 fold, at least 30 fold, at least 40 fold, at least 50 fold, at least 100 fold, at least 1000 fold or at least 10000 fold, including all values in between) in a host cell as compared to a control. In some embodiments, the control is a host cell that does not comprise a heterologous polynucleotide that encodes a cytochrome P450 with reduced activity as compared to a control cytochrome P450 or a host cell that does not comprise a heterologous polynucleotide that reduces cytochrome P450 activity.

### Epoxide Hydrolase Enzymes (EPHs)

Aspects of the present disclosure provide epoxide hydrolase enzymes (EPHs), which may be useful, for example, in the conversion of 24-25 epoxy-cucurbitadienol to 24-25 dihydroxy-cucurbitadienol or in the conversion of 11-hydroxy-24,25-epoxycucurbitadienol to mogrol. EPHs are capable of converting an epoxide into two hydroxyls.

EPHs of the present disclosure may comprise a sequence that is at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100% identical, including all values in between, with a EPH sequence (e.g., nucleic acid or amino acid sequence) in Tables 13 and 14, with a sequence set forth as SEQ ID NO: 268-276, 284-292, 300-301 or 309-310, or to any EPH sequence disclosed in this application or known in the art.

In some embodiments, a recombinant EPH of the present disclosure is capable of promoting hydrolysis of an epoxide in a cucurbitadienol compound (e.g., hydrolysis of the epoxide in 24-25 epoxy-cucurbitadienol). In some embodiments, an EPH of the present disclosure catalyzes the formation of a mogrol precursor (e.g., 24-25 dihydroxy-cucurbitadienol).

It should be appreciated that activity (e.g., specific activity) of an EPH can be measured by any means known to one of ordinary skill in the art. In some embodiments, activity (e.g., specific activity) of an EPH may be measured as the concentration of a mogrol precursor (e.g., 24-25 dihydroxy-cucurbitadienol) or mogrol produced. In some embodiments, a recombinant EPH of the present disclosure will allow production of at least 1-100 μg/L, at least 100-1000 μg/L, at least 1-100 mg/L, at least 100-1000 mg/L, at least 1-10 g/L or at least 10-100 g/L, including all values in between.

In some embodiments, the activity (e.g., specific activity) of an EPH is at least 1.1 fold (e.g., at least 1.3 fold, at least 1.5 fold, at least 1.7 fold, at least 1.9 fold, at least 2 fold, at least 2.5 fold, at least 3 fold, at least 4 fold, at least 5 fold, at least 10 fold, at least 20 fold, at least 30 fold, at least 40 fold, at least 50 fold, or at least 100 fold, including all values in between) greater than that of a control EPH.

### Squalene Epoxidases Enzymes (SQEs)

Aspects of the present disclosure provide squalene epoxidases (SQEs), which are capable of oxidizing a squalene (e.g., squalene or 2-3-oxidosqualene) to produce a squalene epoxide (e.g., 2-3-oxidosqualene or 2-3, 22-23-diepoxysqualene). SQEs may also be referred to as squalene monooxygenases.

SQEs of the present disclosure may comprise a sequence that is at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100% identical, including all values in between, with a SQE sequence (e.g., nucleic acid or amino acid sequence) in Tables 13 and 14, with a sequence set forth as SEQ ID NO: 277-279, 293-295, 303 or 312, or to any SQE sequence disclosed in this application or known in the art.

In some embodiments, an SQE of the present disclosure is capable of promoting formation of an epoxide in a squalene compound (e.g., epoxidation of squalene or 2,3-oxidosqualene). In some embodiments, an SQE of the present disclosure catalyzes the formation of a mogrol precursor (e.g., 2-3-oxidosqualene or 2-3, 22-23-diepoxysqualene).

Activity, such as specific activity, of a recombinant SQE may be measured as the concentration of a mogrol precursor (e.g., 2-3-oxidosqualene or 2-3, 22-23-diepoxysqualene) produced per unit of enzyme per unit of time. In some embodiments, an SQE of the present disclosure has an activity, such as specific activity, of at least 0.0000001 μmol/min/mg (e.g., at least 0.000001 μmol/min/mg, at least 0.00001 μmol/min/mg, at least 0.0001 μmol/min/mg, at least 0.001 μmol/min/mg, at least 0.01 μmol/min/mg, at least 0.1 μmol/min/mg, at least 1 μmol/min/mg, at least 10 μmol/min/mg, or at least 100 μmol/min/mg, including all values in between).

In some embodiments, the activity, such as specific activity, of a SQE is at least 1.1 fold (e.g., at least 1.3 fold, at least 1.5 fold, at least 1.7 fold, at least 1.9 fold, at least 2 fold, at least 2.5 fold, at least 3 fold, at least 4 fold, at least 5 fold, at least 10 fold, at least 20 fold, at least 30 fold, at least 40 fold, at least 50 fold, or at least 100 fold, including all values in between) greater than that of a control SQE.

### Variants

Aspects of the disclosure relate to polynucleotides encoding any of the recombinant polypeptides described, such as lanosterol synthase, acetoacetyl COA synthase, CB5, CDS, UGT, C11 hydroxylase, cytochrome P450 reductase, and EPH, SQE enzymes and any proteins associated with the disclosure. Variants of polynucleotide or amino acid sequences described in this application are also encompassed by the present disclosure. A variant may share at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity with a reference sequence, including all values in between.

Unless otherwise noted, the term “sequence identity,” as known in the art, refers to a relationship between the sequences of two polypeptides or polynucleotides, as determined by sequence comparison (alignment). In some embodiments, sequence identity is determined across the entire length of a sequence, while in other embodiments, sequence identity is determined over a region of a sequence.

Identity can also refer to the degree of sequence relatedness between two sequences as determined by the number of matches between strings of two or more residues (e.g., nucleic acid or amino acid residues). Identity measures the percent of identical matches between the smaller of two or more sequences with gap alignments (if any) addressed by a particular mathematical model, algorithms, or computer program.

Identity of related polypeptides or nucleic acid sequences can be readily calculated by any of the methods known to one of ordinary skill in the art. The “percent identity” of two sequences (e.g., nucleic acid or amino acid sequences) may, for example, be determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST® and XBLAST® programs (version 2.0) of Altschul et al., J. Mol. Biol. 215:403-10, 1990. BLAST® protein searches can be performed, for example, with the XBLAST program, score=50, wordlength=3 to obtain amino acid sequences homologous to the protein molecules of the invention. Where gaps exist between two sequences, Gapped BLAST® can be utilized, for example, as described in Altschul et al., Nucleic Acids Res. 25(17):3389-3402, 1997. When utilizing BLAST® and Gapped BLAST® programs, the default parameters of the respective programs (e.g., XBLAST® and NBLAST®) can be used, or the parameters can be adjusted appropriately as would be understood by one of ordinary skill in the art.

Another local alignment technique which may be used, for example, is based on the Smith-Waterman algorithm (Smith, T. F. & Waterman, M. S. (1981) “Identification of common molecular subsequences.” J. Mol. Biol. 147:195-197). A general global alignment technique which may be used, for example, is the Needleman-Wunsch algorithm (Needleman, S. B. & Wunsch, C. D. (1970) “A general method applicable to the search for similarities in the amino acid sequences of two proteins.” J. Mol. Biol. 48:443-453), which is based on dynamic programming.

More recently, a Fast Optimal Global Sequence Alignment Algorithm (FOGSAA) was developed that purportedly produces global alignment of nucleic acid and amino acid sequences faster than other optimal global alignment methods, including the Needleman-Wunsch algorithm. In some embodiments, the identity of two polypeptides is determined by aligning the two amino acid sequences, calculating the number of identical amino acids, and dividing by the length of one of the amino acid sequences. In some embodiments, the identity of two nucleic acids is determined by aligning the two nucleotide sequences and calculating the number of identical nucleotide and dividing by the length of one of the nucleic acids.

For multiple sequence alignments, computer programs including Clustal Omega (Sievers et al., Mol Syst Biol. 2011 Oct. 11; 7:539) may be used.

In preferred embodiments, a sequence, including a nucleic acid or amino acid sequence, is found to have a specified percent identity to a reference sequence, such as a sequence disclosed in this application and/or recited in the claims when sequence identity is determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993 (e.g., BLAST®, NBLAST®, XBLAST® or Gapped BLAST® programs, using default parameters of the respective programs).

In some embodiments, a sequence, including a nucleic acid or amino acid sequence, is found to have a specified percent identity to a reference sequence, such as a sequence disclosed in this application and/or recited in the claims when sequence identity is determined using the Smith-Waterman algorithm (Smith, T. F. & Waterman, M. S. (1981) “Identification of common molecular subsequences.” J. Mol. Biol. 147: 195-197) or the Needleman-Wunsch algorithm (Needleman, S. B. & Wunsch, C. D. (1970) “A general method applicable to the search for similarities in the amino acid sequences of two proteins.” J. Mol. Biol. 48:443-453).

In some embodiments, a sequence, including a nucleic acid or amino acid sequence, is found to have a specified percent identity to a reference sequence, such as a sequence disclosed in this application and/or recited in the claims when sequence identity is determined using a Fast Optimal Global Sequence Alignment Algorithm (FOGSAA).

In some embodiments, a sequence, including a nucleic acid or amino acid sequence, is found to have a specified percent identity to a reference sequence, such as a sequence disclosed in this application and/or recited in the claims when sequence identity is determined using Clustal Omega (Sievers et al., Mol Syst Biol. 2011 Oct. 11; 7:539).

As used in this application, a residue (such as a nucleic acid residue or an amino acid residue) in sequence “X” is referred to as corresponding to a position or residue (such as a nucleic acid residue or an amino acid residue) “Z” in a different sequence “Y” when the residue in sequence “X” is at the counterpart position of “Z” in sequence “Y” when sequences X and Y are aligned using amino acid sequence alignment tools known in the art.

Variant sequences may be homologous sequences. As used in this application, homologous sequences are sequences (e.g., nucleic acid or amino acid sequences) that share a certain percent identity (e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% percent identity, including all values in between) and include but are not limited to paralogous sequences, orthologous sequences, or sequences arising from convergent evolution. Paralogous sequences arise from duplication of a gene within a genome of a species, while orthologous sequences diverge after a speciation event. Two different species may have evolved independently but may each comprise a sequence that shares a certain percent identity with a sequence from the other species as a result of convergent evolution.

In some embodiments, a polypeptide variant (e.g., lanosterol synthase, acetoacetyl CoA synthase, CB5, CDS, UGT, C11 hydroxylase, cytochrome P450 reductase, EPH, or SQE variant or variant of any protein associated with the disclosure) comprises a domain that shares a secondary structure (e.g., alpha helix, beta sheet) with a reference polypeptide (e.g., a reference lanosterol synthase, acetoacetyl CoA synthase, CB5, CDS, UGT, C11 hydroxylase, cytochrome P450 reductase, EPH, SQE, or any protein associated with the disclosure). In some embodiments, a polypeptide variant (e.g., lanosterol synthase, acetoacetyl CoA synthase, CB5, CDS, UGT, C11 hydroxylase, cytochrome P450 reductase, EPH, or SQE variant or variant of any protein associated with the disclosure) shares a tertiary structure with a reference polypeptide (e.g., a reference lanosterol synthase, acetoacetyl COA synthase, CB5, CDS, UGT, C11 hydroxylase, cytochrome P450 reductase, EPH, SQE, or any protein associated with the disclosure). As a non-limiting example, a variant polypeptide may have low primary sequence identity (e.g., less than 80%, less than 75%, less than 70%, less than 65%, less than 60%, less than 55%, less than 50%, less than 45%, less than 40%, less than 35%, less than 30%, less than 25%, less than 20%, less than 15%, less than 10%, or less than 5% sequence identity) compared to a reference polypeptide, but share one or more secondary structures (e.g., including but not limited to loops, alpha helices, or beta sheets, or have the same tertiary structure as a reference polypeptide. For example, a loop may be located between a beta sheet and an alpha helix, between two alpha helices, or between two beta sheets. Homology modeling may be used to compare two or more tertiary structures.

Mutations can be made in a nucleotide sequence by a variety of methods known to one of ordinary skill in the art. For example, mutations can be made by PCR-directed mutation, site-directed mutagenesis according to the method of Kunkel (Kunkel, Proc. Nat. Acad. Sci. U.S.A. 82: 488-492, 1985), by chemical synthesis of a gene encoding a polypeptide, by gene editing tools, or by insertions, such as insertion of a tag (e.g., a HIS tag or a GFP tag). Mutations can include, for example, substitutions, deletions, and translocations, generated by any method known in the art. Methods for producing mutations may be found in in references such as Molecular Cloning: A Laboratory Manual, J. Sambrook, et al., eds., Fourth Edition, Cold Spring Harbor Laboratory Press, Cold Spring Harbor, New York, 2012, or Current Protocols in Molecular Biology, F. M. Ausubel, et al., eds., John Wiley & Sons, Inc., New York, 2010.

In some embodiments, methods for producing variants include circular permutation (Yu and Lutz, Trends Biotechnol. 2011 January; 29(1):18-25). In circular permutation, the linear primary sequence of a polypeptide can be circularized (e.g., by joining the N-terminal and C-terminal ends of the sequence) and the polypeptide can be severed (“broken”) at a different location. Thus, the linear primary sequence of the new polypeptide may have low sequence identity (e.g., less than 80%, less than 75%, less than 70%, less than 65%, less than 60%, less than 55%, less than 50%, less than 45%, less than 40%, less than 35%, less than 30%, less than 25%, less than 20%, less than 15%, less than 10%, less or less than 5%, including all values in between) as determined by linear sequence alignment methods (e.g., Clustal Omega or BLAST). Topological analysis of the two proteins, however, may reveal that the tertiary structure of the two polypeptides is similar or dissimilar. Without being bound by a particular theory, a variant polypeptide created through circular permutation of a reference polypeptide and with a similar tertiary structure as the reference polypeptide can share similar functional characteristics (e.g., enzymatic activity, enzyme kinetics, substrate specificity or product specificity). In some instances, circular permutation may alter the secondary structure, tertiary structure or quaternary structure and produce a protein with different functional characteristics (e.g., increased or decreased enzymatic activity, different substrate specificity, or different product specificity). Sec, e.g., Yu and Lutz, Trends Biotechnol. 2011 January; 29(1):18-25.

It should be appreciated that in a protein that has undergone circular permutation, the linear amino acid sequence of the protein would differ from a reference protein that has not undergone circular permutation. However, one of ordinary skill in the art would be able to determine which residues in the protein that has undergone circular permutation correspond to residues in the reference protein that has not undergone circular permutation by, for example, aligning the sequences and detecting conserved motifs, and/or by comparing the structures or predicted structures of the proteins, e.g., by homology modeling.

In some embodiments, an algorithm that determines the percent identity between a sequence of interest and a reference sequence described in this application accounts for the presence of circular permutation between the sequences. The presence of circular permutation may be detected using any method known in the art, including, for example, RASPODOM (Weiner et al., Bioinformatics. 2005 Apr. 1; 21(7):932-7). In some embodiments, the presence of circulation permutation is corrected for (e.g., the domains in at least one sequence are rearranged) prior to calculation of the percent identity between a sequence of interest and a sequence described in this application. The claims of this application should be understood to encompass sequences for which percent identity to a reference sequence is calculated after taking into account potential circular permutation of the sequence.

Functional variants of the recombinant lanosterol synthases, acetoacetyl CoA synthases, CB5, CDSs, UGTs, C11 hydroxylases, cytochrome P450 reductases, EPHs, squalene epoxidases, and any other proteins disclosed in this application are also encompassed by the present disclosure. For example, functional variants may bind one or more of the same substrates (e.g., mogrol, mogroside, or precursors thereof) or produce one or more of the same products (e.g., mogrol, mogroside, or precursors thereof). Functional variants may be identified using any method known in the art. For example, the algorithm of Karlin and Altschul Proc. Natl. Acad. Sci. USA 87:2264-68, 1990 described above may be used to identify homologous proteins with known functions.

Putative functional variants may also be identified by searching for polypeptides with functionally annotated domains. Databases including Pfam (Sonnhammer et al., Proteins. 1997 July; 28(3):405-20) may be used to identify polypeptides with a particular domain. For example, among oxidosqualene cyclases, additional CDS enzymes may be identified in some instances by searching for polypeptides with a leucine residue corresponding to position 123 of SEQ ID NO: 256. This leucine residue has been implicated in determining the product specificity of the CDS enzyme; mutation of this residue can, for instance, result in cycloartenol or parkeol as a product (Takase et al., Org Biomol Chem. 2015 Jul. 13(26):7331-6).

Additional UGT enzymes may be identified, for example, by searching for polypeptides with a UDPGT domain (PROSITE accession number PS00375).

Homology modeling may also be used to identify amino acid residues that are amenable to mutation without affecting function. A non-limiting example of such a method may include use of position-specific scoring matrix (PSSM) and an energy minimization protocol. Sec, e.g., Stormo et al., Nucleic Acids Res. 1982 May 11; 10(9):2997-3011.

PSSM may be paired with calculation of a Rosetta energy function, which determines the difference between the wild-type and the single-point mutant. Without being bound by a particular theory, potentially stabilizing mutations are desirable for protein engineering (e.g., production of functional homologs). In some embodiments, a potentially stabilizing mutation has a ΔΔG_{calc} value of less than −0.1 (e.g., less than −0.2, less than −0.3, less than −0.35, less than −0.4, less than −0.45, less than −0.5, less than −0.55, less than −0.6, less than −0.65, less than −0.7, less than −0.75, less than −0.8, less than −0.85, less than −0.9, less than −0.95, or less than −1.0) Rosetta energy units (R.e.u.). See, e.g., Goldenzweig et al., Mol Cell. 2016 Jul. 21; 63(2):337-346. doi: 10.1016/j.molcel.2016.06.012.

In some embodiments, a lanosterol synthase, acetoacetyl CoA synthase, CB5, CDS, UGT, C11 hydroxylase, cytochrome P450 reductase, EPH, or SQE coding sequence or coding sequence of any protein associated with the disclosure comprises a mutation at 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more than 100 positions corresponding to a reference coding sequence. In some embodiments, the lanosterol synthase, acetoacetyl CoA synthase, CB5, CDS, UGT, C11 hydroxylase, cytochrome P450 reductase, EPH, or SQE coding sequence or coding sequence of any protein associated with the disclosure comprises a mutation in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more codons of the coding sequence relative to a reference coding sequence. As will be understood by one of ordinary skill in the art, a mutation within a codon may or may not change the amino acid that is encoded by the codon due to degeneracy of the genetic code. In some embodiments, the one or more mutations in the coding sequence do not alter the amino acid sequence of the coding sequence relative to the amino acid sequence of a reference polypeptide.

In some embodiments, the one or more mutations in a recombinant lanosterol synthase, acetoacetyl CoA synthase, CB5, CDS, UGT, C11 hydroxylase, cytochrome P450 reductase, EPH, or SQE sequence or other recombinant protein sequence associated with the disclosure alter the amino acid sequence of the polypeptide relative to the amino acid sequence of a reference polypeptide. In some embodiments, the one or more mutations alter the amino acid sequence of the recombinant polypeptide relative to the amino acid sequence of a reference polypeptide and alter (enhance or reduce) an activity of the polypeptide relative to the reference polypeptide.

The activity, including specific activity, of any of the recombinant polypeptides described in this application may be measured using methods known in the art. As a non-limiting example, a recombinant polypeptide's activity may be determined by measuring its substrate specificity, product(s) produced, the concentration of product(s) produced, or any combination thereof. As used in this application, “specific activity” of a recombinant polypeptide refers to the amount (e.g., concentration) of a particular product produced for a given amount (e.g., concentration) of the recombinant polypeptide per unit time.

The skilled artisan will also realize that mutations in a recombinant polypeptide coding sequence may result in conservative amino acid substitutions to provide functionally equivalent variants of the foregoing polypeptides, e.g., variants that retain the activities of the polypeptides. As used in this application, a “conservative amino acid substitution” or “conservatively substituted” refers to an amino acid substitution that does not alter the relative charge or size characteristics or functional activity of the protein in which the amino acid substitution is made.

In some instances, an amino acid is characterized by its R group (see, e.g., Table 2). For example, an amino acid may comprise a nonpolar aliphatic R group, a positively charged R group, a negatively charged R group, a nonpolar aromatic R group, or a polar uncharged R group. Non-limiting examples of an amino acid comprising a nonpolar aliphatic R group include alanine, glycine, valine, leucine, methionine, and isoleucine. Non-limiting examples of an amino acid comprising a positively charged R group includes lysine, arginine, and histidine. Non-limiting examples of an amino acid comprising a negatively charged R group include aspartate and glutamate. Non-limiting examples of an amino acid comprising a nonpolar, aromatic R group include phenylalanine, tyrosine, and tryptophan. Non-limiting examples of an amino acid comprising a polar uncharged R group include serine, threonine, cysteine, proline, asparagine, and glutamine.

Non-limiting examples of functionally equivalent variants of polypeptides may include conservative amino acid substitutions in the amino acid sequences of proteins disclosed in this application. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D. Additional non-limiting examples of conservative amino acid substitutions are provided in Table 2.

In some embodiments, 1, 2, 3, 4, 5, 6, 7, 8, 9, 10, 11, 12, 13, 14, 15, 16, 17, 18, 19, 20 or more than 20 residues can be changed when preparing variant polypeptides. In some embodiments, amino acids are replaced by conservative amino acid substitutions.

TABLE 2
Non-limiting Examples of Conservative Amino Acid Substitutions

| Original | | Conservative Amino |
|---|---|---|
| Residue | R Group Type | Acid Substitutions |
| Ala (A) | nonpolar aliphatic R group | Cys, Gly, Ser |
| Arg (R) | positively charged R group | His, Lys |
| Asn (N) | polar uncharged R group | Asp, Gln, Glu |
| Asp (D) | negatively charged R group | Asn, Gln, Glu |
| Cys (C) | polar uncharged R. group | Ala, Ser |
| Gln (Q) | polar uncharged R group | Asn, Asp, Glu |
| Glu (E) | negatively charged R group | Asp, Asp, Gln |
| Gly (G) | nonpolar aliphatic R group | Ala, Ser |
| His (H) | positively charged R. group | Arg, Tyr, Trp |
| Ile (I) | nonpolar aliphatic R group | Leu, Met, Val |
| Leu (L) | nonpolar aliphatic R group | Ile, Met, Val |
| Lys (K) | positively charged R group | Arg, His |
| Met (M) | nonpolar aliphatic R group | Ile, Leu, Phe, Val |
| Pro (P) | polar uncharged R group | |
| Phe (F) | nonpolar aromatic R group | Met, Trp, Tyr |
| Ser (S) | polar uncharged R group | Ala, Gly, Thr |
| Thr (T) | polar uncharged R group | Ala, Asn, Ser |
| Trp (W) | nonpolar aromatic R group | His, Phe, Tyr, Met |
| Tyr (Y) | nonpolar aromatic R group | His, Phe, Trp |
| Val (V) | nonpolar aliphatic R group | Ile, Leu, Met, Thr |

Amino acid substitutions in the amino acid sequence of a polypeptide to produce a recombinant polypeptide variant having a desired property and/or activity can be made by alteration of the coding sequence of the polypeptide. Similarly, conservative amino acid substitutions in the amino acid sequence of a polypeptide to produce functionally equivalent variants of the polypeptide typically are made by alteration of the coding sequence of the recombinant polypeptide (e.g., lanosterol synthase, acetoacetyl CoA synthase, CB5, UGT, CDS, P450, cytochrome P450 reductase, EPH, squalene epoxidase, or any protein associated with the disclosure).

### Expression of Nucleic Acids in Host Cells

Aspects of the present disclosure relate to the recombinant expression of genes encoding proteins, functional modifications and variants thereof, as well as uses relating thereto. For example, the methods described in this application may be used to produce mogrol precursors, mogrol, and/or mogrosides.

The term “heterologous” with respect to a polynucleotide, such as a polynucleotide comprising a gene, is used interchangeably with the term “exogenous” and the term “recombinant” and refers to: a polynucleotide that has been artificially supplied to a biological system; a polynucleotide that has been modified within a biological system; or a polynucleotide whose expression or regulation has been manipulated within a biological system. A heterologous polynucleotide that is introduced into or expressed in a host cell may be a polynucleotide that comes from a different organism or species from the host cell, or may be a synthetic polynucleotide, or may be a polynucleotide that is also endogenously expressed in the same organism or species as the host cell. For example, a polynucleotide that is endogenously expressed in a host cell may be considered heterologous when it is: situated non-naturally in the host cell; expressed recombinantly in the host cell, either stably or transiently; modified within the host cell; selectively edited within the host cell; expressed in a copy number that differs from the naturally occurring copy number within the host cell; or expressed in a non-natural way within the host cell, such as by manipulating regulatory regions that control expression of the polynucleotide. In some embodiments, a heterologous polynucleotide is a polynucleotide that is endogenously expressed in a host cell but whose expression is driven by a promoter that does not naturally regulate expression of the polynucleotide. In other embodiments, a heterologous polynucleotide is a polynucleotide that is endogenously expressed in a host cell and whose expression is driven by a promoter that does naturally regulate expression of the polynucleotide, but the promoter or another regulatory region is modified. In some embodiments, the promoter is recombinantly activated or repressed. For example, gene-editing based techniques may be used to regulate expression of a polynucleotide, including an endogenous polynucleotide, from a promoter, including an endogenous promoter. Sec, e.g., Chavez et al., Nat Methods. 2016 July; 13(7): 563-567. A heterologous polynucleotide may comprise a wild-type sequence or a mutant sequence as compared with a reference polynucleotide sequence.

A nucleic acid encoding any of the recombinant polypeptides, such as lanosterol synthases, acetoacetyl COA synthases, CB5, CDSs, UGTs, C11 hydroxylases, cytochrome P450 reductases, EPHs, SQEs, or any proteins associated with the disclosure, described in this application may be incorporated into any appropriate vector through any method known in the art. For example, the vector may be an expression vector, including but not limited to a viral vector (e.g., a lentiviral, retroviral, adenoviral, or adeno-associated viral vector), any vector suitable for transient expression, any vector suitable for constitutive expression, or any vector suitable for inducible expression (e.g., a galactose-inducible or doxycycline-inducible vector).

In some embodiments, a vector replicates autonomously in the cell. A vector can contain one or more endonuclease restriction sites that are cut by a restriction endonuclease to insert and ligate a nucleic acid containing a gene described in this application to produce a recombinant vector that is able to replicate in a cell. Vectors can be composed of DNA or RNA. Cloning vectors include, but are not limited to: plasmids, fosmids, phagemids, virus genomes and artificial chromosomes. As used in this application, the terms “expression vector” or “expression construct” refer to a nucleic acid construct, generated recombinantly or synthetically, with a series of specified nucleic acid elements that permit transcription of a particular nucleic acid in a host cell, such as a yeast cell. In some embodiments, the nucleic acid sequence of a gene described in this application is inserted into a cloning vector such that it is operably joined to regulatory sequences and, in some embodiments, expressed as an RNA transcript. In some embodiments, the vector contains one or more markers, such as a selectable marker as described in this application, to identify cells transformed or transfected with the recombinant vector. In some embodiments, the nucleic acid sequence of a gene described in this application is codon-optimized. Codon optimization may increase production of the gene product by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100%, including all values in between) relative to a reference sequence that is not codon-optimized.

A coding sequence and a regulatory sequence are said to be “operably joined” or “operably linked” when the coding sequence and the regulatory sequence are covalently linked and the expression or transcription of the coding sequence is under the influence or control of the regulatory sequence. If the coding sequence is to be translated into a functional protein, the coding sequence and the regulatory sequence are said to be operably joined or linked if induction of a promoter in the 5′ regulatory sequence permits the coding sequence to be transcribed and if the nature of the linkage between the coding sequence and the regulatory sequence does not (1) result in the introduction of a frame-shift mutation, (2) interfere with the ability of the promoter region to direct the transcription of the coding sequence, or (3) interfere with the ability of the corresponding RNA transcript to be translated into a protein.

In some embodiments, the nucleic acid encoding any of the proteins described in this application is under the control of regulatory sequences (e.g., enhancer sequences). In some embodiments, a nucleic acid is expressed under the control of a promoter. The promoter can be a native promoter, e.g., the promoter of the gene in its endogenous context, which provides normal regulation of expression of the gene. Alternatively, a promoter can be a promoter that is different from the native promoter of the gene, e.g., the promoter is different from the promoter of the gene in its endogenous context.

In some embodiments, the promoter is a eukaryotic promoter. Non-limiting examples of eukaryotic promoters include TDH3, PGK1, PKC1, PDC1, TEF1, TEF2, RPL18B, SSA1, TDH2, PYK1, TPI1 GAL1, GAL10, GAL7, GAL3, GAL2, MET3, MET25, HXT3, HXT7, ACT1, ADH1, ADH2, CUP1-1, ENO2, and SOD1, as would be known to one of ordinary skill in the art (see, e.g., Addgene website: blog.addgene.org/plasmids-101-the-promoter-region). In some embodiments, the promoter is a prokaryotic promoter (e.g., bacteriophage or bacterial promoter). Non-limiting examples of bacteriophage promoters include Pls1con, T3, T7, SP6, and PL. Non-limiting examples of bacterial promoters include Pbad, PmgrB, Ptrc2, Plac/ara, Ptac, and Pm.

In some embodiments, the promoter is an inducible promoter. As used in this application, an “inducible promoter” is a promoter controlled by the presence or absence of a molecule. Non-limiting examples of inducible promoters include chemically-regulated promoters and physically-regulated promoters. For chemically-regulated promoters, the transcriptional activity can be regulated by one or more compounds, such as alcohol, tetracycline, galactose, a steroid, a metal, or other compounds. For physically-regulated promoters, transcriptional activity can be regulated by a phenomenon such as light or temperature. Non-limiting examples of tetracycline-regulated promoters include anhydrotetracycline (aTc)-responsive promoters and other tetracycline-responsive promoter systems (e.g., a tetracycline repressor protein (tetR), a tetracycline operator sequence (tetO) and a tetracycline transactivator fusion protein (tTA)). Non-limiting examples of steroid-regulated promoters include promoters based on the rat glucocorticoid receptor, human estrogen receptor, moth ecdysone receptors, and promoters from the steroid/retinoid/thyroid receptor superfamily. Non-limiting examples of metal-regulated promoters include promoters derived from metallothionein (proteins that bind and sequester metal ions) genes. Non-limiting examples of pathogenesis-regulated promoters include promoters induced by salicylic acid, ethylene or benzothiadiazole (BTH). Non-limiting examples of temperature/heat-inducible promoters include heat shock promoters. Non-limiting examples of light-regulated promoters include light responsive promoters from plant cells. In certain embodiments, the inducible promoter is a galactose-inducible promoter. In some embodiments, the inducible promoter is induced by one or more physiological conditions (e.g., pH, temperature, radiation, osmotic pressure, saline gradients, cell surface binding, or concentration of one or more extrinsic or intrinsic inducing agents). Non-limiting examples of an extrinsic inducer or inducing agent include amino acids and amino acid analogs, saccharides and polysaccharides, nucleic acids, protein transcriptional activators and repressors, cytokines, toxins, petroleum-based compounds, metal containing compounds, salts, ions, enzyme substrate analogs, hormones or any combination thereof.

In some embodiments, the promoter is a constitutive promoter. As used in this application, a “constitutive promoter” refers to an unregulated promoter that allows continuous transcription of a gene. Non-limiting examples of a constitutive promoter include TDH3, PGK1, PKC1, PDC1, TEF1, TEF2, RPL18B, SSA1, TDH2, PYK1, TPI1, HXT3, HXT7, ACT1, ADH1, ADH2, ENO2, and SOD1.

Other inducible promoters or constitutive promoters known to one of ordinary skill in the art are also contemplated.

Regulatory sequences needed for gene expression may vary between species or cell types, but generally include, as necessary, 5′ non-transcribed and 5′ non-translated sequences involved with the initiation of transcription and translation respectively, such as a TATA box, capping sequence, CAAT sequence, and the like. In particular, such 5′ non-transcribed regulatory sequences will include a promoter region which includes a promoter sequence for transcriptional control of the operably joined gene. Regulatory sequences may also include enhancer sequences or upstream activator sequences. Vectors may include 5′ leader or signal sequences. The regulatory sequence may also include a terminator sequence. In some embodiments, a terminator sequence marks the end of a gene in DNA during transcription. The choice and design of one or more appropriate vectors suitable for inducing expression of one or more genes described in this application in a host cell is within the ability and discretion of one of ordinary skill in the art.

Expression vectors containing the necessary elements for expression are commercially available and known to one of ordinary skill in the art (see, e.g., Sambrook et al., Molecular Cloning: A Laboratory Manual, Fourth Edition, Cold Spring Harbor Laboratory Press, 2012).

In some embodiments, introduction of a polynucleotide, such as a polynucleotide encoding a recombinant polypeptide, into a host cell results in genomic integration of the polynucleotide. In some embodiments, a host cell comprises at least 1 copy, at least 2 copies, at least 3 copies, at least 4 copies, at least 5 copies, at least 6 copies, at least 7 copies, at least 8 copies, at least 9 copies, at least 10 copies, at least 11 copies, at least 12 copies, at least 13 copies, at least 14 copies, at least 15 copies, at least 16 copies, at least 17 copies, at least 18 copies, at least 19 copies, at least 20 copies, at least 21 copies, at least 22 copies, at least 23 copies, at least 24 copies, at least 25 copies, at least 26 copies, at least 27 copies, at least 28 copies, at least 29 copies, at least 30 copies, at least 31 copies, at least 32 copies, at least 33 copies, at least 34 copies, at least 35 copies, at least 36 copies, at least 37 copies, at least 38 copies, at least 39 copies, at least 40 copies, at least 41 copies, at least 42 copies, at least 43 copies, at least 44 copies, at least 45 copies, at least 46 copies, at least 47 copies, at least 48 copies, at least 49 copies, at least 50 copies, at least 60 copies, at least 70 copies, at least 80 copies, at least 90 copies, at least 100 copies, or more, including any values in between, of a polynucleotide sequence, such as a polynucleotide sequence encoding any of the recombinant polypeptides described in this application, in its genome.

### Host Cells

Any of the proteins of the disclosure may be expressed in a host cell. As used in this application, the term “host cell” refers to a cell that can be used to express a polynucleotide, such as a polynucleotide that encodes a protein used in production of mogrol, mogrosides, and precursors thereof.

Any suitable host cell may be used to produce any of the recombinant polypeptides, including lanosterol synthases, acetoacetyl COA synthases, CB5, CDSs, UGTs, C11 hydroxylases, cytochrome P450 reductases, EPHs, SQEs, and other proteins disclosed in this application, including eukaryotic cells or prokaryotic cells. Suitable host cells include, but are not limited to, fungal cells (e.g., yeast cells), bacterial cells (e.g., E. coli cells), algal cells, plant cells, insect cells, and animal cells, including mammalian cells.

Suitable yeast host cells include, but are not limited to: Candida, Hansenula, Saccharomyces (e.g., S. cerevisiae), Schizosaccharomyces, Pichia, Kluyveromyces, and Yarrowia (e.g., Y. lipolytica). In some embodiments, the yeast cell is Hansenula polymorpha, Saccharomyces cerevisiae, Saccaromyces carlsbergensis, Saccharomyces diastaticus, Saccharomyces norbensis, Saccharomyces kluyveri, Schizosaccharomyces pombe, Pichia finlandica, Pichia trehalophila, Pichia kodamae, Pichia membranaefaciens, Pichia opuntiae, Pichia pastoris, Pichia pseudopastoris, Pichia membranifaciens, Komagataella pseudopastoris, Komagataella pastoris, Komagataella kurtzmanii, Komagataella mondaviorum, Pichia thermotolerans, Pichia salictaria, Pichia quercuum, Pichia pijperi, Pichia stipitis, Pichia methanolica, Pichia angusta, Komagataella phaffii, Komagataella pastoris, Kluyveromyces lactis, Candida albicans, Candida boidinii or Yarrowia lipolytica. In some embodiments, the yeast strain is an industrial polyploid yeast strain. Other non-limiting examples of fungal cells include cells obtained from Aspergillus spp., Penicillium spp., Fusarium spp., Rhizopus spp., Acremonium spp., Neurospora spp., Sordaria spp., Magnaporthe spp., Allomyces spp., Ustilago spp., Botrytis spp., and Trichoderma spp.

In certain embodiments, the host cell is an algal cell such as, Chlamydomonas (e.g., C. Reinhardtii) and Phormidium (P. sp. ATCC29409).

In other embodiments, the host cell is a prokaryotic cell. Suitable prokaryotic cells include gram positive, gram negative, and gram-variable bacterial cells. The host cell may be a species of, but not limited to: Agrobacterium, Alicyclobacillus, Anabaena, Anacystis, Acinetobacter, Acidothermus, Arthrobacter, Azobacter, Bacillus, Bifidobacterium, Brevibacterium, Butyrivibrio, Buchnera, Campestris, Campylobacter, Clostridium, Corynebacterium, Chromatium, Coprococcus, Escherichia, Enterococcus, Enterobacter, Erwinia, Fusobacterium, Faecalibacterium, Francisella, Flavobacterium, Geobacillus, Haemophilus, Helicobacter, Klebsiella, Lactobacillus, Lactococcus, Ilyobacter, Micrococcus, Microbacterium, Mesorhizobium, Methylobacterium, Methylobacterium, Mycobacterium, Neisseria, Pantoea, Pseudomonas, Prochlorococcus, Rhodobacter, Rhodopseudomonas, Rhodopseudomonas, Roseburia, Rhodospirillum, Rhodococcus, Scenedesmus, Streptomyces, Streptococcus, Synecoccus, Saccharomonospora, Saccharopolyspora, Staphylococcus, Serratia, Salmonella, Shigella, Thermoanaerobacterium, Tropheryma, Tularensis, Temecula, Thermosynechococcus, Thermococcus, Ureaplasma, Xanthomonas, Xylella, Yersinia, and Zymomonas.

In some embodiments, the bacterial host cell is of the Agrobacterium species (e.g., A. radiobacter, A. rhizogenes, A. rubi), the Arthrobacter species (e.g., A. aurescens, A. citreus, A. globformis, A. hydrocarboglutamicus, A. mysorens, A. nicotianae, A. paraffineus, A. protophonniae, A. roseoparaffinus, A. sulfureus, A. ureafaciens), or the Bacillus species (e.g., B. thuringiensis, B. anthracis, B. megaterium, B. subtilis, B. lentus, B. circulans, B. pumilus, B. lautus, B. coagulans, B. brevis, B. firmus, B. alkaophius, B. licheniformis, B. clausii, B. stearothermophilus, B. halodurans and B. amyloliquefaciens. In particular embodiments, the host cell is an industrial Bacillus strain including but not limited to B. subtilis, B. pumilus, B. licheniformis, B. megaterium, B. clausii, B. stearothermophilus and B. amyloliquefaciens. In some embodiments, the host cell is an industrial Clostridium species (e.g., C. acetobutylicum, C. tetani E88, C. lituseburense, C. saccharobutylicum, C. perfringens, C. beijerinckii). In some embodiments, the host cell is an industrial Corynebacterium species (e.g., C. glutamicum, C. acetoacidophilum). In some embodiments, the host cell is an industrial Escherichia species (e.g., E. coli). In some embodiments, the host cell is an industrial Erwinia species (e.g., E. uredovora, E. carotovora, E. ananas, E. herbicola, E. punctata, E. terreus). In some embodiments, the host cell is an industrial Pantoea species (e.g., P. citrea, P. agglomerans). In some embodiments, the host cell is an industrial Pseudomonas species, (e.g., P. putida, P. aeruginosa, P. mevalonii). In some embodiments, the host cell is an industrial Streptococcus species (e.g., S. equisimiles, S. pyogenes, S. uberis). In some embodiments, the host cell is an industrial Streptomyces species (e.g., S. ambofaciens, S. achromogenes, S. avermitilis, S. coelicolor, S. aureofaciens, S. aureus, S. fungicidicus, S. griseus, S. lividans). In some embodiments, the host cell is an industrial Zymomonas species (e.g., Z. mobilis, Z. lipolytica).

The present disclosure is also suitable for use with a variety of animal cell types, including mammalian cells, for example, human (including 293, HeLa, WI38, PER.C6 and Bowes melanoma cells), mouse (including 3T3, NS0, NS1, Sp2/0), hamster (CHO, BHK), monkey (COS, FRhL, Vero), and hybridoma cell lines.

The present disclosure is also suitable for use with a variety of plant cell types.

The term “cell,” as used in this application, may refer to a single cell or a population of cells, such as a population of cells belonging to the same cell line or strain. Use of the singular term “cell” should not be construed to refer explicitly to a single cell rather than a population of cells.

The host cell may comprise genetic modifications relative to a wild-type counterpart. As a non-limiting example, a host cell (e.g., S. cerevisiae or Y. lipolytica) may be modified to reduce or inactivate one or more of the following genes: hydroxymethylglutaryl-CoA (HMG-CoA) reductase (HMG1), acetyl-CoA C-acetyltransferase (acetoacetyl-CoA thiolase) (ERG10), 3-hydroxy-3-methylglutaryl-CoA (HMG-COA) synthase (ERG13), farnesyl-diphosphate farnesyl transferase (squalene synthase) (ERG9), may be modified to overexpress squalene epoxidase, or may be modified to downregulate lanosterol synthase. In some embodiments, the squalene epoxidase is encoded by an ERG1 gene. In some embodiments, the lanosterol synthase is encoded by an ERG7 gene. In some embodiments, a host cell (e.g., S. cerevisiae) may be modified to reduce or inactivate one or more of the following genes: hydroxymethylglutaryl-CoA (HMG-COA) reductase (HMG1), acetyl-CoA C-acetyltransferase (acetoacetyl-CoA thiolase), 3-hydroxy-3-methylglutaryl-CoA (HMG-CoA) synthase, farnesyl-diphosphate farnesyl transferase (squalene synthase), squalene epoxidase, or lanosterol synthase. In some embodiments, a host cell may be modified to reduce or inactivate the activity of a lanosterol synthase or squalene epoxidase. In some embodiments, a host cell is modified to reduce or eliminate expression of one or more transporter genes, such as PDR1 or PDR3, and/or the glucanase gene EXG1.

Reduced enzyme activity can mean decreased enzyme expression, decreased enzyme stability, decreased enzyme specific activity, and/or a decrease in enzyme function due to interference by another protein, a nucleic acid or a small molecule inhibitor as known in the art.

In some embodiments, a host cell is modified to reduce or inactivate at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 genes.

In some embodiments, a host cell is modified to reduce or inactivate 1, 2, 3, 4, 5, 6, 7, 8, 9, 10, 11, 12, 13, 14, 15, 16, 17, 18, 19, or 20 genes.

Reduction of gene expression and/or gene inactivation may be achieved through any suitable method, including but not limited to deletion of the gene, introduction of a point mutation into the gene, truncation of the gene, introduction of an insertion into the gene, introduction of a tag or fusion into the gene, or selective editing of the gene. For example, polymerase chain reaction (PCR)-based methods may be used (see, e.g., Gardner et al., Methods Mol Biol. 2014; 1205:45-78) or well-known gene-editing techniques may be used. As a non-limiting example, genes may be deleted through gene replacement (e.g., with a marker, including a selection marker). A gene may also be truncated through the use of a transposon system (see, e.g., Poussu et al., Nucleic Acids Res. 2005; 33(12): e104).

A vector encoding any of the recombinant polypeptides described in this application may be introduced into a suitable host cell using any method known in the art. Non-limiting examples of yeast transformation protocols are described in Gietz et al., Yeast transformation can be conducted by the LiAc/SS Carrier DNA/PEG method. Methods Mol Biol. 2006; 313:107-20, which is incorporated by reference in its entirety. Host cells may be cultured under any suitable conditions as would be understood by one of ordinary skill in the art. For example, any media, temperature, and incubation conditions known in the art may be used. For host cells carrying an inducible vector, cells may be cultured with an appropriate inducible agent to promote expression.

Aspects of the present disclosure provide a host cell comprising a mevalonate pathway (or a portion thereof), wherein the expression, level and/or activity of a lanosterol synthase (which converts 2-3-oxido-squalene to lanosterol) is decreased but not abolished. In some embodiments, the activity of a lanosterol synthase is decreased, but not abolished, using any mutation(s) or combination of mutations thereof described herein. In some embodiments, the decrease in lanosterol synthase expression, level, or activity decreases the amount of 2-3-oxido-squalene being converted into lanosterol, and increases the amount of 2-3-oxido-squalene available to be shunted into another pathway and being converted, via one or more enzymatic steps, into one or more compounds of interest, which are therefore produced at a higher level in the cell. In some embodiments, a compound of interest is a mogrol precursor, mogrol, and/or mogroside).

In some embodiments, the host cell further comprises a heterologous nucleic acid encoding an acetoacetyl COA synthase (e.g., an acetoacetyl COA synthase comprising the amino acid sequence provided in SEQ ID NO: 6 and/or encoded by a polynucleotide comprising the sequence provided in SEQ ID NO: 7), which increases synthesis of acetoacetyl-CoA, which is a precursor to 2-3-oxido-squalene.

In some embodiments, the expression, level and/or activity of an enzyme involved in production of the compound of interest is increased; in various embodiments, the enzyme involved in production of the compound of interest is any of: a UDP-glycosyltransferases (UGT) enzyme (e.g., a primary or secondary UGT), a cucurbitadienol synthase (CDS) enzyme, a C11 hydroxylase, an epoxide hydrolase (EPH), and squalene epoxidase (SQE).

In some embodiments, wherein 11-oxo mogrol is not a desired product, the level, expression and/or activity of a cytochrome P450 reductase, which is involved in synthesis of 11-oxo mogrol, is decreased.

In some embodiments, mogrol precursors include but are not limited to: 2,3,22,23-dioxidosqualene, cucurbitadienol, 24, 25-expoxycucurbitadienol, 11-hydroxycucurbitadienol, 11-hydroxy-24,25-epoxycucurbitadienol, 11-hydroxy-cucurbitadienol, 11-oxo-cucurbitadienol, and 24,25-dihydroxycucurbitadienol.

In some embodiments, mogrosides include, but are not limited to: mogroside I-A1 (MIA1), mogroside IE (MIE or MIE), mogroside II-A1 (MIIA1 or M2A1), mogroside II-A2 (MIIA2 or M2A2), mogroside III-A1 (MIIIA1 or M3A1), mogroside II-E (MIIE or M2E), mogroside III (MIII or M3), siamenoside I, mogroside IV (MIV or M4), mogroside IVa (MIVA or M4A), isomogroside IV, mogroside III-E (MIIIE or M3E), mogroside V (MV or M5), mogroside VIA (MVIA), mogroside VIB (MVIB), isomogroside V, mogroside VIa1 (MVIa1), and mogroside VI (MVI or M6). In some embodiments, the mogroside is siamenoside I, which may be referred to as siamenoside or Siam. In some embodiments, the mogroside is MIIIE.

Any of the cells disclosed in this application can be cultured in media of any type (rich or minimal) and any composition prior to, during, and/or after contact and/or integration of a nucleic acid. The conditions of the culture or culturing process can be optimized through routine experimentation as would be understood by one of ordinary skill in the art. In some embodiments, the selected media is supplemented with various components. In some embodiments, the concentration and amount of a supplemental component is optimized. In some embodiments, other aspects of the media and growth conditions (e.g., pH, temperature, etc.) are optimized through routine experimentation. In some embodiments, the frequency that the media is supplemented with one or more supplemental components, and the amount of time that the cell is cultured, is optimized.

Culturing of the cells described in this application can be performed in culture vessels known and used in the art. In some embodiments, an aerated reaction vessel (e.g., a stirred tank reactor) is used to culture the cells. In some embodiments, a bioreactor or fermenter is used to culture the cell. Thus, in some embodiments, the cells are used in fermentation. As used in this application, the terms “bioreactor” and “fermenter” are interchangeably used and refer to an enclosure, or partial enclosure, in which a biological, biochemical and/or chemical reaction takes place, involving a living organism, part of a living organism, or purified proteins. A “large-scale bioreactor” or “industrial-scale bioreactor” is a bioreactor that is used to generate a product on a commercial or quasi-commercial scale. Large scale bioreactors typically have volumes in the range of liters, hundreds of liters, thousands of liters, or more.

Non-limiting examples of bioreactors include: stirred tank fermenters, bioreactors agitated by rotating mixing devices, chemostats, bioreactors agitated by shaking devices, airlift fermenters, packed-bed reactors, fixed-bed reactors, fluidized bed bioreactors, bioreactors employing wave induced agitation, centrifugal bioreactors, roller bottles, and hollow fiber bioreactors, roller apparatuses (for example benchtop, cart-mounted, and/or automated varieties), vertically-stacked plates, spinner flasks, stirring or rocking flasks, shaken multi-well plates, MD bottles, T-flasks, Roux bottles, multiple-surface tissue culture propagators, modified fermenters, and coated beads (e.g., beads coated with serum proteins, nitrocellulose, or carboxymethyl cellulose to prevent cell attachment).

In some embodiments, the bioreactor includes a cell culture system where the cell (e.g., yeast cell) is in contact with moving liquids and/or gas bubbles. In some embodiments, the cell or cell culture is grown in suspension. In other embodiments, the cell or cell culture is attached to a solid phase carrier. Non-limiting examples of a carrier system includes microcarriers (e.g., polymer spheres, microbeads, and microdisks that can be porous or nonporous), cross-linked beads (e.g., dextran) charged with specific chemical groups (e.g., tertiary amine groups), 2D microcarriers including cells trapped in nonporous polymer fibers, 3D carriers (e.g., carrier fibers, hollow fibers, multicartridge reactors, and semi-permeable membranes that can comprising porous fibers), microcarriers having reduced ion exchange capacity, encapsulation cells, capillaries, and aggregates. In some embodiments, carriers are fabricated from materials such as dextran, gelatin, glass, or cellulose.

In some embodiments, industrial-scale processes are operated in continuous, semi-continuous or non-continuous modes. Non-limiting examples of operation modes are batch, fed batch, extended batch, repetitive batch, draw/fill, rotating-wall, spinning flask, and/or perfusion mode of operation. In some embodiments, a bioreactor allows continuous or semi-continuous replenishment of the substrate stock, for example a carbohydrate source and/or continuous or semi-continuous separation of the product, from the bioreactor.

In some embodiments, the bioreactor or fermenter includes a sensor and/or a control system to measure and/or adjust reaction parameters. Non-limiting examples of reaction parameters include biological parameters (e.g., growth rate, cell size, cell number, cell density, cell type, or cell state, etc.), chemical parameters (e.g., pH, redox-potential, concentration of reaction substrate and/or product, concentration of dissolved gases, such as oxygen concentration and CO₂ concentration, nutrient concentrations, metabolite concentrations, concentration of an oligopeptide, concentration of an amino acid, concentration of a vitamin, concentration of a hormone, concentration of an additive, serum concentration, ionic strength, concentration of an ion, relative humidity, molarity, osmolarity, concentration of other chemicals, for example buffering agents, adjuvants, or reaction by-products), physical/mechanical parameters (e.g., density, conductivity, degree of agitation, pressure, and flow rate, shear stress, shear rate, viscosity, color, turbidity, light absorption, mixing rate, conversion rate, as well as thermodynamic parameters, such as temperature, light intensity/quality, etc.). Sensors to measure the parameters described in this application are well known to one of ordinary skill in the relevant mechanical and electronic arts. Control systems to adjust the parameters in a bioreactor based on the inputs from a sensor described in this application are well known to one of ordinary skill in the art in bioreactor engineering.

In some embodiments, the method involves batch fermentation (e.g., shake flask fermentation). General considerations for batch fermentation (e.g., shake flask fermentation) include the level of oxygen and glucose. For example, batch fermentation (e.g., shake flask fermentation) may be oxygen and glucose limited, so in some embodiments, the capability of a strain to perform in a well-designed fed-batch fermentation is underestimated. Also, the final product (e.g., mogrol precursor, mogrol, mogroside precursor, or mogroside) may display some differences from the substrate (e.g., mogrol precursor, mogrol, mogroside precursor, or mogroside) in terms of solubility, toxicity, cellular accumulation and secretion and in some embodiments can have different fermentation kinetics.

Aspects of the present disclosure provide methods of increasing production of a compound of interest, e.g., a mogrol precursor, mogrol, and/or mogroside in a host cell by decreasing but not abolishing lanosterol synthase activity by introducing one or more mutation(s) described herein into lanosterol synthase. In some embodiments, the methods further comprise increasing the expression, level and/or activity of an enzyme involved in synthesis of the compound of interest, e.g., a UDP-glycosyltransferases (UGT) enzyme, a cucurbitadienol synthase (CDS) enzyme, a C11 hydroxylase, an epoxide hydrolase (EPH), and/or a squalene epoxidase (SQE). In some embodiments of the method, wherein 11-oxo mogrol is not a desired product, the level, expression and/or activity of a cytochrome P450 reductase is decreased. In some embodiments of the method, the host cell further comprises a heterologous polynucleotide encoding an acetoacetyl CoA synthase.

The methods described in this application encompass production of the mogrol precursors (e.g., squalene, 2,3-oxidosqualene, or 24-25 epoxy-cucurbitadienol), mogrol, or mogrosides (e.g., MIA1, MIE1, MIIA1, MIIA2, MIIIA1, MIIE, MIII, siamenoside I, mogroside IV, isomogroside IV, MIIIE, MVIA, MVIB, isomogroside V, MVIa1, and mogroside V) using a recombinant cell, cell lysate or isolated recombinant polypeptides (e.g., lanosterol synthase, acetoacetyl CoA synthase, CB5, CDS, UGT, C11 hydroxylase, cytochrome P450 reductase, EPH, squalene epoxidase, and any proteins associated with the disclosure).

Mogrol precursors (e.g., squalene, 2,3-oxidosqualene, or 24-25 epoxy-cucurbitadienol), mogrol, mogrosides (e.g., MIA1, MIE, MIIA1, MIIA2, MIIIA1, MIIE, MIII, siamenoside I, mogroside IV, isomogroside IV, MIIIE, MVIA, MVIB, isomogroside V, MVIa1, and mogroside V) produced by any of the recombinant cells disclosed in this application may be identified and extracted using any method known in the art. Mass spectrometry (e.g., LC-MS, GC-MS) is a non-limiting example of a method for identification and may be used to help extract a compound of interest.

The phraseology and terminology used in this application is for the purpose of description and should not be regarded as limiting. The use of terms such as “including,” “comprising,” “having,” “containing,” “involving,” and/or variations thereof in this application, is meant to encompass the items listed thereafter and equivalents thereof as well as additional items.

The present invention is further illustrated by the following Examples, which in no way should be construed as further limiting. The entire contents of all of the references (including literature references, issued patents, published patent applications, and co pending patent applications) cited throughout this application are hereby expressly incorporated by reference.

### EXAMPLES

### Example 1. Identification of Lanosterol Synthases with Reduced Activity

This Example describes identification of lanosterol synthases with reduced activity. Mutagenic PCR was performed on an ERG7 template, and the PCR mixture was cleaved with BsaI and ligated to pERG7.NatR cleaved with HindIII and NcoI, to create a library of mutants, ranging from low (2-4 mutations per gene), to medium (6-9 mutations per gene), to high (12-20 mutations per gene). Cleavage of these plasmids with PacI and SspI and introduction into a Yarrowia strain (genotype pTEF-HMGt erg7Δ13 [GPR1-1 ERG7 HygR]) yielded plates (grown at 22° C. or 30° C.) of nourseothricin resistant (NatR) transformants that were replica-plated to YNBAc (YNB+30 mM glacial acetic acid) at the appropriate temperature. 372 acetate resistant (AcR) clones were identified and picked to YPD medium, grown at the appropriate temperature, and subsequently inoculated to YPD4 medium, grown for three days at 30° C. and the supernatants assayed for mevalonic acid by LC-RIA. AcR cells are able to grow on media containing acetic acid. At the same time, the clones propagated originally at 22° C. were tested for temperature sensitive growth at 32° C., while those grown at 30° C. were tested for cold sensitivity at 18° C.

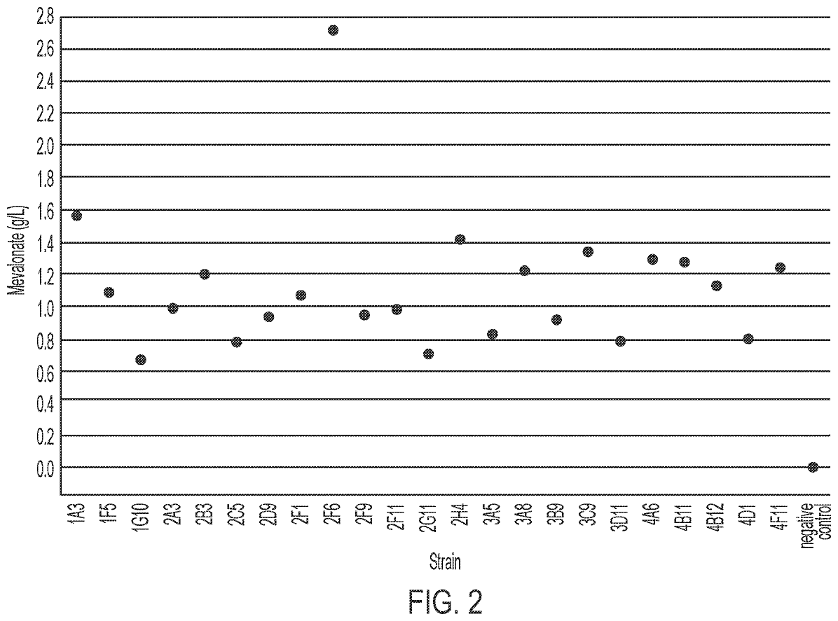

As shown in Table 3 and FIG. 2, nine temperature sensitive (T.s.) and three partially cold sensitive (C.s.) clones were identified that increased mevalonate titer relative to the parent. These strains were 1A3, 2F9, 2F6, 2C5, 2B3, 2A5, 2F1, 3B9, and 3D11. Of the strains tested, 2F6, which harbors the lanosterol synthase set forth in SEQ ID NO: 3, showed the highest mevalonate titer. 4A6 and 4F11 have the same mutations. The strains not labeled as T.s. or C.s. are neither temperature- nor cold-sensitive.

TABLE 3
Lanosterol Synthase Activity as Determined
by Mevalonate Titer in Yarrowia host cells*

| | | Protein | | Type |
|---|---|---|---|---|
| | Mutation(s) relative | SEQ | Mevalonate | of |
| Strain | to SEQ ID NO: 1 | ID NO: | titer (g/L) | Mutant |
| Parent | none | 1 | 0.0 ± 0.0 | |
| 1A3 | R33Q, R193C, D289G, | 331 | 1.6 | T.s. |
| | N295I, S296T, N620S, | | | |
| | and Y736F | | | |
| 1F5 | unknown | | 1.1 | |
| 1G10 | unknown | | 0.7 | |
| 2G11 | unknown | | 0.7 | |
| 2F11 | R184W, L235M, L260R, | 119 | 1 | |
| | and E710Q | | | |
| 2F9 | K47E, L92I, T360S, | 325 | 1 | T.s. |
| | S372P, T444M, and R578P | | | |
| 2D9 | unknown | | 0.9 | |
| 2F6 | D50G, K66R, N94S, G417S, | 3 | 2.7 | T.s. |
| | E617V, and F726L | | | |
| 2C5 | N14Y, N132S, Y145C, | 329 | 0.8 | T.s. |
| | R193H, I286F, L316R, | | | |
| | F432I, E442V, T444S, | | | |
| | I479S, K631R, and T655A | | | |
| 2H4 | F432S, D452G, and I536F | 85 | 1.4 | |
| 2B3 | E287G, K329N, E617V, and | 324 | 1.2 | T.s |
| | F726V | | | |
| 2A5 | E231V, A407V, Q423L, | 323 | 1 | T.s. |
| | A529T, and Y564C | | | |
| 2F1 | V248F, D371V, and G702D | 118 | 1.1 | T.s. |
| 3A5 | L197V, K282I, N314S, | 326 | 0.8 | |
| | P370L, A608T, G638D, and | | | |
| | F650L | | | |
| 3A8 | L491Q, Y586F, and R660H | 120 | 1.2 | |
| 3B9 | G122C, H249L, and K738M | 316 | 0.9 | C.s. |
| 3C9 | P227L, E474V, V559A, and | 318 | 1.3 | |
| | Y564N | | | |
| 3D11 | K85N, G158S, S515L, | 321 | 0.8 | C.s |
| | P526T, Q619L, and | | | |
| | Q742* | | | |
| 4D1 | unknown | | 0.8 | |
| 4A6 | G107D and K631E | 319 | 1.3 | |
| 4B11 | T212I, W213L, N544Y, and | 322 | 1.3 | |
| | V552E | | | |
| 4F11 | G107D and K631E | 319 | 1.3 | |
| 4B12 | I172N, C414S, L560M, and | 84 | 1.1 | |
| | G679S | | | |

*indicates a truncation

Many of the mevalonate-excreting ERG7 alleles also significantly perturbed the steady state levels of other metabolites; 2F6 in particular decreased squalene, and increased oxidosqualene, dioxidosqualene, and ergosterol.

### Example 2. Characterization of an Acetoacetyl COA Synthase that Increases Squalene Production in Yarrowia Host Cells

This Example describes characterization of the effect of an acetoacetyl COA synthase on squalene production in a host cell. An acetoacetyl COA synthase comprising SEQ ID NO: 6 and encoded by SEQ ID NO: 7 was constructed. Various constructs were constructed, each expressing the acetoacetyl COA synthase under the control of a different promoter. The constructs were then randomly inserted into a Yarrowia host cell strain that produced about 17.2 mg/L squalene. As shown in Table 4, the acetoacetyl CoA synthase (represented by SEQ ID NO: 6 and 7) increased squalene titers to about 23.8-33 mg/L.

TABLE 4
Expression of an Acetoacetyl COA Synthase (SEQ ID NO:
6) Under the Control of Various Promoters in Yarrowia

| | | Average | |
|---|---|---|---|
| | Squalene | Squalene | |
| | [mg/L] | [mg/L] | promoter expressing NphT7 |
| Control | 18.9 | 17.2 | — |
| Control | 15.1 | | |
| Control | 17.6 | | |
| NphT7-A | 32.4 | 30.1 | tef Yarrowia alimentaria |
| NphT7-A | 28.9 | | (YAALOS06) |
| NphT7-A | 29.1 | | |
| NphT7-B | 27.5 | 28.6 | act1p (YB392) |
| NphT7-B | 28.7 | | |
| NphT7-B | 29.7 | | |
| NphT7-D | 20.9 | 27.8 | pMDH1 (YB392) |
| NphT7-D | 33.5 | | |
| NphT7-D | 29.1 | | |
| NphT7-F | 31.4 | 32.7 | gapDH Y. porcina (YAPO0S01) |
| NphT7-F | 32.3 | | |
| NphT7-F | 34.3 | | |
| NphT7-G | 35.9 | 33.0 | tef Y. deformans (YADE0S01) |
| NphT7-G | 30.0 | | |
| NphT7-G | 33.1 | | |
| NphT7-H | 23.5 | 24.0 | gapDH C. osloensis (YAOS0S01) |
| NphT7-H | 23.2 | | |
| NphT7-H | 25.4 | | |
| NphT7-I | 19.8 | 23.8 | tef Y. sp. (JCM 30694) |
| NphT7-I | 25.2 | | |
| NphT7-I | 26.6 | | |

Several of the nphT7 cassettes also induced very high mevalonate secretion, up to 5 g/L. which represents a significant fraction of the theoretical yield.

### Example 3. Production of Cucurbitadienol in ERG7 Mutant Host Cells

This Example describes characterization of cucurbitadienol synthases (CDSs) in different Yarrowia host cells comprising mutants of SEQ ID NO: 1.

Acetate resistant (AcR) cells were generated as in Example 1 using pERG7-NatR plasmids that resulted in clones with high mevalonate titers. AcR cells are able to grow on media containing acetic acid. Constructs encoding a particular CDS were inserted randomly into these cells. All strains except for strains 887779 and 870688 express AquAgaCDS16 (SEQ ID NOs: 226 and 327). Strains 887779 and 870688 express SgCDS1 (SEQ ID NOS: 256 and 332). Strains 950910 and 950917 also express NphT7 (SEQ ID NO: 6). The resulting nourseothricin resistant (NatR) isolates were picked and grown in 96-deepwell plates in 0.5 mL YPD medium for two days at 30° C., subcultured into 0.5 mL YPD10 medium for 4 days at 30° C. and then the cultures were assayed for cucurbitadienol by GC-MS. Nourseothricin resistance allows for the selection of cells comprising a heterologous nucleic acid encoding a CDS. Strain 870688 comprising SEQ ID NO: 1 was used as a control.

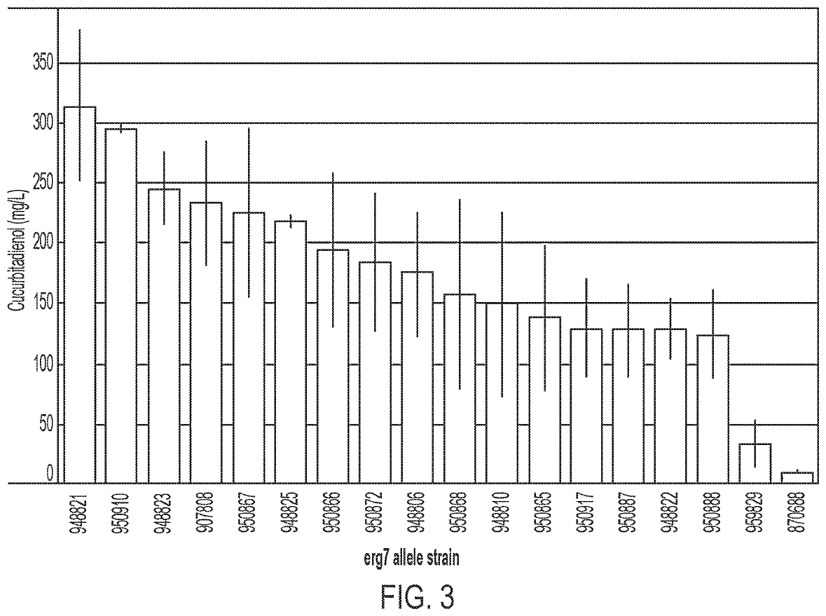

As shown in Table 5 and FIG. 3, cucurbitadienol titers of Yarrowia strains comprising a mutant lanosterol synthase are significantly greater than the strain comprising SEQ ID NO: 1.

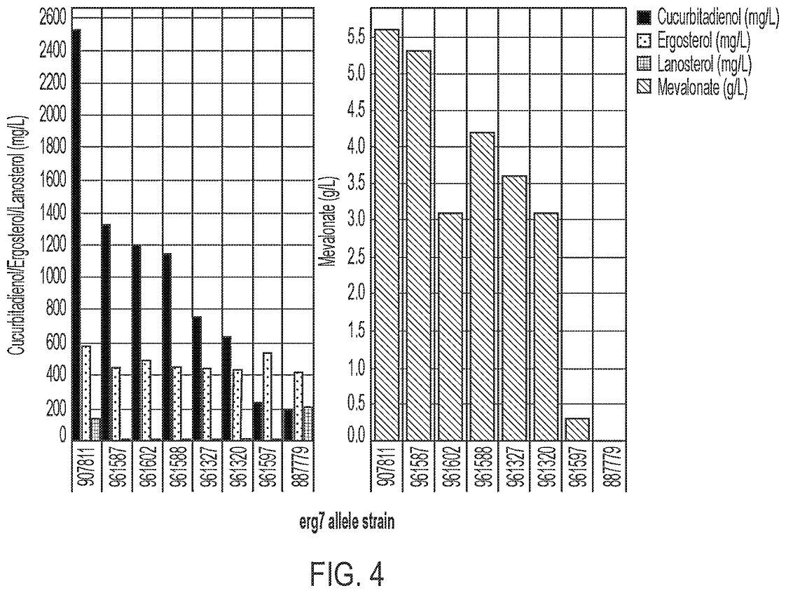

A selection of strains was then run in ambr 250 bioreactors, where cucurbitadienol, ergosterol and lanosterol were assayed by GC-MS and mevalonate by HPLC. Strain 887779 comprising SEQ ID NO: 1 was used as a control. As shown in FIG. 4 and Tables 6A-6B, Yarrowia strains with mutant lanosterol synthase alleles accumulate less lanosterol and more mevalonate and cucurbitadienol relative to a strain comprising the wild-type lanosterol synthase comprising SEQ ID NO: 1.

TABLE 5
Effects of Lanosterol Synthase Mutations on Cucurbitadienol Production in Yarrowia

| | | | | Average Fold |
|---|---|---|---|---|
| | | | | Cucurbitadienol |
| | | | Average | Titer Increase |
| Yarrowia | Lanosterol synthase mutations | Protein | Cucurbitadienol | Relative to |
| Strain | relative to SEQ ID NO: 1 | SEQ ID NO | Titer (mg/L) | Strain 870688 |
| 948821 | K85N and G158S | 86 | 314.7 | 35.6 |
| 950910 | I172N, C414S, L560M, and | 84 | 295.4 | 33.4 |
| | G679S | | | |
| 948823 | I172N, C414S, L560M, and | 84 | 245 | 27.7 |
| | G679S | | | |
| 907808 | R193C, D289G, N295I, | 83 | 233.7 | 26.4 |
| | S296T, N620S, and Y736F | | | |
| 950867 | D80G, P83L, T170A, T198I, | 92 | 225.4 | 25.5 |
| | and A228T | | | |
| 948825 | I172N, C414S, and L560M | 89 | 218.3 | 24.7 |
| 950866 | D371V, K498N, M610I, and | 91 | 194 | 21.9 |
| | G702D | | | |
| 950872 | T360S, S372P, T444M, and | 94 | 184.8 | 20.9 |
| | R578P | | | |
| 948806 | I172N, C414S, L560M, and | 84 | 175 | 19.8 |
| | G679S | | | |
| 950868 | D50G, K66R, N94S, G417S, | 3 | 157.8 | 17.8 |
| | E617V, and F726L | | | |
| 948810 | F432S, D452G, and I536F | 85 | 149.4 | 16.9 |
| 950865 | D371V, M610I, and G702D | 90 | 137.7 | 15.6 |
| 950917 | D50G, K66R, N94S, G417S, | 95 | 129.3 | 14.6 |
| | and E617V | | | |
| 950887 | D50G, K66R, N94S, G417S, | 95 | 128.1 | 14.5 |
| | and E617V | | | |
| 948822 | L197V, K282I, N314S, and | 87 | 127.6 | 14.4 |
| | P370L | | | |
| 950888 | D80G, P83L, T170A, T198I, | 92 | 124.7 | 14.1 |
| | and A228T | | | |
| 959829 | L309F, V344A, T398I, and | 99 | 32.1 | 3.6 |
| | K686E | | | |
| 870688 | N/A (wild-type ERG7 (WT)) | 1 | 8.9 | 1 |

TABLE 6A
Effects of Lanosterol Synthase Mutations on Cucurbitadienol Production in Yarrowia

| | | | | Average Fold |
|---|---|---|---|---|
| | | | | Cucurbitadienol |
| | | | Average | Titer Increase |
| Yarrowia | Lanosterol synthase mutations | Protein | Cucurbitadienol | Relative to |
| Strain | relative to SEQ ID NO: 1 | SEQ ID NO | Titer (mg/L) | Strain 870688 |
| 907811 | D50G, K66R, N94S, G417S, E617V, | 3 | 2522.1 | 13.3 |
| | and F726L | | | |
| 950865 | D371V, M610I, and G702D | 90 | 1327.7 | 7.0 |
| 950872 | T360S, S372P, T444M, and R578P | 94 | 1200.2 | 6.4 |
| 950866 | D371V, K498N, M610I, and G702D | 91 | 1143.8 | 6.1 |
| 948823 | I172N, C414S, L560M, and G679S | 84 | 764.5 | 4.0 |
| 948825 | I172N, C414S, and L560M | 89 | 638.5 | 3.4 |
| 950867 | D80G, P83L, T170A, T198I, and | 92 | 231.2 | 1.2 |
| | A228T | | | |
| 887779 | N/A (wild-type ERG7 (WT)) | 1 | 189.0 | 1.0 |

TABLE 6B
Effects of Lanosterol Synthase Mutations on Ergosterol,
Lanosterol, and Mevalonate Production in Yarrowia

| Yarrowia | Lanosterol synthase mutations | Protein | Ergosterol | Lanosterol | Mevalonate |
|---|---|---|---|---|---|
| Strain | relative to SEQ ID NO: 1 | SEQ ID NO | (mg/L) | (mg/L) | (g/L) |
| 907811 | D50G, K66R, N94S, G417S, E617V, | 3 | 580.6 | 137.4 | 5.57 |
| | and F726L | | | | |
| 950865 | D371V, M610I, and G702D | 90 | 452.2 | 8.2 | 5.29 |
| 950872 | T360S, S372P, T444M, and R578P | 94 | 496.5 | 8.1 | 3.08 |
| 950866 | D371V, K498N, M610I, and G702D | 91 | 455.7 | 10.8 | 4.18 |
| 948823 | I172N, C414S, L560M, and G679S | 84 | 443.5 | 11.9 | 3.6 |
| 948825 | I172N, C414S, and L560M | 89 | 436.6 | 11.1 | 3.09 |
| 950867 | D80G, P83L, T170A, T198I, and | 92 | 537.9 | 8.2 | 0.293 |
| | A228T | | | | |
| 887779 | N/A (wild-type ERG7 (WT)) | 1 | 422.0 | 207.9 | 0 |

### Example 4. Production of Oxidosqualene in Saccharomyces cerevisiae Host Cells with Mutants of SEQ ID NO: 313

This Example describes identification of lanosterol synthases with reduced activity using SEQ ID NO: 313 as a template for mutation.

Three different temperature sensitive lanosterol synthase mutants were tested and host cells comprising each of these lanosterol synthase mutants were analyzed for consumption of glucose and production of oxidosqualene, mevalonate, ergosterol, and ethanol. A parent strain with a native lanosterol synthase (SEQ ID NO: 313) was used as the negative control.

Strain 756247 expressed a lanosterol synthase comprising the protein sequence of SEQ ID NO: 100. The nucleotide sequence encoding SEQ ID NO: 100 comprises the following mutations relative to SEQ ID NO: 8 (mutations in SEQ ID NO: 100 relative to SEQ ID NO: 313 are shown in parenthesis): C361T (P121S), C407T (A136V), G474A (silent), A898G (S300G), A909G (silent), T965G (V322G), A1312G (K438E), T1506A (F502L), T1732C (silent), A1882G (K628E), and T2178G (Y726*—truncation mutation). A silent mutation results in no change in the amino acid sequence.

Strain 756248 expressed a lanosterol synthase comprising the protein sequence of SEQ ID NO: 101. The nucleotide sequence encoding SEQ ID NO: 101 comprises the following mutations relative to SEQ ID NO: 8 (mutations in SEQ ID NO: 101 relative to SEQ ID NO: 313 are shown in parenthesis): C333T (silent), A803G/A804T (K268S), A841G (T281A), T1504C (F502L), C1811A (T604N), G1966A (A656T), and A2078G (E693G).

Strain 756249 expressed a lanosterol synthase comprising the protein sequence of SEQ ID NO: 102. The nucleotide sequence encoding SEQ ID NO: 102 comprises the following mutations relative to SEQ ID NO: 8 (mutations in SEQ ID NO: 102 relative to SEQ ID NO: 313 are shown in parenthesis): A190G (R64G), A358G (I120V), G678T (M226I), T823A (F275I), A997G (T333A), and T1855A (C619S).

To measure 2-3-oxidosqualene production, strains were first grown overnight at 30° C., diluted to a starting OD of 0.2 and grown for an additional 16 h either at 30° C. or 35° C. in triplicates in 96-well deep well plates. Cell culture volumes were 500 μL and the media used in this experiment was YPD (10 g/L Yeast Extract, 20 g/L Peptone and 20 g/L Dextrose). 200 μL of the culture and 400 μL of ethyl acetate containing internal standards (100 μm tridecane and 100 mg/L pregnenolone) were transferred to a 96-well deep well plate containing 100 μL of silica/zirconia beads (0.5 mm dia., Cat.no. 11079105z Biospec) in each well. The plate containing the samples was heat sealed and agitated at 1750 rpm for 5 minutes using a Genogrinder. The plate was then centrifuged for 10 minutes at 4000 rpm at 4° C. to separate the aqueous and organic layers. The plate was then stored at −30° C. for 2 h to freeze the aqueous layer and 100 μL from the top layer was transferred to a glass vial analyzed by a GC-FID. A gas chromatograph (Thermo Scientific Trace 1310) with a TG-5MS column (15 m×0.25 mm×0.25 μm) was used at a flow rate of 1.5 mL/min. The eluents were determined by comparing peak retention times to those of known standard substances, and the amounts were quantified by comparing the peak area of the analyte to the peak area of the standard substance at known concentrations.

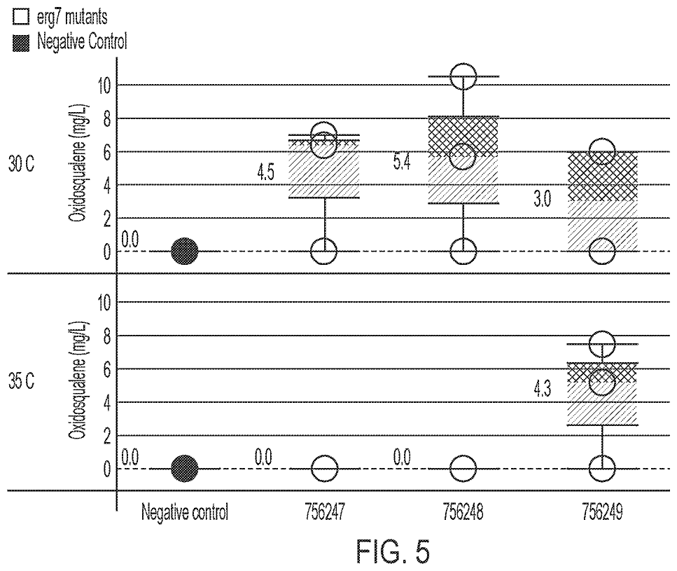

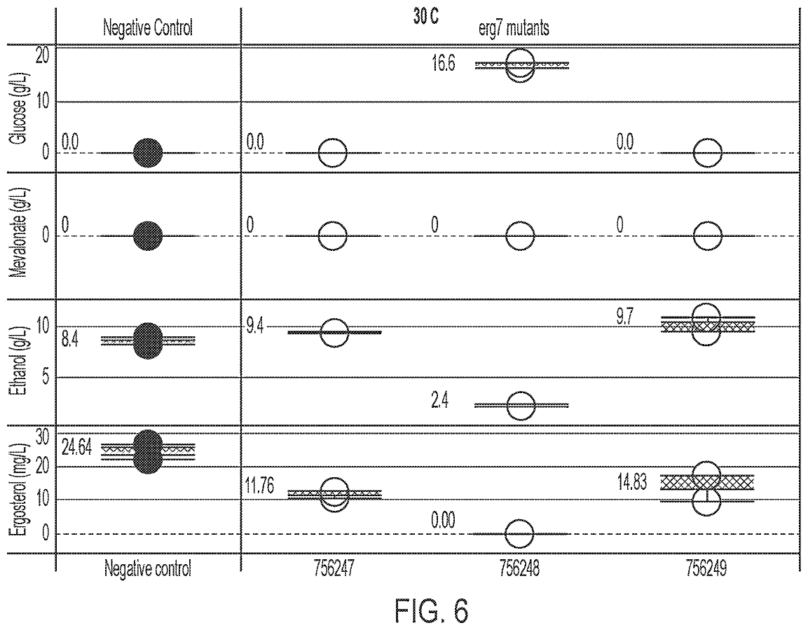

As shown in FIG. 6 and Table 7, at 30° C., Saccharomyces cerevisiae host cells comprising any one of SEQ ID NOs: 100-102 produced less ergosterol than the parent strain (the negative control), indicating that lanosterol synthases comprising any one of SEQ ID NOs: 100-102 were less active and had impaired lanosterol synthase activity compared to a wild-type lanosterol synthase comprising SEQ ID NO: 313 at this temperature. At 30° C., 5-10 mg/L of oxidosqualene was detected in all three lanosterol synthase mutant strains while the control strain did not produce detectable levels of oxidosqualene (FIG. 5 and Table 7). Thus, host cells with decreased lanosterol synthase activity showed increased oxidosqualene production.

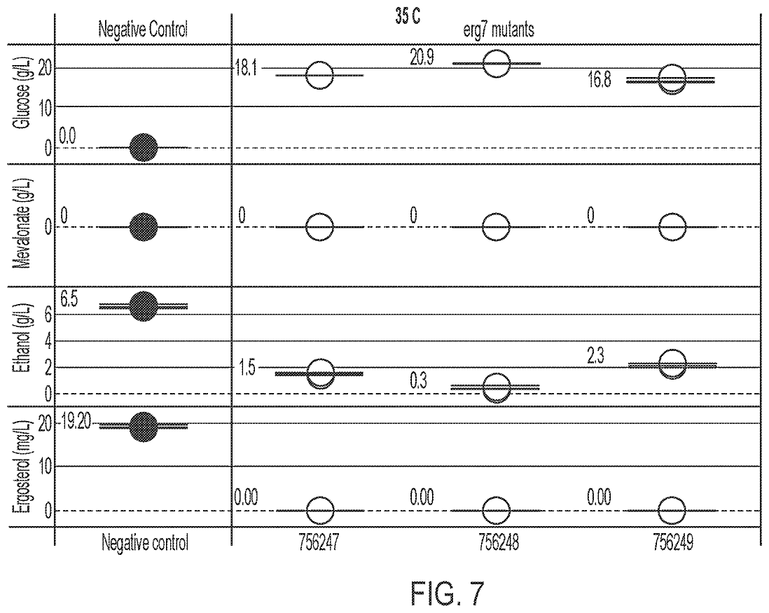

At 35° C., the lanosterol synthase mutant strains were unable to grow or grew minimally compared to the control strain as shown by the residual glucose numbers (FIG. 7 and Table 8). For all strains, the starting glucose concentration was 20 g/L. Without being bound by a particular theory, it is possible that since the lanosterol synthase mutants are temperature sensitive, the cells cannot survive in the absence of a functional lanosterol synthase comprising SEQ ID NO: 313 at higher temperatures. Only strain 756249 accumulated some oxidosqualene at 35° C. The control strain with the native lanosterol synthase gene encoding SEQ ID NO: 313 was able to consume all the glucose at 30° C. and 35° C., but did not produce detectable levels of oxidosqualene. Thus, the results suggest that complete knockout of lanosterol synthase activity is detrimental to these cells.

TABLE 7
Effects of Lanosterol Synthase Mutations Relative to
SEQ ID NO: 313 on Glucose Consumption and Oxidosqualene,
Mevalonate, Ergosterol, and Ethanol Production by
Saccharomyces cerevisiae Host Cells at 30° C.

| Saccharomyces | | Oxidosqua- | Glu- | | Ergos- | Eth- |
|---|---|---|---|---|---|---|
| cerevisiae | | lene | cose | Mevalonate | terol | anol |
| Strain | | (mg/L) | [g/L] | [g/L] | [mg/L] | [g/L] |
| Negative | 1 | 0.00 | 0.04 | 0.00 | 22.29 | 8.98 |
| control | 2 | 0.00 | 0.04 | 0.00 | 26.89 | 8.36 |
| (parent | 3 | 0.00 | 0.04 | 0.00 | 24.75 | 8.42 |
| strain with | | | | | | |
| a wild-type | | | | | | |
| lanosterol | | | | | | |
| synthase) | | | | | | |
| 756247 | 1 | 6.38 | 0.04 | 0.00 | 10.49 | 9.35 |
| | 2 | 7.01 | 0.04 | 0.00 | 12.71 | 9.52 |
| | 3 | 0.00 | 0.09 | 0.00 | 12.08 | 9.44 |
| 756248 | 1 | 5.71 | 16.10 | 0.00 | 0.00 | 2.50 |
| | 2 | 0.00 | 17.00 | 0.00 | 0.00 | 2.26 |
| | 3 | 10.53 | 17.00 | 0.00 | 0.00 | 2.36 |
| 756249 | 1 | 6.05 | 0.04 | 0.00 | 9.51 | 10.90 |
| | 2 | 0.00 | 0.03 | 0.00 | 17.32 | 9.52 |
| | 3 | 0.00 | 0.03 | 0.00 | 17.66 | 9.72 |

TABLE 8
Effects of Lanosterol Synthase Mutations Relative to
SEQ ID NO: 313 on Glucose Consumption and Oxidosqualene,
Mevalonate, Ergosterol, and Ethanol Production by
Saccharomyces cerevisiae Host Cells at 35° C.

| Saccharomyces | | Oxidosqua- | Glu- | | Ergos- | Eth- |
|---|---|---|---|---|---|---|
| cerevisiae | | lene | cose | Mevalonate | terol | anol |
| Strain | | (mg/L) | [g/L] | [g/L] | [mg/L] | [g/L] |
| Negative | 1 | 0.00 | 0.04 | 0.00 | 18.78 | 6.37 |
| control | 2 | 0.00 | 0.04 | 0.00 | 19.35 | 6.54 |
| (parent | 3 | 0.00 | 0.04 | 0.00 | 19.48 | 6.63 |
| strain with | | | | | | |
| a wild-type | | | | | | |
| lanosterol | | | | | | |
| synthase) | | | | | | |
| 756247 | 1 | 0.00 | 18.00 | 0.00 | 0.00 | 1.54 |
| | 2 | 0.00 | 18.10 | 0.00 | 0.00 | 1.48 |
| | 3 | 0.00 | 18.00 | 0.00 | 0.00 | 1.37 |
| 756248 | 1 | 0.00 | 21.00 | 0.00 | 0.00 | 0.53 |
| | 2 | 0.00 | 21.00 | 0.00 | 0.00 | 0.31 |
| | 3 | 0.00 | 20.70 | 0.00 | 0.00 | 0.28 |
| 756249 | 1 | 5.24 | 17.20 | 0.00 | 0.00 | 1.98 |
| | 2 | 7.54 | 16.40 | 0.00 | 0.00 | 2.29 |
| | 3 | 0.00 | 16.40 | 0.00 | 0.00 | 2.26 |

TABLE 9
Non-limiting Examples of Amino Acid
Changes Relative to SEQ ID NO: 1*

| | |
|---|---|
| | Amino acid change |
| Position | relative to SEQ ID NO: 1 |

| | | |
|---|---|---|
| 14 | N14Y | |
| 33 | R33Q | |
| 47 | K47E | |
| 50 | D50G | |
| 66 | K66R | |
| 80 | D80G | |
| 83 | P83L | |
| 85 | K85N | |
| 92 | L92I | |
| 94 | N94S | |
| 107 | G107D | |
| 122 | G122C | |
| 132 | N132S | |
| 145 | Y145C | |
| 158 | G158S | |
| 170 | T170A | |
| 172 | I172N | |
| 184 | R184W | |
| 193 | R193C | R193H |
| 197 | L197V | |
| 198 | T198I | |
| 212 | T212I | |
| 213 | W213L | |
| 227 | P227L | |
| 228 | A228T | |
| 231 | E231V | |
| 235 | L235M | |
| 248 | V248F | |
| 249 | H249L | |
| 260 | L260R | |
| 282 | K282I | |
| 286 | I286F | |
| 287 | E287G | |
| 289 | D289G | |
| 295 | N295I | |
| 296 | S296T | |
| 309 | L309F | |
| 314 | N314S | |
| 316 | L316R | |
| 329 | K329N | |
| 344 | V344A | |
| 360 | T360S | |
| 370 | P370L | |
| 371 | D371V | |
| 372 | S372P | |
| 398 | T398I | |
| 407 | A407V | |
| 414 | C414S | |
| 417 | G417S | |
| 423 | Q423L | |
| 432 | F432I | F432S |
| 437 | R437L | |
| 442 | E442V | |
| 444 | T444M | T444S |
| 452 | D452G | |
| 474 | E474V | |
| 479 | I479S | |
| 491 | L491Q | |
| 498 | K498N | |
| 515 | S515L | |
| 526 | P526T | |
| 529 | A529T | |
| 536 | I536F | |
| 544 | N544Y | |
| 552 | V552E | |
| 559 | V559A | |
| 560 | L560M | |
| 564 | Y564C | Y564N |
| 578 | R578P | |
| 586 | Y586F | |
| 608 | A608T | |
| 610 | M610I | |
| 617 | E617V | |
| 619 | Q619L | |
| 620 | N620S | |
| 631 | K631E | K631R |
| 638 | G638D | |
| 650 | F650L | |
| 655 | T655A | |
| 660 | R660H | |
| 679 | G679S | |
| 686 | K686E | |
| 702 | G702D | |
| 710 | E710Q | |
| 726 | F726L | F726V |
| 736 | Y736F | |
| 738 | K738M | |
| 742 | Q742* | |

*indicates a truncation

TABLE 10
Non-limiting Examples of Amino Acid
Changes Relative to SEQ ID NO: 313*

| | Amino acid change relative |
|---|---|
| Position | to SEQ ID NO: 313 |
| 64 | R64G |
| 120 | I120V |
| 121 | P121S |
| 136 | A136V |
| 226 | M226I |
| 268 | K268S |
| 275 | F275I |
| 281 | T281A |
| 300 | S300G |
| 322 | V322G |
| 333 | T333A |
| 438 | K438E |
| 502 | F502L |
| 604 | T604N |
| 619 | C619S |
| 628 | K628E |
| 656 | A656T |
| 693 | E693G |
| 726 | Y726* |

*indicates a truncation that results in deletion of residues 726-731 in SEQ ID NO: 313

TABLE 11
Non-limiting Examples of Lanosterol Synthase Sequences

| | | Nucleic | | |
|---|---|---|---|---|
| | | Acid | | Protein |
| | | SEQ ID | | SEQ |
| Strain | Nucleotide Sequence | NO | Protein Sequence | ID NO |
| 870688 | ATGGGAATCCACGAAAGTGTGTCGAA | 61 | MGIHESVSKQFAKNGHSKY | 1 |
| | ACAGTTTGCGAAAAACGGACATTCCAA | | RSDRYGLPKTDLRRWTFHA | |
| | GTACCGCAGCGACCGATACGGCTTACC | | SDLGAQWWKYDDTTPLEE | |
| | TAAGACGGATCTGCGACGATGGACGTT | | LEKRATDYVKYSLELPGYA | |
| | CCACGCGTCCGATCTGGGGGCGCAATG | | PVTLDSKPVKNAYEAALKN | |
| | GTGGAAGTATGACGATACCACACCGCT | | WHLFASLQDPDSGAWQSE | |
| | GGAAGAGCTGGAAAAGAGGGCTACCG | | YDGPQFMSIGYVTACYFGG | |
| | ACTACGTCAAATACTCGCTGGAGCTGC | | NEIPTPVKTEMIRYIVNTAH | |
| | CGGGATACGCGCCCGTGACTCTGGACT | | PVDGGWGLHKEDKSTCFG | |
| | CCAAGCCCGTGAAAAATGCCTACGAA | | TSINYVVLRLLGLSRDHPVC | |
| | GCGGCTCTCAAAAACTGGCATCTGTTT | | VKARKTLLTKFGGAINNPH | |
| | GCGTCGCTGCAAGACCCCGACTCCGGC | | WGKTWLSILNLYKWEGVN | |
| | GCGTGGCAGAGCGAATACGACGGACC | | PAPGELWLLPYFVPVHPGR | |
| | GCAGTTCATGAGCATCGGCTATGTGAC | | WWVHTRWIYLAMGYLEA | |
| | GGCGTGCTACTTTGGCGGCAACGAGAT | | AEAQCELTPLLEELRDEIYK | |
| | CCCCACGCCGGTCAAAACCGAAATGAT | | KPYSEIDFSKHCNSISGVDL | |
| | CAGATACATTGTCAACACAGCCCACCC | | YYPHTGLLKFGNALLRRYR | |
| | AGTTGACGGAGGCTGGGGCCTTCACAA | | KFRPQWIKEKVKEEIYNLC | |
| | AGAAGACAAGAGCACCTGCTTCGGTAC | | LREVSNTRHLCLAPVNNAM | |
| | CAGCATCAACTACGTGGTCCTGCGACT | | TSIVMYLHEGPDSANYKKI | |
| | ACTGGGCCTGTCACGGGACCATCCGGT | | AARWPEFLSLNPSGMFMN | |
| | CTGCGTCAAGGCGCGCAAAACGCTGCT | | GTNGLQVWDTAFAVQYAC | |
| | CACCAAGTTTGGCGGCGCCATCAACAA | | VCGFAELPQYQKTIRAAFD | |
| | CCCCCATTGGGGCAAGACCTGGCTGTC | | FLDRSQINEPTEENSYRDDR | |
| | GATTCTCAATCTCTACAAATGGGAGGG | | VGGWPFSTKTQGYPVSDCT | |
| | TGTGAATCCGGCCCCTGGCGAGCTCTG | | AEALKAIIMVQNTPGYEDL | |
| | GCTGTTGCCCTACTTTGTTCCTGTTCAT | | KKQVSDKRKHTAIDLLLGM | |
| | CCGGGCCGATGGTGGGTCCATACCCGG | | QNVGSFEPGSFASYEPIRAS | |
| | TGGATCTACCTTGCCATGGGCTATCTG | | SMLEKINPAEVFGNIMVEY | |
| | GAGGCTGCGGAGGCCCAATGCGAACT | | PYVECTDSVVLGLSYFRKY | |
| | CACTCCGTTGCTGGAGGAGCTCCGAGA | | HDYRNEDVDRAISAAIGYII | |
| | CGAAATCTACAAAAAGCCCTACTCGGA | | REQQPDGGFFGSWGVCYC | |
| | GATTGATTTCTCCAAACATTGCAACTC | | YAHMFAMEALETQNLNYN | |
| | CATCTCCGGAGTCGACCTCTACTATCC | | NCSTVQKACDFLAGYQEA | |
| | CCACACCGGCCTTTTGAAGTTTGGCAA | | DGGWAEDFKSCETQMYVR | |
| | CGCGCTTCTCCGACGATACCGCAAGTT | | GPHSLVVPTAMALLSLMSG | |
| | CAGACCGCAGTGGATCAAAGAAAAGG | | RYPQEDKIHAAARFLMSKQ | |
| | TCAAGGAGGAAATTTACAACTTGTGCC | | MSNGEWLKEEMEGVFNHT | |
| | TTCGAGAGGTTTCCAACACACGACACT | | CAIEYPNYRFYFVMKALGL | |
| | TGTGTCTCGCTCCCGTCAACAATGCCA | | YFKGYCQ | |
| | TGACCTCCATTGTCATGTATCTCCATGA | | | |
| | GGGGCCCGATTCGGCGAATTACAAAA | | | |
| | AGATTGCGGCCCGATGGCCCGAATTTC | | | |
| | TGTCTCTGAATCCGTCGGGAATGTTTA | | | |
| | TGAACGGCACCAACGGTCTCCAGGTCT | | | |
| | GGGATACTGCGTTTGCCGTGCAATACG | | | |
| | CGTGTGTTTGTGGCTTTGCCGAACTTCC | | | |
| | CCAGTACCAGAAGACGATCCGAGCGG | | | |
| | CGTTTGATTTTCTCGATCGGTCCCAGAT | | | |
| | CAACGAGCCGACGGAGGAAAATTCCT | | | |
| | ATCGAGACGACCGCGTCGGAGGATGG | | | |
| | CCCTTTAGTACCAAGACCCAGGGGTAT | | | |
| | CCGGTCTCCGACTGTACTGCCGAGGCT | | | |
| | CTCAAGGCCATCATCATGGTCCAGAAT | | | |
| | ACGCCTGGATACGAGGATCTGAAGAA | | | |
| | ACAAGTGTCTGACAAGCGGAAACACA | | | |
| | CTGCCATCGATCTACTTTTGGGAATGC | | | |
| | AGAACGTGGGCTCGTTTGAACCGGGCT | | | |
| | CTTTCGCCTCCTATGAGCCTATCCGGG | | | |
| | CGTCGTCCATGCTGGAGAAGATCAATC | | | |
| | CGGCCGAGGTGTTTGGAAACATCATGG | | | |
| | TGGAGTATCCGTACGTGGAATGCACTG | | | |
| | ATTCTGTTGTTCTGGGTCTCTCCTACTT | | | |
| | TCGAAAGTACCACGATTACCGCAACGA | | | |
| | AGACGTGGACCGAGCCATCTCTGCTGC | | | |
| | CATTGGATACATTATTCGAGAGCAGCA | | | |
| | GCCTGACGGCGGCTTCTTTGGCTCCTG | | | |
| | GGGCGTGTGCTACTGCTACGCTCACAT | | | |
| | GTTTGCCATGGAGGCTCTGGAGACGCA | | | |
| | GAATCTCAACTATAACAACTGTTCCAC | | | |
| | GGTTCAAAAGGCGTGCGACTTTCTGGC | | | |
| | GGGCTACCAGGAAGCAGATGGAGGCT | | | |
| | GGGCCGAGGACTTTAAGTCGTGCGAGA | | | |
| | CCCAGATGTACGTGCGCGGACCCCATT | | | |
| | CGCTGGTCGTGCCTACTGCCATGGCCC | | | |
| | TGTTGAGTTTGATGAGTGGTCGGTATC | | | |
| | CCCAGGAGGACAAGATTCATGCTGCGG | | | |
| | CCCGGTTTCTCATGAGCAAGCAGATGA | | | |
| | GCAACGGTGAGTGGCTCAAGGAGGAG | | | |
| | ATGGAGGGGGTGTTTAACCATACTTGT | | | |
| | GCCATTGAGTATCCCAACTACCGGTTT | | | |
| | TATTTTGTCATGAAGGCTTTGGGGTTGT | | | |
| | ATTTCAAGGGATATTGCCAGTGA | | | |
| 907808 | ATGGGAATCCACGAAAGTGTGTCGAA | 62 | MGIHESVSKQFAKNGHSKY | 83 |
| | ACAGTTTGCGAAAAACGGACATTCCAA | | RSDRYGLPKTDLRRWTFHA | |
| | GTACCGCAGCGACCGATACGGCTTACC | | SDLGAQWWKYDDTTPLEE | |
| | TAAGACGGATCTGCGACGATGGACGTT | | LEKRATDYVKYSLELPGYA | |
| | CCACGCGTCCGATCTGGGGGCGCAATG | | PVTLDSKPVKNAYEAALKN | |
| | GTGGAAGTATGACGATACCACACCGCT | | WHLFASLQDPDSGAWQSE | |
| | GGAAGAGTTGGAAAAGAGGGCTACCG | | YDGPQFMSIGYVTACYFGG | |
| | ACTACGTCAAATACTCGCTGGAGCTGC | | NEIPTPVKTEMIRYIVNTAH | |
| | CGGGATACGCGCCCGTGACTCTGGACT | | PVDGGWGLHKEDKSTCFG | |
| | CCAAGCCCGTGAAAAATGCCTACGAA | | TSINYVVLRLLGLSRDHPVC | |
| | GCGGCTCTCAAAAACTGGCATCTGTTT | | VKACKTLLTKFGGAINNPH | |
| | GCGTCGCTGCAAGACCCCGACTCCGGC | | WGKTWLSILNLYKWEGVN | |
| | GCATGGCAGTCGGAATACGACGGACC | | PAPGELWLLPYFVPVHPGR | |
| | GCAGTTCATGTCGATCGGTTATGTGAC | | WWVHTRWIYLAMGYLEA | |
| | GGCGTGCTACTTTGGCGGCAACGAGAT | | AEAQCELTPLLEELRDEIYK | |
| | CCCCACGCCGGTCAAAACCGAAATGAT | | KPYSEIGFSKHCITISGVDLY | |
| | CAGATACATTGTCAACACAGCCCACCC | | YPHTGLLKFGNALLRRYRK | |
| | AGTTGACGGAGGCTGGGGCCTTCACAA | | FRPQWIKEKVKEEIYNLCLR | |
| | AGAAGACAAGAGCACCTGTTTCGGTAC | | EVSNTRHLCLAPVNNAMTS | |
| | CAGCATCAACTACGTGGTCCTGCGACT | | IVMYLHEGPDSANYKKIAA | |
| | ACTGGGCCTGTCGCGGGATCATCCGGT | | RWPEFLSLNPSGMFMNGTN | |
| | CTGCGTCAAGGCGTGCAAAACGCTGCT | | GLQVWDTAFAVQYACVCG | |
| | CACCAAGTTTGGCGGCGCCATCAACAA | | FAELPQYQKTIRAAFDFLDR | |
| | CCCCCATTGGGGCAAGACCTGGCTGTC | | SQINEPTEENSYRDDRVGG | |
| | GATTCTCAATCTCTACAAATGGGAGGG | | WPFSTKTQGYPVSDCTAEA | |
| | TGTGAATCCGGCCCCTGGCGAGCTCTG | | LKAIIMVQNTPGYEDLKKQ | |
| | GCTGTTGCCCTACTTTGTTCCTGTTCAT | | VSDKRKHTAIDLLLGMQNV | |
| | CCGGGCCGATGGTGGGTCCATACCCGG | | GSFEPGSFASYEPIRASSML | |
| | TGGATCTACCTTGCCATGGGCTATCTG | | EKINPAEVFGNIMVEYPYV | |
| | GAGGCTGCGGAGGCCCAATGCGAACT | | ECTDSVVLGLSYFRKYHDY | |
| | CACTCCGTTGCTGGAGGAGCTCCGAGA | | RNEDVDRAISAAIGYIIREQ | |
| | CGAAATCTACAAAAAGCCCTACTCGGA | | QPDGGFFGSWGVCYCYAH | |
| | GATTGGTTTCTCCAAACATTGCATCAC | | MFAMEALETQSLNYNNCST | |
| | CATCTCCGGAGTCGACCTCTACTATCC | | VQKACDFLAGYQEADGGW | |
| | CCACACCGGCCTTTTGAAGTTTGGCAA | | AEDFKSCETQMYVRGPHSL | |
| | CGCGCTTCTCCGACGATACCGCAAGTT | | VVPTAMALLSLMSGRYPQE | |
| | CAGACCGCAGTGGATCAAAGAAAAGG | | DKIHAAARFLMSKQMSNG | |
| | TCAAGGAGGAAATTTATAACTTGTGCC | | EWLKEEMEGVFNHTCAIEY | |
| | TTCGAGAGGTTTCCAACACACGACACT | | PNYRFYFVMKALGLFFKGY | |
| | TGTGTCTCGCTCCCGTCAACAATGCCA | | CQ | |
| | TGACCTCCATTGTCATGTATCTCCATGA | | | |
| | GGGGCCCGATTCGGCGAATTACAAAA | | | |
| | AGATTGCGGCCCGATGGCCCGAATTTC | | | |
| | TGTCTCTGAATCCGTCGGGAATGTTTA | | | |
| | TGAACGGCACCAACGGTCTGCAGGTCT | | | |
| | GGGATACTGCGTTTGCCGTGCAATACG | | | |
| | CGTGTGTTTGTGGCTTTGCCGAACTTCC | | | |
| | CCAGTACCAGAAGACGATCCGAGCGG | | | |
| | CGTTTGATTTTCTCGATCGGTCCCAGAT | | | |
| | CAACGAGCCGACGGAGGAAAATTCCT | | | |
| | ATCGAGACGACCGCGTCGGAGGATGG | | | |
| | CCCTTTAGTACCAAGACCCAGGGGTAT | | | |
| | CCAGTCTCCGACTGTACTGCCGAGGCT | | | |
| | CTCAAGGCCATCATCATGGTCCAGAAT | | | |
| | ACGCCTGGATACGAGGATCTGAAGAA | | | |
| | ACAAGTGTCTGACAAGCGGAAACACA | | | |
| | CTGCCATCGATCTACTTTTGGGAATGC | | | |
| | AGAACGTGGGCTCGTTTGAACCGGGCT | | | |
| | CTTTCGCCTCCTATGAGCCTATCCGGG | | | |
| | CGTCGTCCATGCTGGAGAAGATCAATC | | | |
| | CGGCCGAGGTGTTTGGAAACATCATGG | | | |
| | TGGAGTATCCGTACGTGGAATGCACTG | | | |
| | ATTCTGTTGTTCTGGGTCTGTCCTACTT | | | |
| | TCGAAAGTACCACGATTACCGCAACGA | | | |
| | AGACGTGGACCGAGCCATCTCTGCTGC | | | |
| | CATTGGATACATTATTCGAGAGCAGCA | | | |
| | GCCTGACGGCGGCTTCTTTGGCTCCTG | | | |
| | GGGCGTGTGCTACTGCTACGCTCACAT | | | |
| | GTTTGCCATGGAGGCTCTGGAGACGCA | | | |
| | GAGTCTCAACTATAACAACTGTTCCAC | | | |
| | GGTTCAAAAGGCGTGCGACTTTCTGGC | | | |
| | GGGCTACCAGGAAGCAGATGGAGGCT | | | |
| | GGGCCGAGGACTTTAAGTCGTGCGAGA | | | |
| | CTCAGATGTACGTGCGCGGACCCCATT | | | |
| | CGCTGGTCGTGCCTACTGCCATGGCCC | | | |
| | TGTTGAGTTTGATGAGTGGTCGGTATC | | | |
| | CCCAGGAGGACAAGATTCATGCTGCGG | | | |
| | CCCGGTTTCTCATGAGCAAGCAGATGA | | | |
| | GCAACGGTGAGTGGCTCAAGGAGGAG | | | |
| | ATGGAGGGGGTGTTTAACCATACTTGT | | | |
| | GCCATTGAGTATCCCAACTACCGGTTT | | | |
| | TATTTTGTCATGAAGGCTTTGGGGTTGT | | | |
| | TTTTCAAGGGATATTGCCAGTGA | | | |
| 948806 | ATGGGAATCCACGAAAGTGTGTCGAA | 63 | MGIHESVSKQFAKNGHSKY | 84 |
| | ACAGTTTGCGAAAAACGGACATTCCAA | | RSDRYGLPKTDLRRWTFHA | |
| | GTACCGCAGCGACCGATACGGCTTACC | | SDLGAQWWKYDDTTPLEE | |
| | TAAGACGGATCTGCGACGATGGACGTT | | LEKRATDYVKYSLELPGYA | |
| | CCACGCGTCCGATCTGGGGGCGCAATG | | PVTLDSKPVKNAYEAALKN | |
| | GTGGAAGTATGACGATACCACACCGCT | | WHLFASLQDPDSGAWQSE | |
| | GGAAGAGCTGGAAAAGAGGGCTACCG | | YDGPQFMSIGYVTACYFGG | |
| | ACTACGTCAAATACTCGCTGGAGCTGC | | NEIPTPVKTEMIRYIVNTAH | |
| | CGGGATACGCGCCCGTGACTCTGGACT | | PVDGGWGLHKEDKSTCFG | |
| | CCAAGCCCGTGAAAAATGCCTACGAA | | TSNNYVVLRLLGLSRDHPV | |
| | GCGGCTCTCAAAAACTGGCATCTGTTT | | CVKARKTLLTKFGGAINNP | |
| | GCGTCGCTGCAAGACCCCGACTCCGGC | | HWGKTWLSILNLYKWEGV | |
| | GCATGGCAGTCGGAATACGACGGACC | | NPAPGELWLLPYFVPVHPG | |
| | GCAGTTCATGTCGATCGGTTATGTGAC | | RWWVHTRWIYLAMGYLE | |
| | GGCGTGCTACTTTGGCGGCAACGAGAT | | AAEAQCELTPLLEELRDEIY | |
| | CCCCACGCCGGTCAAAACCGAAATGAT | | KKPYSEIDFSKHCNSISGVD | |
| | CAGATACATTGTCAACACAGCCCACCC | | LYYPHTGLLKFGNALLRRY | |
| | AGTTGACGGAGGCTGGGGCCTTCACAA | | RKFRPQWIKEKVKEEIYNL | |
| | AGAAGACAAGAGCACCTGTTTCGGTAC | | CLREVSNTRHLCLAPVNNA | |
| | CAGCAACAACTACGTGGTCCTGCGACT | | MTSIVMYLHEGPDSANYKK | |
| | ACTGGGCCTGTCACGGGATCATCCGGT | | IAARWPEFLSLNPSGMFMN | |
| | CTGCGTCAAGGCGCGCAAAACGCTGCT | | GTNGLQVWDTAFAVQYAS | |
| | CACCAAGTTTGGCGGCGCCATCAACAA | | VCGFAELPQYQKTIRAAFD | |
| | CCCCCATTGGGGCAAGACCTGGCTGTC | | FLDRSQINEPTEENSYRDDR | |
| | GATTCTCAATCTCTACAAATGGGAGGG | | VGGWPFSTKTQGYPVSDCT | |
| | TGTGAATCCGGCCCCTGGCGAGCTCTG | | AEALKAIIMVQNTPGYEDL | |
| | GCTGTTGCCCTACTTTGTTCCTGTTCAT | | KKQVSDKRKHTAIDLLLGM | |
| | CCGGGCCGATGGTGGGTCCATACCCGG | | QNVGSFEPGSFASYEPIRAS | |
| | TGGATCTACCTTGCCATGGGCTATCTG | | SMLEKINPAEVFGNIMVEY | |
| | GAGGCTGCGGAGGCCCAATGCGAACT | | PYVECTDSVVMGLSYFRKY | |
| | CACTCCGTTGCTGGAGGAGCTCCGAGA | | HDYRNEDVDRAISAAIGYII | |
| | CGAAATCTACAAAAAGCCCTACTCGGA | | REQQPDGGFFGSWGVCYC | |
| | GATTGATTTCTCCAAACATTGCAACTC | | YAHMFAMEALETQNLNYN | |
| | CATCTCCGGAGTCGACCTCTACTATCC | | NCSTVQKACDFLAGYQEA | |
| | CCACACCGGCCTTTTGAAGTTTGGCAA | | DGGWAEDFKSCETQMYVR | |
| | CGCGCTTCTCCGACGATACCGCAAGTT | | GPHSLVVPTAMALLSLMSS | |
| | CAGACCGCAGTGGATCAAAGAAAAGG | | RYPQEDKIHAAARFLMSKQ | |
| | TCAAGGAGGAAATTTACAACTTGTGCC | | MSNGEWLKEEMEGVFNHT | |
| | TTCGAGAGGTTTCCAACACACGACACT | | CAIEYPNYRFYFVMKALGL | |
| | TGTGTCTCGCTCCCGTCAACAATGCCA | | YFKGYCQ | |
| | TGACCTCCATTGTCATGTATCTCCATGA | | | |
| | GGGGCCCGATTCGGCGAATTACAAAA | | | |
| | AGATTGCGGCCCGATGGCCCGAATTTC | | | |
| | TGTCTCTGAATCCGTCGGGAATGTTTA | | | |
| | TGAACGGCACCAACGGTCTGCAGGTCT | | | |
| | GGGATACTGCGTTTGCCGTGCAATACG | | | |
| | CGAGTGTTTGTGGCTTTGCCGAACTTC | | | |
| | CCCAGTACCAGAAGACGATCCGAGCG | | | |
| | GCGTTTGATTTTCTCGATCGGTCCCAG | | | |
| | ATCAACGAGCCGACGGAGGAAAATTC | | | |
| | CTATCGAGACGACCGCGTCGGAGGATG | | | |
| | GCCCTTTAGTACCAAGACCCAGGGGTA | | | |
| | TCCAGTCTCCGACTGTACTGCCGAGGC | | | |
| | TCTCAAGGCCATCATCATGGTCCAGAA | | | |
| | TACGCCTGGATACGAGGATCTGAAGAA | | | |
| | ACAAGTGTCTGACAAGCGGAAACACA | | | |
| | CTGCCATCGATCTACTTTTGGGAATGC | | | |
| | AGAACGTGGGCTCGTTTGAACCGGGCT | | | |
| | CTTTCGCCTCCTATGAGCCTATCCGGG | | | |
| | CGTCGTCCATGCTGGAGAAGATCAATC | | | |
| | CGGCCGAGGTGTTTGGAAACATCATGG | | | |
| | TGGAGTATCCGTACGTGGAATGCACTG | | | |
| | ATTCTGTTGTTATGGGTCTGTCCTACTT | | | |
| | TCGAAAGTACCACGATTACCGCAACGA | | | |
| | AGACGTGGACCGAGCCATCTCTGCTGC | | | |
| | CATTGGATACATTATTCGAGAGCAGCA | | | |
| | GCCTGACGGCGGCTTCTTTGGCTCCTG | | | |
| | GGGCGTGTGCTACTGCTACGCTCACAT | | | |
| | GTTTGCCATGGAGGCTCTGGAGACGCA | | | |
| | GAATCTCAACTATAACAACTGTTCCAC | | | |
| | GGTTCAAAAGGCGTGCGACTTTCTGGC | | | |
| | GGGCTACCAGGAAGCAGATGGAGGCT | | | |
| | GGGCCGAGGACTTTAAGTCGTGCGAGA | | | |
| | CTCAGATGTACGTGCGCGGACCCCATT | | | |
| | CGCTGGTCGTGCCTACTGCCATGGCCC | | | |
| | TGTTGAGTTTGATGAGTAGTCGGTATC | | | |
| | CCCAGGAGGACAAGATTCATGCTGCGG | | | |
| | CCCGGTTTCTCATGAGCAAGCAGATGA | | | |
| | GCAACGGTGAGTGGCTCAAGGAGGAG | | | |
| | ATGGAGGGGGTGTTTAACCATACTTGT | | | |
| | GCCATTGAGTATCCCAACTACCGGTTT | | | |
| | TATTTTGTCATGAAGGCTTTGGGGTTGT | | | |
| | ATTTCAAGGGATATTGCCAGTGA | | | |
| 948810 | ATGGGAATCCACGAAAGTGTGTCGAA | 64 | MGIHESVSKQFAKNGHSKY | 85 |
| | ACAGTTTGCGAAAAACGGACATTCCAA | | RSDRYGLPKTDLRRWTFHA | |
| | GTACCGCAGCGACCGATACGGCTTACC | | SDLGAQWWKYDDTTPLEE | |
| | TAAGACGGATCTGCGACGATGGACGTT | | LEKRATDYVKYSLELPGYA | |
| | CCACGCGTCCGATCTGGGGGCGCAATG | | PVTLDSKPVKNAYEAALKN | |
| | GTGGAAGTATGACGATACCACACCGCT | | WHLFASLQDPDSGAWQSE | |
| | GGAAGAGCTGGAAAAGAGGGCTACCG | | YDGPQFMSIGYVTACYFGG | |
| | ACTACGTCAAATACTCGCTGGAGCTGC | | NEIPTPVKTEMIRYIVNTAH | |
| | CGGGATACGCGCCCGTGACTCTGGACT | | PVDGGWGLHKEDKSTCFG | |
| | CCAAGCCCGTGAAAAATGCCTACGAA | | TSINYVVLRLLGLSRDHPVC | |
| | GCGGCTCTCAAAAACTGGCATCTGTTT | | VKARKTLLTKFGGAINNPH | |
| | GCGTCGCTGCAAGACCCCGACTCCGGC | | WGKTWLSILNLYKWEGVN | |
| | GCATGGCAGTCGGAATACGACGGACC | | PAPGELWLLPYFVPVHPGR | |
| | GCAGTTCATGTCGATCGGTTATGTGAC | | WWVHTRWIYLAMGYLEA | |
| | GGCGTGCTACTTTGGCGGCAACGAGAT | | AEAQCELTPLLEELRDEIYK | |
| | CCCCACGCCGGTCAAAACCGAAATGAT | | KPYSEIDFSKHCNSISGVDL | |
| | CAGATACATTGTCAACACAGCCCACCC | | YYPHTGLLKFGNALLRRYR | |
| | AGTTGACGGAGGCTGGGGCCTTCACAA | | KFRPQWIKEKVKEEIYNLC | |
| | AGAAGACAAGAGCACCTGTTTCGGTAC | | LREVSNTRHLCLAPVNNAM | |
| | CAGCATCAACTACGTGGTCCTGCGACT | | TSIVMYLHEGPDSANYKKI | |
| | ACTGGGCCTGTCACGGGATCATCCGGT | | AARWPEFLSLNPSGMFMN | |
| | CTGCGTCAAGGCGCGCAAAACGCTGCT | | GTNGLQVWDTAFAVQYAC | |
| | CACCAAGTTTGGCGGCGCCATCAACAA | | VCGFAELPQYQKTIRAASD | |
| | CCCCCATTGGGGCAAGACCTGGCTGTC | | FLDRSQINEPTEENSYRDGR | |
| | GATTCTCAATCTCTACAAATGGGAGGG | | VGGWPFSTKTQGYPVSDCT | |
| | TGTGAATCCGGCCCCTGGCGAGCTCTG | | AEALKAIIMVQNTPGYEDL | |
| | GCTGTTGCCCTACTTTGTTCCTGTTCAT | | KKQVSDKRKHTAIDLLLGM | |
| | CCGGGCCGATGGTGGGTCCATACCCGG | | QNVGSFEPGSFASYEPIRAS | |
| | TGGATCTACCTTGCCATGGGCTATCTG | | SMLEKFNPAEVFGNIMVEY | |
| | GAGGCTGCGGAGGCCCAATGCGAACT | | PYVECTDSVVLGLSYFRKY | |
| | CACTCCGTTGCTGGAGGAGCTCCGAGA | | HDYRNEDVDRAISAAIGYII | |
| | CGAAATCTACAAAAAGCCCTACTCGGA | | REQQPDGGFFGSWGVCYC | |
| | GATTGATTTCTCCAAACATTGCAACTC | | YAHMFAMEALETQNLNYN | |
| | CATCTCCGGAGTCGACCTCTACTATCC | | NCSTVQKACDFLAGYQEA | |
| | CCACACCGGCCTTTTGAAGTTTGGCAA | | DGGWAEDFKSCETQMYVR | |
| | CGCGCTTCTCCGACGATACCGCAAGTT | | GPHSLVVPTAMALLSLMSG | |
| | CAGACCGCAGTGGATCAAAGAAAAGG | | RYPQEDKIHAAARFLMSKQ | |
| | TCAAGGAGGAAATTTACAACTTGTGCC | | MSNGEWLKEEMEGVFNHT | |
| | TTCGAGAGGTTTCCAACACACGACACT | | CAIEYPNYRFYFVMKALGL | |
| | TGTGTCTCGCTCCCGTCAACAATGCCA | | YFKGYCQ | |
| | TGACCTCCATTGTCATGTATCTCCATGA | | | |
| | GGGGCCCGATTCGGCGAATTACAAAA | | | |
| | AGATTGCGGCCCGATGGCCCGAATTTC | | | |
| | TGTCTCTGAATCCGTCGGGAATGTTTA | | | |
| | TGAACGGCACCAACGGTCTGCAGGTCT | | | |
| | GGGATACTGCGTTTGCCGTGCAATACG | | | |
| | CGTGTGTTTGTGGCTTTGCCGAACTTCC | | | |
| | CCAGTACCAGAAGACGATCCGAGCGG | | | |
| | CGTCTGATTTTCTCGATCGGTCCCAGAT | | | |
| | CAACGAGCCGACGGAGGAAAATTCCT | | | |
| | ATCGAGACGGCCGCGTCGGAGGATGG | | | |
| | CCCTTTAGTACCAAGACCCAGGGGTAT | | | |
| | CCAGTCTCCGACTGTACTGCCGAGGCT | | | |
| | CTCAAGGCCATCATCATGGTCCAGAAT | | | |
| | ACGCCTGGATACGAGGATCTGAAGAA | | | |
| | ACAAGTGTCTGACAAGCGGAAACACA | | | |
| | CTGCCATCGATCTACTTTTGGGAATGC | | | |
| | AGAACGTGGGCTCGTTTGAACCGGGCT | | | |
| | CTTTCGCCTCCTATGAGCCTATCCGGG | | | |
| | CGTCGTCCATGCTGGAGAAGTTCAATC | | | |
| | CGGCCGAGGTGTTTGGAAACATCATGG | | | |
| | TGGAGTATCCGTACGTGGAATGCACTG | | | |
| | ATTCTGTTGTTCTGGGTCTGTCCTACTT | | | |
| | TCGAAAGTACCACGATTACCGCAACGA | | | |
| | AGACGTGGACCGAGCCATCTCTGCTGC | | | |
| | CATTGGATACATTATTCGAGAGCAGCA | | | |
| | GCCTGACGGTGGCTTCTTTGGCTCCTG | | | |
| | GGGCGTGTGCTACTGCTACGCTCACAT | | | |
| | GTTTGCCATGGAGGCTCTGGAGACGCA | | | |
| | GAATCTCAACTATAACAACTGTTCCAC | | | |
| | GGTTCAAAAGGCGTGCGACTTTCTGGC | | | |
| | GGGCTACCAGGAAGCAGATGGAGGCT | | | |
| | GGGCCGAGGACTTTAAGTCGTGCGAGA | | | |
| | CCCAGATGTACGTGCGCGGACCCCATT | | | |
| | CGCTGGTCGTGCCTACTGCCATGGCCC | | | |
| | TGTTGAGTTTGATGAGTGGTCGGTATC | | | |
| | CCCAGGAGGACAAGATTCATGCTGCGG | | | |
| | CCCGGTTTCTCATGAGCAAGCAGATGA | | | |
| | GCAACGGTGAGTGGCTCAAGGAGGAG | | | |
| | ATGGAGGGGGTGTTTAACCATACTTGT | | | |
| | GCCATTGAGTATCCCAACTACCGGTTT | | | |
| | TATTTTGTCATGAAGGCTTTGGGGTTGT | | | |
| | ATTTCAAGGGATATTGCCAGTGA | | | |
| 948821 | ATGGGAATCCACGAAAGTGTGTCGAA | 65 | MGIHESVSKQFAKNGHSKY | 86 |
| | ACAGTTTGCGAAAAACGGACATTCCAA | | RSDRYGLPKTDLRRWTFHA | |
| | GTACCGCAGCGACCGATACGGCTTACC | | SDLGAQWWKYDDTTPLEE | |
| | TAAGACGGATCTGCGACGATGGACGTT | | LEKRATDYVKYSLELPGYA | |
| | CCACGCGTCCGATCTGGGGGCGCAATG | | PVTLDSKPVNNAYEAALKN | |
| | GTGGAAGTATGACGATACCACACCGCT | | WHLFASLQDPDSGAWQSE | |
| | GGAAGAGCTGGAAAAGAGGGCTACCG | | YDGPQFMSIGYVTACYFGG | |
| | ACTACGTCAAATACTCGCTGGAGCTGC | | NEIPTPVKTEMIRYIVNTAH | |
| | CGGGATACGCGCCCGTGACTCTGGACT | | PVDGGWSLHKEDKSTCFGT | |
| | CCAAGCCCGTGAATAATGCCTACGAAG | | SINYVVLRLLGLSRDHPVC | |
| | CGGCTCTCAAAAACTGGCATCTGTTTG | | VKARKTLLTKFGGAINNPH | |
| | CGTCGCTGCAAGACCCCGACTCCGGCG | | WGKTWLSILNLYKWEGVN | |
| | CATGGCAGTCGGAATACGACGGACCG | | PAPGELWLLPYFVPVHPGR | |
| | CAGTTCATGTCGATCGGTTATGTGACG | | WWVHTRWIYLAMGYLEA | |
| | GCATGCTACTTTGGCGGCAACGAGATC | | AEAQCELTPLLEELRDEIYK | |
| | CCCACGCCGGTCAAAACCGAAATGATC | | KPYSEIDFSKHCNSISGVDL | |
| | AGATACATTGTCAACACAGCCCACCCA | | YYPHTGLLKFGNALLRRYR | |
| | GTTGACGGAGGCTGGAGCCTTCACAAA | | KFRPQWIKEKVKEEIYNLC | |
| | GAAGACAAGAGCACCTGTTTCGGTACC | | LREVSNTRHLCLAPVNNAM | |
| | AGCATCAACTACGTGGTCCTGCGACTA | | TSIVMYLHEGPDSANYKKI | |
| | CTGGGCCTGTCACGGGATCATCCGGTC | | AARWPEFLSLNPSGMFMN | |
| | TGCGTCAAGGCGCGCAAAACGCTGCTC | | GTNGLQVWDTAFAVQYAC | |
| | ACCAAGTTTGGCGGCGCCATCAACAAC | | VCGFAELPQYQKTIRAAFD | |
| | CCCCATTGGGGCAAGACCTGGCTGTCG | | FLDRSQINEPTEENSYRDDR | |
| | ATTCTCAATCTCTACAAATGGGAGGGT | | VGGWPFSTKTQGYPVSDCT | |
| | GTGAATCCGGCCCCTGGCGAGCTCTGG | | AEALKAIIMVQNTPGYEDL | |
| | CTGTTGCCCTACTTTGTTCCTGTTCATC | | KKQVSDKRKHTAIDLLLGM | |
| | CGGGCCGATGGTGGGTCCATACCCGGT | | QNVGSFEPGSFASYEPIRAS | |
| | GGATCTACCTTGCCATGGGCTATCTGG | | SMLEKINPAEVFGNIMVEY | |
| | AGGCTGCGGAGGCCCAATGCGAACTC | | PYVECTDSVVLGLSYFRKY | |
| | ACTCCGTTGCTGGAGGAGCTCCGAGAC | | HDYRNEDVDRAISAAIGYII | |
| | GAAATCTACAAAAAGCCCTACTCGGAG | | REQQPDGGFFGSWGVCYC | |
| | ATTGATTTCTCCAAACATTGCAACTCC | | YAHMFAMEALETQNLNYN | |
| | ATCTCCGGAGTCGACCTCTACTATCCC | | NCSTVQKACDFLAGYQEA | |
| | CACACCGGCCTTTTGAAGTTTGGCAAC | | DGGWAEDFKSCETQMYVR | |
| | GCGCTTCTCCGACGATACCGCAAGTTC | | GPHSLVVPTAMALLSLMSG | |
| | AGACCGCAGTGGATCAAAGAAAAGGT | | RYPQEDKIHAAARFLMSKQ | |
| | CAAGGAGGAAATTTACAACTTGTGCCT | | MSNGEWLKEEMEGVFNHT | |
| | TCGAGAGGTTTCCAACACACGACACTT | | CAIEYPNYRFYFVMKALGL | |
| | GTGTCTCGCTCCCGTCAACAATGCCAT | | YFKGYCQ | |
| | GACCTCCATTGTCATGTATCTCCATGA | | | |
| | GGGGCCCGATTCGGCGAATTACAAAA | | | |
| | AGATTGCGGCCCGATGGCCCGAATTTC | | | |
| | TGTCTCTGAATCCGTCGGGAATGTTTA | | | |
| | TGAACGGCACCAACGGTCTCCAGGTCT | | | |
| | GGGATACTGCGTTTGCCGTGCAATACG | | | |
| | CGTGTGTTTGTGGCTTTGCCGAACTTCC | | | |
| | CCAGTACCAGAAGACGATCCGAGCGG | | | |
| | CGTTTGATTTTCTCGATCGGTCCCAGAT | | | |
| | CAACGAGCCGACGGAGGAAAATTCCT | | | |
| | ATCGAGACGACCGCGTCGGAGGATGG | | | |
| | CCCTTTAGTACCAAGACCCAGGGGTAT | | | |
| | CCGGTCTCCGACTGTACTGCCGAGGCT | | | |
| | CTCAAGGCCATCATCATGGTCCAGAAT | | | |
| | ACGCCTGGATACGAGGATCTGAAGAA | | | |
| | ACAAGTGTCTGACAAGCGGAAACACA | | | |
| | CTGCCATCGATCTACTTTTGGGAATGC | | | |
| | AGAACGTGGGCTCGTTTGAACCGGGCT | | | |
| | CTTTCGCCTCCTATGAGCCTATCCGGG | | | |
| | CGTCGTCCATGCTGGAGAAGATCAATC | | | |
| | CGGCCGAGGTGTTTGGAAACATCATGG | | | |
| | TGGAGTATCCGTACGTGGAATGCACTG | | | |
| | ATTCTGTTGTTCTGGGTCTCTCCTACTT | | | |
| | TCGAAAGTACCACGATTACCGCAACGA | | | |
| | AGACGTGGACCGAGCCATCTCTGCTGC | | | |
| | CATTGGATACATTATTCGAGAGCAGCA | | | |
| | GCCTGACGGCGGCTTCTTTGGCTCCTG | | | |
| | GGGCGTGTGCTACTGCTACGCTCACAT | | | |
| | GTTTGCCATGGAGGCTCTGGAGACGCA | | | |
| | GAATCTCAACTATAACAACTGTTCCAC | | | |
| | GGTTCAAAAGGCGTGCGACTTTCTGGC | | | |
| | GGGCTACCAGGAAGCAGATGGAGGCT | | | |
| | GGGCCGAGGACTTTAAGTCGTGCGAGA | | | |
| | CCCAGATGTACGTGCGCGGACCCCATT | | | |
| | CGCTGGTCGTGCCTACTGCCATGGCCC | | | |
| | TGTTGAGTTTGATGAGTGGTCGGTATC | | | |
| | CCCAGGAGGACAAGATTCATGCTGCGG | | | |
| | CCCGGTTTCTCATGAGCAAGCAGATGA | | | |
| | GCAACGGTGAGTGGCTCAAGGAGGAG | | | |
| | ATGGAGGGGGTGTTTAACCATACTTGT | | | |
| | GCCATTGAGTATCCCAACTACCGGTTT | | | |
| | TATTTTGTCATGAAGGCTTTGGGGTTGT | | | |
| | ATTTCAAGGGATATTGCCAGTGA | | | |
| 948822 | ATGGGAATCCACGAAAGTGTGTCGAA | 66 | MGIHESVSKQFAKNGHSKY | 87 |
| | ACAGTTTGCGAAAAACGGACATTCCAA | | RSDRYGLPKTDLRRWTFHA | |
| | GTACCGCAGCGACCGATACGGCTTACC | | SDLGAQWWKYDDTTPLEE | |
| | TAAGACGGATCTGCGACGATGGACGTT | | LEKRATDYVKYSLELPGYA | |
| | CCACGCGTCCGATCTGGGGGCGCAATG | | PVTLDSKPVKNAYEAALKN | |
| | GTGGAAGTATGACGATACCACACCGCT | | WHLFASLQDPDSGAWQSE | |
| | GGAAGAGCTGGAAAAGAGGGCTACCG | | YDGPQFMSIGYVTACYFGG | |
| | ACTACGTCAAATACTCGCTGGAGCTGC | | NEIPTPVKTEMIRYIVNTAH | |
| | CGGGATACGCGCCCGTGACTCTGGACT | | PVDGGWGLHKEDKSTCFG | |
| | CCAAGCCCGTGAAAAATGCCTACGAA | | TSINYVVLRLLGLSRDHPVC | |
| | GCGGCTCTCAAAAACTGGCATCTGTTT | | VKARKTLVTKFGGAINNPH | |
| | GCGTCGCTGCAAGACCCCGACTCCGGC | | WGKTWLSILNLYKWEGVN | |
| | GCATGGCAGTCGGAATACGACGGACC | | PAPGELWLLPYFVPVHPGR | |
| | GCAGTTCATGTCGATCGGTTATGTGAC | | WWVHTRWIYLAMGYLEA | |
| | GGCGTGCTACTTTGGCGGCAACGAGAT | | AEAQCELTPLLEELRDEIYI | |
| | CCCCACGCCGGTCAAAACCGAAATGAT | | KPYSEIDFSKHCNSISGVDL | |
| | CAGATACATTGTCAACACAGCCCACCC | | YYPHTGLLKFGSALLRRYR | |
| | AGTTGACGGAGGCTGGGGCCTTCACAA | | KFRPQWIKEKVKEEIYNLC | |
| | AGAAGACAAGAGCACCTGTTTCGGTAC | | LREVSNTRHLCLAPVNNAM | |
| | CAGCATCAACTACGTGGTCCTGCGACT | | TSIVMYLHEGLDSANYKKI | |
| | ACTGGGCCTGTCACGGGATCATCCGGT | | AARWPEFLSLNPSGMFMN | |
| | CTGCGTCAAGGCGCGCAAAACGCTGGT | | GTNGLQVWDTAFAVQYAC | |
| | CACCAAGTTTGGCGGCGCCATCAACAA | | VCGFAELPQYQKTIRAAFD | |
| | CCCCCATTGGGGCAAGACCTGGCTGTC | | FLDRSQINEPTEENSYRDDR | |
| | GATTCTCAATCTCTACAAATGGGAGGG | | VGGWPFSTKTQGYPVSDCT | |
| | TGTGAATCCGGCCCCTGGCGAGCTCTG | | AEALKAIIMVQNTPGYEDL | |
| | GCTGTTGCCCTACTTTGTTCCTGTTCAT | | KKQVSDKRKHTAIDLLLGM | |
| | CCGGGCCGATGGTGGGTCCATACCCGG | | QNVGSFEPGSFASYEPIRAS | |
| | TGGATCTACCTTGCCATGGGCTATCTG | | SMLEKINPAEVFGNIMVEY | |
| | GAGGCTGCGGAGGCCCAATGCGAACT | | PYVECTDSVVLGLSYFRKY | |
| | CACTCCGTTGCTGGAGGAGCTCCGAGA | | HDYRNEDVDRAISAAIGYII | |
| | CGAAATCTACATAAAGCCCTACTCGGA | | REQQPDGGFFGSWGVCYC | |
| | GATTGATTTCTCCAAACATTGCAACTC | | YAHMFAMEALETQNLNYN | |
| | CATCTCCGGAGTCGACCTCTACTATCC | | NCSTVQKACDFLAGYQEA | |
| | CCACACCGGCCTTTTGAAGTTTGGCAG | | DGGWAEDFKSCETQMYVR | |
| | CGCGCTTCTCCGACGATACCGCAAGTT | | GPHSLVVPTAMALLSLMSG | |
| | CAGACCGCAGTGGATCAAAGAAAAGG | | RYPQEDKIHAAARFLMSKQ | |
| | TCAAGGAGGAAATTTACAACTTGTGCC | | MSNGEWLKEEMEGVFNHT | |
| | TTCGAGAGGTTTCCAACACACGACACT | | CAIEYPNYRFYFVMKALGL | |
| | TGTGTCTCGCTCCCGTCAACAATGCCA | | YFKGYCQ | |
| | TGACCTCCATTGTCATGTATCTCCATGA | | | |
| | GGGGCTCGATTCGGCGAATTACAAAAA | | | |
| | GATTGCGGCCCGATGGCCCGAATTTCT | | | |
| | GTCTCTGAATCCGTCGGGAATGTTTAT | | | |
| | GAACGGCACCAACGGTCTGCAGGTCTG | | | |
| | GGATACTGCGTTTGCCGTGCAATACGC | | | |
| | GTGTGTTTGTGGCTTTGCCGAACTTCCC | | | |
| | CAGTACCAGAAGACGATCCGAGCGGC | | | |
| | GTTTGATTTTCTCGATCGGTCCCAGATC | | | |
| | AACGAGCCGACGGAGGAAAATTCCTA | | | |
| | TCGAGACGACCGCGTCGGAGGATGGC | | | |
| | CCTTTAGTACCAAGACCCAGGGGTATC | | | |
| | CAGTCTCCGACTGTACTGCCGAGGCTC | | | |
| | TCAAGGCCATCATCATGGTCCAGAATA | | | |
| | CGCCTGGATACGAGGATCTGAAGAAA | | | |
| | CAAGTGTCTGACAAGCGGAAACACACT | | | |
| | GCCATCGATCTACTTTTGGGAATGCAG | | | |
| | AACGTGGGCTCGTTTGAACCGGGCTCT | | | |
| | TTCGCCTCCTATGAGCCTATCCGGGCG | | | |
| | TCGTCCATGCTGGAGAAGATCAATCCG | | | |
| | GCCGAGGTGTTTGGAAACATCATGGTG | | | |
| | GAGTATCCGTACGTGGAATGCACTGAT | | | |
| | TCTGTTGTTCTGGGTCTGTCCTACTTTC | | | |
| | GAAAGTACCACGATTACCGCAACGAA | | | |
| | GACGTGGACCGAGCCATCTCTGCTGCC | | | |
| | ATTGGATATATTATTCGAGAGCAGCAG | | | |
| | CCTGACGGCGGCTTCTTTGGCTCCTGG | | | |
| | GGCGTGTGCTACTGCTACGCTCACATG | | | |
| | TTTGCCATGGAGGCTCTGGAGACGCAG | | | |
| | AATCTCAACTATAACAACTGTTCCACG | | | |
| | GTTCAAAAGGCGTGCGACTTTCTGGCG | | | |
| | GGCTACCAGGAAGCAGATGGAGGCTG | | | |
| | GGCCGAGGACTTTAAGTCGTGCGAGAC | | | |
| | CCAGATGTACGTGCGCGGACCCCATTC | | | |
| | GCTGGTCGTGCCTACTGCCATGGCCCT | | | |
| | GTTGAGTTTGATGAGTGGTCGGTATCC | | | |
| | CCAGGAGGACAAGATTCATGCTGCGGC | | | |
| | CCGGTTTCTCATGAGCAAGCAGATGAG | | | |
| | CAACGGTGAGTGGCTCAAGGAGGAGA | | | |
| | TGGAGGGGGTGTTTAACCATACTTGTG | | | |
| | CCATTGAGTATCCCAACTACCGGTTTT | | | |
| | ATTTTGTCATGAAGGCTTTGGGGTTGT | | | |
| | ATTTCAAGGGATATTGCCAGTGA | | | |
| 948823 | ATGGGAATCCACGAAAGTGTGTCGAA | 63 | MGIHESVSKQFAKNGHSKY | 84 |
| | ACAGTTTGCGAAAAACGGACATTCCAA | | RSDRYGLPKTDLRRWTFHA | |
| | GTACCGCAGCGACCGATACGGCTTACC | | SDLGAQWWKYDDTTPLEE | |
| | TAAGACGGATCTGCGACGATGGACGTT | | LEKRATDYVKYSLELPGYA | |
| | CCACGCGTCCGATCTGGGGGCGCAATG | | PVTLDSKPVKNAYEAALKN | |
| | GTGGAAGTATGACGATACCACACCGCT | | WHLFASLQDPDSGAWQSE | |
| | GGAAGAGCTGGAAAAGAGGGCTACCG | | YDGPQFMSIGYVTACYFGG | |
| | ACTACGTCAAATACTCGCTGGAGCTGC | | NEIPTPVKTEMIRYIVNTAH | |
| | CGGGATACGCGCCCGTGACTCTGGACT | | PVDGGWGLHKEDKSTCFG | |
| | CCAAGCCCGTGAAAAATGCCTACGAA | | TSNNYVVLRLLGLSRDHPV | |
| | GCGGCTCTCAAAAACTGGCATCTGTTT | | CVKARKTLLTKFGGAINNP | |
| | GCGTCGCTGCAAGACCCCGACTCCGGC | | HWGKTWLSILNLYKWEGV | |
| | GCATGGCAGTCGGAATACGACGGACC | | NPAPGELWLLPYFVPVHPG | |
| | GCAGTTCATGTCGATCGGTTATGTGAC | | RWWVHTRWIYLAMGYLE | |
| | GGCGTGCTACTTTGGCGGCAACGAGAT | | AAEAQCELTPLLEELRDEIY | |
| | CCCCACGCCGGTCAAAACCGAAATGAT | | KKPYSEIDFSKHCNSISGVD | |
| | CAGATACATTGTCAACACAGCCCACCC | | LYYPHTGLLKFGNALLRRY | |
| | AGTTGACGGAGGCTGGGGCCTTCACAA | | RKFRPQWIKEKVKEEIYNL | |
| | AGAAGACAAGAGCACCTGTTTCGGTAC | | CLREVSNTRHLCLAPVNNA | |
| | CAGCAACAACTACGTGGTCCTGCGACT | | MTSIVMYLHEGPDSANYKK | |
| | ACTGGGCCTGTCACGGGATCATCCGGT | | IAARWPEFLSLNPSGMFMN | |
| | CTGCGTCAAGGCGCGCAAAACGCTGCT | | GTNGLQVWDTAFAVQYAS | |
| | CACCAAGTTTGGCGGCGCCATCAACAA | | VCGFAELPQYQKTIRAAFD | |
| | CCCCCATTGGGGCAAGACCTGGCTGTC | | FLDRSQINEPTEENSYRDDR | |
| | GATTCTCAATCTCTACAAATGGGAGGG | | VGGWPFSTKTQGYPVSDCT | |
| | TGTGAATCCGGCCCCTGGCGAGCTCTG | | AEALKAIIMVQNTPGYEDL | |
| | GCTGTTGCCCTACTTTGTTCCTGTTCAT | | KKQVSDKRKHTAIDLLLGM | |
| | CCGGGCCGATGGTGGGTCCATACCCGG | | QNVGSFEPGSFASYEPIRAS | |
| | TGGATCTACCTTGCCATGGGCTATCTG | | SMLEKINPAEVFGNIMVEY | |
| | GAGGCTGCGGAGGCCCAATGCGAACT | | PYVECTDSVVMGLSYFRKY | |
| | CACTCCGTTGCTGGAGGAGCTCCGAGA | | HDYRNEDVDRAISAAIGYII | |
| | CGAAATCTACAAAAAGCCCTACTCGGA | | REQQPDGGFFGSWGVCYC | |
| | GATTGATTTCTCCAAACATTGCAACTC | | YAHMFAMEALETQNLNYN | |
| | CATCTCCGGAGTCGACCTCTACTATCC | | NCSTVQKACDFLAGYQEA | |
| | CCACACCGGCCTTTTGAAGTTTGGCAA | | DGGWAEDFKSCETQMYVR | |
| | CGCGCTTCTCCGACGATACCGCAAGTT | | GPHSLVVPTAMALLSLMSS | |
| | CAGACCGCAGTGGATCAAAGAAAAGG | | RYPQEDKIHAAARFLMSKQ | |
| | TCAAGGAGGAAATTTACAACTTGTGCC | | MSNGEWLKEEMEGVFNHT | |
| | TTCGAGAGGTTTCCAACACACGACACT | | CAIEYPNYRFYFVMKALGL | |
| | TGTGTCTCGCTCCCGTCAACAATGCCA | | YFKGYCQ | |
| | TGACCTCCATTGTCATGTATCTCCATGA | | | |
| | GGGGCCCGATTCGGCGAATTACAAAA | | | |
| | AGATTGCGGCCCGATGGCCCGAATTTC | | | |
| | TGTCTCTGAATCCGTCGGGAATGTTTA | | | |
| | TGAACGGCACCAACGGTCTGCAGGTCT | | | |
| | GGGATACTGCGTTTGCCGTGCAATACG | | | |
| | CGAGTGTTTGTGGCTTTGCCGAACTTC | | | |
| | CCCAGTACCAGAAGACGATCCGAGCG | | | |
| | GCGTTTGATTTTCTCGATCGGTCCCAG | | | |
| | ATCAACGAGCCGACGGAGGAAAATTC | | | |
| | CTATCGAGACGACCGCGTCGGAGGATG | | | |
| | GCCCTTTAGTACCAAGACCCAGGGGTA | | | |
| | TCCAGTCTCCGACTGTACTGCCGAGGC | | | |
| | TCTCAAGGCCATCATCATGGTCCAGAA | | | |
| | TACGCCTGGATACGAGGATCTGAAGAA | | | |
| | ACAAGTGTCTGACAAGCGGAAACACA | | | |
| | CTGCCATCGATCTACTTTTGGGAATGC | | | |
| | AGAACGTGGGCTCGTTTGAACCGGGCT | | | |
| | CTTTCGCCTCCTATGAGCCTATCCGGG | | | |
| | CGTCGTCCATGCTGGAGAAGATCAATC | | | |
| | CGGCCGAGGTGTTTGGAAACATCATGG | | | |
| | TGGAGTATCCGTACGTGGAATGCACTG | | | |
| | ATTCTGTTGTTATGGGTCTGTCCTACTT | | | |
| | TCGAAAGTACCACGATTACCGCAACGA | | | |
| | AGACGTGGACCGAGCCATCTCTGCTGC | | | |
| | CATTGGATACATTATTCGAGAGCAGCA | | | |
| | GCCTGACGGCGGCTTCTTTGGCTCCTG | | | |
| | GGGCGTGTGCTACTGCTACGCTCACAT | | | |
| | GTTTGCCATGGAGGCTCTGGAGACGCA | | | |
| | GAATCTCAACTATAACAACTGTTCCAC | | | |
| | GGTTCAAAAGGCGTGCGACTTTCTGGC | | | |
| | GGGCTACCAGGAAGCAGATGGAGGCT | | | |
| | GGGCCGAGGACTTTAAGTCGTGCGAGA | | | |
| | CTCAGATGTACGTGCGCGGACCCCATT | | | |
| | CGCTGGTCGTGCCTACTGCCATGGCCC | | | |
| | TGTTGAGTTTGATGAGTAGTCGGTATC | | | |
| | CCCAGGAGGACAAGATTCATGCTGCGG | | | |
| | CCCGGTTTCTCATGAGCAAGCAGATGA | | | |
| | GCAACGGTGAGTGGCTCAAGGAGGAG | | | |
| | ATGGAGGGGGTGTTTAACCATACTTGT | | | |
| | GCCATTGAGTATCCCAACTACCGGTTT | | | |
| | TATTTTGTCATGAAGGCTTTGGGGTTGT | | | |
| | ATTTCAAGGGATATTGCCAGTGA | | | |
| 948825 | ATGGGAATCCACGAAAGTGTGTCGAA | 68 | MGIHESVSKQFAKNGHSKY | 89 |
| | ACAGTTTGCGAAAAACGGACATTCCAA | | RSDRYGLPKTDLRRWTFHA | |
| | GTACCGCAGCGACCGATACGGCTTACC | | SDLGAQWWKYDDTTPLEE | |
| | TAAGACGGATCTGCGACGATGGACGTT | | LEKRATDYVKYSLELPGYA | |
| | CCACGCGTCCGATCTGGGGGCGCAATG | | PVTLDSKPVKNAYEAALKN | |
| | GTGGAAGTATGACGATACCACACCGCT | | WHLFASLQDPDSGAWQSE | |
| | GGAAGAGCTGGAAAAGAGGGCTACCG | | YDGPQFMSIGYVTACYFGG | |
| | ACTACGTCAAATACTCGCTGGAGCTGC | | NEIPTPVKTEMIRYIVNTAH | |
| | CGGGATACGCGCCCGTGACTCTGGACT | | PVDGGWGLHKEDKSTCFG | |
| | CCAAGCCCGTGAAAAATGCCTACGAA | | TSNNYVVLRLLGLSRDHPV | |
| | GCGGCTCTCAAAAACTGGCATCTGTTT | | CVKARKTLLTKFGGAINNP | |
| | GCGTCGCTGCAAGACCCCGACTCCGGC | | HWGKTWLSILNLYKWEGV | |
| | GCATGGCAGTCGGAATACGACGGACC | | NPAPGELWLLPYFVPVHPG | |
| | GCAGTTCATGTCGATCGGTTATGTGAC | | RWWVHTRWIYLAMGYLE | |
| | GGCGTGCTACTTTGGCGGCAACGAGAT | | AAEAQCELTPLLEELRDEIY | |
| | CCCCACGCCGGTCAAAACCGAAATGAT | | KKPYSEIDFSKHCNSISGVD | |
| | CAGATACATTGTCAACACAGCCCACCC | | LYYPHTGLLKFGNALLRRY | |
| | AGTTGACGGAGGCTGGGGCCTTCACAA | | RKFRPQWIKEKVKEEIYNL | |
| | AGAAGACAAGAGCACCTGTTTCGGTAC | | CLREVSNTRHLCLAPVNNA | |
| | CAGCAACAACTACGTGGTCCTGCGACT | | MTSIVMYLHEGPDSANYKK | |
| | ACTGGGCCTGTCACGGGATCATCCGGT | | IAARWPEFLSLNPSGMFMN | |
| | CTGCGTCAAGGCGCGCAAAACGCTGCT | | GTNGLQVWDTAFAVQYAS | |
| | CACCAAGTTTGGCGGCGCCATCAACAA | | VCGFAELPQYQKTIRAAFD | |
| | CCCCCATTGGGGCAAGACCTGGCTGTC | | FLDRSQINEPTEENSYRDDR | |
| | GATTCTCAATCTCTACAAATGGGAGGG | | VGGWPFSTKTQGYPVSDCT | |
| | TGTGAATCCGGCCCCTGGCGAGCTCTG | | AEALKAIIMVQNTPGYEDL | |
| | GCTGTTGCCCTACTTTGTTCCTGTTCAT | | KKQVSDKRKHTAIDLLLGM | |
| | CCGGGCCGATGGTGGGTCCATACCCGG | | QNVGSFEPGSFASYEPIRAS | |
| | TGGATCTACCTTGCCATGGGCTATCTG | | SMLEKINPAEVFGNIMVEY | |
| | GAGGCTGCGGAGGCCCAATGCGAACT | | PYVECTDSVVMGLSYFRKY | |
| | CACTCCGTTGCTGGAGGAGCTCCGAGA | | HDYRNEDVDRAISAAIGYII | |
| | CGAAATCTACAAAAAGCCCTACTCGGA | | REQQPDGGFFGSWGVCYC | |
| | GATTGATTTCTCCAAACATTGCAACTC | | YAHMFAMEALETQNLNYN | |
| | CATCTCCGGAGTCGACCTCTACTATCC | | NCSTVQKACDFLAGYQEA | |
| | CCACACCGGCCTTTTGAAGTTTGGCAA | | DGGWAEDFKSCETQMYVR | |
| | CGCGCTTCTCCGACGATACCGCAAGTT | | GPHSLVVPTAMALLSLMSG | |
| | CAGACCGCAGTGGATCAAAGAAAAGG | | RYPQEDKIHAAARFLMSKQ | |
| | TCAAGGAGGAAATTTACAACTTGTGCC | | MSNGEWLKEEMEGVFNHT | |
| | TTCGAGAGGTTTCCAACACACGACACT | | CAIEYPNYRFYFVMKALGL | |
| | TGTGTCTCGCTCCCGTCAACAATGCCA | | YFKGYCQ | |
| | TGACCTCCATTGTCATGTATCTCCATGA | | | |
| | GGGGCCCGATTCGGCGAATTACAAAA | | | |
| | AGATTGCGGCCCGATGGCCCGAATTTC | | | |
| | TGTCTCTGAATCCGTCGGGAATGTTTA | | | |
| | TGAACGGCACCAACGGTCTGCAGGTCT | | | |
| | GGGATACTGCGTTTGCCGTGCAATACG | | | |
| | CGAGTGTTTGTGGCTTTGCCGAACTTC | | | |
| | CCCAGTACCAGAAGACGATCCGAGCG | | | |
| | GCGTTTGATTTTCTCGATCGGTCCCAG | | | |
| | ATCAACGAGCCGACGGAGGAAAATTC | | | |
| | CTATCGAGACGACCGCGTCGGAGGATG | | | |
| | GCCCTTTAGTACCAAGACCCAGGGGTA | | | |
| | TCCAGTCTCCGACTGTACTGCCGAGGC | | | |
| | TCTCAAGGCCATCATCATGGTCCAGAA | | | |
| | TACGCCTGGATACGAGGATCTGAAGAA | | | |
| | ACAAGTGTCTGACAAGCGGAAACACA | | | |
| | CTGCCATCGATCTACTTTTGGGAATGC | | | |
| | AGAACGTGGGCTCGTTTGAACCGGGCT | | | |
| | CTTTCGCCTCCTATGAGCCTATCCGGG | | | |
| | CGTCGTCCATGCTGGAGAAGATCAATC | | | |
| | CGGCCGAGGTGTTTGGAAACATCATGG | | | |
| | TGGAGTATCCGTACGTGGAATGCACTG | | | |
| | ATTCTGTTGTTATGGGTCTGTCCTACTT | | | |
| | TCGAAAGTACCACGATTACCGCAACGA | | | |
| | AGACGTGGACCGAGCCATCTCTGCTGC | | | |
| | CATTGGATACATTATTCGAGAGCAGCA | | | |
| | GCCTGACGGCGGCTTCTTTGGCTCCTG | | | |
| | GGGCGTGTGCTACTGCTACGCTCACAT | | | |
| | GTTTGCCATGGAGGCTCTGGAGACGCA | | | |
| | GAATCTCAACTATAACAACTGTTCCAC | | | |
| | GGTTCAAAAGGCGTGCGACTTTCTGGC | | | |
| | GGGCTACCAGGAAGCAGATGGAGGCT | | | |
| | GGGCCGAGGACTTTAAGTCGTGCGAGA | | | |
| | CCCAGATGTACGTGCGCGGACCCCATT | | | |
| | CGCTGGTCGTGCCTACTGCCATGGCCC | | | |
| | TGTTGAGTTTGATGAGTGGTCGGTATC | | | |
| | CCCAGGAGGACAAGATTCATGCTGCGG | | | |
| | CCCGGTTTCTCATGAGCAAGCAGATGA | | | |
| | GCAACGGTGAGTGGCTCAAGGAGGAG | | | |
| | ATGGAGGGGGTGTTTAACCATACTTGT | | | |
| | GCCATTGAGTATCCCAACTACCGGTTT | | | |
| | TATTTTGTCATGAAGGCTTTGGGGTTGT | | | |
| | ATTTCAAGGGATATTGCCAGTGA | | | |
| 950865 | ATGGGAATCCACGAAAGTGTGTCGAA | 69 | MGIHESVSKQFAKNGHSKY | 90 |
| | ACAGTTTGCGAAAAACGGACATTCCAA | | RSDRYGLPKTDLRRWTFHA | |
| | GTACCGCAGCGACCGATACGGCTTACC | | SDLGAQWWKYDDTTPLEE | |
| | TAAGACGGATCTGCGACGATGGACGTT | | LEKRATDYVKYSLELPGYA | |
| | CCACGCGTCCGATCTGGGGGCGCAATG | | PVTLDSKPVKNAYEAALKN | |
| | GTGGAAGTATGACGATACCACACCGCT | | WHLFASLQDPDSGAWQSE | |
| | GGAAGAGCTGGAAAAGAGGGCTACCG | | YDGPQFMSIGYVTACYFGG | |
| | ACTACGTCAAATACTCGCTGGAGCTGC | | NEIPTPVKTEMIRYIVNTAH | |
| | CGGGATACGCGCCCGTGACTCTGGACT | | PVDGGWGLHKEDKSTCFG | |
| | CCAAGCCCGTGAAAAATGCCTACGAA | | TSINYVVLRLLGLSRDHPVC | |
| | GCGGCTCTCAAAAACTGGCATCTGTTT | | VKARKTLLTKFGGAINNPH | |
| | GCGTCGCTGCAAGACCCCGACTCCGGC | | WGKTWLSILNLYKWEGVN | |
| | GCGTGGCAGAGCGAATACGACGGACC | | PAPGELWLLPYFVPVHPGR | |
| | GCAGTTCATGAGCATCGGCTATGTGAC | | WWVHTRWIYLAMGYLEA | |
| | GGCGTGCTACTTTGGCGGCAACGAGAT | | AEAQCELTPLLEELRDEIYK | |
| | CCCCACGCCGGTCAAAACCGAAATGAT | | KPYSEIDFSKHCNSISGVDL | |
| | CAGATACATTGTCAACACAGCCCACCC | | YYPHTGLLKFGNALLRRYR | |
| | AGTTGACGGAGGCTGGGGCCTTCACAA | | KFRPQWIKEKVKEEIYNLC | |
| | AGAAGACAAGAGCACCTGCTTCGGTAC | | LREVSNTRHLCLAPVNNAM | |
| | CAGCATCAACTACGTGGTCCTGCGACT | | TSIVMYLHEGPVSANYKKI | |
| | ACTGGGCCTGTCACGGGACCATCCGGT | | AARWPEFLSLNPSGMFMN | |
| | CTGCGTCAAGGCGCGCAAAACGCTGCT | | GTNGLQVWDTAFAVQYAC | |
| | CACCAAGTTTGGCGGCGCCATCAACAA | | VCGFAELPQYQKTIRAAFD | |
| | CCCCCATTGGGGCAAGACCTGGCTGTC | | FLDRSQINEPTEENSYRDDR | |
| | GATTCTCAATCTCTACAAATGGGAGGG | | VGGWPFSTKTQGYPVSDCT | |
| | TGTGAATCCGGCCCCTGGCGAGCTCTG | | AEALKAIIMVQNTPGYEDL | |
| | GCTGTTGCCCTACTTTGTTCCTGTTCAT | | KKQVSDKRKHTAIDLLLGM | |
| | CCGGGCCGATGGTGGGTCCATACCCGG | | QNVGSFEPGSFASYEPIRAS | |
| | TGGATCTACCTTGCCATGGGCTATCTG | | SMLEKINPAEVFGNIMVEY | |
| | GAGGCTGCGGAGGCCCAATGCGAACT | | PYVECTDSVVLGLSYFRKY | |
| | CACTCCGTTGCTGGAGGAGCTCCGAGA | | HDYRNEDVDRAISAAIGYII | |
| | CGAAATCTACAAAAAGCCCTACTCGGA | | REQQPDGGFFGSWGVCYC | |
| | GATTGATTTCTCCAAACATTGCAACTC | | YAHIFAMEALETQNLNYNN | |
| | CATCTCCGGAGTCGACCTCTACTATCC | | CSTVQKACDFLAGYQEAD | |
| | CCACACCGGCCTTTTGAAGTTTGGCAA | | GGWAEDFKSCETQMYVRG | |
| | CGCGCTTCTCCGACGATACCGCAAGTT | | PHSLVVPTAMALLSLMSGR | |
| | CAGACCGCAGTGGATCAAAGAAAAGG | | YPQEDKIHAAARFLMSKQ | |
| | TCAAGGAGGAAATTTACAACTTGTGCC | | MSNDEWLKEEMEGVFNHT | |
| | TTCGAGAGGTTTCCAACACACGACACT | | CAIEYPNYRFYFVMKALGL | |
| | TGTGTCTCGCTCCCGTCAACAATGCCA | | YFKGYCQ | |
| | TGACCTCCATTGTCATGTATCTCCATGA | | | |
| | GGGGCCCGTTTCGGCGAATTACAAAAA | | | |
| | GATTGCGGCCCGATGGCCCGAATTTCT | | | |
| | GTCTCTGAATCCGTCGGGAATGTTTAT | | | |
| | GAACGGCACCAACGGTCTGCAGGTCTG | | | |
| | GGATACTGCGTTTGCCGTGCAATACGC | | | |
| | GTGTGTTTGTGGCTTTGCCGAACTTCCC | | | |
| | CAGTACCAGAAGACGATCCGAGCGGC | | | |
| | GTTTGATTTTCTCGATCGGTCCCAGATC | | | |
| | AACGAGCCGACGGAGGAAAATTCCTA | | | |
| | TCGAGACGACCGCGTCGGAGGATGGC | | | |
| | CCTTTAGTACCAAGACCCAGGGGTATC | | | |
| | CAGTCTCCGACTGTACTGCCGAGGCTC | | | |
| | TCAAGGCCATCATCATGGTCCAGAATA | | | |
| | CGCCTGGATACGAGGATCTGAAGAAA | | | |
| | CAAGTGTCTGACAAGCGGAAACACACT | | | |
| | GCCATCGATCTACTTTTGGGAATGCAG | | | |
| | AACGTGGGCTCGTTTGAACCGGGCTCT | | | |
| | TTCGCCTCCTATGAGCCTATCCGGGCG | | | |
| | TCGTCCATGCTGGAGAAGATCAATCCG | | | |
| | GCCGAGGTGTTTGGAAACATCATGGTG | | | |
| | GAGTATCCGTACGTGGAATGCACTGAT | | | |
| | TCTGTTGTTCTGGGTCTGTCCTACTTTC | | | |
| | GAAAGTACCACGATTACCGCAACGAA | | | |
| | GACGTGGACCGAGCCATCTCTGCTGCC | | | |
| | ATTGGATACATTATTCGAGAGCAGCAG | | | |
| | CCTGACGGCGGCTTCTTTGGCTCCTGG | | | |
| | GGCGTGTGCTACTGCTACGCTCACATA | | | |
| | TTTGCCATGGAGGCTCTGGAGACGCAG | | | |
| | AATCTCAACTATAACAACTGTTCCACG | | | |
| | GTTCAAAAGGCGTGCGACTTTCTGGCG | | | |
| | GGCTACCAGGAAGCAGATGGAGGCTG | | | |
| | GGCCGAGGACTTTAAGTCGTGCGAGAC | | | |
| | TCAGATGTACGTGCGCGGACCCCATTC | | | |
| | GCTGGTCGTGCCTACTGCCATGGCCCT | | | |
| | GTTGAGTTTGATGAGTGGTCGGTATCC | | | |
| | CCAGGAGGACAAGATTCATGCTGCGGC | | | |
| | CCGGTTTCTCATGAGCAAGCAGATGAG | | | |
| | CAACGATGAGTGGCTCAAGGAGGAGA | | | |
| | TGGAGGGGGTGTTTAACCATACTTGTG | | | |
| | CCATTGAGTATCCCAACTACCGGTTTT | | | |
| | ATTTTGTCATGAAGGCTTTGGGGTTGT | | | |
| | ATTTCAAGGGATATTGCCAGTGA | | | |
| 950866 | ATGGGAATCCACGAAAGTGTGTCGAA | 70 | MGIHESVSKQFAKNGHSKY | 91 |
| | ACAGTTTGCGAAAAACGGACATTCCAA | | RSDRYGLPKTDLRRWTFHA | |
| | GTACCGCAGCGACCGATACGGCTTACC | | SDLGAQWWKYDDTTPLEE | |
| | TAAGACGGATCTGCGACGATGGACGTT | | LEKRATDYVKYSLELPGYA | |
| | CCACGCGTCCGATCTGGGGGCGCAATG | | PVTLDSKPVKNAYEAALKN | |
| | GTGGAAGTATGACGATACCACACCGCT | | WHLFASLQDPDSGAWQSE | |
| | GGAAGAGCTGGAAAAGAGGGCTACCG | | YDGPQFMSIGYVTACYFGG | |
| | ACTACGTCAAATACTCGCTGGAGCTGC | | NEIPTPVKTEMIRYIVNTAH | |
| | CGGGATACGCGCCCGTGACTCTGGACT | | PVDGGWGLHKEDKSTCFG | |
| | CCAAGCCCGTGAAAAATGCCTACGAA | | TSINYVVLRLLGLSRDHPVC | |
| | GCGGCTCTCAAAAACTGGCATCTGTTT | | VKARKTLLTKFGGAINNPH | |
| | GCGTCGCTGCAAGACCCCGACTCCGGC | | WGKTWLSILNLYKWEGVN | |
| | GCGTGGCAGAGCGAATACGACGGACC | | PAPGELWLLPYFVPVHPGR | |
| | GCAGTTCATGAGCATCGGCTATGTGAC | | WWVHTRWIYLAMGYLEA | |
| | GGCGTGCTACTTTGGCGGCAACGAGAT | | AEAQCELTPLLEELRDEIYK | |
| | CCCCACGCCGGTCAAAACCGAAATGAT | | KPYSEIDFSKHCNSISGVDL | |
| | CAGATACATTGTCAACACAGCCCACCC | | YYPHTGLLKFGNALLRRYR | |
| | AGTTGACGGAGGCTGGGGCCTTCACAA | | KFRPQWIKEKVKEEIYNLC | |
| | AGAAGACAAGAGCACCTGCTTCGGTAC | | LREVSNTRHLCLAPVNNAM | |
| | CAGCATCAACTACGTGGTCCTGCGACT | | TSIVMYLHEGPVSANYKKI | |
| | ACTGGGCCTGTCACGGGACCATCCGGT | | AARWPEFLSLNPSGMFMN | |
| | CTGCGTCAAGGCGCGCAAAACGCTGCT | | GTNGLQVWDTAFAVQYAC | |
| | CACCAAGTTTGGCGGCGCCATCAACAA | | VCGFAELPQYQKTIRAAFD | |
| | CCCCCATTGGGGCAAGACCTGGCTGTC | | FLDRSQINEPTEENSYRDDR | |
| | GATTCTCAATCTCTACAAATGGGAGGG | | VGGWPFSTKTQGYPVSDCT | |
| | TGTGAATCCGGCCCCTGGCGAGCTCTG | | AEALKAIIMVQNTPGYEDL | |
| | GCTGTTGCCCTACTTTGTTCCTGTTCAT | | KKQVSDNRKHTAIDLLLGM | |
| | CCGGGCCGATGGTGGGTCCATACCCGG | | QNVGSFEPGSFASYEPIRAS | |
| | TGGATCTACCTTGCCATGGGCTATCTG | | SMLEKINPAEVFGNIMVEY | |
| | GAGGCTGCGGAGGCCCAATGCGAACT | | PYVECTDSVVLGLSYFRKY | |
| | CACTCCGTTGCTGGAGGAGCTCCGAGA | | HDYRNEDVDRAISAAIGYII | |
| | CGAAATCTACAAAAAGCCCTACTCGGA | | REQQPDGGFFGSWGVCYC | |
| | GATTGATTTCTCCAAACATTGCAACTC | | YAHIFAMEALETQNLNYNN | |
| | CATCTCCGGAGTCGACCTCTACTATCC | | CSTVQKACDFLAGYQEAD | |
| | CCACACCGGCCTTTTGAAGTTTGGCAA | | GGWAEDFKSCETQMYVRG | |
| | CGCGCTTCTCCGACGATACCGCAAGTT | | PHSLVVPTAMALLSLMSGR | |
| | CAGACCGCAGTGGATCAAAGAAAAGG | | YPQEDKIHAAARFLMSKQ | |
| | TCAAGGAGGAAATTTACAACTTGTGCC | | MSNDEWLKEEMEGVFNHT | |
| | TTCGAGAGGTTTCCAACACACGACACT | | CAIEYPNYRFYFVMKALGL | |
| | TGTGTCTCGCTCCCGTCAACAATGCCA | | YFKGYCQ | |
| | TGACCTCCATTGTCATGTATCTCCATGA | | | |
| | GGGGCCCGTTTCGGCGAATTACAAAAA | | | |
| | GATTGCGGCCCGATGGCCCGAATTTCT | | | |
| | GTCTCTGAATCCGTCGGGAATGTTTAT | | | |
| | GAACGGCACCAACGGTCTGCAGGTCTG | | | |
| | GGATACTGCGTTTGCCGTGCAATACGC | | | |
| | GTGTGTTTGTGGCTTTGCCGAACTTCCC | | | |
| | CAGTACCAGAAGACGATCCGAGCGGC | | | |
| | GTTTGATTTTCTCGATCGGTCCCAGATC | | | |
| | AACGAGCCGACGGAGGAAAATTCCTA | | | |
| | TCGAGACGACCGCGTCGGAGGATGGC | | | |
| | CCTTTAGTACCAAGACCCAGGGGTATC | | | |
| | CAGTCTCCGACTGTACTGCCGAGGCTC | | | |
| | TCAAGGCCATCATCATGGTCCAGAATA | | | |
| | CGCCTGGATACGAGGATCTGAAGAAA | | | |
| | CAAGTGTCTGACAATCGGAAACACACT | | | |
| | GCCATCGATCTACTTTTGGGAATGCAG | | | |
| | AACGTGGGCTCGTTTGAACCGGGCTCT | | | |
| | TTCGCCTCCTATGAGCCTATCCGGGCG | | | |
| | TCGTCCATGCTGGAGAAGATCAATCCG | | | |
| | GCCGAGGTGTTTGGAAACATCATGGTG | | | |
| | GAGTATCCGTACGTGGAATGCACTGAT | | | |
| | TCTGTTGTTCTGGGTCTGTCCTACTTTC | | | |
| | GAAAGTACCACGATTACCGCAACGAA | | | |
| | GACGTGGACCGAGCCATCTCTGCTGCC | | | |
| | ATTGGATACATTATTCGAGAGCAGCAG | | | |
| | CCTGACGGCGGCTTCTTTGGCTCCTGG | | | |
| | GGCGTGTGCTACTGCTACGCTCACATA | | | |
| | TTTGCCATGGAGGCTCTGGAGACGCAG | | | |
| | AATCTCAACTATAACAACTGTTCCACG | | | |
| | GTTCAAAAGGCGTGCGACTTTCTGGCG | | | |
| | GGCTACCAGGAAGCAGATGGAGGCTG | | | |
| | GGCCGAGGACTTTAAGTCGTGCGAGAC | | | |
| | TCAGATGTACGTGCGCGGACCCCATTC | | | |
| | GCTGGTCGTGCCTACTGCCATGGCCCT | | | |
| | GTTGAGTTTGATGAGTGGTCGGTATCC | | | |
| | CCAGGAGGACAAGATTCATGCTGCGGC | | | |
| | CCGGTTTCTCATGAGCAAGCAGATGAG | | | |
| | CAACGATGAGTGGCTCAAGGAGGAGA | | | |
| | TGGAGGGGGTGTTTAACCATACTTGTG | | | |
| | CCATTGAGTATCCCAACTACCGGTTTT | | | |
| | ATTTTGTCATGAAGGCTTTGGGGTTGT | | | |
| | ATTTCAAGGGATATTGCCAGTGA | | | |
| 950867 | ATGGGAATCCACGAAAGTGTGTCGAA | 71 | MGIHESVSKQFAKNGHSKY | 92 |
| | ACAGTTTGCGAAAAACGGACATTCCAA | | RSDRYGLPKTDLRRWTFHA | |
| | GTACCGCAGCGACCGATACGGCTTACC | | SDLGAQWWKYDDTTPLEE | |
| | TAAGACGGATCTGCGACGATGGACGTT | | LEKRATDYVKYSLELPGYA | |
| | CCACGCGTCCGATCTGGGGGCGCAATG | | PVTLGSKLVKNAYEAALKN | |
| | GTGGAAGTATGACGATACCACACCGCT | | WHLFASLQDPDSGAWQSE | |
| | GGAAGAGCTGGAAAAGAGGGCTACCG | | YDGPQFMSIGYVTACYFGG | |
| | ACTACGTCAAATACTCGCTGGAGCTGC | | NEIPTPVKTEMIRYIVNTAH | |
| | CGGGATACGCGCCCGTGACTCTGGGCT | | PVDGGWGLHKEDKSTCFG | |
| | CCAAGCTCGTGAAAAATGCCTACGAAG | | ASINYVVLRLLGLSRDHPV | |
| | CGGCTCTCAAAAACTGGCATCTGTTTG | | CVKARKTLLIKFGGAINNP | |
| | CGTCGCTGCAAGACCCCGACTCCGGCG | | HWGKTWLSILNLYKWEGV | |
| | CATGGCAGTCGGAATACGACGGACCG | | NPTPGELWLLPYFVPVHPG | |
| | CAGTTCATGTCGATCGGTTATGTGACG | | RWWVHTRWIYLAMGYLE | |
| | GCGTGCTACTTTGGCGGCAACGAGATC | | AAEAQCELTPLLEELRDEIY | |
| | CCCACGCCGGTCAAAACCGAAATGATC | | KKPYSEIDFSKHCNSISGVD | |
| | AGATACATTGTCAACACAGCCCACCCA | | LYYPHTGLLKFGNALLRRY | |
| | GTTGACGGAGGCTGGGGCCTTCACAAA | | RKFRPQWIKEKVKEEIYNL | |
| | GAAGACAAGAGCACCTGTTTCGGTGCC | | CLREVSNTRHLCLAPVNNA | |
| | AGCATCAACTACGTGGTCCTGCGACTA | | MTSIVMYLHEGPDSANYKK | |
| | CTGGGCCTGTCACGGGATCATCCGGTC | | IAARWPEFLSLNPSGMFMN | |
| | TGCGTCAAGGCGCGCAAAACGCTGCTC | | GTNGLQVWDTAFAVQYAC | |
| | ATCAAGTTTGGCGGCGCCATCAACAAC | | VCGFAELPQYQKTIRAAFD | |
| | CCCCATTGGGGCAAGACCTGGCTGTCG | | FLDRSQINEPTEENSYRDDR | |
| | ATTCTCAATCTCTACAAATGGGAGGGT | | VGGWPFSTKTQGYPVSDCT | |
| | GTGAATCCGACCCCTGGCGAGCTCTGG | | AEALKAIIMVQNTPGYEDL | |
| | CTGTTGCCCTACTTTGTTCCTGTTCATC | | KKQVSDKRKHTAIDLLLGM | |
| | CGGGCCGATGGTGGGTCCATACCCGGT | | QNVGSFEPGSFASYEPIRAS | |
| | GGATCTACCTTGCCATGGGCTATCTGG | | SMLEKINPAEVFGNIMVEY | |
| | AGGCTGCGGAGGCCCAATGCGAACTC | | PYVECTDSVVLGLSYFRKY | |
| | ACTCCGTTGCTGGAGGAGCTCCGAGAC | | HDYRNEDVDRAISAAIGYII | |
| | GAAATCTACAAAAAGCCCTACTCGGAG | | REQQPDGGFFGSWGVCYC | |
| | ATTGATTTCTCCAAACATTGCAACTCC | | YAHMFAMEALETQNLNYN | |
| | ATCTCCGGAGTCGACCTCTACTATCCC | | NCSTVQKACDFLAGYQEA | |
| | CACACCGGCCTTTTGAAGTTTGGCAAC | | DGGWAEDFKSCETQMYVR | |
| | GCGCTTCTCCGACGATACCGCAAGTTC | | GPHSLVVPTAMALLSLMSG | |
| | AGACCGCAGTGGATCAAAGAAAAGGT | | RYPQEDKIHAAARFLMSKQ | |
| | CAAGGAGGAAATTTACAACTTGTGCCT | | MSNGEWLKEEMEGVFNHT | |
| | TCGAGAGGTTTCCAACACACGACACTT | | CAIEYPNYRFYFVMKALGL | |
| | GTGTCTCGCTCCCGTCAACAATGCCAT | | YFKGYCQ | |
| | GACCTCCATTGTCATGTATCTCCATGA | | | |
| | GGGGCCCGATTCGGCGAATTACAAAA | | | |
| | AGATTGCGGCCCGATGGCCCGAATTTC | | | |
| | TGTCTCTGAATCCGTCGGGAATGTTTA | | | |
| | TGAACGGCACCAACGGTCTGCAGGTCT | | | |
| | GGGATACTGCGTTTGCCGTGCAATACG | | | |
| | CGTGTGTTTGTGGCTTTGCCGAACTTCC | | | |
| | CCAGTACCAGAAGACGATCCGAGCGG | | | |
| | CGTTTGATTTTCTCGATCGGTCCCAGAT | | | |
| | CAACGAGCCGACGGAGGAAAATTCCT | | | |
| | ATCGAGACGACCGCGTCGGAGGATGG | | | |
| | CCCTTTAGTACCAAGACCCAGGGGTAT | | | |
| | CCAGTCTCCGACTGTACTGCCGAGGCT | | | |
| | CTCAAGGCCATCATCATGGTCCAGAAT | | | |
| | ACGCCTGGATACGAGGATCTGAAGAA | | | |
| | ACAAGTGTCTGACAAGCGGAAACACA | | | |
| | CTGCCATCGATCTACTTTTGGGAATGC | | | |
| | AGAACGTGGGCTCGTTTGAACCGGGCT | | | |
| | CTTTCGCCTCCTATGAGCCTATCCGGG | | | |
| | CGTCGTCCATGCTGGAGAAGATCAATC | | | |
| | CGGCCGAGGTGTTTGGAAACATCATGG | | | |
| | TGGAGTATCCGTACGTGGAATGCACTG | | | |
| | ATTCTGTTGTTCTGGGTCTGTCCTACTT | | | |
| | TCGAAAGTACCACGATTACCGCAACGA | | | |
| | AGACGTGGACCGAGCCATCTCTGCTGC | | | |
| | CATTGGATACATTATTCGAGAGCAGCA | | | |
| | GCCTGACGGCGGCTTCTTTGGCTCCTG | | | |
| | GGGCGTGTGCTACTGCTACGCTCACAT | | | |
| | GTTTGCCATGGAGGCTCTGGAGACGCA | | | |
| | GAATCTCAACTATAACAACTGTTCCAC | | | |
| | GGTTCAAAAGGCGTGCGACTTTCTGGC | | | |
| | GGGCTACCAGGAAGCAGATGGAGGCT | | | |
| | GGGCCGAGGACTTTAAGTCGTGCGAGA | | | |
| | CTCAGATGTACGTGCGCGGACCCCATT | | | |
| | CGCTGGTCGTGCCTACTGCCATGGCCC | | | |
| | TGTTGAGTTTGATGAGTGGTCGGTATC | | | |
| | CCCAGGAGGACAAGATTCATGCTGCGG | | | |
| | CCCGGTTTCTCATGAGCAAGCAGATGA | | | |
| | GCAACGGTGAGTGGCTCAAGGAGGAG | | | |
| | ATGGAGGGGGTGTTTAACCATACTTGT | | | |
| | GCCATTGAGTATCCCAACTACCGGTTT | | | |
| | TATTTTGTCATGAAGGCTTTGGGGTTGT | | | |
| | ATTTCAAGGGATATTGCCAGTGA | | | |
| 950868 | ATGGGAATCCACGAAAGTGTGTCGAA | 4 | MGIHESVSKQFAKNGHSKY | 3 |
| | ACAGTTTGCGAAAAACGGACATTCCAA | | RSDRYGLPKTDLRRWTFHA | |
| | GTACCGCAGCGACCGATACGGCTTACC | | SDLGAQWWKYDGTTPLEE | |
| | TAAGACGGATCTGCGACGATGGACGTT | | LEKRATDYVRYSLELPGYA | |
| | CCACGCGTCCGATCTGGGGGCGCAATG | | PVTLDSKPVKNAYEAALKS | |
| | GTGGAAGTATGACGGTACCACACCGCT | | WHLFASLQDPDSGAWQSE | |
| | GGAAGAGCTGGAAAAGAGGGCTACCG | | YDGPQFMSIGYVTACYFGG | |
| | ACTACGTCAGATACTCGCTGGAGCTGC | | NEIPTPVKTEMIRYIVNTAH | |
| | CGGGATACGCGCCCGTGACTCTGGACT | | PVDGGWGLHKEDKSTCFG | |
| | CCAAGCCCGTGAAAAATGCCTACGAA | | TSINYVVLRLLGLSRDHPVC | |
| | GCGGCTCTCAAAAGCTGGCATCTGTTT | | VKARKTLLTKFGGAINNPH | |
| | GCGTCGCTGCAAGACCCCGACTCCGGC | | WGKTWLSILNLYKWEGVN | |
| | GCATGGCAGTCGGAATACGACGGACC | | PAPGELWLLPYFVPVHPGR | |
| | GCAGTTCATGTCGATCGGTTATGTGAC | | WWVHTRWIYLAMGYLEA | |
| | GGCGTGCTACTTTGGCGGCAACGAGAT | | AEAQCELTPLLEELRDEIYK | |
| | CCCCACGCCGGTCAAAACCGAAATGAT | | KPYSEIDFSKHCNSISGVDL | |
| | CAGATACATTGTCAACACAGCCCACCC | | YYPHTGLLKFGNALLRRYR | |
| | AGTTGACGGAGGCTGGGGCCTTCACAA | | KFRPQWIKEKVKEEIYNLC | |
| | AGAAGACAAGAGCACCTGTTTCGGTAC | | LREVSNTRHLCLAPVNNAM | |
| | CAGCATCAACTACGTGGTCCTGCGACT | | TSIVMYLHEGPDSANYKKI | |
| | ACTGGGCCTGTCACGGGATCATCCGGT | | AARWPEFLSLNPSGMFMN | |
| | CTGCGTCAAGGCGCGCAAAACGCTGCT | | GTNGLQVWDTAFAVQYAC | |
| | CACCAAGTTTGGCGGCGCCATCAACAA | | VCSFAELPQYQKTIRAAFDF | |
| | CCCCCATTGGGGCAAGACCTGGCTGTC | | LDRSQINEPTEENSYRDDRV | |
| | GATTCTCAATCTCTACAAATGGGAGGG | | GGWPFSTKTQGYPVSDCTA | |
| | TGTGAATCCGGCCCCTGGCGAGCTCTG | | EALKAIIMVQNTPGYEDLK | |
| | GCTGTTGCCCTACTTTGTTCCTGTTCAT | | KQVSDKRKHTAIDLLLGMQ | |
| | CCGGGCCGATGGTGGGTCCATACCCGG | | NVGSFEPGSFASYEPIRASS | |
| | TGGATCTACCTTGCCATGGGCTATCTG | | MLEKINPAEVFGNIMVEYP | |
| | GAGGCTGCGGAGGCCCAATGCGAACT | | YVECTDSVVLGLSYFRKYH | |
| | CACTCCGTTGCTGGAGGAGCTCCGAGA | | DYRNEDVDRAISAAIGYIIR | |
| | CGAAATCTACAAAAAGCCCTACTCGGA | | EQQPDGGFFGSWGVCYCY | |
| | GATTGATTTCTCCAAACATTGCAACTC | | AHMFAMEALVTQNLNYNN | |
| | CATCTCCGGAGTCGACCTCTACTATCC | | CSTVQKACDFLAGYQEAD | |
| | CCACACCGGCCTTTTGAAGTTTGGCAA | | GGWAEDFKSCETQMYVRG | |
| | CGCGCTTCTCCGACGATACCGCAAGTT | | PHSLVVPTAMALLSLMSGR | |
| | CAGACCGCAGTGGATCAAAGAAAAGG | | YPQEDKIHAAARFLMSKQ | |
| | TCAAGGAGGAAATTTACAACTTGTGCC | | MSNGEWLKEEMEGVFNHT | |
| | TTCGAGAGGTTTCCAACACACGACACT | | CAIEYPNYRLYFVMKALGL | |
| | TGTGTCTCGCTCCCGTCAACAATGCCA | | YFKGYCQ | |
| | TGACCTCCATTGTCATGTATCTCCATGA | | | |
| | GGGGCCCGATTCGGCGAATTACAAAA | | | |
| | AGATTGCGGCCCGATGGCCCGAATTTC | | | |
| | TGTCTCTGAATCCGTCGGGAATGTTTA | | | |
| | TGAACGGCACCAACGGTCTGCAGGTCT | | | |
| | GGGATACTGCGTTTGCCGTGCAATACG | | | |
| | CGTGTGTTTGTAGCTTTGCCGAACTTCC | | | |
| | CCAGTACCAGAAGACGATCCGAGCGG | | | |
| | CGTTTGATTTTCTCGATCGGTCCCAGAT | | | |
| | CAACGAGCCGACGGAGGAAAATTCCT | | | |
| | ATCGAGACGACCGCGTCGGAGGATGG | | | |
| | CCCTTTAGTACCAAGACCCAGGGGTAT | | | |
| | CCAGTCTCCGACTGTACTGCCGAGGCT | | | |
| | CTCAAGGCCATCATCATGGTCCAGAAT | | | |
| | ACGCCTGGATACGAGGATCTGAAGAA | | | |
| | ACAAGTGTCTGACAAGCGGAAACACA | | | |
| | CTGCCATCGATCTACTTTTGGGAATGC | | | |
| | AGAACGTGGGCTCGTTTGAACCGGGCT | | | |
| | CTTTCGCCTCCTATGAGCCTATCCGGG | | | |
| | CGTCGTCCATGCTGGAGAAGATCAATC | | | |
| | CGGCCGAGGTGTTTGGAAACATCATGG | | | |
| | TGGAGTATCCGTACGTGGAATGCACTG | | | |
| | ATTCTGTTGTTCTGGGTCTGTCCTACTT | | | |
| | TCGAAAGTACCACGATTACCGCAACGA | | | |
| | AGACGTGGACCGAGCCATCTCTGCTGC | | | |
| | CATCGGATACATTATTCGAGAGCAGCA | | | |
| | GCCTGACGGTGGCTTCTTTGGCTCCTG | | | |
| | GGGCGTGTGCTACTGCTACGCTCACAT | | | |
| | GTTTGCCATGGAGGCTCTGGTGACGCA | | | |
| | GAATCTCAACTATAACAACTGTTCCAC | | | |
| | GGTTCAAAAGGCGTGCGACTTTCTGGC | | | |
| | GGGCTACCAGGAAGCAGATGGAGGCT | | | |
| | GGGCCGAGGACTTTAAGTCGTGCGAGA | | | |
| | CTCAGATGTACGTGCGCGGACCCCATT | | | |
| | CGCTGGTCGTGCCTACTGCCATGGCCC | | | |
| | TGTTGAGTTTGATGAGTGGTCGGTATC | | | |
| | CCCAGGAGGACAAGATTCATGCTGCGG | | | |
| | CCCGGTTTCTCATGAGCAAGCAGATGA | | | |
| | GCAACGGTGAGTGGCTCAAGGAGGAG | | | |
| | ATGGAGGGGGTGTTTAACCATACTTGT | | | |
| | GCCATTGAGTATCCCAACTACCGGTTA | | | |
| | TATTTTGTCATGAAGGCTTTGGGGTTGT | | | |
| | ATTTCAAGGGATATTGCCAGTGA | | | |
| 950872 | ATGGGAATCCACGAAAGTGTGTCGAA | 73 | MGIHESVSKQFAKNGHSKY | 94 |
| | ACAGTTTGCGAAAAACGGACATTCCAA | | RSDRYGLPKTDLRRWTFHA | |
| | GTACCGCAGCGACCGATACGGCTTACC | | SDLGAQWWKYDDTTPLEE | |
| | TAAGACGGATCTGCGACGATGGACGTT | | LEKRATDYVKYSLELPGYA | |
| | CCACGCGTCCGATCTGGGGGCGCAATG | | PVTLDSKPVKNAYEAALKN | |
| | GTGGAAGTATGACGATACCACACCGCT | | WHLFASLQDPDSGAWQSE | |
| | GGAAGAGCTGGAAAAGAGGGCTACCG | | YDGPQFMSIGYVTACYFGG | |
| | ACTACGTCAAATACTCGCTGGAGCTGC | | NEIPTPVKTEMIRYIVNTAH | |
| | CGGGATACGCGCCCGTGACTCTGGACT | | PVDGGWGLHKEDKSTCFG | |
| | CCAAGCCCGTGAAAAATGCCTACGAA | | TSINYVVLRLLGLSRDHPVC | |
| | GCGGCTCTCAAAAACTGGCATCTGTTT | | VKARKTLLTKFGGAINNPH | |
| | GCGTCGCTGCAAGACCCCGACTCCGGC | | WGKTWLSILNLYKWEGVN | |
| | GCGTGGCAGAGCGAATACGACGGACC | | PAPGELWLLPYFVPVHPGR | |
| | GCAGTTCATGAGCATCGGCTATGTGAC | | WWVHTRWIYLAMGYLEA | |
| | GGCGTGCTACTTTGGCGGCAACGAGAT | | AEAQCELTPLLEELRDEIYK | |
| | CCCCACGCCGGTCAAAACCGAAATGAT | | KPYSEIDFSKHCNSISGVDL | |
| | CAGATACATTGTCAACACAGCCCACCC | | YYPHTGLLKFGNALLRRYR | |
| | AGTTGACGGAGGCTGGGGCCTTCACAA | | KFRPQWIKEKVKEEIYNLC | |
| | AGAAGACAAGAGCACCTGCTTCGGTAC | | LREVSNTRHLCLAPVNNAM | |
| | CAGCATCAACTACGTGGTCCTGCGACT | | SSIVMYLHEGPDPANYKKI | |
| | ACTGGGCCTGTCACGGGATCATCCGGT | | AARWPEFLSLNPSGMFMN | |
| | CTGCGTCAAGGCGCGCAAAACGCTGCT | | GTNGLQVWDTAFAVQYAC | |
| | CACCAAGTTTGGCGGCGCCATCAACAA | | VCGFAELPQYQKTIRAAFD | |
| | CCCCCATTGGGGCAAGACCTGGCTGTC | | FLDRSQINEPMEENSYRDD | |
| | GATTCTCAATCTCTACAAATGGGAGGG | | RVGGWPFSTKTQGYPVSDC | |
| | TGTGAATCCGGCCCCTGGCGAGCTCTG | | TAEALKAIIMVQNTPGYED | |
| | GCTGTTGCCCTACTTTGTTCCTGTTCAT | | LKKQVSDKRKHTAIDLLLG | |
| | CCGGGTCGATGGTGGGTCCATACCCGG | | MQNVGSFEPGSFASYEPIRA | |
| | TGGATCTACCTTGCCATGGGCTATCTG | | SSMLEKINPAEVFGNIMVE | |
| | GAGGCTGCGGAGGCCCAATGCGAACT | | YPYVECTDSVVLGLSYFRK | |
| | CACTCCGTTGCTGGAGGAGCTCCGAGA | | YHDYRNEDVDPAISAAIGYI | |
| | CGAAATCTACAAAAAGCCCTACTCGGA | | IREQQPDGGFFGSWGVCYC | |
| | GATTGATTTCTCCAAACATTGCAACTC | | YAHMFAMEALETQNLNYN | |
| | CATCTCCGGAGTCGACCTCTACTATCC | | NCSTVQKACDFLAGYQEA | |
| | CCACACCGGCCTTTTGAAGTTTGGCAA | | DGGWAEDFKSCETQMYVR | |
| | CGCGCTTCTCCGACGATACCGCAAGTT | | GPHSLVVPTAMALLSLMSG | |
| | CAGACCGCAGTGGATCAAAGAAAAGG | | RYPQEDKIHAAARFLMSKQ | |
| | TCAAGGAGGAAATTTACAACTTGTGCC | | MSNGEWLKEEMEGVFNHT | |
| | TTCGAGAGGTTTCCAACACACGACACT | | CAIEYPNYRFYFVMKALGL | |
| | TGTGTCTCGCTCCCGTCAACAATGCCA | | YFKGYCQ | |
| | TGTCCTCCATTGTCATGTATCTCCATGA | | | |
| | GGGGCCCGATCCGGCGAATTACAAAA | | | |
| | AGATTGCGGCCCGATGGCCCGAATTTC | | | |
| | TGTCTCTGAATCCGTCGGGAATGTTTA | | | |
| | TGAACGGCACCAACGGTCTGCAGGTCT | | | |
| | GGGATACTGCGTTTGCCGTGCAATACG | | | |
| | CGTGTGTTTGTGGCTTTGCCGAACTTCC | | | |
| | CCAGTACCAGAAGACGATCCGAGCGG | | | |
| | CGTTTGATTTTCTCGATCGGTCCCAGAT | | | |
| | CAACGAGCCGATGGAGGAAAATTCCT | | | |
| | ATCGAGACGACCGCGTCGGAGGATGG | | | |
| | CCCTTTAGTACCAAGACCCAGGGGTAT | | | |
| | CCAGTCTCCGACTGTACTGCCGAGGCT | | | |
| | CTCAAGGCCATCATCATGGTCCAGAAT | | | |
| | ACACCTGGATACGAGGATCTGAAGAA | | | |
| | ACAAGTGTCTGACAAGCGGAAACACA | | | |
| | CTGCCATCGATCTACTTTTGGGAATGC | | | |
| | AGAACGTGGGCTCGTTTGAACCGGGCT | | | |
| | CTTTCGCCTCCTATGAGCCTATCCGGG | | | |
| | CGTCGTCCATGCTGGAGAAGATCAATC | | | |
| | CGGCCGAGGTGTTTGGAAACATCATGG | | | |
| | TGGAGTATCCGTACGTGGAATGCACTG | | | |
| | ATTCTGTTGTTCTGGGTCTCTCCTACTT | | | |
| | TCGAAAGTACCACGATTACCGCAACGA | | | |
| | AGACGTGGACCCAGCCATCTCTGCTGC | | | |
| | CATTGGATACATTATTCGAGAGCAGCA | | | |
| | GCCTGACGGCGGCTTCTTTGGCTCCTG | | | |
| | GGGCGTGTGCTACTGCTACGCTCACAT | | | |
| | GTTTGCCATGGAGGCTCTGGAGACGCA | | | |
| | GAATCTCAACTATAACAACTGTTCCAC | | | |
| | GGTTCAAAAGGCGTGCGACTTTCTGGC | | | |
| | GGGCTACCAGGAAGCAGATGGAGGCT | | | |
| | GGGCCGAGGACTTTAAGTCGTGCGAGA | | | |
| | CCCAGATGTACGTGCGCGGACCCCATT | | | |
| | CGCTGGTCGTGCCTACTGCCATGGCCC | | | |
| | TGTTGAGTTTGATGAGTGGTCGGTATC | | | |
| | CCCAGGAGGACAAGATTCATGCTGCGG | | | |
| | CCCGGTTTCTCATGAGCAAGCAGATGA | | | |
| | GCAACGGTGAGTGGCTCAAGGAGGAG | | | |
| | ATGGAGGGGGTGTTTAACCATACTTGT | | | |
| | GCCATTGAGTATCCCAACTACCGGTTT | | | |
| | TATTTTGTCATGAAGGCTTTGGGGTTGT | | | |
| | ATTTCAAGGGATATTGCCAGTGA | | | |
| 950887 | ATGGGAATCCACGAAAGTGTGTCGAA | 74 | MGIHESVSKQFAKNGHSKY | 95 |
| | ACAGTTTGCGAAAAACGGACATTCCAA | | RSDRYGLPKTDLRRWTFHA | |
| | GTACCGCAGCGACCGATACGGCTTACC | | SDLGAQWWKYDGTTPLEE | |
| | TAAGACGGATCTGCGACGATGGACGTT | | LEKRATDYVRYSLELPGYA | |
| | CCACGCGTCCGATCTGGGGGCGCAATG | | PVTLDSKPVKNAYEAALKS | |
| | GTGGAAGTATGACGGTACCACACCGCT | | WHLFASLQDPDSGAWQSE | |
| | GGAAGAGCTGGAAAAGAGGGCTACCG | | YDGPQFMSIGYVTACYFGG | |
| | ACTACGTCAGATACTCGCTGGAGCTGC | | NEIPTPVKTEMIRYIVNTAH | |
| | CGGGATACGCGCCCGTGACTCTGGACT | | PVDGGWGLHKEDKSTCFG | |
| | CCAAGCCCGTGAAAAATGCCTACGAA | | TSINYVVLRLLGLSRDHPVC | |
| | GCGGCTCTCAAAAGCTGGCATCTGTTT | | VKARKTLLTKFGGAINNPH | |
| | GCGTCGCTGCAAGACCCCGACTCCGGC | | WGKTWLSILNLYKWEGVN | |
| | GCATGGCAGTCGGAATACGACGGACC | | PAPGELWLLPYFVPVHPGR | |
| | GCAGTTCATGTCGATCGGTTATGTGAC | | WWVHTRWIYLAMGYLEA | |
| | GGCGTGCTACTTTGGCGGCAACGAGAT | | AEAQCELTPLLEELRDEIYK | |
| | CCCCACGCCGGTCAAAACCGAAATGAT | | KPYSEIDFSKHCNSISGVDL | |
| | CAGATACATTGTCAACACAGCCCACCC | | YYPHTGLLKFGNALLRRYR | |
| | AGTTGACGGAGGCTGGGGCCTTCACAA | | KFRPQWIKEKVKEEIYNLC | |
| | AGAAGACAAGAGCACCTGTTTCGGTAC | | LREVSNTRHLCLAPVNNAM | |
| | CAGCATCAACTACGTGGTCCTGCGACT | | TSIVMYLHEGPDSANYKKI | |
| | ACTGGGCCTGTCACGGGATCATCCGGT | | AARWPEFLSLNPSGMFMN | |
| | CTGCGTCAAGGCGCGCAAAACGCTGCT | | GTNGLQVWDTAFAVQYAC | |
| | CACCAAGTTTGGCGGCGCCATCAACAA | | VCSFAELPQYQKTIRAAFDF | |
| | CCCCCATTGGGGCAAGACCTGGCTGTC | | LDRSQINEPTEENSYRDDRV | |
| | GATTCTCAATCTCTACAAATGGGAGGG | | GGWPFSTKTQGYPVSDCTA | |
| | TGTGAATCCGGCCCCTGGCGAGCTCTG | | EALKAIIMVQNTPGYEDLK | |
| | GCTGTTGCCCTACTTTGTTCCTGTTCAT | | KQVSDKRKHTAIDLLLGMQ | |
| | CCGGGCCGATGGTGGGTCCATACCCGG | | NVGSFEPGSFASYEPIRASS | |
| | TGGATCTACCTTGCCATGGGCTATCTG | | MLEKINPAEVFGNIMVEYP | |
| | GAGGCTGCGGAGGCCCAATGCGAACT | | YVECTDSVVLGLSYFRKYH | |
| | CACTCCGTTGCTGGAGGAGCTCCGAGA | | DYRNEDVDRAISAAIGYIIR | |
| | CGAAATCTACAAAAAGCCCTACTCGGA | | EQQPDGGFFGSWGVCYCY | |
| | GATTGATTTCTCCAAACATTGCAACTC | | AHMFAMEALVTQNLNYNN | |
| | CATCTCCGGAGTCGACCTCTACTATCC | | CSTVQKACDFLAGYQEAD | |
| | CCACACCGGCCTTTTGAAGTTTGGCAA | | GGWAEDFKSCETQMYVRG | |
| | CGCGCTTCTCCGACGATACCGCAAGTT | | PHSLVVPTAMALLSLMSGR | |
| | CAGACCGCAGTGGATCAAAGAAAAGG | | YPQEDKIHAAARFLMSKQ | |
| | TCAAGGAGGAAATTTACAACTTGTGCC | | MSNGEWLKEEMEGVFNHT | |
| | TTCGAGAGGTTTCCAACACACGACACT | | CAIEYPNYRFYFVMKALGL | |
| | TGTGTCTCGCTCCCGTCAACAATGCCA | | YFKGYCQ | |
| | TGACCTCCATTGTCATGTATCTCCATGA | | | |
| | GGGGCCCGATTCGGCGAATTACAAAA | | | |
| | AGATTGCGGCCCGATGGCCCGAATTTC | | | |
| | TGTCTCTGAATCCGTCGGGAATGTTTA | | | |
| | TGAACGGCACCAACGGTCTGCAGGTCT | | | |
| | GGGATACTGCGTTTGCCGTGCAATACG | | | |
| | CGTGTGTTTGTAGCTTTGCCGAACTTCC | | | |
| | CCAGTACCAGAAGACGATCCGAGCGG | | | |
| | CGTTTGATTTTCTCGATCGGTCCCAGAT | | | |
| | CAACGAGCCGACGGAGGAAAATTCCT | | | |
| | ATCGAGACGACCGCGTCGGAGGATGG | | | |
| | CCCTTTAGTACCAAGACCCAGGGGTAT | | | |
| | CCAGTCTCCGACTGTACTGCCGAGGCT | | | |
| | CTCAAGGCCATCATCATGGTCCAGAAT | | | |
| | ACGCCTGGATACGAGGATCTGAAGAA | | | |
| | ACAAGTGTCTGACAAGCGGAAACACA | | | |
| | CTGCCATCGATCTACTTTTGGGAATGC | | | |
| | AGAACGTGGGCTCGTTTGAACCGGGCT | | | |
| | CTTTCGCCTCCTATGAGCCTATCCGGG | | | |
| | CGTCGTCCATGCTGGAGAAGATCAATC | | | |
| | CGGCCGAGGTGTTTGGAAACATCATGG | | | |
| | TGGAGTATCCGTACGTGGAATGCACTG | | | |
| | ATTCTGTTGTTCTGGGTCTGTCCTACTT | | | |
| | TCGAAAGTACCACGATTACCGCAACGA | | | |
| | AGACGTGGACCGAGCCATCTCTGCTGC | | | |
| | CATCGGATACATTATTCGAGAGCAGCA | | | |
| | GCCTGACGGTGGCTTCTTTGGCTCCTG | | | |
| | GGGCGTGTGCTACTGCTACGCTCACAT | | | |
| | GTTTGCCATGGAGGCTCTGGTGACGCA | | | |
| | GAATCTCAACTATAACAACTGTTCCAC | | | |
| | GGTTCAAAAGGCGTGCGACTTTCTGGC | | | |
| | GGGCTACCAGGAAGCAGATGGAGGCT | | | |
| | GGGCCGAGGACTTTAAGTCGTGCGAGA | | | |
| | CTCAGATGTACGTGCGCGGACCCCATT | | | |
| | CGCTGGTCGTGCCTACTGCCATGGCCC | | | |
| | TGTTGAGTTTGATGAGTGGTCGGTATC | | | |
| | CCCAGGAGGACAAGATTCATGCTGCGG | | | |
| | CCCGGTTTCTCATGAGCAAGCAGATGA | | | |
| | GCAACGGTGAGTGGCTCAAGGAGGAG | | | |
| | ATGGAGGGGGTGTTTAACCATACTTGT | | | |
| | GCCATTGAGTATCCCAACTACCGGTTT | | | |
| | TATTTTGTCATGAAGGCTTTGGGGTTGT | | | |
| | ATTTCAAGGGATATTGCCAGTGA | | | |
| 950888 | ATGGGAATCCACGAAAGTGTGTCGAA | 71 | MGIHESVSKQFAKNGHSKY | 92 |
| | ACAGTTTGCGAAAAACGGACATTCCAA | | RSDRYGLPKTDLRRWTFHA | |
| | GTACCGCAGCGACCGATACGGCTTACC | | SDLGAQWWKYDDTTPLEE | |
| | TAAGACGGATCTGCGACGATGGACGTT | | LEKRATDYVKYSLELPGYA | |
| | CCACGCGTCCGATCTGGGGGCGCAATG | | PVTLGSKLVKNAYEAALKN | |
| | GTGGAAGTATGACGATACCACACCGCT | | WHLFASLQDPDSGAWQSE | |
| | GGAAGAGCTGGAAAAGAGGGCTACCG | | YDGPQFMSIGYVTACYFGG | |
| | ACTACGTCAAATACTCGCTGGAGCTGC | | NEIPTPVKTEMIRYIVNTAH | |
| | CGGGATACGCGCCCGTGACTCTGGGCT | | PVDGGWGLHKEDKSTCFG | |
| | CCAAGCTCGTGAAAAATGCCTACGAAG | | ASINYVVLRLLGLSRDHPV | |
| | CGGCTCTCAAAAACTGGCATCTGTTTG | | CVKARKTLLIKFGGAINNP | |
| | CGTCGCTGCAAGACCCCGACTCCGGCG | | HWGKTWLSILNLYKWEGV | |
| | CATGGCAGTCGGAATACGACGGACCG | | NPTPGELWLLPYFVPVHPG | |
| | CAGTTCATGTCGATCGGTTATGTGACG | | RWWVHTRWIYLAMGYLE | |
| | GCGTGCTACTTTGGCGGCAACGAGATC | | AAEAQCELTPLLEELRDEIY | |
| | CCCACGCCGGTCAAAACCGAAATGATC | | KKPYSEIDFSKHCNSISGVD | |
| | AGATACATTGTCAACACAGCCCACCCA | | LYYPHTGLLKFGNALLRRY | |
| | GTTGACGGAGGCTGGGGCCTTCACAAA | | RKFRPQWIKEKVKEEIYNL | |
| | GAAGACAAGAGCACCTGTTTCGGTGCC | | CLREVSNTRHLCLAPVNNA | |
| | AGCATCAACTACGTGGTCCTGCGACTA | | MTSIVMYLHEGPDSANYKK | |
| | CTGGGCCTGTCACGGGATCATCCGGTC | | IAARWPEFLSLNPSGMFMN | |
| | TGCGTCAAGGCGCGCAAAACGCTGCTC | | GTNGLQVWDTAFAVQYAC | |
| | ATCAAGTTTGGCGGCGCCATCAACAAC | | VCGFAELPQYQKTIRAAFD | |
| | CCCCATTGGGGCAAGACCTGGCTGTCG | | FLDRSQINEPTEENSYRDDR | |
| | ATTCTCAATCTCTACAAATGGGAGGGT | | VGGWPFSTKTQGYPVSDCT | |
| | GTGAATCCGACCCCTGGCGAGCTCTGG | | AEALKAIIMVQNTPGYEDL | |
| | CTGTTGCCCTACTTTGTTCCTGTTCATC | | KKQVSDKRKHTAIDLLLGM | |
| | CGGGCCGATGGTGGGTCCATACCCGGT | | QNVGSFEPGSFASYEPIRAS | |
| | GGATCTACCTTGCCATGGGCTATCTGG | | SMLEKINPAEVFGNIMVEY | |
| | AGGCTGCGGAGGCCCAATGCGAACTC | | PYVECTDSVVLGLSYFRKY | |
| | ACTCCGTTGCTGGAGGAGCTCCGAGAC | | HDYRNEDVDRAISAAIGYII | |
| | GAAATCTACAAAAAGCCCTACTCGGAG | | REQQPDGGFFGSWGVCYC | |
| | ATTGATTTCTCCAAACATTGCAACTCC | | YAHMFAMEALETQNLNYN | |
| | ATCTCCGGAGTCGACCTCTACTATCCC | | NCSTVQKACDFLAGYQEA | |
| | CACACCGGCCTTTTGAAGTTTGGCAAC | | DGGWAEDFKSCETQMYVR | |
| | GCGCTTCTCCGACGATACCGCAAGTTC | | GPHSLVVPTAMALLSLMSG | |
| | AGACCGCAGTGGATCAAAGAAAAGGT | | RYPQEDKIHAAARFLMSKQ | |
| | CAAGGAGGAAATTTACAACTTGTGCCT | | MSNGEWLKEEMEGVFNHT | |
| | TCGAGAGGTTTCCAACACACGACACTT | | CAIEYPNYRFYFVMKALGL | |
| | GTGTCTCGCTCCCGTCAACAATGCCAT | | YFKGYCQ | |
| | GACCTCCATTGTCATGTATCTCCATGA | | | |
| | GGGGCCCGATTCGGCGAATTACAAAA | | | |
| | AGATTGCGGCCCGATGGCCCGAATTTC | | | |
| | TGTCTCTGAATCCGTCGGGAATGTTTA | | | |
| | TGAACGGCACCAACGGTCTGCAGGTCT | | | |
| | GGGATACTGCGTTTGCCGTGCAATACG | | | |
| | CGTGTGTTTGTGGCTTTGCCGAACTTCC | | | |
| | CCAGTACCAGAAGACGATCCGAGCGG | | | |
| | CGTTTGATTTTCTCGATCGGTCCCAGAT | | | |
| | CAACGAGCCGACGGAGGAAAATTCCT | | | |
| | ATCGAGACGACCGCGTCGGAGGATGG | | | |
| | CCCTTTAGTACCAAGACCCAGGGGTAT | | | |
| | CCAGTCTCCGACTGTACTGCCGAGGCT | | | |
| | CTCAAGGCCATCATCATGGTCCAGAAT | | | |
| | ACGCCTGGATACGAGGATCTGAAGAA | | | |
| | ACAAGTGTCTGACAAGCGGAAACACA | | | |
| | CTGCCATCGATCTACTTTTGGGAATGC | | | |
| | AGAACGTGGGCTCGTTTGAACCGGGCT | | | |
| | CTTTCGCCTCCTATGAGCCTATCCGGG | | | |
| | CGTCGTCCATGCTGGAGAAGATCAATC | | | |
| | CGGCCGAGGTGTTTGGAAACATCATGG | | | |
| | TGGAGTATCCGTACGTGGAATGCACTG | | | |
| | ATTCTGTTGTTCTGGGTCTGTCCTACTT | | | |
| | TCGAAAGTACCACGATTACCGCAACGA | | | |
| | AGACGTGGACCGAGCCATCTCTGCTGC | | | |
| | CATTGGATACATTATTCGAGAGCAGCA | | | |
| | GCCTGACGGCGGCTTCTTTGGCTCCTG | | | |
| | GGGCGTGTGCTACTGCTACGCTCACAT | | | |
| | GTTTGCCATGGAGGCTCTGGAGACGCA | | | |
| | GAATCTCAACTATAACAACTGTTCCAC | | | |
| | GGTTCAAAAGGCGTGCGACTTTCTGGC | | | |
| | GGGCTACCAGGAAGCAGATGGAGGCT | | | |
| | GGGCCGAGGACTTTAAGTCGTGCGAGA | | | |
| | CTCAGATGTACGTGCGCGGACCCCATT | | | |
| | CGCTGGTCGTGCCTACTGCCATGGCCC | | | |
| | TGTTGAGTTTGATGAGTGGTCGGTATC | | | |
| | CCCAGGAGGACAAGATTCATGCTGCGG | | | |
| | CCCGGTTTCTCATGAGCAAGCAGATGA | | | |
| | GCAACGGTGAGTGGCTCAAGGAGGAG | | | |
| | ATGGAGGGGGTGTTTAACCATACTTGT | | | |
| | GCCATTGAGTATCCCAACTACCGGTTT | | | |
| | TATTTTGTCATGAAGGCTTTGGGGTTGT | | | |
| | ATTTCAAGGGATATTGCCAGTGA | | | |
| 950910 | ATGGGAATCCACGAAAGTGTGTCGAA | 63 | MGIHESVSKQFAKNGHSKY | 84 |
| | ACAGTTTGCGAAAAACGGACATTCCAA | | RSDRYGLPKTDLRRWTFHA | |
| | GTACCGCAGCGACCGATACGGCTTACC | | SDLGAQWWKYDDTTPLEE | |
| | TAAGACGGATCTGCGACGATGGACGTT | | LEKRATDYVKYSLELPGYA | |
| | CCACGCGTCCGATCTGGGGGCGCAATG | | PVTLDSKPVKNAYEAALKN | |
| | GTGGAAGTATGACGATACCACACCGCT | | WHLFASLQDPDSGAWQSE | |
| | GGAAGAGCTGGAAAAGAGGGCTACCG | | YDGPQFMSIGYVTACYFGG | |
| | ACTACGTCAAATACTCGCTGGAGCTGC | | NEIPTPVKTEMIRYIVNTAH | |
| | CGGGATACGCGCCCGTGACTCTGGACT | | PVDGGWGLHKEDKSTCFG | |
| | CCAAGCCCGTGAAAAATGCCTACGAA | | TSNNYVVLRLLGLSRDHPV | |
| | GCGGCTCTCAAAAACTGGCATCTGTTT | | CVKARKTLLTKFGGAINNP | |
| | GCGTCGCTGCAAGACCCCGACTCCGGC | | HWGKTWLSILNLYKWEGV | |
| | GCATGGCAGTCGGAATACGACGGACC | | NPAPGELWLLPYFVPVHPG | |
| | GCAGTTCATGTCGATCGGTTATGTGAC | | RWWVHTRWIYLAMGYLE | |
| | GGCGTGCTACTTTGGCGGCAACGAGAT | | AAEAQCELTPLLEELRDEIY | |
| | CCCCACGCCGGTCAAAACCGAAATGAT | | KKPYSEIDFSKHCNSISGVD | |
| | CAGATACATTGTCAACACAGCCCACCC | | LYYPHTGLLKFGNALLRRY | |
| | AGTTGACGGAGGCTGGGGCCTTCACAA | | RKFRPQWIKEKVKEEIYNL | |
| | AGAAGACAAGAGCACCTGTTTCGGTAC | | CLREVSNTRHLCLAPVNNA | |
| | CAGCAACAACTACGTGGTCCTGCGACT | | MTSIVMYLHEGPDSANYKK | |
| | ACTGGGCCTGTCACGGGATCATCCGGT | | IAARWPEFLSLNPSGMFMN | |
| | CTGCGTCAAGGCGCGCAAAACGCTGCT | | GTNGLQVWDTAFAVQYAS | |
| | CACCAAGTTTGGCGGCGCCATCAACAA | | VCGFAELPQYQKTIRAAFD | |
| | CCCCCATTGGGGCAAGACCTGGCTGTC | | FLDRSQINEPTEENSYRDDR | |
| | GATTCTCAATCTCTACAAATGGGAGGG | | VGGWPFSTKTQGYPVSDCT | |
| | TGTGAATCCGGCCCCTGGCGAGCTCTG | | AEALKAIIMVQNTPGYEDL | |
| | GCTGTTGCCCTACTTTGTTCCTGTTCAT | | KKQVSDKRKHTAIDLLLGM | |
| | CCGGGCCGATGGTGGGTCCATACCCGG | | QNVGSFEPGSFASYEPIRAS | |
| | TGGATCTACCTTGCCATGGGCTATCTG | | SMLEKINPAEVFGNIMVEY | |
| | GAGGCTGCGGAGGCCCAATGCGAACT | | PYVECTDSVVMGLSYFRKY | |
| | CACTCCGTTGCTGGAGGAGCTCCGAGA | | HDYRNEDVDRAISAAIGYII | |
| | CGAAATCTACAAAAAGCCCTACTCGGA | | REQQPDGGFFGSWGVCYC | |
| | GATTGATTTCTCCAAACATTGCAACTC | | YAHMFAMEALETQNLNYN | |
| | CATCTCCGGAGTCGACCTCTACTATCC | | NCSTVQKACDFLAGYQEA | |
| | CCACACCGGCCTTTTGAAGTTTGGCAA | | DGGWAEDFKSCETQMYVR | |
| | CGCGCTTCTCCGACGATACCGCAAGTT | | GPHSLVVPTAMALLSLMSS | |
| | CAGACCGCAGTGGATCAAAGAAAAGG | | RYPQEDKIHAAARFLMSKQ | |
| | TCAAGGAGGAAATTTACAACTTGTGCC | | MSNGEWLKEEMEGVFNHT | |
| | TTCGAGAGGTTTCCAACACACGACACT | | CAIEYPNYRFYFVMKALGL | |
| | TGTGTCTCGCTCCCGTCAACAATGCCA | | YFKGYCQ | |
| | TGACCTCCATTGTCATGTATCTCCATGA | | | |
| | GGGGCCCGATTCGGCGAATTACAAAA | | | |
| | AGATTGCGGCCCGATGGCCCGAATTTC | | | |
| | TGTCTCTGAATCCGTCGGGAATGTTTA | | | |
| | TGAACGGCACCAACGGTCTGCAGGTCT | | | |
| | GGGATACTGCGTTTGCCGTGCAATACG | | | |
| | CGAGTGTTTGTGGCTTTGCCGAACTTC | | | |
| | CCCAGTACCAGAAGACGATCCGAGCG | | | |
| | GCGTTTGATTTTCTCGATCGGTCCCAG | | | |
| | ATCAACGAGCCGACGGAGGAAAATTC | | | |
| | CTATCGAGACGACCGCGTCGGAGGATG | | | |
| | GCCCTTTAGTACCAAGACCCAGGGGTA | | | |
| | TCCAGTCTCCGACTGTACTGCCGAGGC | | | |
| | TCTCAAGGCCATCATCATGGTCCAGAA | | | |
| | TACGCCTGGATACGAGGATCTGAAGAA | | | |
| | ACAAGTGTCTGACAAGCGGAAACACA | | | |
| | CTGCCATCGATCTACTTTTGGGAATGC | | | |
| | AGAACGTGGGCTCGTTTGAACCGGGCT | | | |
| | CTTTCGCCTCCTATGAGCCTATCCGGG | | | |
| | CGTCGTCCATGCTGGAGAAGATCAATC | | | |
| | CGGCCGAGGTGTTTGGAAACATCATGG | | | |
| | TGGAGTATCCGTACGTGGAATGCACTG | | | |
| | ATTCTGTTGTTATGGGTCTGTCCTACTT | | | |
| | TCGAAAGTACCACGATTACCGCAACGA | | | |
| | AGACGTGGACCGAGCCATCTCTGCTGC | | | |
| | CATTGGATACATTATTCGAGAGCAGCA | | | |
| | GCCTGACGGCGGCTTCTTTGGCTCCTG | | | |
| | GGGCGTGTGCTACTGCTACGCTCACAT | | | |
| | GTTTGCCATGGAGGCTCTGGAGACGCA | | | |
| | GAATCTCAACTATAACAACTGTTCCAC | | | |
| | GGTTCAAAAGGCGTGCGACTTTCTGGC | | | |
| | GGGCTACCAGGAAGCAGATGGAGGCT | | | |
| | GGGCCGAGGACTTTAAGTCGTGCGAGA | | | |
| | CTCAGATGTACGTGCGCGGACCCCATT | | | |
| | CGCTGGTCGTGCCTACTGCCATGGCCC | | | |
| | TGTTGAGTTTGATGAGTAGTCGGTATC | | | |
| | CCCAGGAGGACAAGATTCATGCTGCGG | | | |
| | CCCGGTTTCTCATGAGCAAGCAGATGA | | | |
| | GCAACGGTGAGTGGCTCAAGGAGGAG | | | |
| | ATGGAGGGGGTGTTTAACCATACTTGT | | | |
| | GCCATTGAGTATCCCAACTACCGGTTT | | | |
| | TATTTTGTCATGAAGGCTTTGGGGTTGT | | | |
| | ATTTCAAGGGATATTGCCAGTGA | | | |
| 950917 | ATGGGAATCCACGAAAGTGTGTCGAA | 74 | MGIHESVSKQFAKNGHSKY | 95 |
| | ACAGTTTGCGAAAAACGGACATTCCAA | | RSDRYGLPKTDLRRWTFHA | |
| | GTACCGCAGCGACCGATACGGCTTACC | | SDLGAQWWKYDGTTPLEE | |
| | TAAGACGGATCTGCGACGATGGACGTT | | LEKRATDYVRYSLELPGYA | |
| | CCACGCGTCCGATCTGGGGGCGCAATG | | PVTLDSKPVKNAYEAALKS | |
| | GTGGAAGTATGACGGTACCACACCGCT | | WHLFASLQDPDSGAWQSE | |
| | GGAAGAGCTGGAAAAGAGGGCTACCG | | YDGPQFMSIGYVTACYFGG | |
| | ACTACGTCAGATACTCGCTGGAGCTGC | | NEIPTPVKTEMIRYIVNTAH | |
| | CGGGATACGCGCCCGTGACTCTGGACT | | PVDGGWGLHKEDKSTCFG | |
| | CCAAGCCCGTGAAAAATGCCTACGAA | | TSINYVVLRLLGLSRDHPVC | |
| | GCGGCTCTCAAAAGCTGGCATCTGTTT | | VKARKTLLTKFGGAINNPH | |
| | GCGTCGCTGCAAGACCCCGACTCCGGC | | WGKTWLSILNLYKWEGVN | |
| | GCATGGCAGTCGGAATACGACGGACC | | PAPGELWLLPYFVPVHPGR | |
| | GCAGTTCATGTCGATCGGTTATGTGAC | | WWVHTRWIYLAMGYLEA | |
| | GGCGTGCTACTTTGGCGGCAACGAGAT | | AEAQCELTPLLEELRDEIYK | |
| | CCCCACGCCGGTCAAAACCGAAATGAT | | KPYSEIDFSKHCNSISGVDL | |
| | CAGATACATTGTCAACACAGCCCACCC | | YYPHTGLLKFGNALLRRYR | |
| | AGTTGACGGAGGCTGGGGCCTTCACAA | | KFRPQWIKEKVKEEIYNLC | |
| | AGAAGACAAGAGCACCTGTTTCGGTAC | | LREVSNTRHLCLAPVNNAM | |
| | CAGCATCAACTACGTGGTCCTGCGACT | | TSIVMYLHEGPDSANYKKI | |
| | ACTGGGCCTGTCACGGGATCATCCGGT | | AARWPEFLSLNPSGMFMN | |
| | CTGCGTCAAGGCGCGCAAAACGCTGCT | | GTNGLQVWDTAFAVQYAC | |
| | CACCAAGTTTGGCGGCGCCATCAACAA | | VCSFAELPQYQKTIRAAFDF | |
| | CCCCCATTGGGGCAAGACCTGGCTGTC | | LDRSQINEPTEENSYRDDRV | |
| | GATTCTCAATCTCTACAAATGGGAGGG | | GGWPFSTKTQGYPVSDCTA | |
| | TGTGAATCCGGCCCCTGGCGAGCTCTG | | EALKAIIMVQNTPGYEDLK | |
| | GCTGTTGCCCTACTTTGTTCCTGTTCAT | | KQVSDKRKHTAIDLLLGMQ | |
| | CCGGGCCGATGGTGGGTCCATACCCGG | | NVGSFEPGSFASYEPIRASS | |
| | TGGATCTACCTTGCCATGGGCTATCTG | | MLEKINPAEVFGNIMVEYP | |
| | GAGGCTGCGGAGGCCCAATGCGAACT | | YVECTDSVVLGLSYFRKYH | |
| | CACTCCGTTGCTGGAGGAGCTCCGAGA | | DYRNEDVDRAISAAIGYIIR | |
| | CGAAATCTACAAAAAGCCCTACTCGGA | | EQQPDGGFFGSWGVCYCY | |
| | GATTGATTTCTCCAAACATTGCAACTC | | AHMFAMEALVTQNLNYNN | |
| | CATCTCCGGAGTCGACCTCTACTATCC | | CSTVQKACDFLAGYQEAD | |
| | CCACACCGGCCTTTTGAAGTTTGGCAA | | GGWAEDFKSCETQMYVRG | |
| | CGCGCTTCTCCGACGATACCGCAAGTT | | PHSLVVPTAMALLSLMSGR | |
| | CAGACCGCAGTGGATCAAAGAAAAGG | | YPQEDKIHAAARFLMSKQ | |
| | TCAAGGAGGAAATTTACAACTTGTGCC | | MSNGEWLKEEMEGVFNHT | |
| | TTCGAGAGGTTTCCAACACACGACACT | | CAIEYPNYRFYFVMKALGL | |
| | TGTGTCTCGCTCCCGTCAACAATGCCA | | YFKGYCQ | |
| | TGACCTCCATTGTCATGTATCTCCATGA | | | |
| | GGGGCCCGATTCGGCGAATTACAAAA | | | |
| | AGATTGCGGCCCGATGGCCCGAATTTC | | | |
| | TGTCTCTGAATCCGTCGGGAATGTTTA | | | |
| | TGAACGGCACCAACGGTCTGCAGGTCT | | | |
| | GGGATACTGCGTTTGCCGTGCAATACG | | | |
| | CGTGTGTTTGTAGCTTTGCCGAACTTCC | | | |
| | CCAGTACCAGAAGACGATCCGAGCGG | | | |
| | CGTTTGATTTTCTCGATCGGTCCCAGAT | | | |
| | CAACGAGCCGACGGAGGAAAATTCCT | | | |
| | ATCGAGACGACCGCGTCGGAGGATGG | | | |
| | CCCTTTAGTACCAAGACCCAGGGGTAT | | | |
| | CCAGTCTCCGACTGTACTGCCGAGGCT | | | |
| | CTCAAGGCCATCATCATGGTCCAGAAT | | | |
| | ACGCCTGGATACGAGGATCTGAAGAA | | | |
| | ACAAGTGTCTGACAAGCGGAAACACA | | | |
| | CTGCCATCGATCTACTTTTGGGAATGC | | | |
| | AGAACGTGGGCTCGTTTGAACCGGGCT | | | |
| | CTTTCGCCTCCTATGAGCCTATCCGGG | | | |
| | CGTCGTCCATGCTGGAGAAGATCAATC | | | |
| | CGGCCGAGGTGTTTGGAAACATCATGG | | | |
| | TGGAGTATCCGTACGTGGAATGCACTG | | | |
| | ATTCTGTTGTTCTGGGTCTGTCCTACTT | | | |
| | TCGAAAGTACCACGATTACCGCAACGA | | | |
| | AGACGTGGACCGAGCCATCTCTGCTGC | | | |
| | CATCGGATACATTATTCGAGAGCAGCA | | | |
| | GCCTGACGGTGGCTTCTTTGGCTCCTG | | | |
| | GGGCGTGTGCTACTGCTACGCTCACAT | | | |
| | GTTTGCCATGGAGGCTCTGGTGACGCA | | | |
| | GAATCTCAACTATAACAACTGTTCCAC | | | |
| | GGTTCAAAAGGCGTGCGACTTTCTGGC | | | |
| | GGGCTACCAGGAAGCAGATGGAGGCT | | | |
| | GGGCCGAGGACTTTAAGTCGTGCGAGA | | | |
| | CTCAGATGTACGTGCGCGGACCCCATT | | | |
| | CGCTGGTCGTGCCTACTGCCATGGCCC | | | |
| | TGTTGAGTTTGATGAGTGGTCGGTATC | | | |
| | CCCAGGAGGACAAGATTCATGCTGCGG | | | |
| | CCCGGTTTCTCATGAGCAAGCAGATGA | | | |
| | GCAACGGTGAGTGGCTCAAGGAGGAG | | | |
| | ATGGAGGGGGTGTTTAACCATACTTGT | | | |
| | GCCATTGAGTATCCCAACTACCGGTTT | | | |
| | TATTTTGTCATGAAGGCTTTGGGGTTGT | | | |
| | ATTTCAAGGGATATTGCCAGTGA | | | |
| 959829 | ATGGGAATCCACGAAAGTGTGTCGAA | 78 | MGIHESVSKQFAKNGHSKY | 99 |
| | ACAGTTTGCGAAAAACGGACATTCCAA | | RSDRYGLPKTDLRRWTFHA | |
| | GTACCGCAGCGACCGATACGGCTTACC | | SDLGAQWWKYDDTTPLEE | |
| | TAAGACGGATCTGCGACGATGGACGTT | | LEKRATDYVKYSLELPGYA | |
| | CCACGCGTCCGATCTGGGGGCGCAATG | | PVTLDSKPVKNAYEAALKN | |
| | GTGGAAGTATGACGATACCACACCGCT | | WHLFASLQDPDSGAWQSE | |
| | GGAAGAGCTGGAAAAGAGGGCTACCG | | YDGPQFMSIGYVTACYFGG | |
| | ACTACGTCAAATACTCGCTGGAGCTGC | | NEIPTPVKTEMIRYIVNTAH | |
| | CGGGATACGCGCCCGTGACTCTGGACT | | PVDGGWGLHKEDKSTCFG | |
| | CCAAGCCCGTGAAAAATGCCTACGAA | | TSINYVVLRLLGLSRDHPVC | |
| | GCGGCTCTCAAAAACTGGCATCTGTTT | | VKARKTLLTKFGGAINNPH | |
| | GCGTCGCTGCAAGACCCCGACTCCGGC | | WGKTWLSILNLYKWEGVN | |
| | GCATGGCAGTCGGAATACGACGGACC | | PAPGELWLLPYFVPVHPGR | |
| | GCAGTTCATGTCGATCGGTTATGTGAC | | WWVHTRWIYLAMGYLEA | |
| | GGCGTGCTACTTTGGCGGCAACGAGAT | | AEAQCELTPLLEELRDEIYK | |
| | CCCCACGCCGGTCAAAACCGAAATGAT | | KPYSEIDFSKHCNSISGVDL | |
| | CAGATACATTGTCAACACAGCCCACCC | | YYPHTGFLKFGNALLRRYR | |
| | AGTTGACGGAGGCTGGGGCCTTCACAA | | KFRPQWIKEKVKEEIYNLC | |
| | AGAAGACAAGAGCACCTGTTTCGGTAC | | LREASNTRHLCLAPVNNAM | |
| | CAGCATCAACTACGTGGTCCTGCGACT | | TSIVMYLHEGPDSANYKKI | |
| | ACTGGGCCTGTCACGGGATCATCCGGT | | AARWPEFLSLNPSGMFMN | |
| | CTGCGTCAAGGCGCGCAAAACGCTGCT | | GINGLQVWDTAFAVQYAC | |
| | CACCAAGTTTGGCGGCGCCATCAACAA | | VCGFAELPQYQKTIRAAFD | |
| | CCCCCATTGGGGCAAGACCTGGCTGTC | | FLDRSQINEPTEENSYRDDR | |
| | GATTCTCAATCTCTACAAATGGGAGGG | | VGGWPFSTKTQGYPVSDCT | |
| | TGTGAATCCGGCCCCTGGCGAGCTCTG | | AEALKAIIMVQNTPGYEDL | |
| | GCTGTTGCCCTACTTTGTTCCTGTTCAT | | KKQVSDKRKHTAIDLLLGM | |
| | CCGGGCCGATGGTGGGTCCATACCCGG | | QNVGSFEPGSFASYEPIRAS | |
| | TGGATCTACCTTGCCATGGGCTATCTG | | SMLEKINPAEVFGNIMVEY | |
| | GAGGCTGCGGAGGCCCAATGCGAACT | | PYVECTDSVVLGLSYFRKY | |
| | CACTCCGTTGCTGGAGGAGCTCCGAGA | | HDYRNEDVDRAISAAIGYII | |
| | TGAAATCTACAAAAAGCCCTACTCGGA | | REQQPDGGFFGSWGVCYC | |
| | GATTGATTTCTCCAAACATTGCAACTC | | YAHMFAMEALETQNLNYN | |
| | CATCTCCGGAGTCGACCTCTACTACCC | | NCSTVQKACDFLAGYQEA | |
| | CCACACCGGCTTTTTGAAGTTTGGCAA | | DGGWAEDFKSCETQMYVR | |
| | CGCGCTTCTCCGACGATACCGCAAGTT | | GPHSLVVPTAMALLSLMSG | |
| | CAGACCGCAGTGGATCAAAGAAAAGG | | RYPQEDEIHAAARFLMSKQ | |
| | TCAAGGAGGAAATTTACAACTTGTGCC | | MSNGEWLKEEMEGVFNHT | |
| | TTCGAGAGGCTTCCAACACACGACACT | | CAIEYPNYRFYFVMKALGL | |
| | TGTGTCTCGCTCCCGTCAACAATGCCA | | YFKGYCQ | |
| | TGACCTCCATTGTCATGTATCTCCATGA | | | |
| | GGGGCCCGATTCGGCGAATTACAAAA | | | |
| | AGATTGCGGCCCGATGGCCCGAATTTC | | | |
| | TGTCTCTGAATCCGTCGGGAATGTTTA | | | |
| | TGAACGGCATCAACGGTCTGCAGGTCT | | | |
| | GGGATACTGCGTTTGCCGTGCAATACG | | | |
| | CGTGTGTTTGTGGCTTTGCCGAACTTCC | | | |
| | CCAGTACCAGAAGACGATCCGAGCGG | | | |
| | CGTTTGATTTTCTCGATCGGTCCCAGAT | | | |
| | CAACGAGCCGACGGAGGAAAATTCCT | | | |
| | ATCGAGACGACCGCGTCGGAGGATGG | | | |
| | CCCTTTAGTACCAAGACCCAGGGGTAT | | | |
| | CCAGTCTCCGACTGTACTGCCGAGGCT | | | |
| | CTCAAGGCCATCATCATGGTCCAGAAT | | | |
| | ACGCCTGGATACGAGGATCTGAAGAA | | | |
| | ACAAGTATCTGACAAGCGGAAACACA | | | |
| | CTGCCATCGATCTACTTTTGGGAATGC | | | |
| | AGAACGTGGGCTCGTTTGAACCGGGCT | | | |
| | CTTTCGCCTCCTATGAGCCTATCCGGG | | | |
| | CGTCGTCCATGCTGGAGAAGATCAATC | | | |
| | CGGCCGAGGTGTTTGGAAACATCATGG | | | |
| | TGGAGTATCCGTACGTGGAATGCACTG | | | |
| | ATTCTGTTGTTCTGGGTCTGTCCTACTT | | | |
| | TCGAAAGTACCACGATTACCGTAACGA | | | |
| | AGACGTGGACCGAGCCATCTCTGCTGC | | | |
| | CATTGGATACATTATTCGAGAGCAGCA | | | |
| | GCCTGACGGCGGCTTCTTTGGCTCCTG | | | |
| | GGGCGTGTGCTACTGCTACGCTCACAT | | | |
| | GTTTGCCATGGAGGCTCTGGAGACGCA | | | |
| | GAATCTCAACTATAACAACTGTTCCAC | | | |
| | GGTTCAAAAGGCGTGCGACTTTCTGGC | | | |
| | AGGTTACCAGGAAGCAGATGGAGGCT | | | |
| | GGGCCGAGGACTTTAAGTCGTGCGAGA | | | |
| | CTCAGATGTACGTGCGCGGACCCCATT | | | |
| | CGCTGGTCGTGCCTACTGCCATGGCCC | | | |
| | TGTTGAGTTTGATGAGTGGTCGGTATC | | | |
| | CCCAGGAGGACGAGATTCATGCTGCGG | | | |
| | CCCGGTTTCTCATGAGCAAGCAGATGA | | | |
| | GCAACGGTGAGTGGCTCAAGGAGGAG | | | |
| | ATGGAGGGGGTGTTTAACCATACTTGT | | | |
| | GCCATTGAGTATCCCAACTACCGGTTT | | | |
| | TATTTTGTCATGAAGGCTTTGGGGTTGT | | | |
| | ATTTCAAGGGATATTGCCAGTGA | | | |
| Parent | ATGACAGAATTTTATTCTGACACAATC | 79 | MTEFYSDTIGLPKTDPRLW | 313 |
| strain | GGTCTACCAAAGACAGATCCACGTCTT | | RLRTDELGRESWEYLTPQQ | |
| from | TGGAGACTGAGAACTGATGAGCTAGG | | AANDPPSTFTQWLLQDPKF | |
| Example | CCGAGAAAGCTGGGAATATTTAACCCC | | PQPHPERNKHSPDFSAFDA | |
| 4 | TCAGCAAGCCGCAAACGACCCACCATC | | CHNGASFFKLLQEPDSGIFP | |
| | CACTTTCACGCAGTGGCTTCTTCAAGA | | CQYKGPMFMTIGYVAVNYI | |
| | TCCCAAATTTCCTCAACCTCATCCAGA | | AGIEIPEHERIELIRYIVNTA | |
| | AAGAAATAAGCATTCACCAGATTTTTC | | HPVDGGWGLHSVDKSTVF | |
| | AGCCTTCGATGCGTGTCATAATGGTGC | | GTVLNYVILRLLGLPKDHP | |
| | ATCTTTTTTCAAACTGCTTCAAGAGCCT | | VCAKARSTLLRLGGAIGSP | |
| | GACTCAGGTATTTTTCCGTGTCAATAT | | HWGKIWLSALNLYKWEGV | |
| | AAAGGACCCATGTTCATGACAATCGGT | | NPAPPETWLLPYSLPMHPG | |
| | TACGTAGCCGTAAACTATATCGCCGGT | | RWWVHTRGVYIPVSYLSLV | |
| | ATTGAAATTCCTGAGCATGAGAGAATA | | KFSCPMTPLLEELRNEIYTK | |
| | GAATTAATTAGATACATCGTCAATACA | | PFDKINFSKNRNTVCGVDL | |
| | GCACATCCGGTTGATGGTGGCTGGGGT | | YYPHSTTLNIANSLVVFYEK | |
| | CTACATTCTGTTGACAAATCCACCGTG | | YLRNRFIYSLSKKKVYDLIK | |
| | TTTGGTACAGTATTGAACTATGTAATCT | | TELQNTDSLCIAPVNQAFC | |
| | TACGTTTATTGGGTCTACCCAAGGACC | | ALVTLIEEGVDSEAFQRLQ | |
| | ACCCGGTTTGCGCCAAGGCAAGAAGC | | YRFKDALFHGPQGMTIMGT | |
| | ACATTGTTAAGGTTAGGCGGTGCTATT | | NGVQTWDCAFAIQYFFVA | |
| | GGATCCCCTCACTGGGGAAAAATTTGG | | GLAERPEFYNTIVSAYKFLC | |
| | CTAAGTGCACTAAACTTGTATAAATGG | | HAQFDTECVPGSYRDKRKG | |
| | GAAGGTGTGAACCCTGCCCCTCCTGAA | | AWGFSTKTQGYTVADCTA | |
| | ACTTGGTTACTTCCATATTCACTGCCCA | | EAIKAIIMVKNSPVFSEVHH | |
| | TGCATCCGGGGAGATGGTGGGTTCATA | | MISSERLFEGIDVLLNLQNI | |
| | CTAGAGGTGTTTACATTCCGGTCAGTT | | GSFEYGSFATYEKIKAPLA | |
| | ACCTGTCATTGGTCAAATTTTCTTGCCC | | METLNPAEVFGNIMVEYPY | |
| | AATGACTCCTCTTCTTGAAGAACTGAG | | VECTDSSVLGLTYFHKYFD | |
| | GAATGAAATTTACACTAAACCGTTTGA | | YRKEEIRTRIRIAIEFIKKSQL | |
| | CAAGATTAACTTCTCCAAGAACAGGAA | | PDGSWYGSWGICFTYAGM | |
| | TACCGTATGTGGAGTAGACCTATATTA | | FALEALHTVGETYENSSTV | |
| | CCCCCATTCTACTACTTTGAATATTGCG | | RKGCDFLVSKQMKDGGWG | |
| | AACAGCCTTGTAGTATTTTACGAAAAA | | ESMKSSELHSYVDSEKSLV | |
| | TACCTAAGAAACCGGTTCATTTACTCT | | VQTAWALIALLFAEYPNKE | |
| | CTATCCAAGAAGAAGGTTTATGATCTA | | VIDRGIDLLKNRQEESGEW | |
| | ATCAAAACGGAGTTACAGAATACTGAT | | KFESVEGVFNHSCAIEYPSY | |
| | TCCTTGTGTATAGCACCTGTTAACCAG | | RFLFPIKALGMYSRAYETH | |
| | GCGTTTTGCGCACTTGTCACTCTTATTG | | TL | |
| | AAGAAGGGGTAGACTCGGAAGCGTTC | | | |
| | CAGCGTCTCCAATATAGGTTCAAGGAT | | | |
| | GCATTGTTCCATGGTCCACAGGGTATG | | | |
| | ACCATTATGGGAACAAATGGTGTGCAA | | | |
| | ACCTGGGATTGTGCGTTTGCCATTCAA | | | |
| | TACTTTTTCGTCGCAGGCCTCGCAGAA | | | |
| | AGACCTGAATTCTATAACACAATTGTC | | | |
| | TCTGCCTATAAATTCTTGTGTCATGCTC | | | |
| | AATTTGACACCGAGTGCGTTCCAGGTA | | | |
| | GTTATAGGGATAAGAGAAAGGGGGCT | | | |
| | TGGGGCTTCTCAACAAAAACACAGGGC | | | |
| | TATACAGTGGCAGATTGCACTGCAGAA | | | |
| | GCAATTAAAGCCATCATCATGGTGAAA | | | |
| | AACTCTCCCGTCTTTAGTGAAGTACAC | | | |
| | CATATGATTAGCAGTGAACGTTTATTT | | | |
| | GAAGGCATTGATGTGTTATTGAACCTA | | | |
| | CAAAACATCGGATCTTTTGAATATGGT | | | |
| | TCCTTTGCAACCTATGAAAAAATCAAG | | | |
| | GCCCCACTAGCAATGGAAACCTTGAAT | | | |
| | CCTGCTGAAGTTTTTGGTAACATAATG | | | |
| | GTAGAATACCCATACGTGGAATGTACT | | | |
| | GATTCATCCGTTCTGGGGTTGACATAT | | | |
| | TTTCACAAGTACTTCGACTATAGGAAA | | | |
| | GAGGAAATACGTACACGCATCAGAAT | | | |
| | CGCCATCGAATTCATAAAAAAATCTCA | | | |
| | ATTACCAGATGGAAGTTGGTATGGAAG | | | |
| | CTGGGGTATTTGTTTTACATATGCCGGT | | | |
| | ATGTTTGCATTGGAGGCATTACACACC | | | |
| | GTGGGGGAGACCTATGAGAATTCCTCA | | | |
| | ACGGTAAGAAAAGGTTGCGACTTCTTG | | | |
| | GTCAGTAAACAGATGAAGGATGGCGG | | | |
| | TTGGGGGGAATCAATGAAGTCCAGTGA | | | |
| | ATTACATAGTTATGTGGATAGTGAAAA | | | |
| | ATCGCTAGTCGTTCAAACCGCATGGGC | | | |
| | GCTAATTGCACTTCTTTTCGCTGAATAT | | | |
| | CCTAATAAAGAAGTCATCGACCGCGGT | | | |
| | ATTGACCTTTTAAAAAATAGACAAGAA | | | |
| | GAATCCGGGGAATGGAAATTTGAAAG | | | |
| | TGTAGAAGGTGTTTTCAACCACTCTTG | | | |
| | TGCAATTGAATACCCAAGTTATCGATT | | | |
| | CTTATTCCCTATTAAGGCATTAGGTAT | | | |
| | GTACAGCAGGGCATATGAAACACATA | | | |
| | CGCTTTAA | | | |
| 756247 | ATGACAGAATTTTATTCTGACACAATC | 80 | MTEFYSDTIGLPKTDPRLW | 100 |
| | GGTCTACCAAAGACAGATCCACGTCTT | | RLRTDELGRESWEYLTPQQ | |
| | TGGAGACTGAGAACTGATGAGCTAGG | | AANDPPSTFTQWLLQDPKF | |
| | CCGAGAAAGCTGGGAATATTTAACCCC | | PQPHPERNKHSPDFSAFDA | |
| | TCAGCAAGCCGCAAACGACCCACCATC | | CHNGASFFKLLQEPDSGIFP | |
| | CACTTTCACGCAGTGGCTTCTTCAAGA | | CQYKGPMFMTIGYVAVNYI | |
| | TCCCAAATTTCCTCAACCTCATCCAGA | | AGIEISEHERIELIRYIVNTV | |
| | AAGAAATAAGCATTCACCAGATTTTTC | | HPVDGGWGLHSVDKSTVF | |
| | AGCCTTCGATGCGTGTCATAATGGTGC | | GTVLNYVILRLLGLPKDHP | |
| | ATCTTTTTTCAAACTGCTTCAAGAGCCT | | VCAKARSTLLRLGGAIGSP | |
| | GACTCAGGTATTTTTCCGTGTCAATAT | | HWGKIWLSALNLYKWEGV | |
| | AAAGGACCCATGTTCATGACAATCGGT | | NPAPPETWLLPYSLPMHPG | |
| | TACGTAGCCGTAAACTATATCGCCGGT | | RWWVHTRGVYIPVSYLSLV | |
| | ATTGAAATTTCTGAGCATGAGAGAATA | | KFSCPMTPLLEELRNEIYTK | |
| | GAATTAATTAGATACATCGTCAATACA | | PFDKINFSKNRNTVCGVDL | |
| | GTACATCCGGTTGATGGTGGCTGGGGT | | YYPHSTTLNIANGLVVFYE | |
| | CTACATTCTGTTGACAAATCCACCGTG | | KYLRNRFIYSLSKKKGYDLI | |
| | TTTGGTACAGTATTAAACTATGTAATCT | | KTELQNTDSLCIAPVNQAF | |
| | TACGTTTATTGGGTCTACCCAAGGACC | | CALVTLIEEGVDSEAFQRLQ | |
| | ACCCGGTTTGCGCCAAGGCAAGAAGC | | YRFKDALFHGPQGMTIMGT | |
| | ACATTGTTAAGGTTAGGCGGTGCTATT | | NGVQTWDCAFAIQYFFVA | |
| | GGATCCCCTCACTGGGGAAAAATTTGG | | GLAERPEFYNTIVSAYKFLC | |
| | CTAAGTGCACTAAACTTGTATAAATGG | | HAQFDTECVPGSYRDERKG | |
| | GAAGGTGTGAACCCTGCCCCTCCTGAA | | AWGFSTKTQGYTVADCTA | |
| | ACTTGGTTACTTCCATATTCACTGCCCA | | EAIKAIIMVKNSPVFSEVHH | |
| | TGCATCCGGGGAGATGGTGGGTTCATA | | MISSERLFEGIDVLLNLQNI | |
| | CTAGAGGTGTTTACATTCCGGTCAGTT | | GSLEYGSFATYEKIKAPLA | |
| | ACCTGTCATTGGTCAAATTTTCTTGCCC | | METLNPAEVFGNIMVEYPY | |
| | AATGACTCCTCTTCTTGAAGAACTGAG | | VECTDSSVLGLTYFHKYFD | |
| | GAATGAAATTTACACTAAACCGTTTGA | | YRKEEIRTRIRIAIEFIKKSQL | |
| | CAAGATTAACTTCTCCAAGAACAGGAA | | PDGSWYGSWGICFTYAGM | |
| | TACCGTATGTGGAGTAGACCTATATTA | | FALEALHTVGETYENSSTV | |
| | CCCCCATTCTACTACTTTGAATATTGCG | | RKGCDFLVSKQMEDGGWG | |
| | AACGGCCTTGTAGTGTTTTACGAAAAA | | ESMKSSELHSYVDSEKSLV | |
| | TACCTAAGAAACCGGTTCATTTACTCT | | VQTAWALIALLFAEYPNKE | |
| | CTATCCAAGAAGAAGGGTTATGATCTA | | VIDRGIDLLKNRQEESGEW | |
| | ATCAAAACGGAGTTACAGAATACTGAT | | KFESVEGVFNHSCAIEYPSY | |
| | TCCTTGTGTATAGCACCTGTTAACCAG | | RFLFPIKALGMYSRA | |
| | GCGTTTTGCGCACTTGTCACTCTTATTG | | | |
| | AAGAAGGGGTAGACTCGGAAGCGTTC | | | |
| | CAGCGTCTCCAATATAGGTTCAAGGAT | | | |
| | GCATTGTTCCATGGTCCACAGGGTATG | | | |
| | ACCATTATGGGAACAAATGGTGTGCAA | | | |
| | ACCTGGGATTGTGCGTTTGCCATTCAA | | | |
| | TACTTTTTCGTCGCAGGCCTCGCAGAA | | | |
| | AGACCTGAATTCTATAACACAATTGTC | | | |
| | TCTGCCTATAAATTCTTGTGTCATGCTC | | | |
| | AATTTGACACCGAGTGCGTTCCAGGTA | | | |
| | GTTATAGGGATGAGAGAAAGGGGGCT | | | |
| | TGGGGCTTCTCAACAAAAACACAGGGC | | | |
| | TATACAGTGGCAGATTGCACTGCAGAA | | | |
| | GCAATTAAAGCCATCATCATGGTGAAA | | | |
| | AACTCTCCCGTCTTTAGTGAAGTACAC | | | |
| | CATATGATTAGCAGTGAACGTTTATTT | | | |
| | GAAGGCATTGATGTGTTATTGAACCTA | | | |
| | CAAAACATCGGATCTTTAGAATATGGT | | | |
| | TCCTTTGCAACCTATGAAAAAATCAAG | | | |
| | GCCCCACTAGCAATGGAAACCTTGAAT | | | |
| | CCTGCTGAAGTTTTTGGTAACATAATG | | | |
| | GTAGAATACCCATACGTGGAATGTACT | | | |
| | GATTCATCCGTTCTGGGGTTGACATAT | | | |
| | TTTCACAAGTACTTCGACTATAGGAAA | | | |
| | GAGGAAATACGTACACGCATCAGAAT | | | |
| | CGCCATCGAATTCATAAAAAAATCTCA | | | |
| | ACTACCAGATGGAAGTTGGTATGGAAG | | | |
| | CTGGGGTATTTGTTTTACATATGCCGGT | | | |
| | ATGTTTGCATTGGAGGCATTACACACC | | | |
| | GTGGGGGAGACCTATGAGAATTCCTCA | | | |
| | ACGGTAAGAAAAGGTTGCGACTTCTTG | | | |
| | GTCAGTAAACAGATGGAGGATGGCGG | | | |
| | TTGGGGGGAATCAATGAAGTCCAGTGA | | | |
| | ATTACATAGTTATGTGGATAGTGAAAA | | | |
| | ATCGCTAGTCGTTCAAACCGCATGGGC | | | |
| | GCTAATTGCACTTCTTTTCGCTGAATAT | | | |
| | CCTAATAAAGAAGTCATCGACCGCGGT | | | |
| | ATTGACCTTTTAAAAAATAGACAAGAA | | | |
| | GAATCCGGGGAATGGAAATTTGAAAG | | | |
| | TGTAGAAGGTGTTTTCAACCACTCTTG | | | |
| | TGCAATTGAATACCCAAGTTATCGATT | | | |
| | CTTATTCCCTATTAAGGCATTAGGTAT | | | |
| | GTACAGCAGGGCATAG | | | |
| 756248 | ATGACAGAATTTTATTCTGACACAATC | 81 | MTEFYSDTIGLPKTDPRLW | 101 |
| | GGTCTACCAAAGACAGATCCACGTCTT | | RLRTDELGRESWEYLTPQQ | |
| | TGGAGACTGAGAACTGATGAGCTAGG | | AANDPPSTFTQWLLQDPKF | |
| | CCGAGAAAGCTGGGAATATTTAACCCC | | PQPHPERNKHSPDFSAFDA | |
| | TCAGCAAGCCGCAAACGACCCACCATC | | CHNGASFFKLLQEPDSGIFP | |
| | CACTTTCACGCAGTGGCTTCTTCAAGA | | CQYKGPMFMTIGYVAVNYI | |
| | TCCCAAATTTCCTCAACCTCATCCAGA | | AGIEIPEHERIELIRYIVNTA | |
| | AAGAAATAAGCATTCACCAGATTTTTC | | HPVDGGWGLHSVDKSTVF | |
| | AGCCTTCGATGCGTGTCATAATGGTGC | | GTVLNYVILRLLGLPKDHP | |
| | ATCTTTTTTCAAACTGCTTCAAGAGCCT | | VCAKARSTLLRLGGAIGSP | |
| | GACTCAGGTATTTTTCCGTGTCAATAT | | HWGKIWLSALNLYKWEGV | |
| | AAAGGACCCATGTTCATGACAATCGGT | | NPAPPETWLLPYSLPMHPG | |
| | TACGTAGCTGTAAACTATATCGCCGGT | | RWWVHTRGVYIPVSYLSLV | |
| | ATTGAAATTCCTGAGCATGAGAGAATA | | KFSCPMTPLLEELRNEIYTS | |
| | GAATTAATTAGATACATCGTCAATACA | | PFDKINFSKNRNAVCGVDL | |
| | GCACATCCGGTTGATGGTGGCTGGGGT | | YYPHSTTLNIANSLVVFYEK | |
| | CTACATTCTGTTGACAAATCCACCGTG | | YLRNRFIYSLSKKKVYDLIK | |
| | TTTGGTACAGTATTGAACTATGTAATCT | | TELQNTDSLCIAPVNQAFC | |
| | TACGTTTATTGGGTCTACCCAAGGACC | | ALVTLIEEGVDSEAFQRLQ | |
| | ACCCGGTTTGCGCCAAGGCAAGAAGC | | YRFKDALFHGPQGMTIMGT | |
| | ACATTGTTAAGGTTAGGCGGTGCTATT | | NGVQTWDCAFAIQYFFVA | |
| | GGATCCCCTCACTGGGGAAAAATTTGG | | GLAERPEFYNTIVSAYKFLC | |
| | CTAAGTGCACTAAACTTGTATAAATGG | | HAQFDTECVPGSYRDKRKG | |
| | GAAGGTGTGAACCCTGCCCCTCCTGAA | | AWGFSTKTQGYTVADCTA | |
| | ACTTGGTTACTTCCATATTCACTGCCCA | | EAIKAIIMVKNSPVFSEVHH | |
| | TGCATCCGGGGAGATGGTGGGTTCATA | | MISSERLFEGIDVLLNLQNI | |
| | CTAGAGGTGTTTACATTCCGGTCAGTT | | GSLEYGSFATYEKIKAPLA | |
| | ACCTGTCATTGGTCAAATTTTCTTGCCC | | METLNPAEVFGNIMVEYPY | |
| | AATGACTCCTCTTCTTGAAGAACTGAG | | VECTDSSVLGLTYFHKYFD | |
| | GAATGAAATTTACACTAGTCCGTTTGA | | YRKEEIRTRIRIAIEFIKKSQL | |
| | CAAGATTAACTTCTCCAAGAACAGGAA | | PDGSWYGSWGICFTYAGM | |
| | TGCCGTATGTGGAGTAGACCTATATTA | | FALEALHNVGETYENSSTV | |
| | CCCCCATTCTACTACTTTGAATATTGCG | | RKGCDFLVSKQMKDGGWG | |
| | AACAGCCTTGTAGTATTTTACGAAAAA | | ESMKSSELHSYVDSEKSLV | |
| | TACCTAAGAAACCGGTTCATTTACTCT | | VQTTWALIALLFAEYPNKE | |
| | CTATCCAAGAAGAAGGTTTATGATCTA | | VIDRGIDLLKNRQEESGEW | |
| | ATCAAAACGGAGTTACAGAATACTGAT | | KFGSVEGVFNHSCAIEYPSY | |
| | TCCTTGTGTATAGCACCTGTTAACCAG | | RFLFPIKALGMYSRAYETH | |
| | GCGTTTTGCGCACTTGTCACTCTTATTG | | TL | |
| | AAGAAGGGGTAGACTCGGAAGCGTTC | | | |
| | CAGCGTCTCCAATATAGGTTCAAGGAT | | | |
| | GCATTGTTCCATGGTCCACAGGGTATG | | | |
| | ACCATTATGGGAACAAATGGTGTGCAA | | | |
| | ACCTGGGATTGTGCGTTTGCCATTCAA | | | |
| | TACTTTTTCGTCGCAGGCCTCGCAGAA | | | |
| | AGACCTGAATTCTATAACACAATTGTC | | | |
| | TCTGCCTATAAATTCTTGTGTCATGCTC | | | |
| | AATTTGACACCGAGTGCGTTCCAGGTA | | | |
| | GTTATAGGGATAAGAGAAAGGGGGCT | | | |
| | TGGGGCTTCTCAACAAAAACACAGGGC | | | |
| | TATACAGTGGCAGATTGCACTGCAGAA | | | |
| | GCAATTAAAGCCATCATCATGGTGAAA | | | |
| | AACTCTCCCGTCTTTAGTGAAGTACAC | | | |
| | CATATGATTAGCAGTGAACGTTTATTT | | | |
| | GAAGGCATTGATGTGTTATTGAACCTA | | | |
| | CAAAACATCGGATCTCTTGAATATGGT | | | |
| | TCCTTTGCAACCTATGAAAAAATCAAG | | | |
| | GCCCCACTAGCAATGGAAACCTTGAAT | | | |
| | CCTGCTGAAGTTTTTGGTAACATAATG | | | |
| | GTAGAATACCCATACGTGGAATGTACT | | | |
| | GATTCATCCGTTCTGGGGTTGACATAT | | | |
| | TTTCACAAGTACTTCGACTATAGGAAA | | | |
| | GAGGAAATACGTACACGCATCAGAAT | | | |
| | CGCCATCGAATTCATAAAAAAATCTCA | | | |
| | ATTACCAGATGGAAGTTGGTATGGAAG | | | |
| | CTGGGGTATTTGTTTTACATATGCCGGT | | | |
| | ATGTTTGCATTGGAGGCATTACACAAC | | | |
| | GTGGGGGAGACCTATGAGAATTCCTCA | | | |
| | ACGGTAAGAAAAGGTTGCGACTTCTTG | | | |
| | GTCAGTAAACAGATGAAGGATGGCGG | | | |
| | TTGGGGGGAATCAATGAAGTCCAGTGA | | | |
| | ATTACATAGTTATGTGGATAGTGAAAA | | | |
| | ATCGCTAGTCGTTCAAACCACATGGGC | | | |
| | GCTAATTGCACTTCTTTTCGCTGAATAT | | | |
| | CCTAATAAAGAAGTCATCGACCGCGGT | | | |
| | ATTGACCTTTTAAAAAATAGACAAGAA | | | |
| | GAATCCGGGGAATGGAAATTTGGAAG | | | |
| | TGTAGAAGGTGTTTTCAACCACTCTTG | | | |
| | TGCAATTGAATACCCAAGTTATCGATT | | | |
| | CTTATTCCCTATTAAGGCATTAGGTAT | | | |
| | GTACAGCAGGGCATATGAAACACATA | | | |
| | CGCTTTAA | | | |
| 756249 | ATGACAGAATTTTATTCTGACACAATC | 82 | MTEFYSDTIGLPKTDPRLW | 102 |
| | GGTCTACCAAAGACAGATCCACGTCTT | | RLRTDELGRESWEYLTPQQ | |
| | TGGAGACTGAGAACTGATGAGCTAGG | | AANDPPSTFTQWLLQDPKF | |
| | CCGAGAAAGCTGGGAATATTTAACCCC | | PQPHPEGNKHSPDFSAFDA | |
| | TCAGCAAGCCGCAAACGACCCACCATC | | CHNGASFFKLLQEPDSGIFP | |
| | CACTTTCACGCAGTGGCTTCTTCAAGA | | CQYKGPMFMTIGYVAVNYI | |
| | TCCCAAATTTCCTCAACCTCATCCAGA | | AGIEVPEHERIELIRYIVNTA | |
| | AGGAAATAAGCATTCACCAGATTTTTC | | HPVDGGWGLHSVDKSTVF | |
| | AGCCTTCGATGCGTGTCATAATGGTGC | | GTVLNYVILRLLGLPKDHP | |
| | ATCTTTTTTCAAACTGCTTCAAGAGCCT | | VCAKARSTLLRLGGAIGSP | |
| | GACTCAGGTATTTTTCCGTGTCAATAT | | HWGKIWLSALNLYKWEGV | |
| | AAAGGACCCATGTTCATGACAATCGGT | | NPAPPETWLLPYSLPIHPGR | |
| | TACGTAGCCGTAAACTATATCGCCGGT | | WWVHTRGVYIPVSYLSLV | |
| | ATTGAAGTTCCTGAGCATGAGAGAATA | | KFSCPMTPLLEELRNEIYTK | |
| | GAATTAATTAGATACATCGTCAATACA | | PFDKINISKNRNTVCGVDLY | |
| | GCACATCCGGTTGATGGTGGCTGGGGT | | YPHSTTLNIANSLVVFYEKY | |
| | CTACATTCTGTTGACAAATCCACCGTG | | LRNRFIYSLSKKKVYDLIKT | |
| | TTTGGTACAGTATTGAACTATGTAATCT | | ELQNADSLCIAPVNQAFCA | |
| | TACGTTTATTGGGTCTACCCAAGGACC | | LVTLIEEGVDSEAFQRLQYR | |
| | ACCCGGTTTGCGCCAAGGCAAGAAGC | | FKDALFHGPQGMTIMGTNG | |
| | ACATTGTTAAGGTTAGGCGGTGCTATT | | VQTWDCAFAIQYFFVAGLA | |
| | GGATCCCCTCACTGGGGAAAAATTTGG | | ERPEFYNTIVSAYKFLCHAQ | |
| | CTAAGTGCACTAAACTTGTATAAATGG | | FDTECVPGSYRDKRKGAW | |
| | GAAGGTGTGAACCCTGCCCCTCCTGAA | | GFSTKTQGYTVADCTAEAI | |
| | ACTTGGTTACTTCCATATTCACTGCCCA | | KAIIMVKNSPVFSEVHHMIS | |
| | TTCATCCGGGGAGATGGTGGGTTCATA | | SERLFEGIDVLLNLQNIGSF | |
| | CTAGAGGTGTTTACATTCCGGTCAGTT | | EYGSFATYEKIKAPLAMET | |
| | ACCTGTCATTGGTCAAATTTTCTTGCCC | | LNPAEVFGNIMVEYPYVEC | |
| | AATGACTCCTCTTCTTGAAGAACTGAG | | TDSSVLGLTYFHKYFDYRK | |
| | GAATGAAATTTACACTAAACCGTTTGA | | EEIRTRIRIAIEFIKKSQLPDG | |
| | CAAGATTAACATCTCCAAGAACAGGA | | SWYGSWGICFTYAGMFAL | |
| | ATACCGTATGTGGAGTAGACCTATATT | | EALHTVGETYENSSTVRKG | |
| | ACCCCCATTCTACTACTTTGAATATTGC | | SDFLVSKQMKDGGWGESM | |
| | GAACAGCCTTGTAGTATTTTACGAAAA | | KSSELHSYVDSEKSLVVQT | |
| | ATACCTAAGAAACCGGTTCATTTACTC | | AWALIALLFAEYPNKEVID | |
| | TCTATCCAAGAAGAAGGTTTATGATCT | | RGIDLLKNRQEESGEWKFE | |
| | AATCAAAACGGAGTTACAGAATGCTG | | SVEGVFNHSCAIEYPSYRFL | |
| | ATTCCTTGTGTATAGCACCTGTTAACC | | FPIKALGMYSRAYETHTL | |
| | AGGCGTTTTGCGCACTTGTCACTCTTAT | | | |
| | TGAAGAAGGGGTAGACTCGGAAGCGT | | | |
| | TCCAGCGTCTCCAATATAGGTTCAAGG | | | |
| | ATGCATTGTTCCATGGTCCACAGGGTA | | | |
| | TGACCATTATGGGAACAAATGGTGTGC | | | |
| | AAACCTGGGATTGTGCGTTTGCCATTC | | | |
| | AATACTTTTTCGTCGCAGGCCTCGCAG | | | |
| | AAAGACCTGAATTCTATAACACAATTG | | | |
| | TCTCTGCCTATAAATTCTTGTGTCATGC | | | |
| | TCAATTTGACACCGAGTGCGTTCCAGG | | | |
| | TAGTTATAGGGATAAGAGAAAGGGGG | | | |
| | CTTGGGGCTTCTCAACAAAAACACAGG | | | |
| | GCTATACAGTGGCAGATTGCACTGCAG | | | |
| | AAGCAATTAAAGCCATCATCATGGTGA | | | |
| | AAAACTCTCCCGTCTTTAGTGAAGTAC | | | |
| | ACCATATGATTAGCAGTGAACGTTTAT | | | |
| | TTGAAGGCATTGATGTGTTATTGAACC | | | |
| | TACAAAACATCGGATCTTTTGAATATG | | | |
| | GTTCCTTTGCAACCTATGAAAAAATCA | | | |
| | AGGCCCCACTAGCAATGGAAACCTTGA | | | |
| | ATCCTGCTGAAGTTTTTGGTAACATAA | | | |
| | TGGTAGAATACCCATACGTGGAATGTA | | | |
| | CTGATTCATCCGTTCTGGGGTTGACAT | | | |
| | ATTTTCACAAGTACTTCGACTATAGGA | | | |
| | AAGAGGAAATACGTACACGCATCAGA | | | |
| | ATCGCCATCGAATTCATAAAAAAATCT | | | |
| | CAATTACCAGATGGAAGTTGGTATGGA | | | |
| | AGCTGGGGTATTTGTTTTACATATGCC | | | |
| | GGTATGTTTGCATTGGAGGCATTACAC | | | |
| | ACCGTGGGGGAGACCTATGAGAATTCC | | | |
| | TCAACGGTAAGAAAAGGTAGCGACTTC | | | |
| | TTGGTCAGTAAACAGATGAAGGATGGC | | | |
| | GGTTGGGGGGAATCAATGAAGTCCAGT | | | |
| | GAATTACATAGTTATGTGGATAGTGAA | | | |
| | AAATCGCTAGTCGTTCAAACCGCATGG | | | |
| | GCGCTAATTGCACTTCTTTTCGCTGAAT | | | |
| | ATCCTAATAAAGAAGTCATCGACCGCG | | | |
| | GTATTGACCTTTTAAAAAATAGACAAG | | | |
| | AAGAATCCGGGGAATGGAAATTTGAA | | | |
| | AGTGTAGAAGGTGTTTTCAACCACTCT | | | |
| | TGTGCAATTGAATACCCAAGTTATCGA | | | |
| | TTCTTATTCCCTATTAAGGCATTAGGTA | | | |
| | TGTACAGCAGGGCATATGAAACACATA | | | |
| | CGCTTTAA | | | |
| N/A | ATGGGAATCCACGAAAGTGTGTCGAA | 2 | MGIHESVSKQFAKNGHSKY | 1 |
| (Wild- | ACAGTTTGCGAAAAACGGACATTCCAA | | RSDRYGLPKTDLRRWTFHA | |
| type | GTACCGCAGCGACCGATACGGCTTACC | | SDLGAQWWKYDDTTPLEE | |
| ERG7) | TAAGACGGATCTGCGACGATGGACGTT | | LEKRATDYVKYSLELPGYA | |
| | CCACGCGTCCGATCTGGGGGCGCAATG | | PVTLDSKPVKNAYEAALKN | |
| | GTGGAAGTATGACGATACCACACCGCT | | WHLFASLQDPDSGAWQSE | |
| | GGAAGAGCTGGAAAAGAGGGCTACCG | | YDGPQFMSIGYVTACYFGG | |
| | ACTACGTCAAATACTCGCTGGAGCTGC | | NEIPTPVKTEMIRYIVNTAH | |
| | CGGGATACGCGCCCGTGACTCTGGACT | | PVDGGWGLHKEDKSTCFG | |
| | CCAAGCCCGTGAAAAATGCCTACGAA | | TSINYVVLRLLGLSRDHPVC | |
| | GCGGCTCTCAAAAACTGGCATCTGTTT | | VKARKTLLTKFGGAINNPH | |
| | GCGTCGCTGCAAGACCCCGACTCCGGC | | WGKTWLSILNLYKWEGVN | |
| | GCATGGCAGTCGGAATACGACGGACC | | PAPGELWLLPYFVPVHPGR | |
| | GCAGTTCATGTCGATCGGTTATGTGAC | | WWVHTRWIYLAMGYLEA | |
| | GGCGTGCTACTTTGGCGGCAACGAGAT | | AEAQCELTPLLEELRDEIYK | |
| | CCCCACGCCGGTCAAAACCGAAATGAT | | KPYSEIDFSKHCNSISGVDL | |
| | CAGATACATTGTCAACACAGCCCACCC | | YYPHTGLLKFGNALLRRYR | |
| | AGTTGACGGAGGCTGGGGCCTTCACAA | | KFRPQWIKEKVKEEIYNLC | |
| | AGAAGACAAGAGCACCTGTTTCGGTAC | | LREVSNTRHLCLAPVNNAM | |
| | CAGCATCAACTACGTGGTCCTGCGACT | | TSIVMYLHEGPDSANYKKI | |
| | ACTGGGCCTGTCACGGGATCATCCGGT | | AARWPEFLSLNPSGMFMN | |
| | CTGCGTCAAGGCGCGCAAAACGCTGCT | | GTNGLQVWDTAFAVQYAC | |
| | CACCAAGTTTGGCGGCGCCATCAACAA | | VCGFAELPQYQKTIRAAFD | |
| | CCCCCATTGGGGCAAGACCTGGCTGTC | | FLDRSQINEPTEENSYRDDR | |
| | GATTCTCAATCTCTACAAATGGGAGGG | | VGGWPFSTKTQGYPVSDCT | |
| | TGTGAATCCGGCCCCTGGCGAGCTCTG | | AEALKAIIMVQNTPGYEDL | |
| | GCTGTTGCCCTACTTTGTTCCTGTTCAT | | KKQVSDKRKHTAIDLLLGM | |
| | CCGGGCCGATGGTGGGTCCATACCCGG | | QNVGSFEPGSFASYEPIRAS | |
| | TGGATCTACCTTGCCATGGGCTATCTG | | SMLEKINPAEVFGNIMVEY | |
| | GAGGCTGCGGAGGCCCAATGCGAACT | | PYVECTDSVVLGLSYFRKY | |
| | CACTCCGTTGCTGGAGGAGCTCCGAGA | | HDYRNEDVDRAISAAIGYII | |
| | CGAAATCTACAAAAAGCCCTACTCGGA | | REQQPDGGFFGSWGVCYC | |
| | GATTGATTTCTCCAAACATTGCAACTC | | YAHMFAMEALETQNLNYN | |
| | CATCTCCGGAGTCGACCTCTACTATCC | | NCSTVQKACDFLAGYQEA | |
| | CCACACCGGCCTTTTGAAGTTTGGCAA | | DGGWAEDFKSCETQMYVR | |
| | CGCGCTTCTCCGACGATACCGCAAGTT | | GPHSLVVPTAMALLSLMSG | |
| | CAGACCGCAGTGGATCAAAGAAAAGG | | RYPQEDKIHAAARFLMSKQ | |
| | TCAAGGAGGAAATTTACAACTTGTGCC | | MSNGEWLKEEMEGVFNHT | |
| | TTCGAGAGGTTTCCAACACACGACACT | | CAIEYPNYRFYFVMKALGL | |
| | TGTGTCTCGCTCCCGTCAACAATGCCA | | YFKGYCQ | |
| | TGACCTCCATTGTCATGTATCTCCATGA | | | |
| | GGGGCCCGATTCGGCGAATTACAAAA | | | |
| | AGATTGCGGCCCGATGGCCCGAATTTC | | | |
| | TGTCTCTGAATCCGTCGGGAATGTTTA | | | |
| | TGAACGGCACCAACGGTCTGCAGGTCT | | | |
| | GGGATACTGCGTTTGCCGTGCAATACG | | | |
| | CGTGTGTTTGTGGCTTTGCCGAACTTCC | | | |
| | CCAGTACCAGAAGACGATCCGAGCGG | | | |
| | CGTTTGATTTTCTCGATCGGTCCCAGAT | | | |
| | CAACGAGCCGACGGAGGAAAATTCCT | | | |
| | ATCGAGACGACCGCGTCGGAGGATGG | | | |
| | CCCTTTAGTACCAAGACCCAGGGGTAT | | | |
| | CCAGTCTCCGACTGTACTGCCGAGGCT | | | |
| | CTCAAGGCCATCATCATGGTCCAGAAT | | | |
| | ACGCCTGGATACGAGGATCTGAAGAA | | | |
| | ACAAGTGTCTGACAAGCGGAAACACA | | | |
| | CTGCCATCGATCTACTTTTGGGAATGC | | | |
| | AGAACGTGGGCTCGTTTGAACCGGGCT | | | |
| | CTTTCGCCTCCTATGAGCCTATCCGGG | | | |
| | CGTCGTCCATGCTGGAGAAGATCAATC | | | |
| | CGGCCGAGGTGTTTGGAAACATCATGG | | | |
| | TGGAGTATCCGTACGTGGAATGCACTG | | | |
| | ATTCTGTTGTTCTGGGTCTGTCCTACTT | | | |
| | TCGAAAGTACCACGATTACCGCAACGA | | | |
| | AGACGTGGACCGAGCCATCTCTGCTGC | | | |
| | CATTGGATACATTATTCGAGAGCAGCA | | | |
| | GCCTGACGGCGGCTTCTTTGGCTCCTG | | | |
| | GGGCGTGTGCTACTGCTACGCTCACAT | | | |
| | GTTTGCCATGGAGGCTCTGGAGACGCA | | | |
| | GAATCTCAACTATAACAACTGTTCCAC | | | |
| | GGTTCAAAAGGCGTGCGACTTTCTGGC | | | |
| | GGGCTACCAGGAAGCAGATGGAGGCT | | | |
| | GGGCCGAGGACTTTAAGTCGTGCGAGA | | | |
| | CTCAGATGTACGTGCGCGGACCCCATT | | | |
| | CGCTGGTCGTGCCTACTGCCATGGCCC | | | |
| | TGTTGAGTTTGATGAGTGGTCGGTATC | | | |
| | CCCAGGAGGACAAGATTCATGCTGCGG | | | |
| | CCCGGTTTCTCATGAGCAAGCAGATGA | | | |
| | GCAACGGTGAGTGGCTCAAGGAGGAG | | | |
| | ATGGAGGGGGTGTTTAACCATACTTGT | | | |
| | GCCATTGAGTATCCCAACTACCGGTTT | | | |
| | TATTTTGTCATGAAGGCTTTGGGGTTGT | | | |
| | ATTTCAAGGGATATTGCCAGTGA | | | |
| 2F1 | ATGGGAATCCACGAAAGTGTGTCGAA | 103 | MGIHESVSKQFAKNGHSKY | 118 |
| | ACAGTTTGCGAAAAACGGACATTCCAA | | RSDRYGLPKTDLRRWTFHA | |
| | GTACCGCAGCGACCGATACGGCTTACC | | SDLGAQWWKYDDTTPLEE | |
| | TAAGACGGATCTGCGACGATGGACGTT | | LEKRATDYVKYSLELPGYA | |
| | CCACGCGTCCGATCTGGGGGCGCAATG | | PVTLDSKPVKNAYEAALKN | |
| | GTGGAAGTATGACGATACCACACCGCT | | WHLFASLQDPDSGAWQSE | |
| | GGAAGAGCTGGAAAAGAGGGCTACCG | | YDGPQFMSIGYVTACYFGG | |
| | ACTACGTCAAATACTCGCTGGAGCTGC | | NEIPTPVKTEMIRYIVNTAH | |
| | CGGGATACGCGCCCGTGACTCTGGACT | | PVDGGWGLHKEDKSTCFG | |
| | CCAAGCCCGTGAAAAATGCCTACGAA | | TSINYVVLRLLGLSRDHPVC | |
| | GCGGCTCTCAAAAACTGGCATCTGTTT | | VKARKTLLTKFGGAINNPH | |
| | GCGTCGCTGCAAGACCCCGACTCCGGC | | WGKTWLSILNLYKWEGVN | |
| | GCATGGCAGTCGGAATACGACGGACC | | PAPGELWLLPYFVPVHPGR | |
| | GCAGTTCATGTCGATCGGTTATGTGAC | | WWFHTRWIYLAMGYLEAA | |
| | GGCGTGCTACTTTGGCGGCAACGAGAT | | EAQCELTPLLEELRDEIYKK | |
| | CCCCACGCCGGTCAAAACCGAAATGAT | | PYSEIDFSKHCNSISGVDLY | |
| | CAGATACATTGTCAACACAGCCCACCC | | YPHTGLLKFGNALLRRYRK | |
| | AGTTGACGGAGGCTGGGGCCTTCACAA | | FRPQWIKEKVKEEIYNLCLR | |
| | AGAAGACAAGAGCACCTGTTTCGGTAC | | EVSNTRHLCLAPVNNAMTS | |
| | CAGCATCAACTACGTGGTCCTGCGACT | | IVMYLHEGPVSANYKKIAA | |
| | ACTGGGCCTGTCACGGGATCATCCGGT | | RWPEFLSLNPSGMFMNGTN | |
| | CTGCGTCAAGGCGCGCAAAACGCTGCT | | GLQVWDTAFAVQYACVCG | |
| | CACCAAGTTTGGCGGCGCCATCAACAA | | FAELPQYQKTIRAAFDFLDR | |
| | CCCCCATTGGGGCAAGACCTGGCTGTC | | SQINEPTEENSYRDDRVGG | |
| | GATTCTCAATCTCTACAAATGGGAGGG | | WPFSTKTQGYPVSDCTAEA | |
| | TGTGAATCCGGCCCCTGGCGAGCTCTG | | LKAIIMVQNTPGYEDLKKQ | |
| | GCTGTTGCCCTACTTTGTTCCTGTTCAT | | VSDKRKHTAIDLLLGMQNV | |
| | CCGGGCCGATGGTGGTTCCATACCCGG | | GSFEPGSFASYEPIRASSML | |
| | TGGATCTACCTTGCCATGGGCTATCTG | | EKINPAEVFGNIMVEYPYV | |
| | GAGGCTGCGGAGGCCCAATGCGAACT | | ECTDSVVLGLSYFRKYHDY | |
| | CACTCCGTTGCTGGAGGAGCTCCGAGA | | RNEDVDRAISAAIGYIIREQ | |
| | CGAAATCTACAAAAAGCCCTACTCGGA | | QPDGGFFGSWGVCYCYAH | |
| | GATTGATTTCTCCAAACATTGCAACTC | | MFAMEALETQNLNYNNCS | |
| | CATCTCCGGAGTCGACCTCTACTATCC | | TVQKACDFLAGYQEADGG | |
| | CCACACCGGCCTTTTGAAGTTTGGCAA | | WAEDFKSCETQMYVRGPH | |
| | CGCGCTTCTCCGACGATACCGCAAGTT | | SLVVPTAMALLSLMSGRYP | |
| | CAGACCGCAGTGGATCAAAGAAAAGG | | QEDKIHAAARFLMSKQMS | |
| | TCAAGGAGGAAATTTACAACTTGTGCC | | NDEWLKEEMEGVFNHTCAI | |
| | TTCGAGAGGTTTCCAACACACGACACT | | EYPNYRFYFVMKALGLYFK | |
| | TGTGTCTCGCTCCCGTCAACAATGCCA | | GYCQ | |
| | TGACCTCCATTGTCATGTATCTCCATGA | | | |
| | GGGGCCCGTTTCGGCGAATTACAAAAA | | | |
| | GATTGCGGCCCGATGGCCCGAATTTCT | | | |
| | GTCTCTGAATCCGTCGGGAATGTTTAT | | | |
| | GAACGGCACCAACGGTCTGCAGGTCTG | | | |
| | GGATACTGCGTTTGCCGTGCAATACGC | | | |
| | GTGTGTTTGTGGCTTTGCCGAACTTCCC | | | |
| | CAGTACCAGAAGACGATCCGAGCGGC | | | |
| | GTTTGATTTTCTCGATCGGTCCCAGATC | | | |
| | AACGAGCCGACGGAGGAAAATTCCTA | | | |
| | TCGAGACGACCGCGTCGGAGGATGGC | | | |
| | CCTTTAGTACCAAGACCCAGGGGTATC | | | |
| | CAGTCTCCGACTGTACTGCCGAGGCTC | | | |
| | TCAAGGCCATCATCATGGTCCAGAATA | | | |
| | CGCCTGGATACGAGGATCTGAAGAAA | | | |
| | CAAGTGTCTGACAAGCGGAAACACACT | | | |
| | GCCATCGATCTACTTTTGGGAATGCAG | | | |
| | AACGTGGGCTCGTTTGAACCGGGCTCT | | | |
| | TTCGCCTCCTATGAGCCTATCCGGGCG | | | |
| | TCGTCCATGCTGGAGAAGATCAATCCG | | | |
| | GCCGAGGTGTTTGGAAACATCATGGTG | | | |
| | GAGTATCCGTACGTGGAATGCACTGAT | | | |
| | TCTGTTGTTCTGGGTCTGTCCTACTTTC | | | |
| | GAAAGTACCACGATTACCGCAACGAA | | | |
| | GACGTGGACCGAGCCATCTCTGCTGCC | | | |
| | ATTGGATACATTATTCGAGAGCAGCAG | | | |
| | CCTGACGGCGGCTTCTTTGGCTCCTGG | | | |
| | GGCGTGTGCTACTGCTACGCTCACATG | | | |
| | TTTGCCATGGAGGCTCTGGAGACGCAG | | | |
| | AATCTCAACTATAACAACTGTTCCACG | | | |
| | GTTCAAAAGGCGTGCGACTTTCTGGCG | | | |
| | GGCTACCAGGAAGCAGATGGAGGCTG | | | |
| | GGCCGAGGACTTTAAGTCGTGCGAGAC | | | |
| | TCAGATGTACGTGCGCGGACCCCATTC | | | |
| | GCTGGTCGTGCCTACTGCCATGGCCCT | | | |
| | GTTGAGTTTGATGAGTGGTCGGTATCC | | | |
| | CCAGGAGGACAAGATTCATGCTGCGGC | | | |
| | CCGGTTTCTCATGAGCAAGCAGATGAG | | | |
| | CAACGATGAGTGGCTCAAGGAGGAGA | | | |
| | TGGAGGGGGTGTTTAACCATACTTGTG | | | |
| | CCATTGAGTATCCCAACTACCGGTTTT | | | |
| | ATTTTGTCATGAAGGCTTTGGGGTTGT | | | |
| | ATTTCAAGGGATATTGCCAGTGA | | | |
| 2F11 | ATGGGAATCCACGAAAGTGTGTCGAA | 104 | MGIHESVSKQFAKNGHSKY | 119 |
| | ACAGTTTGCGAAAAACGGACATTCCAA | | RSDRYGLPKTDLRRWTFHA | |
| | GTACCGCAGCGACCGATACGGCTTACC | | SDLGAQWWKYDDTTPLEE | |
| | TAAGACGGATCTGCGACGATGGACGTT | | LEKRATDYVKYSLELPGYA | |
| | CCACGCGTCCGATCTGGGGGCGCAATG | | PVTLDSKPVKNAYEAALKN | |
| | GTGGAAGTATGACGATACCACACCGCT | | WHLFASLQDPDSGAWQSE | |
| | GGAAGAGCTGGAAAAGAGGGCTACCG | | YDGPQFMSIGYVTACYFGG | |
| | ACTACGTCAAATACTCGCTGGAGCTGC | | NEIPTPVKTEMIRYIVNTAH | |
| | CGGGATACGCGCCCGTGACTCTGGACT | | PVDGGWGLHKEDKSTCFG | |
| | CCAAGCCCGTGAAAAATGCCTACGAA | | TSINYVVLRLLGLSWDHPV | |
| | GCGGCTCTCAAAAACTGGCATCTGTTT | | CVKARKTLLTKFGGAINNP | |
| | GCGTCGCTGCAAGACCCCGACTCCGGC | | HWGKTWLSILNLYKWEGV | |
| | GCATGGCAGTCGGAATACGACGGACC | | NPAPGELWLMPYFVPVHPG | |
| | GCAGTTCATGTCGATCGGTTATGTGAC | | RWWVHTRWIYLAMGYRE | |
| | GGCGTGCTACTTTGGCGGCAACGAGAT | | AAEAQCELTPLLEELRDEIY | |
| | CCCCACGCCGGTCAAAACCGAAATGAT | | KKPYSEIDFSKHCNSISGVD | |
| | CAGATACATTGTCAACACAGCCCACCC | | LYYPHTGLLKFGNALLRRY | |
| | AGTTGACGGAGGCTGGGGCCTTCACAA | | RKFRPQWIKEKVKEEIYNL | |
| | AGAAGACAAGAGCACCTGTTTCGGTAC | | CLREVSNTRHLCLAPVNNA | |
| | CAGCATCAACTACGTGGTCCTGCGACT | | MTSIVMYLHEGPDSANYKK | |
| | ACTGGGCCTGTCATGGGATCATCCGGT | | IAARWPEFLSLNPSGMFMN | |
| | CTGCGTCAAGGCGCGCAAAACGCTGCT | | GTNGLQVWDTAFAVQYAC | |
| | CACCAAGTTTGGCGGCGCCATCAACAA | | VCGFAELPQYQKTIRAAFD | |
| | CCCCCATTGGGGCAAGACCTGGCTGTC | | FLDRSQINEPTEENSYRDDR | |
| | GATTCTCAATCTCTACAAATGGGAGGG | | VGGWPFSTKTQGYPVSDCT | |
| | TGTGAATCCGGCCCCTGGCGAGCTCTG | | AEALKAIIMVQNTPGYEDL | |
| | GCTGATGCCCTACTTTGTTCCTGTTCAT | | KKQVSDKRKHTAIDLLLGM | |
| | CCGGGCCGATGGTGGGTCCATACCCGG | | QNVGSFEPGSFASYEPIRAS | |
| | TGGATCTACCTTGCCATGGGCTATCGG | | SMLEKINPAEVFGNIMVEY | |
| | GAGGCTGCGGAGGCCCAATGCGAACT | | PYVECTDSVVLGLSYFRKY | |
| | CACTCCGTTGCTGGAGGAGCTCCGAGA | | HDYRNEDVDRAISAAIGYII | |
| | CGAAATCTACAAAAAGCCCTACTCGGA | | REQQPDGGFFGSWGVCYC | |
| | GATTGATTTCTCCAAACATTGCAACTC | | YAHMFAMEALETQNLNYN | |
| | CATCTCCGGAGTCGACCTCTACTATCC | | NCSTVQKACDFLAGYQEA | |
| | CCACACCGGCCTTTTGAAGTTTGGCAA | | DGGWAEDFKSCETQMYVR | |
| | CGCGCTTCTCCGACGATACCGCAAGTT | | GPHSLVVPTAMALLSLMSG | |
| | CAGACCGCAGTGGATCAAAGAAAAGG | | RYPQEDKIHAAARFLMSKQ | |
| | TCAAGGAGGAAATTTACAACTTGTGCC | | MSNGEWLKEEMQGVFNHT | |
| | TTCGAGAGGTTTCCAACACACGACACT | | CAIEYPNYRFYFVMKALGL | |
| | TGTGTCTCGCTCCCGTCAACAATGCCA | | YFKGYCQ | |
| | TGACCTCCATTGTCATGTATCTCCATGA | | | |
| | GGGGCCCGATTCGGCGAATTACAAAA | | | |
| | AGATTGCGGCCCGATGGCCCGAATTTC | | | |
| | TGTCTCTGAATCCGTCGGGAATGTTTA | | | |
| | TGAACGGCACCAACGGTCTGCAGGTCT | | | |
| | GGGATACTGCGTTTGCCGTGCAATACG | | | |
| | CGTGTGTTTGTGGCTTTGCCGAACTTCC | | | |
| | CCAGTACCAGAAGACGATCCGAGCGG | | | |
| | CGTTTGATTTTCTCGATCGGTCCCAGAT | | | |
| | CAACGAGCCGACGGAGGAAAATTCCT | | | |
| | ATCGAGACGACCGCGTCGGAGGATGG | | | |
| | CCCTTTAGTACCAAGACCCAGGGGTAT | | | |
| | CCAGTCTCCGACTGTACTGCCGAGGCT | | | |
| | CTCAAGGCCATCATCATGGTCCAGAAT | | | |
| | ACGCCTGGATACGAGGATCTGAAGAA | | | |
| | ACAAGTGTCTGACAAGCGGAAACACA | | | |
| | CTGCCATCGATCTACTTTTGGGAATGC | | | |
| | AGAACGTGGGCTCGTTTGAACCGGGCT | | | |
| | CTTTCGCCTCCTATGAGCCTATCCGGG | | | |
| | CGTCGTCCATGCTGGAGAAGATCAATC | | | |
| | CGGCCGAGGTGTTTGGAAACATCATGG | | | |
| | TGGAGTATCCGTACGTGGAATGCACTG | | | |
| | ATTCTGTTGTTCTGGGTCTGTCCTACTT | | | |
| | TCGAAAGTACCACGATTACCGCAACGA | | | |
| | AGACGTGGACCGAGCCATCTCTGCTGC | | | |
| | CATTGGATACATTATTCGAGAGCAGCA | | | |
| | GCCTGACGGCGGCTTCTTTGGCTCCTG | | | |
| | GGGCGTGTGCTACTGCTACGCTCACAT | | | |
| | GTTTGCCATGGAGGCTCTGGAGACGCA | | | |
| | GAATCTCAACTATAACAACTGTTCCAC | | | |
| | GGTTCAAAAGGCGTGCGACTTTCTGGC | | | |
| | GGGCTACCAGGAAGCAGATGGAGGCT | | | |
| | GGGCCGAGGACTTTAAGTCGTGCGAGA | | | |
| | CTCAGATGTACGTGCGCGGACCCCATT | | | |
| | CGCTGGTCGTGCCTACTGCCATGGCCC | | | |
| | TGTTGAGTTTGATGAGTGGTCGGTATC | | | |
| | CCCAGGAGGACAAGATTCATGCTGCGG | | | |
| | CCCGGTTTCTCATGAGCAAGCAGATGA | | | |
| | GCAACGGTGAGTGGCTCAAGGAGGAG | | | |
| | ATGCAGGGGGTGTTTAACCATACTTGT | | | |
| | GCCATTGAGTATCCCAACTACCGGTTT | | | |
| | TATTTTGTCATGAAGGCTTTGGGGTTGT | | | |
| | ATTTCAAGGGATATTGCCAGTGA | | | |
| 3A8 | ATGGGAATCCACGAAAGTGTGTCGAA | 105 | MGIHESVSKQFAKNGHSKY | 120 |
| | ACAGTTTGCGAAAAACGGACATTCCAA | | RSDRYGLPKTDLRRWTFHA | |
| | GTACCGCAGCGACCGATACGGCTTACC | | SDLGAQWWKYDDTTPLEE | |
| | TAAGACGGATCTGCGACGATGGACGTT | | LEKRATDYVKYSLELPGYA | |
| | CCACGCGTCCGATCTGGGGGCGCAATG | | PVTLDSKPVKNAYEAALKN | |
| | GTGGAAGTATGACGATACCACACCGCT | | WHLFASLQDPDSGAWQSE | |
| | GGAAGAGCTGGAAAAGAGGGCTACCG | | YDGPQFMSIGYVTACYFGG | |
| | ACTACGTCAAATACTCGCTGGAGCTGC | | NEIPTPVKTEMIRYIVNTAH | |
| | CGGGATACGCGCCCGTGACTCTGGACT | | PVDGGWGLHKEDKSTCFG | |
| | CCAAGCCCGTGAAAAATGCCTACGAA | | TSINYVVLRLLGLSRDHPVC | |
| | GCGGCTCTCAAAAACTGGCATCTGTTT | | VKARKTLLTKFGGAINNPH | |
| | GCGTCGCTGCAAGACCCCGACTCCGGC | | WGKTWLSILNLYKWEGVN | |
| | GCATGGCAGTCGGAATACGACGGACC | | PAPGELWLLPYFVPVHPGR | |
| | GCAGTTCATGTCGATCGGTTATGTGAC | | WWVHTRWIYLAMGYLEA | |
| | GGCGTGCTACTTTGGCGGCAACGAGAT | | AEAQCELTPLLEELRDEIYK | |
| | CCCCACGCCGGTCAAAACCGAAATGAT | | KPYSEIDFSKHCNSISGVDL | |
| | CAGATACATTGTCAACACAGCCCACCC | | YYPHTGLLKFGNALLRRYR | |
| | AGTTGACGGAGGCTGGGGCCTTCACAA | | KFRPQWIKEKVKEEIYNLC | |
| | AGAAGACAAGAGCACCTGTTTCGGTAC | | LREVSNTRHLCLAPVNNAM | |
| | CAGCATCAACTACGTGGTCCTGCGACT | | TSIVMYLHEGPDSANYKKI | |
| | ACTGGGCCTGTCACGGGATCATCCGGT | | AARWPEFLSLNPSGMFMN | |
| | CTGCGTCAAGGCGCGCAAAACGCTGCT | | GTNGLQVWDTAFAVQYAC | |
| | CACCAAGTTTGGCGGCGCCATCAACAA | | VCGFAELPQYQKTIRAAFD | |
| | CCCCCATTGGGGCAAGACCTGGCTGTC | | FLDRSQINEPTEENSYRDDR | |
| | GATTCTCAATCTCTACAAATGGGAGGG | | VGGWPFSTKTQGYPVSDCT | |
| | TGTGAATCCGGCCCCTGGCGAGCTCTG | | AEALKAIIMVQNTPGYEDQ | |
| | GCTGTTGCCCTACTTTGTTCCTGTTCAT | | KKQVSDKRKHTAIDLLLGM | |
| | CCGGGCCGATGGTGGGTCCACACCCGG | | QNVGSFEPGSFASYEPIRAS | |
| | TGGATCTACCTTGCCATGGGCTATCTG | | SMLEKINPAEVFGNIMVEY | |
| | GAGGCTGCGGAGGCCCAATGCGAACT | | PYVECTDSVVLGLSYFRKY | |
| | CACTCCGTTGCTGGAGGAGCTCCGAGA | | HDYRNEDVDRAISAAIGFII | |
| | CGAAATCTACAAAAAGCCCTACTCGGA | | REQQPDGGFFGSWGVCYC | |
| | GATTGATTTCTCCAAACATTGCAACTC | | YAHMFAMEALETQNLNYN | |
| | CATCTCCGGAGTCGACCTCTACTATCC | | NCSTVQKACDFLAGYQEA | |
| | CCACACCGGCCTTTTGAAGTTTGGCAA | | DGGWAEDFKSCETQMYVH | |
| | CGCGCTTCTCAGACGATACCGCAAGTT | | GPHSLVVPTAMALLSLMSG | |
| | CAGACCGCAGTGGATCAAAGAAAAGG | | RYPQEDKIHAAARFLMSKQ | |
| | TCAAGGAGGAAATTTACAACTTGTGCC | | MSNGEWLKEEMEGVFNHT | |
| | TTCGAGAGGTTTCCAACACACGACACT | | CAIEYPNYRFYFVMKALGL | |
| | TGTGTCTCGCTCCCGTCAACAATGCCA | | YFKGYCQ | |
| | TGACCTCCATTGTCATGTATCTCCATGA | | | |
| | GGGGCCCGATTCGGCGAATTACAAAA | | | |
| | AGATTGCGGCCCGATGGCCCGAATTTC | | | |
| | TGTCTCTGAATCCGTCGGGAATGTTTA | | | |
| | TGAACGGCACCAACGGTCTGCAGGTCT | | | |
| | GGGATACTGCGTTTGCCGTGCAATACG | | | |
| | CGTGTGTTTGTGGCTTTGCCGAACTTCC | | | |
| | CCAGTACCAGAAGACGATCCGAGCGG | | | |
| | CGTTTGATTTTCTCGATCGGTCCCAGAT | | | |
| | CAACGAGCCGACGGAGGAAAATTCCT | | | |
| | ATCGAGACGACCGCGTCGGAGGATGG | | | |
| | CCCTTTAGTACCAAGACCCAGGGGTAT | | | |
| | CCAGTCTCCGACTGTACTGCCGAGGCT | | | |
| | CTCAAGGCCATCATCATGGTCCAGAAT | | | |
| | ACGCCTGGATACGAGGATCAGAAGAA | | | |
| | ACAAGTGTCTGACAAGCGGAAACACA | | | |
| | CTGCCATCGATCTACTTTTGGGAATGC | | | |
| | AGAACGTGGGCTCGTTTGAACCGGGCT | | | |
| | CTTTCGCCTCCTATGAGCCTATCCGGG | | | |
| | CGTCGTCCATGCTGGAGAAGATCAATC | | | |
| | CGGCCGAGGTGTTTGGAAACATCATGG | | | |
| | TGGAGTATCCGTACGTGGAATGCACTG | | | |
| | ATTCTGTTGTTCTGGGTCTGTCCTACTT | | | |
| | TCGAAAGTACCACGATTACCGCAACGA | | | |
| | AGACGTGGACCGAGCCATCTCTGCTGC | | | |
| | CATTGGATTCATTATTCGAGAGCAGCA | | | |
| | GCCTGACGGCGGCTTCTTTGGCTCCTG | | | |
| | GGGCGTGTGCTACTGCTACGCTCACAT | | | |
| | GTTTGCCATGGAGGCTCTGGAGACGCA | | | |
| | GAATCTCAACTATAACAACTGTTCCAC | | | |
| | GGTTCAAAAGGCGTGCGACTTTCTGGC | | | |
| | GGGCTACCAGGAAGCAGATGGAGGCT | | | |
| | GGGCCGAGGACTTTAAGTCGTGCGAGA | | | |
| | CTCAGATGTACGTGCACGGACCCCATT | | | |
| | CGCTGGTCGTGCCTACTGCCATGGCCC | | | |
| | TGTTGAGTTTGATGAGTGGTCGGTATC | | | |
| | CCCAGGAGGACAAGATTCATGCTGCGG | | | |
| | CCCGGTTTCTCATGAGCAAGCAGATGA | | | |
| | GCAACGGTGAGTGGCTCAAGGAGGAG | | | |
| | ATGGAGGGGGTGTTTAACCATACTTGT | | | |
| | GCCATTGAGTATCCCAACTACCGGTTT | | | |
| | TATTTTGTCATGAAGGCTTTGGGGTTGT | | | |
| | ATTTCAAGGGATATTGCCAGTGA | | | |
| 3B9 | ATGGGAATCCACGAAAGTGTGTCGAA | 106 | MGIHESVSKQFAKNGHSKY | 316 |
| | ACAGTTTGCGAAAAACGGACATTCCAA | | RSDRYGLPKTDLRRWTFHA | |
| | GTACCGCAGCGACCGATACGGCTTACC | | SDLGAQWWKYDDTTPLEE | |
| | TAAGACGGATCTGCGACGATGGACGTT | | LEKRATDYVKYSLELPGYA | |
| | CCACGCGTCCGATCTGGGGGCGCAATG | | PVTLDSKPVKNAYEAALKN | |
| | GTGGAAGTATGACGATACCACACCGCT | | WHLFASLQDPDSGAWQSE | |
| | GGAAGAGCTGGAAAAGAGGGCTACTG | | YDGPQFMSICYVTACYFGG | |
| | ACTACGTCAAATACTCGCTGGAGCTGC | | NEIPTPVKTEMIRYIVNTAH | |
| | CGGGATACGCGCCCGTGACTCTGGACT | | PVDGGWGLHKEDKSTCFG | |
| | CCAAGCCCGTGAAGAATGCCTACGAA | | TSINYVVLRLLGLSRDHPVC | |
| | GCGGCTCTCAAAAACTGGCATCTGTTT | | VKARKTLLTKFGGAINNPH | |
| | GCGTCGCTGCAAGACCCCGACTCCGGC | | WGKTWLSILNLYKWEGVN | |
| | GCATGGCAGTCGGAATACGACGGACC | | PAPGELWLLPYFVPVHPGR | |
| | GCAGTTCATGTCGATCTGTTATGTGAC | | WWVLTRWIYLAMGYLEAA | |
| | GGCGTGCTACTTTGGCGGCAACGAGAT | | EAQCELTPLLEELRDEIYKK | |
| | CCCCACGCCGGTCAAAACCGAAATGAT | | PYSEIDFSKHCNSISGVDLY | |
| | CAGATACATTGTCAACACAGCCCACCC | | YPHTGLLKFGNALLRRYRK | |
| | AGTTGACGGAGGCTGGGGCCTTCACAA | | FRPQWIKEKVKEEIYNLCLR | |
| | AGAAGACAAGAGCACCTGTTTCGGTAC | | EVSNTRHLCLAPVNNAMTS | |
| | CAGCATCAACTACGTGGTCCTGCGACT | | IVMYLHEGPDSANYKKIAA | |
| | ACTGGGCCTGTCACGGGATCATCCGGT | | RWPEFLSLNPSGMFMNGTN | |
| | CTGCGTCAAGGCGCGCAAAACGCTGCT | | GLQVWDTAFAVQYACVCG | |
| | CACCAAGTTTGGCGGCGCCATCAACAA | | FAELPQYQKTIRAAFDFLDR | |
| | CCCCCATTGGGGCAAGACCTGGCTGTC | | SQINEPTEENSYRDDRVGG | |
| | GATTCTCAATCTCTACAAATGGGAGGG | | WPFSTKTQGYPVSDCTAEA | |
| | TGTGAATCCGGCCCCTGGCGAGCTCTG | | LKAIIMVQNTPGYEDLKKQ | |
| | GCTGTTGCCCTACTTTGTTCCTGTTCAT | | VSDKRKHTAIDLLLGMQNV | |
| | CCGGGCCGATGGTGGGTCCTTACCCGG | | GSFEPGSFASYEPIRASSML | |
| | TGGATCTACCTTGCCATGGGCTATCTG | | EKINPAEVFGNIMVEYPYV | |
| | GAGGCTGCGGAGGCCCAATGCGAACT | | ECTDSVVLGLSYFRKYHDY | |
| | CACTCCGTTGCTGGAGGAGCTCCGAGA | | RNEDVDRAISAAIGYIIREQ | |
| | CGAAATCTACAAAAAGCCCTACTCGGA | | QPDGGFFGSWGVCYCYAH | |
| | GATTGATTTCTCCAAACATTGCAACTC | | MFAMEALETQNLNYNNCS | |
| | CATCTCCGGAGTCGACCTCTACTATCC | | TVQKACDFLAGYQEADGG | |
| | CCACACCGGCCTTTTGAAGTTTGGCAA | | WAEDFKSCETQMYVRGPH | |
| | CGCGCTTCTCCGACGATACCGCAAGTT | | SLVVPTAMALLSLMSGRYP | |
| | CAGACCGCAGTGGATCAAAGAAAAGG | | QEDKIHAAARFLMSKQMS | |
| | TCAAGGAGGAAATTTACAACTTGTGCC | | NGEWLKEEMEGVFNHTCAI | |
| | TTCGAGAGGTTTCCAACACACGACACT | | EYPNYRFYFVMKALGLYF | |
| | TGTGTCTCGCTCCCGTCAACAATGCCA | | MGYCQ | |
| | TGACCTCCATTGTCATGTATCTCCATGA | | | |
| | GGGGCCCGATTCGGCGAATTACAAAA | | | |
| | AGATTGCGGCCCGATGGCCCGAATTTC | | | |
| | TGTCTCTGAATCCGTCGGGAATGTTTA | | | |
| | TGAACGGCACCAACGGTCTGCAGGTCT | | | |
| | GGGATACTGCGTTTGCCGTGCAATACG | | | |
| | CGTGTGTTTGTGGCTTTGCCGAACTTCC | | | |
| | CCAGTACCAGAAGACGATCCGAGCGG | | | |
| | CGTTTGATTTTCTCGATCGGTCCCAGAT | | | |
| | CAACGAGCCGACGGAGGAAAATTCCT | | | |
| | ATCGAGACGACCGCGTCGGAGGATGG | | | |
| | CCCTTTAGTACCAAGACCCAGGGGTAT | | | |
| | CCAGTCTCCGACTGTACTGCCGAGGCT | | | |
| | CTCAAGGCCATCATCATGGTCCAGAAT | | | |
| | ACGCCTGGATACGAGGATCTGAAGAA | | | |
| | ACAAGTGTCTGACAAGCGGAAACACA | | | |
| | CTGCCATCGATCTACTTTTGGGAATGC | | | |
| | AGAACGTGGGCTCGTTTGAACCGGGCT | | | |
| | CTTTCGCCTCCTATGAGCCTATCCGGG | | | |
| | CGTCGTCCATGCTGGAGAAGATCAATC | | | |
| | CGGCCGAGGTGTTTGGAAACATCATGG | | | |
| | TGGAGTATCCGTACGTGGAATGCACTG | | | |
| | ATTCTGTTGTTCTGGGTCTGTCCTACTT | | | |
| | TCGAAAGTACCACGATTACCGCAACGA | | | |
| | AGACGTGGACCGAGCCATCTCTGCTGC | | | |
| | CATTGGATACATTATTCGAGAGCAGCA | | | |
| | GCCTGACGGCGGCTTCTTTGGCTCCTG | | | |
| | GGGCGTGTGCTACTGCTACGCTCACAT | | | |
| | GTTTGCCATGGAGGCTCTGGAGACGCA | | | |
| | GAATCTCAACTATAACAACTGTTCCAC | | | |
| | GGTTCAAAAGGCGTGCGACTTTCTGGC | | | |
| | GGGCTACCAGGAAGCAGATGGAGGCT | | | |
| | GGGCCGAGGACTTTAAGTCGTGCGAGA | | | |
| | CTCAGATGTACGTGCGCGGACCCCATT | | | |
| | CGCTGGTCGTGCCTACTGCCATGGCCC | | | |
| | TGTTGAGTTTGATGAGTGGTCGGTATC | | | |
| | CCCAGGAGGACAAGATTCATGCTGCGG | | | |
| | CCCGGTTTCTCATGAGCAAGCAGATGA | | | |
| | GCAACGGTGAGTGGCTCAAGGAGGAG | | | |
| | ATGGAGGGGGTGTTTAACCATACTTGT | | | |
| | GCCATTGAGTATCCCAACTACCGGTTT | | | |
| | TATTTTGTCATGAAGGCTTTGGGGTTGT | | | |
| | ATTTCATGGGATATTGCCAGTGA | | | |
| 3B9b | ATGGGAATCCACGAAAGTGTGTCGAA | 107 | MGIHESVSKQFAKNGHSKY | 317 |
| | ACAGTTTGCGAAAAACGGACATTCCAA | | RSDRYGLPKTDLRRWTFHA | |
| | GTACCGCAGCGACCGATACGGCTTACC | | SDLGAQWWKYDDTTPLEE | |
| | TAAGACGGATCTGCGACGATGGACGTT | | LEKRATDYVKYSLELPGYA | |
| | CCACGCGTCCGATCTGGGGGCGCAATG | | PVTLDSKPVKNAYEAALKN | |
| | GTGGAAGTATGACGATACCACACCGCT | | WHLFASLQDPDSGAWQSE | |
| | GGAAGAGCTGGAAAAGAGGGCTACTG | | YDGPQFMSICYVTACYFGG | |
| | ACTACGTCAAATACTCGCTGGAGCTGC | | NEIPTPVKTEMIRYIVNTAH | |
| | CGGGATACGCGCCCGTGACTCTGGACT | | PVDGGWGLHKEDKSTCFG | |
| | CCAAGCCCGTGAAGAATGCCTACGAA | | TSINYVVLRLLGLSRDHPVC | |
| | GCGGCTCTCAAAAACTGGCATCTGTTT | | VKARKTLLTKFGGAINNPH | |
| | GCGTCGCTGCAAGACCCCGACTCCGGC | | WGKTWLSILNLYKWEGVN | |
| | GCATGGCAGTCGGAATACGACGGACC | | PAPGELWLLPYFVPVHPGR | |
| | GCAGTTCATGTCGATCTGTTATGTGAC | | WWVLTRWIYLAMGYLEAA | |
| | GGCGTGCTACTTTGGCGGCAACGAGAT | | EAQCELTPLLEELRDEIYKK | |
| | CCCCACGCCGGTCAAAACCGAAATGAT | | PYSEIDFSKHCNSISGVDLY | |
| | CAGATACATTGTCAACACAGCCCACCC | | YPHTGLLKFGNALLRRYRK | |
| | AGTTGACGGAGGCTGGGGCCTTCACAA | | FRPQWIKEKVKEEIYNLCLR | |
| | AGAAGACAAGAGCACCTGTTTCGGTAC | | EVSNTRHLCLAPVNNAMTS | |
| | CAGCATCAACTACGTGGTCCTGCGACT | | IVMYLHEGPDSANYKKIAA | |
| | ACTGGGCCTGTCACGGGATCATCCGGT | | RWPEFLSLNPSGMFMNGTN | |
| | CTGCGTCAAGGCGCGCAAAACGCTGCT | | GLQVWDTAFAVQYACVCG | |
| | CACCAAGTTTGGCGGCGCCATCAACAA | | FAELPQYQKTIRAAFDFLDL | |
| | CCCCCATTGGGGCAAGACCTGGCTGTC | | SQINEPTEENSYRDDRVGG | |
| | GATTCTCAATCTCTACAAATGGGAGGG | | WPFSTKTQGYPVSDCTAEA | |
| | TGTGAATCCGGCCCCTGGCGAGCTCTG | | LKAIIMVQNTPGYEDLKKQ | |
| | GCTGTTGCCCTACTTTGTTCCTGTTCAT | | VSDKRKHTAIDLLLGMQNV | |
| | CCGGGCCGATGGTGGGTCCTTACCCGG | | GSFEPGSFASYEPIRASSML | |
| | TGGATCTACCTTGCCATGGGCTATCTG | | EKINPAEVFGNIMVEYPYV | |
| | GAGGCTGCGGAGGCCCAATGCGAACT | | ECTDSVVLGLSYFRKYHDY | |
| | CACTCCGTTGCTGGAGGAGCTCCGAGA | | RNEDVDRAISAAIGYIIREQ | |
| | CGAAATCTACAAAAAGCCCTACTCGGA | | QPDGGFFGSWGVCYCYAH | |
| | GATTGATTTCTCCAAACATTGCAACTC | | MFAMEALETQNLNYNNCS | |
| | CATCTCCGGAGTCGACCTCTACTATCC | | TVQKACDFLAGYQEADGG | |
| | CCACACCGGCCTTTTGAAGTTTGGCAA | | WAEDFKSCETQMYVRGPH | |
| | CGCGCTTCTCCGACGATACCGCAAGTT | | SLVVPTAMALLSLMSGRYP | |
| | CAGACCGCAGTGGATCAAAGAAAAGG | | QEDKIHAAARFLMSKQMS | |
| | TCAAGGAGGAAATTTACAACTTGTGCC | | NGEWLKEEMEGVFNHTCAI | |
| | TTCGAGAGGTTTCCAACACACGACACT | | EYPNYRFYFVMKALGLYF | |
| | TGTGTCTCGCTCCCGTCAACAATGCCA | | MGYCQ | |
| | TGACCTCCATTGTCATGTATCTCCATGA | | | |
| | GGGGCCCGATTCGGCGAATTACAAAA | | | |
| | AGATTGCGGCCCGATGGCCCGAATTTC | | | |
| | TGTCTCTGAATCCGTCGGGAATGTTTA | | | |
| | TGAACGGCACCAACGGTCTGCAGGTCT | | | |
| | GGGATACTGCGTTTGCCGTGCAATACG | | | |
| | CGTGTGTTTGTGGCTTTGCCGAACTTCC | | | |
| | CCAGTACCAGAAGACGATCCGAGCGG | | | |
| | CGTTTGATTTTCTCGATCTGTCCCAGAT | | | |
| | CAACGAGCCGACGGAGGAAAATTCCT | | | |
| | ATCGAGACGACCGCGTCGGAGGATGG | | | |
| | CCCTTTAGTACCAAGACCCAGGGGTAT | | | |
| | CCAGTCTCCGACTGTACTGCCGAGGCT | | | |
| | CTCAAGGCCATCATCATGGTCCAGAAT | | | |
| | ACGCCTGGATACGAGGATCTGAAGAA | | | |
| | ACAAGTGTCTGACAAGCGGAAACACA | | | |
| | CTGCCATCGATCTACTTTTGGGAATGC | | | |
| | AGAACGTGGGCTCGTTTGAACCGGGCT | | | |
| | CTTTCGCCTCCTATGAGCCTATCCGGG | | | |
| | CGTCGTCCATGCTGGAGAAGATCAATC | | | |
| | CGGCCGAGGTGTTTGGAAACATCATGG | | | |
| | TGGAGTATCCGTACGTTGAATGCACTG | | | |
| | ATTCTGTTGTTCTGGGTCTGTCCTACTT | | | |
| | TCGAAAGTACCACGATTACCGCAACGA | | | |
| | AGACGTGGACCGAGCCATCTCTGCTGC | | | |
| | CATTGGATACATTATTCGAGAGCAGCA | | | |
| | GCCTGACGGCGGCTTCTTTGGCTCCTG | | | |
| | GGGCGTGTGCTACTGCTACGCTCACAT | | | |
| | GTTTGCCATGGAGGCTCTGGAGACGCA | | | |
| | GAATCTCAACTATAACAACTGTTCCAC | | | |
| | GGTTCAAAAGGCGTGCGACTTTCTGGC | | | |
| | GGGCTACCAGGAAGCAGATGGAGGCT | | | |
| | GGGCCGAGGACTTTAAGTCGTGCGAGA | | | |
| | CTCAGATGTACGTGCGCGGACCCCATT | | | |
| | CGCTGGTCGTGCCTACTGCCATGGCCC | | | |
| | TGTTGAGTTTGATGAGTGGTCGGTATC | | | |
| | CCCAGGAGGACAAGATTCATGCTGCGG | | | |
| | CCCGGTTTCTCATGAGCAAGCAGATGA | | | |
| | GCAACGGTGAGTGGCTCAAGGAGGAG | | | |
| | ATGGAGGGGGTGTTTAACCATACTTGT | | | |
| | GCCATTGAGTATCCCAACTACCGGTTT | | | |
| | TATTTTGTCATGAAGGCTTTGGGGTTGT | | | |
| | ATTTCATGGGATATTGCCAGTGA | | | |
| 3C9 | ATGGGAATCCACGAAAGTGTGTCGAA | 108 | MGIHESVSKQFAKNGHSKY | 318 |
| | ACAGTTTGCGAAAAACGGACATTCCAA | | RSDRYGLPKTDLRRWTFHA | |
| | GTACCGCAGCGACCGATACGGCTTACC | | SDLGAQWWKYDDTTPLEE | |
| | TAAGACGGATCTGCGACGATGGACGTT | | LEKRATDYVKYSLELPGYA | |
| | CCACGCGTCCGATCTGGGGGCGCAATG | | PVTLDSKPVKNAYEAALKN | |
| | GTGGAAGTATGACGATACCACACCGCT | | WHLFASLQDPDSGAWQSE | |
| | GGAAGAGCTGGAAAAGAGGGCTACCG | | YDGPQFMSIGYVTACYFGG | |
| | ACTACGTCAAATACTCGCTGGAGCTGC | | NEIPTPVKTEMIRYIVNTAH | |
| | CGGGATACGCGCCCGTGACTCTGGACT | | PVDGGWGLHKEDKSTCFG | |
| | CCAAGCCCGTGAAAAATGCCTACGAA | | TSINYVVLRLLGLSRDHPVC | |
| | GCGGCTCTCAAAAACTGGCATCTGTTT | | VKARKTLLTKFGGAINNPH | |
| | GCGTCGCTGCAAGACCCCGACTCCGGC | | WGKTWLSILNLYKWEGVN | |
| | GCATGGCAGTCGGAATACGACGGACC | | LAPGELWLLPYFVPVHPGR | |
| | GCAGTTCATGTCGATCGGTTATGTGAC | | WWVHTRWIYLAMGYLEA | |
| | GGCGTGCTACTTTGGCGGCAACGAGAT | | AEAQCELTPLLEELRDEIYK | |
| | CCCCACGCCGGTCAAAACCGAAATGAT | | KPYSEIDFSKHCNSISGVDL | |
| | CAGATACATTGTCAACACAGCCCACCC | | YYPHTGLLKFGNALLRRYR | |
| | AGTTGACGGAGGCTGGGGCCTTCACAA | | KFRPQWIKEKVKEEIYNLC | |
| | AGAAGACAAGAGCACCTGTTTCGGTAC | | LREVSNTRHLCLAPVNNAM | |
| | CAGCATCAACTACGTGGTCCTGCGACT | | TSIVMYLHEGPDSANYKKI | |
| | ACTGGGCCTGTCACGGGATCATCCGGT | | AARWPEFLSLNPSGMFMN | |
| | CTGCGTCAAGGCGCGCAAAACGCTGCT | | GTNGLQVWDTAFAVQYAC | |
| | CACCAAGTTTGGCGGCGCTATCAACAA | | VCGFAELPQYQKTIRAAFD | |
| | CCCCCATTGGGGCAAGACCTGGCTGTC | | FLDRSQINEPTEENSYRDDR | |
| | GATTCTCAATCTCTACAAATGGGAGGG | | VGGWPFSTKTQGYPVSDCT | |
| | TGTGAATCTGGCCCCTGGCGAGCTCTG | | AVALKAIIMVQNTPGYEDL | |
| | GCTGTTGCCCTACTTTGTTCCTGTTCAT | | KKQVSDKRKHTAIDLLLGM | |
| | CCGGGCCGATGGTGGGTCCATACCCGG | | QNVGSFEPGSFASYEPIRAS | |
| | TGGATCTACCTTGCCATGGGCTATCTG | | SMLEKINPAEVFGNIMVEY | |
| | GAGGCTGCGGAGGCCCAATGCGAACT | | PYVECTDSVALGLSNFRKY | |
| | CACTCCGTTGCTGGAGGAGCTCCGAGA | | HDYRNEDVDRAISAAIGYII | |
| | CGAAATCTACAAAAAGCCCTACTCGGA | | REQQPDGGFFGSWGVCYC | |
| | GATTGATTTCTCCAAACATTGCAACTC | | YAHMFAMEALETQNLNYN | |
| | CATCTCCGGAGTCGACCTCTACTATCC | | NCSTVQKACDFLAGYQEA | |
| | CCACACCGGCCTTTTGAAGTTTGGCAA | | DGGWAEDFKSCETQMYVR | |
| | CGCGCTTCTCCGACGATACCGCAAGTT | | GPHSLVVPTAMALLSLMSG | |
| | CAGACCGCAGTGGATCAAAGAAAAGG | | RYPQEDKIHAAARFLMSKQ | |
| | TCAAGGAGGAAATTTACAACTTGTGCC | | MSNGEWLKEEMEGVFNHT | |
| | TTCGAGAGGTTTCCAACACACGACACT | | CAIEYPNYRFYFVMKALGL | |
| | TGTGTCTCGCTCCCGTCAACAATGCCA | | YFKGYCQ | |
| | TGACCTCCATTGTCATGTATCTCCATGA | | | |
| | GGGGCCCGATTCGGCGAATTACAAAA | | | |
| | AGATTGCGGCCCGATGGCCCGAATTTC | | | |
| | TGTCTCTGAATCCGTCGGGAATGTTTA | | | |
| | TGAACGGCACCAACGGTCTGCAGGTCT | | | |
| | GGGATACTGCGTTTGCCGTGCAATACG | | | |
| | CGTGTGTTTGTGGCTTTGCCGAACTTCC | | | |
| | CCAGTACCAGAAGACGATCCGAGCGG | | | |
| | CGTTTGATTTTCTCGATCGGTCCCAGAT | | | |
| | CAACGAGCCGACGGAAGAAAATTCCT | | | |
| | ATCGAGACGACCGCGTCGGAGGATGG | | | |
| | CCCTTTAGTACCAAGACCCAGGGGTAT | | | |
| | CCAGTCTCCGACTGTACTGCCGTGGCT | | | |
| | CTCAAGGCCATCATCATGGTCCAGAAT | | | |
| | ACGCCTGGATACGAGGATCTGAAGAA | | | |
| | ACAAGTGTCTGACAAGCGGAAACACA | | | |
| | CTGCCATCGATCTACTTTTGGGAATGC | | | |
| | AGAACGTGGGCTCGTTTGAACCGGGCT | | | |
| | CTTTCGCCTCCTATGAGCCTATCCGGG | | | |
| | CGTCGTCCATGCTGGAGAAGATCAATC | | | |
| | CGGCCGAGGTGTTTGGAAACATCATGG | | | |
| | TGGAGTATCCGTACGTGGAATGCACTG | | | |
| | ATTCTGTTGCTCTGGGTCTGTCCAACTT | | | |
| | TCGAAAGTACCACGATTACCGCAACGA | | | |
| | AGACGTGGACCGAGCCATCTCTGCTGC | | | |
| | CATTGGATACATTATTCGAGAGCAGCA | | | |
| | GCCTGACGGCGGCTTCTTTGGCTCCTG | | | |
| | GGGCGTGTGCTACTGCTACGCTCACAT | | | |
| | GTTTGCCATGGAGGCTCTGGAGACGCA | | | |
| | GAATCTCAACTATAACAACTGTTCCAC | | | |
| | GGTTCAAAAGGCGTGCGACTTTCTGGC | | | |
| | GGGCTACCAGGAAGCAGATGGAGGCT | | | |
| | GGGCCGAGGACTTTAAGTCGTGCGAGA | | | |
| | CTCAGATGTACGTGCGCGGACCCCATT | | | |
| | CGCTGGTCGTGCCTACTGCCATGGCCC | | | |
| | TGTTGAGTTTGATGAGTGGTCGGTATC | | | |
| | CCCAGGAGGACAAGATTCATGCTGCGG | | | |
| | CCCGGTTTCTCATGAGCAAGCAGATGA | | | |
| | GCAACGGTGAGTGGCTCAAGGAGGAG | | | |
| | ATGGAGGGGGTGTTTAACCATACTTGT | | | |
| | GCCATTGAGTATCCCAACTACCGGTTT | | | |
| | TATTTTGTCATGAAGGCTTTGGGGTTGT | | | |
| | ATTTCAAGGGATATTGCCAGTGA | | | |
| 4A6 | ATGGGAATCCACGAAAGTGTGTCGAA | 109 | MGIHESVSKQFAKNGHSKY | 319 |
| | ACAGTTTGCGAAAAACGGACATTCCAA | | RSDRYGLPKTDLRRWTFHA | |
| | GTACCGCAGCGACCGATACGGCTTACC | | SDLGAQWWKYDDTTPLEE | |
| | TAAGACGGATCTGCGACGATGGACGTT | | LEKRATDYVKYSLELPGYA | |
| | CCACGCGTCCGATCTGGGGGCGCAATG | | PVTLDSKPVKNAYEAALKN | |
| | GTGGAAGTATGACGATACCACACCGCT | | WHLFASLQDPDSDAWQSE | |
| | GGAAGAGCTGGAAAAGAGGGCTACCG | | YDGPQFMSIGYVTACYFGG | |
| | ACTACGTCAAATACTCGCTGGAGCTGC | | NEIPTPVKTEMIRYIVNTAH | |
| | CGGGATACGCGCCCGTGACTCTGGACT | | PVDGGWGLHKEDKSTCFG | |
| | CCAAGCCCGTGAAAAATGCCTACGAA | | TSINYVVLRLLGLSRDHPVC | |
| | GCGGCTCTCAAAAACTGGCATCTGTTT | | VKARKTLLTKFGGAINNPH | |
| | GCGTCGCTGCAAGACCCCGACTCCGAC | | WGKTWLSILNLYKWEGVN | |
| | GCATGGCAGTCGGAATACGACGGACC | | PAPGELWLLPYFVPVHPGR | |
| | GCAGTTCATGTCGATCGGTTATGTGAC | | WWVHTRWIYLAMGYLEA | |
| | GGCGTGCTACTTTGGCGGCAACGAGAT | | AEAQCELTPLLEELRDEIYK | |
| | CCCCACGCCGGTCAAAACCGAAATGAT | | KPYSEIDFSKHCNSISGVDL | |
| | CAGATACATTGTCAACACAGCCCACCC | | YYPHTGLLKFGNALLRRYR | |
| | AGTTGACGGAGGCTGGGGCCTTCACAA | | KFRPQWIKEKVKEEIYNLC | |
| | AGAAGACAAGAGCACCTGTTTCGGTAC | | LREVSNTRHLCLAPVNNAM | |
| | CAGCATCAACTACGTGGTCCTGCGACT | | TSIVMYLHEGPDSANYKKI | |
| | ACTGGGCCTGTCACGGGATCATCCGGT | | AARWPEFLSLNPSGMFMN | |
| | CTGCGTCAAGGCGCGCAAAACGCTGCT | | GTNGLQVWDTAFAVQYAC | |
| | CACCAAGTTTGGCGGCGCCATCAACAA | | VCGFAELPQYQKTIRAAFD | |
| | CCCCCATTGGGGCAAGACCTGGCTGTC | | FLDRSQINEPTEENSYRDDR | |
| | GATTCTCAATCTCTACAAATGGGAGGG | | VGGWPFSTKTQGYPVSDCT | |
| | TGTGAATCCGGCCCCTGGCGAGCTCTG | | AEALKAIIMVQNTPGYEDL | |
| | GCTGTTGCCCTACTTTGTTCCTGTTCAT | | KKQVSDKRKHTAIDLLLGM | |
| | CCGGGCCGATGGTGGGTCCATACCCGG | | QNVGSFEPGSFASYEPIRAS | |
| | TGGATCTACCTTGCCATGGGCTATCTG | | SMLEKINPAEVFGNIMVEY | |
| | GAGGCTGCGGAGGCCCAATGCGAACT | | PYVECTDSVVLGLSYFRKY | |
| | CACTCCGTTGCTGGAGGAGCTCCGAGA | | HDYRNEDVDRAISAAIGYII | |
| | CGAAATCTACAAAAAGCCCTACTCGGA | | REQQPDGGFFGSWGVCYC | |
| | GATTGATTTCTCCAAACATTGCAACTC | | YAHMFAMEALETQNLNYN | |
| | CATCTCCGGAGTCGACCTCTACTATCC | | NCSTVQEACDFLAGYQEAD | |
| | CCACACCGGCCTTTTGAAGTTTGGCAA | | GGWAEDFKSCETQMYVRG | |
| | CGCGCTTCTCCGACGATACCGCAAGTT | | PHSLVVPTAMALLSLMSGR | |
| | CAGACCGCAGTGGATCAAAGAAAAGG | | YPQEDKIHAAARFLMSKQ | |
| | TCAAGGAGGAAATTTACAACTTGTGCC | | MSNGEWLKEEMEGVFNHT | |
| | TTCGAGAGGTTTCCAACACACGACACT | | CAIEYPNYRFYFVMKALGL | |
| | TGTGTCTCGCTCCCGTCAACAATGCCA | | YFKGYCQ | |
| | TGACCTCCATTGTCATGTATCTCCATGA | | | |
| | GGGGCCCGATTCGGCGAATTACAAAA | | | |
| | AGATTGCGGCCCGATGGCCCGAATTTC | | | |
| | TGTCTCTGAATCCGTCGGGAATGTTTA | | | |
| | TGAACGGCACCAACGGTCTGCAGGTCT | | | |
| | GGGATACTGCGTTTGCCGTGCAATACG | | | |
| | CGTGTGTTTGTGGCTTTGCCGAACTTCC | | | |
| | CCAGTACCAGAAGACGATCCGAGCGG | | | |
| | CGTTTGATTTTCTCGATCGGTCCCAGAT | | | |
| | CAACGAGCCGACGGAGGAAAATTCCT | | | |
| | ATCGAGACGACCGCGTCGGAGGATGG | | | |
| | CCCTTTAGTACCAAGACCCAGGGGTAT | | | |
| | CCAGTCTCCGACTGTACTGCCGAGGCT | | | |
| | CTCAAGGCCATCATCATGGTCCAGAAT | | | |
| | ACGCCTGGATACGAGGATCTGAAGAA | | | |
| | ACAAGTGTCTGACAAGCGGAAACACA | | | |
| | CTGCCATCGATCTACTTTTGGGAATGC | | | |
| | AGAACGTGGGCTCGTTTGAACCGGGCT | | | |
| | CTTTCGCCTCCTATGAGCCTATCCGGG | | | |
| | CGTCGTCCATGCTGGAGAAGATCAATC | | | |
| | CGGCCGAGGTGTTTGGAAACATCATGG | | | |
| | TGGAGTATCCGTACGTGGAATGCACTG | | | |
| | ATTCTGTTGTTCTGGGTCTGTCCTACTT | | | |
| | TCGAAAGTACCACGATTACCGCAACGA | | | |
| | AGACGTGGACCGAGCCATCTCTGCTGC | | | |
| | CATTGGATACATTATTCGAGAGCAGCA | | | |
| | GCCTGACGGCGGCTTCTTTGGCTCCTG | | | |
| | GGGCGTGTGCTACTGCTACGCTCACAT | | | |
| | GTTTGCCATGGAGGCTCTGGAGACGCA | | | |
| | GAATCTCAACTATAACAACTGTTCCAC | | | |
| | GGTTCAAGAGGCGTGCGACTTTCTGGC | | | |
| | GGGCTACCAGGAAGCAGATGGAGGCT | | | |
| | GGGCCGAGGACTTTAAGTCGTGCGAGA | | | |
| | CTCAGATGTACGTGCGCGGACCCCATT | | | |
| | CGCTGGTCGTGCCTACTGCCATGGCCC | | | |
| | TGTTGAGTTTGATGAGTGGTCGGTATC | | | |
| | CCCAGGAGGACAAGATTCATGCTGCGG | | | |
| | CCCGGTTTCTCATGAGCAAGCAGATGA | | | |
| | GCAACGGTGAGTGGCTCAAGGAGGAG | | | |
| | ATGGAGGGGGTGTTTAACCATACTTGT | | | |
| | GCCATTGAGTATCCCAACTACCGGTTT | | | |
| | TATTTTGTCATGAAGGCTTTGGGGTTGT | | | |
| | ATTTCAAGGGATATTGCCAGTGA | | | |
| 4F11 | ATGGGAATCCACGAAAGTGTGTCGAA | 109 | MGIHESVSKQFAKNGHSKY | 319 |
| | ACAGTTTGCGAAAAACGGACATTCCAA | | RSDRYGLPKTDLRRWTFHA | |
| | GTACCGCAGCGACCGATACGGCTTACC | | SDLGAQWWKYDDTTPLEE | |
| | TAAGACGGATCTGCGACGATGGACGTT | | LEKRATDYVKYSLELPGYA | |
| | CCACGCGTCCGATCTGGGGGCGCAATG | | PVTLDSKPVKNAYEAALKN | |
| | GTGGAAGTATGACGATACCACACCGCT | | WHLFASLQDPDSDAWQSE | |
| | GGAAGAGCTGGAAAAGAGGGCTACCG | | YDGPQFMSIGYVTACYFGG | |
| | ACTACGTCAAATACTCGCTGGAGCTGC | | NEIPTPVKTEMIRYIVNTAH | |
| | CGGGATACGCGCCCGTGACTCTGGACT | | PVDGGWGLHKEDKSTCFG | |
| | CCAAGCCCGTGAAAAATGCCTACGAA | | TSINYVVLRLLGLSRDHPVC | |
| | GCGGCTCTCAAAAACTGGCATCTGTTT | | VKARKTLLTKFGGAINNPH | |
| | GCGTCGCTGCAAGACCCCGACTCCGAC | | WGKTWLSILNLYKWEGVN | |
| | GCATGGCAGTCGGAATACGACGGACC | | PAPGELWLLPYFVPVHPGR | |
| | GCAGTTCATGTCGATCGGTTATGTGAC | | WWVHTRWIYLAMGYLEA | |
| | GGCGTGCTACTTTGGCGGCAACGAGAT | | AEAQCELTPLLEELRDEIYK | |
| | CCCCACGCCGGTCAAAACCGAAATGAT | | KPYSEIDFSKHCNSISGVDL | |
| | CAGATACATTGTCAACACAGCCCACCC | | YYPHTGLLKFGNALLRRYR | |
| | AGTTGACGGAGGCTGGGGCCTTCACAA | | KFRPQWIKEKVKEEIYNLC | |
| | AGAAGACAAGAGCACCTGTTTCGGTAC | | LREVSNTRHLCLAPVNNAM | |
| | CAGCATCAACTACGTGGTCCTGCGACT | | TSIVMYLHEGPDSANYKKI | |
| | ACTGGGCCTGTCACGGGATCATCCGGT | | AARWPEFLSLNPSGMFMN | |
| | CTGCGTCAAGGCGCGCAAAACGCTGCT | | GTNGLQVWDTAFAVQYAC | |
| | CACCAAGTTTGGCGGCGCCATCAACAA | | VCGFAELPQYQKTIRAAFD | |
| | CCCCCATTGGGGCAAGACCTGGCTGTC | | FLDRSQINEPTEENSYRDDR | |
| | GATTCTCAATCTCTACAAATGGGAGGG | | VGGWPFSTKTQGYPVSDCT | |
| | TGTGAATCCGGCCCCTGGCGAGCTCTG | | AEALKAIIMVQNTPGYEDL | |
| | GCTGTTGCCCTACTTTGTTCCTGTTCAT | | KKQVSDKRKHTAIDLLLGM | |
| | CCGGGCCGATGGTGGGTCCATACCCGG | | QNVGSFEPGSFASYEPIRAS | |
| | TGGATCTACCTTGCCATGGGCTATCTG | | SMLEKINPAEVFGNIMVEY | |
| | GAGGCTGCGGAGGCCCAATGCGAACT | | PYVECTDSVVLGLSYFRKY | |
| | CACTCCGTTGCTGGAGGAGCTCCGAGA | | HDYRNEDVDRAISAAIGYII | |
| | CGAAATCTACAAAAAGCCCTACTCGGA | | REQQPDGGFFGSWGVCYC | |
| | GATTGATTTCTCCAAACATTGCAACTC | | YAHMFAMEALETQNLNYN | |
| | CATCTCCGGAGTCGACCTCTACTATCC | | NCSTVQEACDFLAGYQEAD | |
| | CCACACCGGCCTTTTGAAGTTTGGCAA | | GGWAEDFKSCETQMYVRG | |
| | CGCGCTTCTCCGACGATACCGCAAGTT | | PHSLVVPTAMALLSLMSGR | |
| | CAGACCGCAGTGGATCAAAGAAAAGG | | YPQEDKIHAAARFLMSKQ | |
| | TCAAGGAGGAAATTTACAACTTGTGCC | | MSNGEWLKEEMEGVFNHT | |
| | TTCGAGAGGTTTCCAACACACGACACT | | CAIEYPNYRFYFVMKALGL | |
| | TGTGTCTCGCTCCCGTCAACAATGCCA | | YFKGYCQ | |
| | TGACCTCCATTGTCATGTATCTCCATGA | | | |
| | GGGGCCCGATTCGGCGAATTACAAAA | | | |
| | AGATTGCGGCCCGATGGCCCGAATTTC | | | |
| | TGTCTCTGAATCCGTCGGGAATGTTTA | | | |
| | TGAACGGCACCAACGGTCTGCAGGTCT | | | |
| | GGGATACTGCGTTTGCCGTGCAATACG | | | |
| | CGTGTGTTTGTGGCTTTGCCGAACTTCC | | | |
| | CCAGTACCAGAAGACGATCCGAGCGG | | | |
| | CGTTTGATTTTCTCGATCGGTCCCAGAT | | | |
| | CAACGAGCCGACGGAGGAAAATTCCT | | | |
| | ATCGAGACGACCGCGTCGGAGGATGG | | | |
| | CCCTTTAGTACCAAGACCCAGGGGTAT | | | |
| | CCAGTCTCCGACTGTACTGCCGAGGCT | | | |
| | CTCAAGGCCATCATCATGGTCCAGAAT | | | |
| | ACGCCTGGATACGAGGATCTGAAGAA | | | |
| | ACAAGTGTCTGACAAGCGGAAACACA | | | |
| | CTGCCATCGATCTACTTTTGGGAATGC | | | |
| | AGAACGTGGGCTCGTTTGAACCGGGCT | | | |
| | CTTTCGCCTCCTATGAGCCTATCCGGG | | | |
| | CGTCGTCCATGCTGGAGAAGATCAATC | | | |
| | CGGCCGAGGTGTTTGGAAACATCATGG | | | |
| | TGGAGTATCCGTACGTGGAATGCACTG | | | |
| | ATTCTGTTGTTCTGGGTCTGTCCTACTT | | | |
| | TCGAAAGTACCACGATTACCGCAACGA | | | |
| | AGACGTGGACCGAGCCATCTCTGCTGC | | | |
| | CATTGGATACATTATTCGAGAGCAGCA | | | |
| | GCCTGACGGCGGCTTCTTTGGCTCCTG | | | |
| | GGGCGTGTGCTACTGCTACGCTCACAT | | | |
| | GTTTGCCATGGAGGCTCTGGAGACGCA | | | |
| | GAATCTCAACTATAACAACTGTTCCAC | | | |
| | GGTTCAAGAGGCGTGCGACTTTCTGGC | | | |
| | GGGCTACCAGGAAGCAGATGGAGGCT | | | |
| | GGGCCGAGGACTTTAAGTCGTGCGAGA | | | |
| | CTCAGATGTACGTGCGCGGACCCCATT | | | |
| | CGCTGGTCGTGCCTACTGCCATGGCCC | | | |
| | TGTTGAGTTTGATGAGTGGTCGGTATC | | | |
| | CCCAGGAGGACAAGATTCATGCTGCGG | | | |
| | CCCGGTTTCTCATGAGCAAGCAGATGA | | | |
| | GCAACGGTGAGTGGCTCAAGGAGGAG | | | |
| | ATGGAGGGGGTGTTTAACCATACTTGT | | | |
| | GCCATTGAGTATCCCAACTACCGGTTT | | | |
| | TATTTTGTCATGAAGGCTTTGGGGTTGT | | | |
| | ATTTCAAGGGATATTGCCAGTGA | | | |
| 3D11 | ATGGGAATCCACGAAAGTGTGTCGAA | 111 | MGIHESVSKQFAKNGHSKY | 321 |
| | ACAGTTTGCGAAAAACGGACATTCCAA | | RSDRYGLPKTDLRRWTFHA | |
| | GTACCGCAGCGACCGATACGGCTTACC | | SDLGAQWWKYDDTTPLEE | |
| | TAAGACGGATCTGCGACGATGGACGTT | | LEKRATDYVKYSLELPGYA | |
| | CCACGCGTCCGATCTGGGGGCGCAATG | | PVTLDSKPVNNAYEAALKN | |
| | GTGGAAGTATGACGATACCACACCGCT | | WHLFASLQDPDSGAWQSE | |
| | GGAAGAGCTGGAAAAGAGGGCTACCG | | YDGPQFMSIGYVTACYFGG | |
| | ACTACGTCAAATACTCGCTGGAGCTGC | | NEIPTPVKTEMIRYIVNTAH | |
| | CGGGATACGCGCCCGTGACTCTGGACT | | PVDGGWSLHKEDKSTCFGT | |
| | CCAAGCCCGTGAATAATGCCTACGAAG | | SINYVVLRLLGLSRDHPVC | |
| | CGGCTCTCAAAAACTGGCATCTGTTTG | | VKARKTLLTKFGGAINNPH | |
| | CGTCGCTGCAAGACCCCGACTCCGGCG | | WGKTWLSILNLYKWEGVN | |
| | CATGGCAGTCGGAATACGACGGACCG | | PAPGELWLLPYFVPVHPGR | |
| | CAGTTCATGTCGATCGGTTATGTGACG | | WWVHTRWIYLAMGYLEA | |
| | GCATGCTACTTTGGCGGCAACGAGATC | | AEAQCELTPLLEELRDEIYK | |
| | CCCACGCCGGTCAAAACCGAAATGATC | | KPYSEIDFSKHCNSISGVDL | |
| | AGATACATTGTCAACACAGCCCACCCA | | YYPHTGLLKFGNALLRRYR | |
| | GTTGACGGAGGCTGGAGCCTTCACAAA | | KFRPQWIKEKVKEEIYNLC | |
| | GAAGACAAGAGCACCTGTTTCGGTACC | | LREVSNTRHLCLAPVNNAM | |
| | AGCATCAACTACGTGGTCCTGCGACTA | | TSIVMYLHEGPDSANYKKI | |
| | CTGGGCCTGTCACGGGATCATCCGGTC | | AARWPEFLSLNPSGMFMN | |
| | TGCGTCAAGGCGCGCAAAACGCTGCTC | | GTNGLQVWDTAFAVQYAC | |
| | ACCAAGTTTGGCGGCGCCATCAACAAC | | VCGFAELPQYQKTIRAAFD | |
| | CCCCATTGGGGCAAGACCTGGCTGTCG | | FLDRSQINEPTEENSYRDDR | |
| | ATTCTCAATCTCTACAAATGGGAGGGT | | VGGWPFSTKTQGYPVSDCT | |
| | GTGAATCCGGCCCCTGGCGAGCTCTGG | | AEALKAIIMVQNTPGYEDL | |
| | CTGTTGCCCTACTTTGTTCCTGTTCATC | | KKQVSDKRKHTAIDLLLGM | |
| | CGGGCCGATGGTGGGTCCATACCCGGT | | QNVGLFEPGSFASYETIRAS | |
| | GGATCTACCTTGCCATGGGCTATCTGG | | SMLEKINPAEVFGNIMVEY | |
| | AGGCTGCGGAGGCCCAATGCGAACTC | | PYVECTDSVVLGLSYFRKY | |
| | ACTCCGTTGCTGGAGGAGCTCCGAGAC | | HDYRNEDVDRAISAAIGYII | |
| | GAAATCTACAAAAAGCCCTACTCGGAG | | REQQPDGGFFGSWGVCYC | |
| | ATTGATTTCTCCAAACATTGCAACTCC | | YAHMFAMEALETLNLNYN | |
| | ATCTCCGGAGTCGACCTCTACTATCCC | | NCSTVQKACDFLAGYQEA | |
| | CACACCGGCCTTTTGAAGTTTGGCAAC | | DGGWAEDFKSCETQMYVR | |
| | GCGCTTCTCCGACGATACCGCAAGTTC | | GPHSLVVPTAMALLSLMSG | |
| | AGACCGCAGTGGATCAAAGAAAAGGT | | RYPQEDKIHAAARFLMSKQ | |
| | CAAGGAGGAAATTTACAACTTGTGCCT | | MSNGEWLKEEMEGVFNHT | |
| | TCGAGAGGTTTCCAACACACGACACTT | | CAIEYPNYRFYFVMKALGL | |
| | GTGTCTCGCTCCCGTCAACAATGCCAT | | YFKGYC | |
| | GACCTCCATTGTCATGTATCTCCATGA | | | |
| | GGGGCCCGATTCGGCGAATTACAAAA | | | |
| | AGATTGCGGCCCGATGGCCCGAATTTC | | | |
| | TGTCTCTGAATCCGTCGGGAATGTTTA | | | |
| | TGAACGGCACCAACGGTCTGCAGGTCT | | | |
| | GGGATACTGCGTTTGCCGTGCAATACG | | | |
| | CGTGTGTTTGTGGCTTTGCCGAACTTCC | | | |
| | CCAGTACCAGAAGACGATCCGAGCGG | | | |
| | CGTTTGATTTTCTCGATCGGTCCCAGAT | | | |
| | CAACGAGCCGACGGAGGAAAATTCCT | | | |
| | ATCGAGACGACCGCGTCGGAGGATGG | | | |
| | CCCTTTAGTACCAAGACCCAGGGGTAT | | | |
| | CCAGTCTCCGACTGTACTGCCGAGGCT | | | |
| | CTCAAGGCCATCATCATGGTCCAGAAT | | | |
| | ACGCCTGGATACGAGGATCTGAAGAA | | | |
| | ACAAGTGTCTGACAAGCGGAAACACA | | | |
| | CTGCCATCGATCTACTTTTGGGAATGC | | | |
| | AGAACGTGGGCTTGTTTGAACCGGGCT | | | |
| | CTTTCGCCTCCTATGAGACTATCCGGG | | | |
| | CGTCGTCCATGCTGGAGAAGATCAATC | | | |
| | CGGCCGAGGTGTTTGGAAACATCATGG | | | |
| | TGGAGTATCCGTACGTGGAATGCACTG | | | |
| | ATTCTGTTGTTCTGGGTCTGTCCTACTT | | | |
| | TCGAAAGTACCACGATTACCGCAACGA | | | |
| | AGACGTGGACCGAGCCATCTCTGCTGC | | | |
| | CATTGGATACATTATTCGAGAGCAGCA | | | |
| | GCCTGACGGCGGCTTCTTTGGCTCCTG | | | |
| | GGGCGTGTGCTACTGCTACGCTCACAT | | | |
| | GTTTGCCATGGAGGCTCTGGAGACGCT | | | |
| | GAATCTCAACTATAACAACTGTTCCAC | | | |
| | GGTTCAAAAGGCGTGCGACTTTCTGGC | | | |
| | GGGCTACCAGGAAGCAGATGGAGGCT | | | |
| | GGGCCGAGGACTTTAAGTCGTGCGAGA | | | |
| | CTCAGATGTACGTGCGCGGACCCCATT | | | |
| | CGCTGGTCGTGCCTACTGCCATGGCCC | | | |
| | TGTTGAGTTTGATGAGTGGTCGGTATC | | | |
| | CCCAGGAGGACAAGATTCATGCTGCGG | | | |
| | CCCGGTTTCTCATGAGCAAGCAGATGA | | | |
| | GCAACGGTGAGTGGCTCAAGGAGGAG | | | |
| | ATGGAGGGGGTGTTTAACCATACTTGT | | | |
| | GCCATTGAGTATCCCAACTACCGGTTT | | | |
| | TATTTTGTCATGAAGGCTTTGGGGTTGT | | | |
| | ATTTCAAGGGATATTGCTAG | | | |
| 4B11 | ATGGGAATCCACGAAAGTGTGTCGAA | 112 | MGIHESVSKQFAKNGHSKY | 322 |
| | ACAGTTTGCGAAAAACGGACATTCCAA | | RSDRYGLPKTDLRRWTFHA | |
| | GTACCGCAGCGACCGATACGGCTTACC | | SDLGAQWWKYDDTTPLEE | |
| | TAAGACGGATCTGCGACGATGGACGTT | | LEKRATDYVKYSLELPGYA | |
| | CCACGCGTCCGATCTGGGGGCGCAATG | | PVTLDSKPVKNAYEAALKN | |
| | GTGGAAGTATGACGATACCACACCGCT | | WHLFASLQDPDSGAWQSE | |
| | GGAAGAGCTGGAAAAGAGGGCTACCG | | YDGPQFMSIGYVTACYFGG | |
| | ACTACGTCAAATACTCGCTGGAGCTGC | | NEIPTPVKTEMIRYIVNTAH | |
| | CGGGATACGCGCCCGTGACTCTGGACT | | PVDGGWGLHKEDKSTCFG | |
| | CCAAGCCCGTGAAAAATGCCTACGAA | | TSINYVVLRLLGLSRDHPVC | |
| | GCGGCTCTCAAAAACTGGCATCTGTTT | | VKARKTLLTKFGGAINNPH | |
| | GCGTCGCTGCAAGACCCCGACTCCGGC | | WGKILLSILNLYKWEGVNP | |
| | GCATGGCAGTCGGAATACGACGGACC | | APGELWLLPYFVPVHPGRW | |
| | GCAGTTCATGTCGATCGGTTATGTGAC | | WVHTRWIYLAMGYLEAAE | |
| | GGCATGCTACTTTGGCGGCAACGAGAT | | AQCELTPLLEELRDEIYKKP | |
| | CCCCACGCCGGTCAAAACTGAAATGAT | | YSEIDFSKHCNSISGVDLYY | |
| | CAGATACATTGTCAACACAGCCCACCC | | PHTGLLKFGNALLRRYRKF | |
| | AGTTGACGGAGGCTGGGGCCTTCACAA | | RPQWIKEKVKEEIYNLCLR | |
| | AGAAGACAAGAGCACCTGTTTCGGTAC | | EVSNTRHLCLAPVNNAMTS | |
| | CAGCATCAACTACGTGGTCCTGCGACT | | IVMYLHEGPDSANYKKIAA | |
| | ACTGGGCCTGTCACGGGATCATCCGGT | | RWPEFLSLNPSGMFMNGTN | |
| | CTGCGTCAAGGCGCGCAAAACGCTGCT | | GLQVWDTAFAVQYACVCG | |
| | CACCAAGTTTGGCGGCGCCATCAACAA | | FAELPQYQKTIRAAFDFLDR | |
| | CCCCCATTGGGGCAAGATCTTGCTGTC | | SQINEPTEENSYRDDRVGG | |
| | GATTCTCAATCTCTACAAATGGGAGGG | | WPFSTKTQGYPVSDCTAEA | |
| | TGTGAATCCGGCCCCTGGCGAGCTCTG | | LKAIIMVQNTPGYEDLKKQ | |
| | GCTGTTGCCCTACTTTGTTCCTGTTCAT | | VSDKRKHTAIDLLLGMQNV | |
| | CCGGGCCGATGGTGGGTCCATACCCGG | | GSFEPGSFASYEPIRASSML | |
| | TGGATCTACCTTGCCATGGGCTATCTG | | EKINPAEVFGYIMVEYPYEE | |
| | GAGGCTGCGGAGGCCCAATGCGAACT | | CTDSVVLGLSYFRKYHDYR | |
| | CACTCCGTTGCTGGAGGAGCTCCGAGA | | NEDVDRAISAAIGYIIREQQ | |
| | CGAAATCTACAAAAAGCCCTACTCGGA | | PDGGFFGSWGVCYCYAHM | |
| | GATTGATTTCTCCAAACATTGCAACTC | | FAMEALETQNLNYNNCSTV | |
| | CATCTCCGGAGTCGACCTCTACTATCC | | QKACDFLAGYQEADGGWA | |
| | CCACACCGGCCTTTTGAAGTTTGGCAA | | EDFKSCETQMYVRGPHSLV | |
| | CGCGCTTCTCCGACGATACCGCAAGTT | | VPTAMALLSLMSGRYPQED | |
| | CAGACCGCAGTGGATCAAAGAAAAGG | | KIHAAARFLMSKQMSNGE | |
| | TCAAGGAGGAAATTTACAACTTGTGCC | | WLKEEMEGVFNHTCAIEYP | |
| | TTCGAGAGGTTTCCAACACACGACACT | | NYRFYFVMKALGLYFKGY | |
| | TGTGTCTCGCTCCCGTCAACAATGCCA | | CQ | |
| | TGACCTCCATTGTCATGTATCTCCATGA | | | |
| | GGGGCCCGATTCGGCGAATTACAAAA | | | |
| | AGATTGCGGCCCGATGGCCCGAATTTC | | | |
| | TGTCTCTGAATCCGTCGGGAATGTTTA | | | |
| | TGAACGGCACCAACGGTCTGCAGGTCT | | | |
| | GGGATACTGCGTTTGCCGTGCAATACG | | | |
| | CGTGTGTTTGTGGCTTTGCCGAACTTCC | | | |
| | CCAGTACCAGAAGACGATCCGAGCGG | | | |
| | CGTTTGATTTTCTCGATCGGTCCCAGAT | | | |
| | CAACGAGCCGACGGAGGAAAATTCCT | | | |
| | ATCGAGACGACCGCGTCGGAGGATGG | | | |
| | CCCTTTAGTACCAAGACCCAGGGGTAT | | | |
| | CCAGTCTCCGACTGTACTGCCGAGGCT | | | |
| | CTCAAGGCCATCATCATGGTCCAGAAT | | | |
| | ACGCCTGGATACGAGGATCTGAAGAA | | | |
| | ACAAGTGTCTGACAAGCGGAAACACA | | | |
| | CTGCCATCGATCTACTTTTGGGAATGC | | | |
| | AGAACGTGGGCTCGTTTGAACCGGGCT | | | |
| | CTTTCGCCTCCTATGAGCCTATCCGGG | | | |
| | CGTCGTCCATGCTGGAGAAGATCAATC | | | |
| | CGGCCGAGGTGTTTGGATACATCATGG | | | |
| | TGGAGTATCCGTACGAGGAATGCACTG | | | |
| | ATTCTGTTGTTCTGGGTCTGTCCTACTT | | | |
| | TCGAAAGTACCACGATTACCGCAACGA | | | |
| | AGACGTGGACCGAGCCATCTCTGCTGC | | | |
| | CATTGGATACATTATTCGAGAGCAGCA | | | |
| | GCCTGACGGCGGCTTCTTTGGCTCCTG | | | |
| | GGGCGTGTGCTACTGCTACGCTCACAT | | | |
| | GTTTGCCATGGAGGCTCTGGAGACGCA | | | |
| | GAATCTCAACTATAACAACTGTTCCAC | | | |
| | AGTTCAAAAGGCGTGCGACTTTCTGGC | | | |
| | GGGCTACCAGGAAGCAGATGGAGGCT | | | |
| | GGGCCGAGGACTTTAAGTCGTGCGAGA | | | |
| | CTCAGATGTACGTGCGCGGACCCCATT | | | |
| | CGCTGGTCGTGCCTACTGCCATGGCCC | | | |
| | TGTTGAGTTTGATGAGTGGTCGGTATC | | | |
| | CCCAGGAGGACAAGATTCATGCTGCGG | | | |
| | CCCGGTTTCTCATGAGCAAGCAGATGA | | | |
| | GCAACGGTGAGTGGCTCAAGGAGGAG | | | |
| | ATGGAGGGGGTGTTTAACCATACTTGT | | | |
| | GCCATTGAGTATCCCAACTACCGGTTT | | | |
| | TATTTTGTCATGAAGGCTTTGGGGTTGT | | | |
| | ATTTCAAGGGATATTGCCAGTGA | | | |
| 4B12 | ATGGGAATCCACGAAAGTGTGTCGAA | 63 | MGIHESVSKQFAKNGHSKY | 84 |
| | ACAGTTTGCGAAAAACGGACATTCCAA | | RSDRYGLPKTDLRRWTFHA | |
| | GTACCGCAGCGACCGATACGGCTTACC | | SDLGAQWWKYDDTTPLEE | |
| | TAAGACGGATCTGCGACGATGGACGTT | | LEKRATDYVKYSLELPGYA | |
| | CCACGCGTCCGATCTGGGGGCGCAATG | | PVTLDSKPVKNAYEAALKN | |
| | GTGGAAGTATGACGATACCACACCGCT | | WHLFASLQDPDSGAWQSE | |
| | GGAAGAGCTGGAAAAGAGGGCTACCG | | YDGPQFMSIGYVTACYFGG | |
| | ACTACGTCAAATACTCGCTGGAGCTGC | | NEIPTPVKTEMIRYIVNTAH | |
| | CGGGATACGCGCCCGTGACTCTGGACT | | PVDGGWGLHKEDKSTCFG | |
| | CCAAGCCCGTGAAAAATGCCTACGAA | | TSNNYVVLRLLGLSRDHPV | |
| | GCGGCTCTCAAAAACTGGCATCTGTTT | | CVKARKTLLTKFGGAINNP | |
| | GCGTCGCTGCAAGACCCCGACTCCGGC | | HWGKTWLSILNLYKWEGV | |
| | GCATGGCAGTCGGAATACGACGGACC | | NPAPGELWLLPYFVPVHPG | |
| | GCAGTTCATGTCGATCGGTTATGTGAC | | RWWVHTRWIYLAMGYLE | |
| | GGCGTGCTACTTTGGCGGCAACGAGAT | | AAEAQCELTPLLEELRDEIY | |
| | CCCCACGCCGGTCAAAACCGAAATGAT | | KKPYSEIDFSKHCNSISGVD | |
| | CAGATACATTGTCAACACAGCCCACCC | | LYYPHTGLLKFGNALLRRY | |
| | AGTTGACGGAGGCTGGGGCCTTCACAA | | RKFRPQWIKEKVKEEIYNL | |
| | AGAAGACAAGAGCACCTGTTTCGGTAC | | CLREVSNTRHLCLAPVNNA | |
| | CAGCAACAACTACGTGGTCCTGCGACT | | MTSIVMYLHEGPDSANYKK | |
| | ACTGGGCCTGTCACGGGATCATCCGGT | | IAARWPEFLSLNPSGMFMN | |
| | CTGCGTCAAGGCGCGCAAAACGCTGCT | | GTNGLQVWDTAFAVQYAS | |
| | CACCAAGTTTGGCGGCGCCATCAACAA | | VCGFAELPQYQKTIRAAFD | |
| | CCCCCATTGGGGCAAGACCTGGCTGTC | | FLDRSQINEPTEENSYRDDR | |
| | GATTCTCAATCTCTACAAATGGGAGGG | | VGGWPFSTKTQGYPVSDCT | |
| | TGTGAATCCGGCCCCTGGCGAGCTCTG | | AEALKAIIMVQNTPGYEDL | |
| | GCTGTTGCCCTACTTTGTTCCTGTTCAT | | KKQVSDKRKHTAIDLLLGM | |
| | CCGGGCCGATGGTGGGTCCATACCCGG | | QNVGSFEPGSFASYEPIRAS | |
| | TGGATCTACCTTGCCATGGGCTATCTG | | SMLEKINPAEVFGNIMVEY | |
| | GAGGCTGCGGAGGCCCAATGCGAACT | | PYVECTDSVVMGLSYFRKY | |
| | CACTCCGTTGCTGGAGGAGCTCCGAGA | | HDYRNEDVDRAISAAIGYII | |
| | CGAAATCTACAAAAAGCCCTACTCGGA | | REQQPDGGFFGSWGVCYC | |
| | GATTGATTTCTCCAAACATTGCAACTC | | YAHMFAMEALETQNLNYN | |
| | CATCTCCGGAGTCGACCTCTACTATCC | | NCSTVQKACDFLAGYQEA | |
| | CCACACCGGCCTTTTGAAGTTTGGCAA | | DGGWAEDFKSCETQMYVR | |
| | CGCGCTTCTCCGACGATACCGCAAGTT | | GPHSLVVPTAMALLSLMSS | |
| | CAGACCGCAGTGGATCAAAGAAAAGG | | RYPQEDKIHAAARFLMSKQ | |
| | TCAAGGAGGAAATTTACAACTTGTGCC | | MSNGEWLKEEMEGVFNHT | |
| | TTCGAGAGGTTTCCAACACACGACACT | | CAIEYPNYRFYFVMKALGL | |
| | TGTGTCTCGCTCCCGTCAACAATGCCA | | YFKGYCQ | |
| | TGACCTCCATTGTCATGTATCTCCATGA | | | |
| | GGGGCCCGATTCGGCGAATTACAAAA | | | |
| | AGATTGCGGCCCGATGGCCCGAATTTC | | | |
| | TGTCTCTGAATCCGTCGGGAATGTTTA | | | |
| | TGAACGGCACCAACGGTCTGCAGGTCT | | | |
| | GGGATACTGCGTTTGCCGTGCAATACG | | | |
| | CGAGTGTTTGTGGCTTTGCCGAACTTC | | | |
| | CCCAGTACCAGAAGACGATCCGAGCG | | | |
| | GCGTTTGATTTTCTCGATCGGTCCCAG | | | |
| | ATCAACGAGCCGACGGAGGAAAATTC | | | |
| | CTATCGAGACGACCGCGTCGGAGGATG | | | |
| | GCCCTTTAGTACCAAGACCCAGGGGTA | | | |
| | TCCAGTCTCCGACTGTACTGCCGAGGC | | | |
| | TCTCAAGGCCATCATCATGGTCCAGAA | | | |
| | TACGCCTGGATACGAGGATCTGAAGAA | | | |
| | ACAAGTGTCTGACAAGCGGAAACACA | | | |
| | CTGCCATCGATCTACTTTTGGGAATGC | | | |
| | AGAACGTGGGCTCGTTTGAACCGGGCT | | | |
| | CTTTCGCCTCCTATGAGCCTATCCGGG | | | |
| | CGTCGTCCATGCTGGAGAAGATCAATC | | | |
| | CGGCCGAGGTGTTTGGAAACATCATGG | | | |
| | TGGAGTATCCGTACGTGGAATGCACTG | | | |
| | ATTCTGTTGTTATGGGTCTGTCCTACTT | | | |
| | TCGAAAGTACCACGATTACCGCAACGA | | | |
| | AGACGTGGACCGAGCCATCTCTGCTGC | | | |
| | CATTGGATACATTATTCGAGAGCAGCA | | | |
| | GCCTGACGGCGGCTTCTTTGGCTCCTG | | | |
| | GGGCGTGTGCTACTGCTACGCTCACAT | | | |
| | GTTTGCCATGGAGGCTCTGGAGACGCA | | | |
| | GAATCTCAACTATAACAACTGTTCCAC | | | |
| | GGTTCAAAAGGCGTGCGACTTTCTGGC | | | |
| | GGGCTACCAGGAAGCAGATGGAGGCT | | | |
| | GGGCCGAGGACTTTAAGTCGTGCGAGA | | | |
| | CTCAGATGTACGTGCGCGGACCCCATT | | | |
| | CGCTGGTCGTGCCTACTGCCATGGCCC | | | |
| | TGTTGAGTTTGATGAGTAGTCGGTATC | | | |
| | CCCAGGAGGACAAGATTCATGCTGCGG | | | |
| | CCCGGTTTCTCATGAGCAAGCAGATGA | | | |
| | GCAACGGTGAGTGGCTCAAGGAGGAG | | | |
| | ATGGAGGGGGTGTTTAACCATACTTGT | | | |
| | GCCATTGAGTATCCCAACTACCGGTTT | | | |
| | TATTTTGTCATGAAGGCTTTGGGGTTGT | | | |
| | ATTTCAAGGGATATTGCCAGTGA | | | |
| 2A5 | ATGGGAATCCACGAAAGTGTGTCGAA | 113 | MGIHESVSKQFAKNGHSKY | 323 |
| | ACAGTTTGCGAAAAACGGACATTCCAA | | RSDRYGLPKTDLRRWTFHA | |
| | GTACCGCAGCGACCGATACGGCTTACC | | SDLGAQWWKYDDTTPLEE | |
| | TAAGACGGATCTGCGACGATGGACGTT | | LEKRATDYVKYSLELPGYA | |
| | CCACGCGTCCGATCTGGGGGCGCAATG | | PVTLDSKPVKNAYEAALKN | |
| | GTGGAAGTATGACGATACCACACCGCT | | WHLFASLQDPDSGAWQSE | |
| | GGAAGAGCTGGAAAAGAGGGCTACCG | | YDGPQFMSIGYVTACYFGG | |
| | ACTACGTCAAATACTCGCTGGAGCTGC | | NEIPTPVKTEMIRYIVNTAH | |
| | CGGGATACGCGCCCGTGACTCTGGACT | | PVDGGWGLHKEDKSTCFG | |
| | CCAAGCCCGTGAAAAATGCCTACGAA | | TSINYVVLRLLGLSRDHPVC | |
| | GCGGCTCTCAAAAACTGGCATCTGTTT | | VKARKTLLTKFGGAINNPH | |
| | GCGTCGCTGCAAGACCCCGACTCCGGC | | WGKTWLSILNLYKWEGVN | |
| | GCATGGCAGTCGGAATACGACGGACC | | PAPGVLWLLPYFVPVHPGR | |
| | GCAGTTCATGTCGATCGGTTATGTGAC | | WWVHTRWIYLAMGYLEA | |
| | GGCGTGCTACTTTGGCGGCAACGAGAT | | AEAQCELTPLLEELRDEIYK | |
| | CCCCACGCCGGTCAAAACCGAAATGAT | | KPYSEIDFSKHCNSISGVDL | |
| | CAGATACATTGTCAACACAGCCCACCC | | YYPHTGLLKFGNALLRRYR | |
| | AGTTGACGGAGGCTGGGGCCTTCACAA | | KFRPQWIKEKVKEEIYNLC | |
| | AGAAGACAAGAGCACCTGTTTCGGTAC | | LREVSNTRHLCLAPVNNAM | |
| | CAGCATCAACTACGTGGTCCTGCGACT | | TSIVMYLHEGPDSANYKKI | |
| | ACTGGGCCTGTCACGGGATCATCCGGT | | AARWPEFLSLNPSGMFMN | |
| | CTGCGTCAAGGCGCGCAAAACGCTGCT | | GTNGLQVWDTVFAVQYAC | |
| | CACCAAGTTTGGCGGCGCCATCAACAA | | VCGFAELPLYQKTIRAAFDF | |
| | CCCCCATTGGGGCAAGACCTGGCTGTC | | LDRSQINEPTEENSYRDDRV | |
| | GATTCTCAATCTCTACAAATGGGAGGG | | GGWPFSTKTQGYPVSDCTA | |
| | TGTGAATCCGGCCCCTGGCGTGCTCTG | | EALKAIIMVQNTPGYEDLK | |
| | GCTGTTGCCCTACTTTGTTCCTGTTCAT | | KQVSDKRKHTAIDLLLGMQ | |
| | CCGGGCCGATGGTGGGTCCATACCCGG | | NVGSFEPGSFASYEPIRTSS | |
| | TGGATCTACCTTGCCATGGGCTATCTG | | MLEKINPAEVFGNIMVEYP | |
| | GAGGCTGCGGAGGCCCAATGCGAACT | | YVECTDSVVLGLSCFRKYH | |
| | CACTCCGTTGCTGGAGGAGCTCCGAGA | | DYRNEDVDRAISAAIGYIIR | |
| | CGAAATCTACAAAAAGCCCTACTCGGA | | EQQPDGGFFGSWGVCYCY | |
| | GATTGATTTCTCCAAACATTGCAACTC | | AHMFAMEALETQNLNYNN | |
| | CATCTCCGGAGTCGACCTCTACTATCC | | CSTVQKACDFLAGYQEAD | |
| | CCACACCGGCCTTTTGAAGTTTGGCAA | | GGWAEDFKSCETQMYVRG | |
| | CGCGCTTCTCCGACGATACCGCAAGTT | | PHSLVVPTAMALLSLMSGR | |
| | CAGACCGCAGTGGATCAAAGAAAAGG | | YPQEDKIHAAARFLMSKQ | |
| | TCAAGGAGGAAATTTACAACTTGTGCC | | MSNGEWLKEEMEGVFNHT | |
| | TTCGAGAGGTTTCCAACACACGACACT | | CAIEYPNYRFYFVMKALGL | |
| | TGTGTCTCGCTCCCGTCAACAATGCCA | | YFKGYCQ | |
| | TGACCTCCATTGTCATGTATCTCCATGA | | | |
| | GGGGCCCGATTCGGCGAATTACAAAA | | | |
| | AGATTGCGGCCCGATGGCCCGAATTTC | | | |
| | TGTCTCTGAATCCGTCGGGAATGTTTA | | | |
| | TGAACGGCACCAACGGTCTGCAGGTCT | | | |
| | GGGATACTGTGTTTGCCGTGCAATACG | | | |
| | CGTGTGTTTGTGGCTTTGCCGAACTTCC | | | |
| | CCTGTACCAGAAGACGATCCGAGCGGC | | | |
| | GTTTGATTTTCTCGATCGGTCCCAGATC | | | |
| | AACGAGCCGACGGAGGAAAATTCCTA | | | |
| | TCGAGACGACCGCGTCGGAGGATGGC | | | |
| | CCTTTAGTACCAAGACCCAGGGGTATC | | | |
| | CAGTCTCCGACTGTACTGCCGAGGCTC | | | |
| | TCAAGGCCATCATCATGGTCCAGAATA | | | |
| | CGCCTGGATACGAGGATCTGAAGAAA | | | |
| | CAAGTGTCTGACAAGCGGAAACACACT | | | |
| | GCCATCGATCTACTTTTGGGAATGCAG | | | |
| | AACGTGGGCTCGTTTGAACCGGGCTCT | | | |
| | TTCGCCTCCTATGAGCCTATCCGGACG | | | |
| | TCGTCCATGCTGGAGAAGATCAATCCG | | | |
| | GCCGAGGTGTTTGGAAACATCATGGTG | | | |
| | GAGTATCCGTACGTGGAATGCACTGAT | | | |
| | TCTGTTGTTCTGGGTCTGTCCTGCTTTC | | | |
| | GAAAGTACCACGATTACCGCAACGAA | | | |
| | GACGTGGACCGAGCCATCTCTGCTGCC | | | |
| | ATTGGATACATTATTCGAGAGCAGCAG | | | |
| | CCTGACGGCGGCTTCTTTGGCTCCTGG | | | |
| | GGCGTGTGCTACTGCTACGCTCACATG | | | |
| | TTTGCCATGGAGGCTCTGGAGACGCAG | | | |
| | AATCTCAACTATAACAACTGTTCCACG | | | |
| | GTTCAAAAGGCGTGCGACTTTCTGGCG | | | |
| | GGCTACCAGGAAGCAGATGGAGGCTG | | | |
| | GGCCGAGGACTTTAAGTCGTGCGAGAC | | | |
| | TCAGATGTACGTGCGCGGACCCCATTC | | | |
| | GCTGGTCGTGCCTACTGCCATGGCCCT | | | |
| | GTTGAGTTTGATGAGTGGTCGGTATCC | | | |
| | CCAGGAGGACAAGATTCATGCTGCGGC | | | |
| | CCGGTTTCTCATGAGCAAGCAGATGAG | | | |
| | CAACGGTGAGTGGCTCAAGGAGGAGA | | | |
| | TGGAGGGGGTGTTTAACCATACTTGTG | | | |
| | CCATTGAGTATCCCAACTACCGGTTTT | | | |
| | ATTTTGTCATGAAGGCTTTGGGGTTGT | | | |
| | ATTTCAAGGGATATTGCCAGTGA | | | |
| 2B3 | ATGGGAATCCACGAAAGTGTGTCGAA | 114 | MGIHESVSKQFAKNGHSKY | 324 |
| | ACAGTTTGCGAAAAACGGACATTCCAA | | RSDRYGLPKTDLRRWTFHA | |
| | GTACCGCAGCGACCGATACGGCTTACC | | SDLGAQWWKYDDTTPLEE | |
| | TAAGACGGATCTGCGACGATGGACGTT | | LEKRATDYVKYSLELPGYA | |
| | CCACGCGTCCGATCTGGGGGCGCAATG | | PVTLDSKPVKNAYEAALKN | |
| | GTGGAAGTATGACGATACCACACCGCT | | WHLFASLQDPDSGAWQSE | |
| | GGAAGAGCTGGAAAAGAGGGCTACCG | | YDGPQFMSIGYVTACYFGG | |
| | ACTACGTCAAATACTCGCTGGAGCTGC | | NEIPTPVKTEMIRYIVNTAH | |
| | CGGGATACGCGCCCGTGACTCTGGACT | | PVDGGWGLHKEDKSTCFG | |
| | CCAAGCCCGTGAAAAATGCCTACGAA | | TSINYVVLRLLGLSRDHPVC | |
| | GCGGCTCTCAAAAACTGGCATCTGTTT | | VKARKTLLTKFGGAINNPH | |
| | GCGTCGCTGCAAGACCCCGACTCAGGC | | WGKTWLSILNLYKWEGVN | |
| | GCATGGCAGTCGGAATACGACGGACC | | PAPGELWLLPYFVPVHPGR | |
| | GCAGTTCATGTCGATCGGTTATGTGAC | | WWVHTRWIYLAMGYLEA | |
| | GGCGTGCTACTTTGGCGGCAACGAGAT | | AEAQCELTPLLEELRDEIYK | |
| | CCCCACGCCGGTCAAAACCGAAATGAT | | KPYSGIDFSKHCNSISGVDL | |
| | CAGATACATTGTCAACACAGCCCACCC | | YYPHTGLLKFGNALLRRYR | |
| | AGTTGACGGAGGCTGGGGCCTTCACAA | | KFRPQWINEKVKEEIYNLC | |
| | AGAAGACAAGAGCACCTGTTTCGGTAC | | LREVSNTRHLCLAPVNNAM | |
| | CAGCATCAACTACGTGGTCCTGCGACT | | TSIVMYLHEGPDSANYKKI | |
| | ACTGGGCCTGTCACGGGATCATCCGGT | | AARWPEFLSLNPSGMFMN | |
| | CTGCGTCAAGGCGCGCAAAACGCTGCT | | GTNGLQVWDTAFAVQYAC | |
| | CACCAAGTTTGGCGGCGCCATCAACAA | | VCGFAELPQYQKTIRAAFD | |
| | CCCCCATTGGGGCAAGACCTGGCTGTC | | FLDRSQINEPTEENSYRDDR | |
| | GATTCTCAATCTCTACAAATGGGAGGG | | VGGWPFSTKTQGYPVSDCT | |
| | TGTGAATCCGGCCCCTGGCGAGCTCTG | | AEALKAIIMVQNTPGYEDL | |
| | GCTGTTGCCCTACTTTGTTCCTGTTCAT | | KKQVSDKRKHTAIDLLLGM | |
| | CCGGGCCGATGGTGGGTCCATACCCGG | | QNVGSFEPGSFASYEPIRAS | |
| | TGGATCTACCTTGCCATGGGCTATCTG | | SMLEKINPAEVFGNIMVEY | |
| | GAGGCTGCGGAGGCCCAATGCGAACT | | PYVECTDSVVLGLSYFRKY | |
| | CACTCCGTTGCTGGAGGAGCTCCGAGA | | HDYRNEDVDRAISAAIGYII | |
| | CGAAATCTACAAAAAGCCCTACTCGGG | | REQQPDGGFFGSWGVCYC | |
| | GATTGATTTCTCCAAACATTGCAACTC | | YAHMFAMEALVTQNLNYN | |
| | CATCTCCGGAGTCGACCTCTACTATCC | | NCSTVQKACDFLAGYQEA | |
| | CCACACCGGCCTTTTGAAGTTTGGCAA | | DGGWAEDFKSCETQMYVR | |
| | CGCGCTTCTCCGACGATACCGCAAGTT | | GPHSLVVPTAMALLSLMSG | |
| | CAGACCGCAGTGGATCAATGAAAAGG | | RYPQEDKIHAAARFLMSKQ | |
| | TCAAGGAGGAAATTTACAACTTGTGCC | | MSNGEWLKEEMEGVFNHT | |
| | TTCGAGAGGTTTCCAACACACGACACT | | CAIEYPNYRVYFVMKALGL | |
| | TGTGTCTCGCTCCCGTCAACAATGCCA | | YFKGYCQ | |
| | TGACCTCCATTGTCATGTATCTCCATGA | | | |
| | GGGGCCCGATTCGGCGAATTACAAAA | | | |
| | AGATTGCGGCCCGATGGCCCGAATTTC | | | |
| | TGTCTCTGAATCCGTCGGGAATGTTTA | | | |
| | TGAACGGCACCAACGGTCTGCAGGTCT | | | |
| | GGGATACTGCGTTTGCCGTGCAATACG | | | |
| | CGTGTGTTTGTGGCTTTGCCGAACTTCC | | | |
| | CCAGTACCAGAAGACGATCCGAGCGG | | | |
| | CGTTTGATTTTCTCGATCGGTCCCAGAT | | | |
| | CAACGAGCCGACGGAGGAAAATTCCT | | | |
| | ATCGAGACGACCGCGTCGGAGGATGG | | | |
| | CCCTTTAGTACCAAGACCCAGGGGTAT | | | |
| | CCAGTCTCCGACTGTACTGCCGAGGCT | | | |
| | CTCAAGGCCATCATCATGGTCCAGAAT | | | |
| | ACGCCTGGATACGAGGATCTGAAGAA | | | |
| | ACAAGTGTCTGACAAGCGGAAACACA | | | |
| | CTGCCATCGATCTACTTTTGGGAATGC | | | |
| | AGAACGTGGGCTCGTTTGAACCGGGCT | | | |
| | CTTTCGCCTCCTATGAGCCTATCCGGG | | | |
| | CGTCGTCCATGCTGGAGAAGATCAATC | | | |
| | CGGCCGAGGTGTTTGGAAACATCATGG | | | |
| | TGGAGTATCCGTACGTGGAATGCACTG | | | |
| | ATTCTGTTGTTCTGGGTCTGTCCTACTT | | | |
| | TCGAAAGTACCACGATTACCGCAACGA | | | |
| | AGACGTGGACCGAGCCATCTCTGCTGC | | | |
| | CATTGGATACATTATTCGAGAGCAGCA | | | |
| | GCCTGACGGCGGCTTCTTTGGCTCCTG | | | |
| | GGGCGTGTGCTACTGCTACGCTCACAT | | | |
| | GTTTGCCATGGAGGCTCTGGTGACGCA | | | |
| | GAATCTCAACTATAACAACTGTTCCAC | | | |
| | GGTTCAAAAGGCGTGCGACTTTCTGGC | | | |
| | GGGCTACCAGGAAGCAGATGGAGGCT | | | |
| | GGGCCGAGGACTTTAAGTCGTGCGAGA | | | |
| | CTCAGATGTACGTGCGCGGACCCCATT | | | |
| | CGCTGGTCGTGCCTACTGCCATGGCCC | | | |
| | TGTTGAGTTTGATGAGTGGTCGGTATC | | | |
| | CCCAGGAGGACAAGATTCATGCTGCGG | | | |
| | CCCGGTTTCTCATGAGCAAGCAGATGA | | | |
| | GCAACGGTGAGTGGCTCAAGGAGGAG | | | |
| | ATGGAGGGGGTGTTTAACCATACTTGT | | | |
| | GCCATTGAGTATCCCAACTACCGGGTT | | | |
| | TATTTTGTCATGAAGGCTTTGGGGTTGT | | | |
| | ATTTCAAGGGATATTGCCAGTGA | | | |
| 2F9 | ATGGGAATCCACGAAAGTGTGTCGAA | 115 | MGIHESVSKQFAKNGHSKY | 325 |
| | ACAGTTTGCGAAAAACGGACATTCCAA | | RSDRYGLPKTDLRRWTFHA | |
| | GTACCGCAGCGACCGATACGGCTTACC | | SDLGAQWWEYDDTTPLEE | |
| | TAAGACGGATCTGCGACGATGGACGTT | | LEKRATDYVKYSLELPGYA | |
| | CCACGCGTCCGATCTGGGGGCGCAATG | | PVTLDSKPVKNAYEAAIKN | |
| | GTGGGAGTATGACGATACCACACCGCT | | WHLFASLQDPDSGAWQSE | |
| | GGAAGAGCTGGAAAAGAGGGCTACCG | | YDGPQFMSIGYVTACYFGG | |
| | ACTACGTCAAATACTCGCTGGAGCTGC | | NEIPTPVKTEMIRYIVNTAH | |
| | CGGGATACGCGCCCGTGACTCTGGACT | | PVDGGWGLHKEDKSTCFG | |
| | CCAAGCCCGTGAAAAATGCCTACGAA | | TSINYVVLRLLGLSRDHPVC | |
| | GCGGCTATCAAAAACTGGCATCTGTTT | | VKARKTLLTKFGGAINNPH | |
| | GCGTCGCTGCAAGACCCCGACTCCGGC | | WGKTWLSILNLYKWEGVN | |
| | GCATGGCAGTCGGAATACGACGGACC | | PAPGELWLLPYFVPVHPGR | |
| | GCAGTTCATGTCGATCGGTTATGTGAC | | WWVHTRWIYLAMGYLEA | |
| | GGCGTGCTACTTTGGCGGCAACGAGAT | | AEAQCELTPLLEELRDEIYK | |
| | CCCCACGCCGGTCAAAACCGAAATGAT | | KPYSEIDFSKHCNSISGVDL | |
| | CAGATACATTGTCAACACAGCCCACCC | | YYPHTGLLKFGNALLRRYR | |
| | AGTTGACGGAGGCTGGGGCCTTCACAA | | KFRPQWIKEKVKEEIYNLC | |
| | AGAAGACAAGAGCACCTGTTTCGGTAC | | LREVSNTRHLCLAPVNNAM | |
| | CAGCATCAACTACGTGGTCCTGCGACT | | SSIVMYLHEGPDPANYKKI | |
| | ACTGGGCCTGTCACGGGATCATCCGGT | | AARWPEFLSLNPSGMFMN | |
| | CTGCGTCAAGGCGCGCAAAACGCTGCT | | GTNGLQVWDTAFAVQYAC | |
| | CACCAAGTTTGGCGGCGCCATCAACAA | | VCGFAELPQYQKTIRAAFD | |
| | CCCCCATTGGGGCAAGACCTGGCTGTC | | FLDRSQINEPMEENSYRDD | |
| | GATTCTCAATCTCTACAAATGGGAGGG | | RVGGWPFSTKTQGYPVSDC | |
| | TGTGAATCCGGCCCCTGGCGAGCTCTG | | TAEALKAIIMVQNTPGYED | |
| | GCTGTTGCCCTACTTTGTTCCTGTTCAT | | LKKQVSDKRKHTAIDLLLG | |
| | CCGGGTCGATGGTGGGTCCATACCCGG | | MQNVGSFEPGSFASYEPIRA | |
| | TGGATCTACCTTGCCATGGGCTATCTG | | SSMLEKINPAEVFGNIMVE | |
| | GAGGCTGCGGAGGCCCAATGCGAACT | | YPYVECTDSVVLGLSYFRK | |
| | CACTCCGTTGCTGGAGGAGCTCCGAGA | | YHDYRNEDVDPAISAAIGYI | |
| | CGAAATCTACAAAAAGCCCTACTCGGA | | IREQQPDGGFFGSWGVCYC | |
| | GATTGATTTCTCCAAACATTGCAACTC | | YAHMFAMEALETQNLNYN | |
| | CATCTCCGGAGTCGACCTCTACTATCC | | NCSTVQKACDFLAGYQEA | |
| | CCACACCGGCCTTTTGAAGTTTGGCAA | | DGGWAEDFKSCETQMYVR | |
| | CGCGCTTCTCCGACGATACCGCAAGTT | | GPHSLVVPTAMALLSLMSG | |
| | CAGACCGCAGTGGATCAAAGAAAAGG | | RYPQEDKIHAAARFLMSKQ | |
| | TCAAGGAGGAAATTTACAACTTGTGCC | | MSNGEWLKEEMEGVFNHT | |
| | TTCGAGAGGTTTCCAACACACGACACT | | CAIEYPNYRFYFVMKALGL | |
| | TGTGTCTCGCTCCCGTCAACAATGCCA | | YFKGYCQ | |
| | TGTCCTCCATTGTCATGTATCTCCATGA | | | |
| | GGGGCCCGATCCGGCGAATTACAAAA | | | |
| | AGATTGCGGCCCGATGGCCCGAATTTC | | | |
| | TGTCTCTGAATCCGTCGGGAATGTTTA | | | |
| | TGAACGGCACCAACGGTCTGCAGGTCT | | | |
| | GGGATACTGCGTTTGCCGTGCAATACG | | | |
| | CGTGTGTTTGTGGCTTTGCCGAACTTCC | | | |
| | CCAGTACCAGAAGACGATCCGAGCGG | | | |
| | CGTTTGATTTTCTCGATCGGTCCCAGAT | | | |
| | CAACGAGCCGATGGAGGAAAATTCCT | | | |
| | ATCGAGACGACCGCGTCGGAGGATGG | | | |
| | CCCTTTAGTACCAAGACCCAGGGGTAT | | | |
| | CCAGTCTCCGACTGTACTGCCGAGGCT | | | |
| | CTCAAGGCCATCATCATGGTCCAGAAT | | | |
| | ACACCTGGATACGAGGATCTGAAGAA | | | |
| | ACAAGTGTCTGACAAGCGGAAACACA | | | |
| | CTGCCATCGATCTACTTTTGGGAATGC | | | |
| | AGAACGTGGGCTCGTTTGAACCGGGCT | | | |
| | CTTTCGCCTCCTATGAGCCTATCCGGG | | | |
| | CGTCGTCCATGCTGGAGAAGATCAATC | | | |
| | CGGCCGAGGTGTTTGGAAACATCATGG | | | |
| | TGGAGTATCCGTACGTGGAATGCACTG | | | |
| | ATTCTGTTGTTCTGGGTCTGTCCTACTT | | | |
| | TCGAAAGTACCACGATTACCGCAACGA | | | |
| | AGACGTGGACCCAGCCATCTCTGCTGC | | | |
| | CATTGGATACATTATTCGAGAGCAGCA | | | |
| | GCCTGACGGCGGCTTCTTTGGCTCCTG | | | |
| | GGGCGTGTGCTACTGCTACGCTCACAT | | | |
| | GTTTGCCATGGAGGCTCTGGAGACGCA | | | |
| | GAATCTCAACTATAACAACTGTTCCAC | | | |
| | GGTTCAAAAGGCGTGCGACTTTCTGGC | | | |
| | GGGCTACCAGGAAGCAGATGGAGGCT | | | |
| | GGGCCGAGGACTTTAAGTCGTGCGAGA | | | |
| | CTCAGATGTACGTGCGCGGACCCCATT | | | |
| | CGCTGGTCGTGCCTACTGCCATGGCCC | | | |
| | TGTTGAGTTTGATGAGTGGTCGGTATC | | | |
| | CCCAGGAGGACAAGATTCATGCTGCGG | | | |
| | CCCGGTTTCTCATGAGCAAGCAGATGA | | | |
| | GCAACGGTGAGTGGCTCAAGGAGGAG | | | |
| | ATGGAGGGGGTGTTTAACCATACTTGT | | | |
| | GCCATTGAGTATCCCAACTACCGGTTT | | | |
| | TATTTTGTCATGAAGGCTTTGGGGTTGT | | | |
| | ATTTCAAGGGATATTGCCAGTGA | | | |
| 1A3 | ATGGGAATCCACGAAAGTGTGTCGAA | 330 | MGIHESVSKQFAKNGHSKY | 331 |
| | ACAGTTTGCGAAAAACGGACATTCCAA | | RSDRYGLPKTDLRQWTFHA | |
| | GTACCGCAGCGACCGATACGGCTTACC | | SDLGAQWWKYDDTTPLEE | |
| | TAAGACGGATCTGCGACAATGGACGTT | | LEKRATDYVKYSLELPGYA | |
| | CCACGCGTCCGATCTGGGGGCGCAATG | | PVTLDSKPVKNAYEAALKN | |
| | GTGGAAGTATGACGATACCACACCGCT | | WHLFASLQDPDSGAWQSE | |
| | GGAAGAGTTGGAAAAGAGGGCTACCG | | YDGPQFMSIGYVTACYFGG | |
| | ACTACGTCAAATACTCGCTGGAGCTGC | | NEIPTPVKTEMIRYIVNTAH | |
| | CGGGATACGCGCCCGTGACTCTGGACT | | PVDGGWGLHKEDKSTCFG | |
| | CCAAGCCCGTGAAAAATGCCTACGAA | | TSINYVVLRLLGLSRDHPVC | |
| | GCGGCTCTCAAAAACTGGCATCTGTTT | | VKACKTLLTKFGGAINNPH | |
| | GCGTCGCTGCAAGACCCCGACTCCGGC | | WGKTWLSILNLYKWEGVN | |
| | GCATGGCAGTCGGAATACGACGGACC | | PAPGELWLLPYFVPVHPGR | |
| | GCAGTTCATGTCGATCGGTTATGTGAC | | WWVHTRWIYLAMGYLEA | |
| | GGCGTGCTACTTTGGCGGCAACGAGAT | | AEAQCELTPLLEELRDEIYK | |
| | CCCCACGCCGGTCAAAACCGAAATGAT | | KPYSEIGFSKHCITISGVDLY | |
| | CAGATACATTGTCAACACAGCCCACCC | | YPHTGLLKFGNALLRRYRK | |
| | AGTTGACGGAGGCTGGGGCCTTCACAA | | FRPQWIKEKVKEEIYNLCLR | |
| | AGAAGACAAGAGCACCTGTTTCGGTAC | | EVSNTRHLCLAPVNNAMTS | |
| | CAGCATCAACTACGTGGTCCTGCGACT | | IVMYLHEGPDSANYKKIAA | |
| | ACTGGGCCTGTCGCGGGATCATCCGGT | | RWPEFLSLNPSGMFMNGTN | |
| | CTGCGTCAAGGCGTGCAAAACGCTGCT | | GLQVWDTAFAVQYACVCG | |
| | CACCAAGTTTGGCGGCGCCATCAACAA | | FAELPQYQKTIRAAFDFLDR | |
| | CCCCCATTGGGGCAAGACCTGGCTGTC | | SQINEPTEENSYRDDRVGG | |
| | GATTCTCAATCTCTACAAATGGGAGGG | | WPFSTKTQGYPVSDCTAEA | |
| | TGTGAATCCGGCCCCTGGCGAGCTCTG | | LKAIIMVQNTPGYEDLKKQ | |
| | GCTGTTGCCCTACTTTGTTCCTGTTCAT | | VSDKRKHTAIDLLLGMQNV | |
| | CCGGGCCGATGGTGGGTCCATACCCGG | | GSFEPGSFASYEPIRASSML | |
| | TGGATCTACCTTGCCATGGGCTATCTG | | EKINPAEVFGNIMVEYPYV | |
| | GAGGCTGCGGAGGCCCAATGCGAACT | | ECTDSVVLGLSYFRKYHDY | |
| | CACTCCGTTGCTGGAGGAGCTCCGAGA | | RNEDVDRAISAAIGYIIREQ | |
| | CGAAATCTACAAAAAGCCCTACTCGGA | | QPDGGFFGSWGVCYCYAH | |
| | GATTGGTTTCTCCAAACATTGCATCAC | | MFAMEALETQSLNYNNCST | |
| | CATCTCCGGAGTCGACCTCTACTATCC | | VQKACDFLAGYQEADGGW | |
| | CCACACCGGCCTTTTGAAGTTTGGCAA | | AEDFKSCETQMYVRGPHSL | |
| | CGCGCTTCTCCGACGATACCGCAAGTT | | VVPTAMALLSLMSGRYPQE | |
| | CAGACCGCAGTGGATCAAAGAAAAGG | | DKIHAAARFLMSKQMSNG | |
| | TCAAGGAGGAAATTTATAACTTGTGCC | | EWLKEEMEGVFNHTCAIEY | |
| | TTCGAGAGGTTTCCAACACACGACACT | | PNYRFYFVMKALGLFFKGY | |
| | TGTGTCTCGCTCCCGTCAACAATGCCA | | CQ | |
| | TGACCTCCATTGTCATGTATCTCCATGA | | | |
| | GGGGCCCGATTCGGCGAATTACAAAA | | | |
| | AGATTGCGGCCCGATGGCCCGAATTTC | | | |
| | TGTCTCTGAATCCGTCGGGAATGTTTA | | | |
| | TGAACGGCACCAACGGTCTGCAGGTCT | | | |
| | GGGATACTGCGTTTGCCGTGCAATACG | | | |
| | CGTGTGTTTGTGGCTTTGCCGAACTTCC | | | |
| | CCAGTACCAGAAGACGATCCGAGCGG | | | |
| | CGTTTGATTTTCTCGATCGGTCCCAGAT | | | |
| | CAACGAGCCGACGGAGGAAAATTCCT | | | |
| | ATCGAGACGACCGCGTCGGAGGATGG | | | |
| | CCCTTTAGTACCAAGACCCAGGGGTAT | | | |
| | CCAGTCTCCGACTGTACTGCCGAGGCT | | | |
| | CTCAAGGCCATCATCATGGTCCAGAAT | | | |
| | ACGCCTGGATACGAGGATCTGAAGAA | | | |
| | ACAAGTGTCTGACAAGCGGAAACACA | | | |
| | CTGCCATCGATCTACTTTTGGGAATGC | | | |
| | AGAACGTGGGCTCGTTTGAACCGGGCT | | | |
| | CTTTCGCCTCCTATGAGCCTATCCGGG | | | |
| | CGTCGTCCATGCTGGAGAAGATCAATC | | | |
| | CGGCCGAGGTGTTTGGAAACATCATGG | | | |
| | TGGAGTATCCGTACGTGGAATGCACTG | | | |
| | ATTCTGTTGTTCTGGGTCTGTCCTACTT | | | |
| | TCGAAAGTACCACGATTACCGCAACGA | | | |
| | AGACGTGGACCGAGCCATCTCTGCTGC | | | |
| | CATTGGATACATTATTCGAGAGCAGCA | | | |
| | GCCTGACGGCGGCTTCTTTGGCTCCTG | | | |
| | GGGCGTGTGCTACTGCTACGCTCACAT | | | |
| | GTTTGCCATGGAGGCTCTGGAGACGCA | | | |
| | GAGTCTCAACTATAACAACTGTTCCAC | | | |
| | GGTTCAAAAGGCGTGCGACTTTCTGGC | | | |
| | GGGCTACCAGGAAGCAGATGGAGGCT | | | |
| | GGGCCGAGGACTTTAAGTCGTGCGAGA | | | |
| | CTCAGATGTACGTGCGCGGACCCCATT | | | |
| | CGCTGGTCGTGCCTACTGCCATGGCCC | | | |
| | TGTTGAGTTTGATGAGTGGTCGGTATC | | | |
| | CCCAGGAGGACAAGATTCATGCTGCGG | | | |
| | CCCGGTTTCTCATGAGCAAGCAGATGA | | | |
| | GCAACGGTGAGTGGCTCAAGGAGGAG | | | |
| | ATGGAGGGGGTGTTTAACCATACTTGT | | | |
| | GCCATTGAGTATCCCAACTACCGGTTT | | | |
| | TATTTTGTCATGAAGGCTTTGGGGTTGT | | | |
| | TTTTCAAGGGATATTGCCAGTGA | | | |
| 2H4 | ATGGGAATCCACGAAAGTGTGTCGAA | 116 | MGIHESVSKQFAKNGHSKY | 85 |
| | ACAGTTTGCGAAAAACGGACATTCCAA | | RSDRYGLPKTDLRRWTFHA | |
| | GTACCGCAGCGACCGATACGGCTTACC | | SDLGAQWWKYDDTTPLEE | |
| | TAAGACGGATCTGCGACGATGGACGTT | | LEKRATDYVKYSLELPGYA | |
| | CCACGCGTCCGATCTGGGGGCGCAATG | | PVTLDSKPVKNAYEAALKN | |
| | GTGGAAGTATGACGATACCACACCGCT | | WHLFASLQDPDSGAWQSE | |
| | GGAAGAGCTGGAAAAGAGGGCTACCG | | YDGPQFMSIGYVTACYFGG | |
| | ACTACGTCAAATACTCGCTGGAGCTGC | | NEIPTPVKTEMIRYIVNTAH | |
| | CGGGATACGCGCCCGTGACTCTGGACT | | PVDGGWGLHKEDKSTCFG | |
| | CCAAGCCCGTGAAAAATGCCTACGAA | | TSINYVVLRLLGLSRDHPVC | |
| | GCGGCTCTCAAAAACTGGCATCTGTTT | | VKARKTLLTKFGGAINNPH | |
| | GCGTCGCTGCAAGACCCCGACTCCGGC | | WGKTWLSILNLYKWEGVN | |
| | GCATGGCAGTCGGAATACGACGGACC | | PAPGELWLLPYFVPVHPGR | |
| | GCAGTTCATGTCGATCGGTTATGTGAC | | WWVHTRWIYLAMGYLEA | |
| | GGCGTGCTACTTTGGCGGCAACGAGAT | | AEAQCELTPLLEELRDEIYK | |
| | CCCCACGCCGGTCAAAACCGAAATGAT | | KPYSEIDFSKHCNSISGVDL | |
| | CAGATACATTGTCAACACAGCCCACCC | | YYPHTGLLKFGNALLRRYR | |
| | AGTTGACGGAGGCTGGGGCCTTCACAA | | KFRPQWIKEKVKEEIYNLC | |
| | AGAAGACAAGAGCACCTGTTTCGGTAC | | LREVSNTRHLCLAPVNNAM | |
| | CAGCATCAACTACGTGGTCCTGCGACT | | TSIVMYLHEGPDSANYKKI | |
| | ACTGGGCCTGTCACGGGATCATCCGGT | | AARWPEFLSLNPSGMFMN | |
| | CTGCGTCAAGGCGCGCAAAACGCTGCT | | GTNGLQVWDTAFAVQYAC | |
| | CACCAAGTTTGGCGGCGCCATCAACAA | | VCGFAELPQYQKTIRAASD | |
| | CCCCCATTGGGGCAAGACCTGGCTGTC | | FLDRSQINEPTEENSYRDGR | |
| | GATTCTCAATCTCTACAAATGGGAGGG | | VGGWPFSTKTQGYPVSDCT | |
| | TGTGAATCCGGCCCCTGGCGAGCTCTG | | AEALKAIIMVQNTPGYEDL | |
| | GCTGTTGCCCTACTTTGTTCCTGTTCAT | | KKQVSDKRKHTAIDLLLGM | |
| | CCGGGCCGATGGTGGGTCCATACCCGG | | QNVGSFEPGSFASYEPIRAS | |
| | TGGATCTACCTTGCCATGGGCTATCTG | | SMLEKFNPAEVFGNIMVEY | |
| | GAGGCTGCGGAGGCCCAATGCGAACT | | PYVECTDSVVLGLSYFRKY | |
| | CACTCCGTTGCTGGAGGAGCTCCGAGA | | HDYRNEDVDRAISAAIGYII | |
| | CGAAATCTACAAAAAGCCCTACTCGGA | | REQQPDGGFFGSWGVCYC | |
| | GATTGATTTCTCCAAACATTGCAACTC | | YAHMFAMEALETQNLNYN | |
| | CATCTCCGGAGTCGACCTCTACTATCC | | NCSTVQKACDFLAGYQEA | |
| | CCACACCGGCCTTTTGAAGTTTGGCAA | | DGGWAEDFKSCETQMYVR | |
| | CGCGCTTCTCCGACGATACCGCAAGTT | | GPHSLVVPTAMALLSLMSG | |
| | CAGACCGCAGTGGATCAAAGAAAAGG | | RYPQEDKIHAAARFLMSKQ | |
| | TCAAGGAGGAAATTTACAACTTGTGCC | | MSNGEWLKEEMEGVFNHT | |
| | TTCGAGAGGTTTCCAACACACGACACT | | CAIEYPNYRFYFVMKALGL | |
| | TGTGTCTCGCTCCCGTCAACAATGCCA | | YFKGYCQ | |
| | TGACCTCCATTGTCATGTATCTCCATGA | | | |
| | GGGGCCCGATTCGGCGAATTACAAAA | | | |
| | AGATTGCGGCCCGATGGCCCGAATTTC | | | |
| | TGTCTCTGAATCCGTCGGGAATGTTTA | | | |
| | TGAACGGCACCAACGGTCTGCAGGTCT | | | |
| | GGGATACTGCGTTTGCCGTGCAATACG | | | |
| | CGTGTGTTTGTGGCTTTGCCGAACTTCC | | | |
| | CCAGTACCAGAAGACGATCCGAGCGG | | | |
| | CGTCTGATTTTCTCGATCGGTCCCAGAT | | | |
| | CAACGAGCCGACGGAGGAAAATTCCT | | | |
| | ATCGAGACGGCCGCGTCGGAGGATGG | | | |
| | CCCTTTAGTACCAAGACCCAGGGGTAT | | | |
| | CCAGTCTCCGACTGTACTGCCGAGGCT | | | |
| | CTCAAGGCCATCATCATGGTCCAGAAT | | | |
| | ACGCCTGGATACGAGGATCTGAAGAA | | | |
| | ACAAGTGTCTGACAAGCGGAAACACA | | | |
| | CTGCCATCGATCTACTTTTGGGAATGC | | | |
| | AGAACGTGGGCTCGTTTGAACCGGGCT | | | |
| | CTTTCGCCTCCTATGAGCCTATCCGGG | | | |
| | CGTCGTCCATGCTGGAGAAGTTCAATC | | | |
| | CGGCCGAGGTGTTTGGAAACATCATGG | | | |
| | TGGAGTATCCGTACGTGGAATGCACTG | | | |
| | ATTCTGTTGTTCTGGGTCTGTCCTACTT | | | |
| | TCGAAAGTACCACGATTACCGCAACGA | | | |
| | AGACGTGGACCGAGCCATCTCTGCTGC | | | |
| | CATTGGATACATTATTCGAGAGCAGCA | | | |
| | GCCTGACGGTGGCTTCTTTGGCTCCTG | | | |
| | GGGCGTGTGCTACTGCTACGCTCACAT | | | |
| | GTTTGCCATGGAGGCTCTGGAGACGCA | | | |
| | GAATCTCAACTATAACAACTGTTCCAC | | | |
| | GGTTCAAAAGGCGTGCGACTTTCTGGC | | | |
| | GGGCTACCAGGAAGCAGATGGAGGCT | | | |
| | GGGCCGAGGACTTTAAGTCGTGCGAGA | | | |
| | CTCAGATGTACGTGCGCGGACCCCATT | | | |
| | CGCTGGTCGTGCCTACTGCCATGGCCC | | | |
| | TGTTGAGTTTGATGAGTGGTCGGTATC | | | |
| | CCCAGGAGGACAAGATTCATGCTGCGG | | | |
| | CCCGGTTTCTCATGAGCAAGCAGATGA | | | |
| | GCAACGGTGAGTGGCTCAAGGAGGAG | | | |
| | ATGGAGGGGGTGTTTAACCATACTTGT | | | |
| | GCCATTGAGTATCCCAACTACCGGTTT | | | |
| | TATTTTGTCATGAAGGCTTTGGGGTTGT | | | |
| | ATTTCAAGGGATATTGCCAGTGA | | | |
| 2F6 | ATGGGAATCCACGAAAGTGTGTCGAA | 4 | MGIHESVSKQFAKNGHSKY | 3 |
| | ACAGTTTGCGAAAAACGGACATTCCAA | | RSDRYGLPKTDLRRWTFHA | |
| | GTACCGCAGCGACCGATACGGCTTACC | | SDLGAQWWKYDGTTPLEE | |
| | TAAGACGGATCTGCGACGATGGACGTT | | LEKRATDYVRYSLELPGYA | |
| | CCACGCGTCCGATCTGGGGGCGCAATG | | PVTLDSKPVKNAYEAALKS | |
| | GTGGAAGTATGACGGTACCACACCGCT | | WHLFASLQDPDSGAWQSE | |
| | GGAAGAGCTGGAAAAGAGGGCTACCG | | YDGPQFMSIGYVTACYFGG | |
| | ACTACGTCAGATACTCGCTGGAGCTGC | | NEIPTPVKTEMIRYIVNTAH | |
| | CGGGATACGCGCCCGTGACTCTGGACT | | PVDGGWGLHKEDKSTCFG | |
| | CCAAGCCCGTGAAAAATGCCTACGAA | | TSINYVVLRLLGLSRDHPVC | |
| | GCGGCTCTCAAAAGCTGGCATCTGTTT | | VKARKTLLTKFGGAINNPH | |
| | GCGTCGCTGCAAGACCCCGACTCCGGC | | WGKTWLSILNLYKWEGVN | |
| | GCATGGCAGTCGGAATACGACGGACC | | PAPGELWLLPYFVPVHPGR | |
| | GCAGTTCATGTCGATCGGTTATGTGAC | | WWVHTRWIYLAMGYLEA | |
| | GGCGTGCTACTTTGGCGGCAACGAGAT | | AEAQCELTPLLEELRDEIYK | |
| | CCCCACGCCGGTCAAAACCGAAATGAT | | KPYSEIDFSKHCNSISGVDL | |
| | CAGATACATTGTCAACACAGCCCACCC | | YYPHTGLLKFGNALLRRYR | |
| | AGTTGACGGAGGCTGGGGCCTTCACAA | | KFRPQWIKEKVKEEIYNLC | |
| | AGAAGACAAGAGCACCTGTTTCGGTAC | | LREVSNTRHLCLAPVNNAM | |
| | CAGCATCAACTACGTGGTCCTGCGACT | | TSIVMYLHEGPDSANYKKI | |
| | ACTGGGCCTGTCACGGGATCATCCGGT | | AARWPEFLSLNPSGMFMN | |
| | CTGCGTCAAGGCGCGCAAAACGCTGCT | | GTNGLQVWDTAFAVQYAC | |
| | CACCAAGTTTGGCGGCGCCATCAACAA | | VCSFAELPQYQKTIRAAFDF | |
| | CCCCCATTGGGGCAAGACCTGGCTGTC | | LDRSQINEPTEENSYRDDRV | |
| | GATTCTCAATCTCTACAAATGGGAGGG | | GGWPFSTKTQGYPVSDCTA | |
| | TGTGAATCCGGCCCCTGGCGAGCTCTG | | EALKAIIMVQNTPGYEDLK | |
| | GCTGTTGCCCTACTTTGTTCCTGTTCAT | | KQVSDKRKHTAIDLLLGMQ | |
| | CCGGGCCGATGGTGGGTCCATACCCGG | | NVGSFEPGSFASYEPIRASS | |
| | TGGATCTACCTTGCCATGGGCTATCTG | | MLEKINPAEVFGNIMVEYP | |
| | GAGGCTGCGGAGGCCCAATGCGAACT | | YVECTDSVVLGLSYFRKYH | |
| | CACTCCGTTGCTGGAGGAGCTCCGAGA | | DYRNEDVDRAISAAIGYIIR | |
| | CGAAATCTACAAAAAGCCCTACTCGGA | | EQQPDGGFFGSWGVCYCY | |
| | GATTGATTTCTCCAAACATTGCAACTC | | AHMFAMEALVTQNLNYNN | |
| | CATCTCCGGAGTCGACCTCTACTATCC | | CSTVQKACDFLAGYQEAD | |
| | CCACACCGGCCTTTTGAAGTTTGGCAA | | GGWAEDFKSCETQMYVRG | |
| | CGCGCTTCTCCGACGATACCGCAAGTT | | PHSLVVPTAMALLSLMSGR | |
| | CAGACCGCAGTGGATCAAAGAAAAGG | | YPQEDKIHAAARFLMSKQ | |
| | TCAAGGAGGAAATTTACAACTTGTGCC | | MSNGEWLKEEMEGVFNHT | |
| | TTCGAGAGGTTTCCAACACACGACACT | | CAIEYPNYRLYFVMKALGL | |
| | TGTGTCTCGCTCCCGTCAACAATGCCA | | YFKGYCQ | |
| | TGACCTCCATTGTCATGTATCTCCATGA | | | |
| | GGGGCCCGATTCGGCGAATTACAAAA | | | |
| | AGATTGCGGCCCGATGGCCCGAATTTC | | | |
| | TGTCTCTGAATCCGTCGGGAATGTTTA | | | |
| | TGAACGGCACCAACGGTCTGCAGGTCT | | | |
| | GGGATACTGCGTTTGCCGTGCAATACG | | | |
| | CGTGTGTTTGTAGCTTTGCCGAACTTCC | | | |
| | CCAGTACCAGAAGACGATCCGAGCGG | | | |
| | CGTTTGATTTTCTCGATCGGTCCCAGAT | | | |
| | CAACGAGCCGACGGAGGAAAATTCCT | | | |
| | ATCGAGACGACCGCGTCGGAGGATGG | | | |
| | CCCTTTAGTACCAAGACCCAGGGGTAT | | | |
| | CCAGTCTCCGACTGTACTGCCGAGGCT | | | |
| | CTCAAGGCCATCATCATGGTCCAGAAT | | | |
| | ACGCCTGGATACGAGGATCTGAAGAA | | | |
| | ACAAGTGTCTGACAAGCGGAAACACA | | | |
| | CTGCCATCGATCTACTTTTGGGAATGC | | | |
| | AGAACGTGGGCTCGTTTGAACCGGGCT | | | |
| | CTTTCGCCTCCTATGAGCCTATCCGGG | | | |
| | CGTCGTCCATGCTGGAGAAGATCAATC | | | |
| | CGGCCGAGGTGTTTGGAAACATCATGG | | | |
| | TGGAGTATCCGTACGTGGAATGCACTG | | | |
| | ATTCTGTTGTTCTGGGTCTGTCCTACTT | | | |
| | TCGAAAGTACCACGATTACCGCAACGA | | | |
| | AGACGTGGACCGAGCCATCTCTGCTGC | | | |
| | CATCGGATACATTATTCGAGAGCAGCA | | | |
| | GCCTGACGGTGGCTTCTTTGGCTCCTG | | | |
| | GGGCGTGTGCTACTGCTACGCTCACAT | | | |
| | GTTTGCCATGGAGGCTCTGGTGACGCA | | | |
| | GAATCTCAACTATAACAACTGTTCCAC | | | |
| | GGTTCAAAAGGCGTGCGACTTTCTGGC | | | |
| | GGGCTACCAGGAAGCAGATGGAGGCT | | | |
| | GGGCCGAGGACTTTAAGTCGTGCGAGA | | | |
| | CTCAGATGTACGTGCGCGGACCCCATT | | | |
| | CGCTGGTCGTGCCTACTGCCATGGCCC | | | |
| | TGTTGAGTTTGATGAGTGGTCGGTATC | | | |
| | CCCAGGAGGACAAGATTCATGCTGCGG | | | |
| | CCCGGTTTCTCATGAGCAAGCAGATGA | | | |
| | GCAACGGTGAGTGGCTCAAGGAGGAG | | | |
| | ATGGAGGGGGTGTTTAACCATACTTGT | | | |
| | GCCATTGAGTATCCCAACTACCGGTTA | | | |
| | TATTTTGTCATGAAGGCTTTGGGGTTGT | | | |
| | ATTTCAAGGGATATTGCCAGTGA | | | |
| 3A5 | ATGGGAATCCACGAAAGTGTGTCGAA | 117 | MGIHESVSKQFAKNGHSKY | 326 |
| | ACAGTTTGCGAAAAACGGACATTCCAA | | RSDRYGLPKTDLRRWTFHA | |
| | GTACCGCAGCGACCGATACGGCTTACC | | SDLGAQWWKYDDTTPLEE | |
| | TAAGACGGATCTGCGACGATGGACGTT | | LEKRATDYVKYSLELPGYA | |
| | CCACGCGTCCGATCTGGGGGCGCAATG | | PVTLDSKPVKNAYEAALKN | |
| | GTGGAAGTATGACGATACCACACCGCT | | WHLFASLQDPDSGAWQSE | |
| | GGAAGAGCTGGAAAAGAGGGCTACCG | | YDGPQFMSIGYVTACYFGG | |
| | ACTACGTCAAATACTCGCTGGAGCTGC | | NEIPTPVKTEMIRYIVNTAH | |
| | CGGGATACGCGCCCGTGACTCTGGACT | | PVDGGWGLHKEDKSTCFG | |
| | CCAAGCCCGTGAAAAATGCCTACGAA | | TSINYVVLRLLGLSRDHPVC | |
| | GCGGCTCTCAAAAACTGGCATCTGTTT | | VKARKTLVTKFGGAINNPH | |
| | GCGTCGCTGCAAGACCCCGACTCCGGC | | WGKTWLSILNLYKWEGVN | |
| | GCATGGCAGTCGGAATACGACGGACC | | PAPGELWLLPYFVPVHPGR | |
| | GCAGTTCATGTCGATCGGTTATGTGAC | | WWVHTRWIYLAMGYLEA | |
| | GGCGTGCTACTTTGGCGGCAACGAGAT | | AEAQCELTPLLEELRDEIYI | |
| | CCCCACGCCGGTCAAAACCGAAATGAT | | KPYSEIDFSKHCNSISGVDL | |
| | CAGATACATTGTCAACACAGCCCACCC | | YYPHTGLLKFGSALLRRYR | |
| | AGTTGACGGAGGCTGGGGCCTTCACAA | | KFRPQWIKEKVKEEIYNLC | |
| | AGAAGACAAGAGCACCTGTTTCGGTAC | | LREVSNTRHLCLAPVNNAM | |
| | CAGCATCAACTACGTGGTCCTGCGACT | | TSIVMYLHEGLDSANYKKI | |
| | ACTGGGCCTGTCACGGGATCATCCGGT | | AARWPEFLSLNPSGMFMN | |
| | CTGCGTCAAGGCGCGCAAAACGCTGGT | | GTNGLQVWDTAFAVQYAC | |
| | CACCAAGTTTGGCGGCGCCATCAACAA | | VCGFAELPQYQKTIRAAFD | |
| | CCCCCATTGGGGCAAGACCTGGCTGTC | | FLDRSQINEPTEENSYRDDR | |
| | GATTCTCAATCTCTACAAATGGGAGGG | | VGGWPFSTKTQGYPVSDCT | |
| | TGTGAATCCGGCCCCTGGCGAGCTCTG | | AEALKAIIMVQNTPGYEDL | |
| | GCTGTTGCCCTACTTTGTTCCTGTTCAT | | KKQVSDKRKHTAIDLLLGM | |
| | CCGGGCCGATGGTGGGTCCATACCCGG | | QNVGSFEPGSFASYEPIRAS | |
| | TGGATCTACCTTGCCATGGGCTATCTG | | SMLEKINPAEVFGNIMVEY | |
| | GAGGCTGCGGAGGCCCAATGCGAACT | | PYVECTDSVVLGLSYFRKY | |
| | CACTCCGTTGCTGGAGGAGCTCCGAGA | | HDYRNEDVDRAISAAIGYII | |
| | CGAAATCTACATAAAGCCCTACTCGGA | | REQQPDGGFFGSWGVCYC | |
| | GATTGATTTCTCCAAACATTGCAACTC | | YTHMFAMEALETQNLNYN | |
| | CATCTCCGGAGTCGACCTCTACTATCC | | NCSTVQKACDFLADYQEA | |
| | CCACACCGGCCTTTTGAAGTTTGGCAG | | DGGWAEDLKSCETQMYVR | |
| | CGCGCTTCTCCGACGATACCGCAAGTT | | GPHSLVVPTAMALLSLMSG | |
| | CAGACCGCAGTGGATCAAAGAAAAGG | | RYPQEDKIHAAARFLMSKQ | |
| | TCAAGGAGGAAATTTACAACTTGTGCC | | MSNGEWLKEEMEGVFNHT | |
| | TTCGAGAGGTTTCCAACACACGACACT | | CAIEYPNYRFYFVMKALGL | |
| | TGTGTCTCGCTCCCGTCAACAATGCCA | | YFKGYCQ | |
| | TGACCTCCATTGTCATGTATCTCCATGA | | | |
| | GGGGCTCGATTCGGCGAATTACAAAAA | | | |
| | GATTGCGGCCCGATGGCCCGAATTTCT | | | |
| | GTCTCTGAATCCGTCGGGAATGTTTAT | | | |
| | GAACGGCACCAACGGTCTGCAGGTCTG | | | |
| | GGATACTGCGTTTGCCGTGCAATACGC | | | |
| | GTGTGTTTGTGGCTTTGCCGAACTTCCC | | | |
| | CAGTACCAGAAGACGATCCGAGCGGC | | | |
| | GTTTGATTTTCTCGATCGGTCCCAGATC | | | |
| | AACGAGCCGACGGAGGAAAATTCCTA | | | |
| | TCGAGACGACCGCGTCGGAGGATGGC | | | |
| | CCTTTAGTACCAAGACCCAGGGGTATC | | | |
| | CAGTCTCCGACTGTACTGCCGAGGCTC | | | |
| | TCAAGGCCATCATCATGGTCCAGAATA | | | |
| | CGCCTGGATACGAGGATCTGAAGAAA | | | |
| | CAAGTGTCTGACAAGCGGAAACACACT | | | |
| | GCCATCGATCTACTTTTGGGAATGCAG | | | |
| | AACGTGGGCTCGTTTGAACCGGGCTCT | | | |
| | TTCGCCTCCTATGAGCCTATCCGGGCG | | | |
| | TCGTCCATGCTGGAGAAGATCAATCCG | | | |
| | GCCGAGGTGTTTGGAAACATCATGGTG | | | |
| | GAGTATCCGTACGTGGAATGCACTGAT | | | |
| | TCTGTTGTTCTGGGTCTGTCCTACTTTC | | | |
| | GAAAGTACCACGATTACCGCAACGAA | | | |
| | GACGTGGACCGAGCCATCTCTGCTGCC | | | |
| | ATTGGATATATTATTCGAGAGCAGCAG | | | |
| | CCTGACGGCGGCTTCTTTGGCTCCTGG | | | |
| | GGCGTGTGCTACTGCTACACTCACATG | | | |
| | TTTGCCATGGAGGCTCTGGAGACGCAG | | | |
| | AATCTCAACTATAACAACTGTTCCACG | | | |
| | GTTCAAAAGGCGTGCGACTTTCTGGCG | | | |
| | GACTACCAGGAAGCAGATGGAGGCTG | | | |
| | GGCCGAGGACCTTAAGTCGTGCGAGAC | | | |
| | TCAGATGTACGTGCGCGGACCCCATTC | | | |
| | GCTGGTCGTGCCTACTGCCATGGCCCT | | | |
| | GTTGAGTTTGATGAGTGGTCGGTATCC | | | |
| | CCAGGAGGACAAGATTCATGCTGCGGC | | | |
| | CCGGTTTCTCATGAGCAAGCAGATGAG | | | |
| | CAACGGTGAGTGGCTCAAGGAGGAGA | | | |
| | TGGAGGGGGTGTTTAACCATACTTGTG | | | |
| | CCATTGAGTATCCCAACTACCGGTTTT | | | |
| | ATTTTGTCATGAAGGCTTTGGGGTTGT | | | |
| | ATTTCAAGGGATATTGCCAGTGA | | | |
| 2C5 | ATGGGAATCCACGAAAGTGTGTCGAA | 328 | MGIHESVSKQFAKYGHSKY | 329 |
| | ACAGTTTGCGAAATACGGACATTCCAA | | RSDRYGLPKTDLRRWTFHA | |
| | GTACCGCAGCGACCGATACGGCTTACC | | SDLGAQWWKYDDTTPLEE | |
| | TAAGACGGATCTGCGACGATGGACGTT | | LEKRATDYVKYSLELPGYA | |
| | CCACGCGTCCGATCTGGGGGCGCAATG | | PVTLDSKPVKNAYEAALKN | |
| | GTGGAAGTATGACGATACCACACCGCT | | WHLFASLQDPDSGAWQSE | |
| | GGAAGAGCTGGAAAAGAGGGCTACCG | | YDGPQFMSIGYVTACYFGG | |
| | ACTACGTCAAATACTCGCTGGAGCTGC | | SEIPTPVKTEMIRCIVNTAHP | |
| | CGGGATACGCGCCCGTGACTCTGGACT | | VDGGWGLHKEDKSTCFGT | |
| | CCAAGCCCGTGAAAAATGCCTACGAA | | SINYVVLRLLGLSRDHPVC | |
| | GCGGCTCTCAAAAACTGGCATCTGTTT | | VKAHKTLLTKFGGAINNPH | |
| | GCGTCGCTGCAAGACCCCGACTCCGGC | | WGKTWLSILNLYKWEGVN | |
| | GCATGGCAGTCGGAATACGACGGACC | | PAPGELWLLPYFVPVHPGR | |
| | GCAGTTCATGTCGATCGGTTATGTGAC | | WWVHTRWIYLAMGYLEA | |
| | GGCGTGCTACTTTGGCGGCAGCGAGAT | | AEAQCELTPLLEELRDEIYK | |
| | CCCCACGCCGGTCAAAACCGAAATGAT | | KPYSEFDFSKHCNSISGVDL | |
| | CAGATGCATTGTCAACACAGCCCACCC | | YYPHTGLLKFGNARLRRYR | |
| | AGTTGACGGAGGCTGGGGCCTTCATAA | | KFRPQWIKEKVKEEIYNLC | |
| | AGAAGACAAGAGCACCTGTTTCGGTAC | | LREVSNTRHLCLAPVNNAM | |
| | CAGCATCAACTACGTGGTCCTGCGACT | | TSIVMYLHEGPDSANYKKI | |
| | ACTGGGCCTGTCACGGGATCATCCGGT | | AARWPEFLSLNPSGMFMN | |
| | CTGCGTCAAGGCGCACAAAACGCTGCT | | GTNGLQVWDTAFAVQYAC | |
| | CACCAAGTTTGGCGGCGCCATCAACAA | | VCGFAELPQYQKTIRAAIDF | |
| | CCCCCATTGGGGCAAGACCTGGCTGTC | | LDRSQINVPSEENSYRDDR | |
| | GATTCTCAATCTCTACAAATGGGAGGG | | VGGWPFSTKTQGYPVSDCT | |
| | TGTGAATCCGGCCCCTGGCGAGCTCTG | | AEALKASIMVQNTPGYEDL | |
| | GCTGTTGCCCTACTTTGTTCCTGTTCAT | | KKQVSDKRKHTAIDLLLGM | |
| | CCGGGCCGATGGTGGGTCCATACCCGG | | QNVGSFEPGSFASYEPIRAS | |
| | TGGATCTACCTTGCCATGGGCTATCTG | | SMLEKINPAEVFGNIMVEY | |
| | GAGGCTGCGGAGGCCCAATGCGAACT | | PYVECTDSVVLGLSYFRKY | |
| | CACTCCGTTGCTGGAGGAGCTCCGAGA | | HDYRNEDVDRAISAAIGYII | |
| | CGAAATCTACAAAAAGCCCTACTCGGA | | REQQPDGGFFGSWGVCYC | |
| | GTTTGATTTCTCCAAACATTGCAACTCC | | YAHMFAMEALETQNLNYN | |
| | ATCTCCGGAGTCGACCTCTACTATCCC | | NCSTVQRACDFLAGYQEA | |
| | CACACCGGCCTTTTGAAGTTTGGCAAC | | DGGWAEDFKSCEAQMYVR | |
| | GCGCGTCTCCGACGATACCGCAAGTTC | | GPHSLVVPTAMALLSLMSG | |
| | AGACCGCAGTGGATCAAAGAAAAGGT | | RYPQEDKIHAAARFLMSKQ | |
| | CAAGGAGGAAATTTACAACTTGTGCCT | | MSNGEWLKEEMEGVFNHT | |
| | TCGAGAGGTTTCCAACACACGACACTT | | CAIEYPNYRFYFVMKALGL | |
| | GTGTCTCGCTCCCGTCAACAATGCCAT | | YFKGYCQ | |
| | GACCTCCATTGTCATGTATCTCCATGA | | | |
| | GGGGCCCGATTCGGCGAATTACAAAA | | | |
| | AGATTGCGGCCCGATGGCCCGAATTTC | | | |
| | TGTCTCTGAATCCGTCGGGAATGTTTA | | | |
| | TGAACGGCACCAACGGTCTGCAGGTCT | | | |
| | GGGATACTGCGTTTGCCGTGCAATACG | | | |
| | CGTGTGTTTGTGGCTTTGCCGAACTTCC | | | |
| | CCAGTACCAGAAGACGATCCGAGCGG | | | |
| | CGATTGATTTTCTCGATCGGTCCCAGA | | | |
| | TCAACGTGCCGTCGGAGGAAAATTCCT | | | |
| | ATCGAGACGACCGCGTCGGAGGATGG | | | |
| | CCCTTTAGTACCAAGACCCAGGGGTAT | | | |
| | CCAGTCTCCGACTGTACTGCCGAGGCT | | | |
| | CTCAAGGCCAGCATCATGGTCCAGAAT | | | |
| | ACGCCTGGATACGAGGATCTGAAGAA | | | |
| | ACAAGTGTCTGACAAGCGGAAACACA | | | |
| | CTGCCATCGATCTACTTTTGGGAATGC | | | |
| | AGAACGTGGGCTCGTTTGAACCGGGCT | | | |
| | CTTTCGCCTCCTATGAGCCTATCCGGG | | | |
| | CGTCGTCCATGCTGGAGAAGATCAATC | | | |
| | CGGCCGAGGTGTTTGGAAACATCATGG | | | |
| | TGGAGTATCCGTACGTGGAATGCACTG | | | |
| | ATTCTGTTGTTCTGGGTCTGTCCTACTT | | | |
| | TCGAAAGTACCACGATTACCGCAACGA | | | |
| | AGACGTGGACCGAGCCATCTCTGCTGC | | | |
| | CATTGGATACATTATTCGAGAGCAGCA | | | |
| | GCCTGACGGCGGCTTCTTTGGCTCCTG | | | |
| | GGGCGTGTGCTACTGCTACGCTCACAT | | | |
| | GTTTGCCATGGAGGCTCTGGAGACGCA | | | |
| | GAATCTCAACTATAACAACTGTTCCAC | | | |
| | GGTTCAAAGGGCGTGCGACTTTCTGGC | | | |
| | GGGCTACCAGGAAGCAGATGGAGGCT | | | |
| | GGGCCGAGGACTTTAAGTCGTGCGAGG | | | |
| | CTCAGATGTACGTGCGCGGACCCCATT | | | |
| | CGCTGGTCGTGCCTACTGCCATGGCCC | | | |
| | TGTTGAGTTTGATGAGTGGTCGGTATC | | | |
| | CCCAGGAGGACAAGATTCATGCTGCGG | | | |
| | CCCGGTTTCTCATGAGCAAGCAGATGA | | | |
| | GCAATGGTGAGTGGCTCAAGGAGGAG | | | |
| | ATGGAGGGGGTGTTTAACCATACTTGT | | | |
| | GCCATTGAGTATCCCAACTACCGGTTT | | | |
| | TATTTTGTCATGAAGGCTTTGGGGTTGT | | | |
| | ATTTCAAGGGATATTGCCAGTGA | | | |
| 887779 | ATGGGAATCCACGAAAGTGTGTCGAA | 61 | MGIHESVSKQFAKNGHSKY | 1 |
| | ACAGTTTGCGAAAAACGGACATTCCAA | | RSDRYGLPKTDLRRWTFHA | |
| | GTACCGCAGCGACCGATACGGCTTACC | | SDLGAQWWKYDDTTPLEE | |
| | TAAGACGGATCTGCGACGATGGACGTT | | LEKRATDYVKYSLELPGYA | |
| | CCACGCGTCCGATCTGGGGGCGCAATG | | PVTLDSKPVKNAYEAALKN | |
| | GTGGAAGTATGACGATACCACACCGCT | | WHLFASLQDPDSGAWQSE | |
| | GGAAGAGCTGGAAAAGAGGGCTACCG | | YDGPQFMSIGYVTACYFGG | |
| | ACTACGTCAAATACTCGCTGGAGCTGC | | NEIPTPVKTEMIRYIVNTAH | |
| | CGGGATACGCGCCCGTGACTCTGGACT | | PVDGGWGLHKEDKSTCFG | |
| | CCAAGCCCGTGAAAAATGCCTACGAA | | TSINYVVLRLLGLSRDHPVC | |
| | GCGGCTCTCAAAAACTGGCATCTGTTT | | VKARKTLLTKFGGAINNPH | |
| | GCGTCGCTGCAAGACCCCGACTCCGGC | | WGKTWLSILNLYKWEGVN | |
| | GCGTGGCAGAGCGAATACGACGGACC | | PAPGELWLLPYFVPVHPGR | |
| | GCAGTTCATGAGCATCGGCTATGTGAC | | WWVHTRWIYLAMGYLEA | |
| | GGCGTGCTACTTTGGCGGCAACGAGAT | | AEAQCELTPLLEELRDEIYK | |
| | CCCCACGCCGGTCAAAACCGAAATGAT | | KPYSEIDFSKHCNSISGVDL | |
| | CAGATACATTGTCAACACAGCCCACCC | | YYPHTGLLKFGNALLRRYR | |
| | AGTTGACGGAGGCTGGGGCCTTCACAA | | KFRPQWIKEKVKEEIYNLC | |
| | AGAAGACAAGAGCACCTGCTTCGGTAC | | LREVSNTRHLCLAPVNNAM | |
| | CAGCATCAACTACGTGGTCCTGCGACT | | TSIVMYLHEGPDSANYKKI | |
| | ACTGGGCCTGTCACGGGACCATCCGGT | | AARWPEFLSLNPSGMFMN | |
| | CTGCGTCAAGGCGCGCAAAACGCTGCT | | GTNGLQVWDTAFAVQYAC | |
| | CACCAAGTTTGGCGGCGCCATCAACAA | | VCGFAELPQYQKTIRAAFD | |
| | CCCCCATTGGGGCAAGACCTGGCTGTC | | FLDRSQINEPTEENSYRDDR | |
| | GATTCTCAATCTCTACAAATGGGAGGG | | VGGWPFSTKTQGYPVSDCT | |
| | TGTGAATCCGGCCCCTGGCGAGCTCTG | | AEALKAIIMVQNTPGYEDL | |
| | GCTGTTGCCCTACTTTGTTCCTGTTCAT | | KKQVSDKRKHTAIDLLLGM | |
| | CCGGGCCGATGGTGGGTCCATACCCGG | | QNVGSFEPGSFASYEPIRAS | |
| | TGGATCTACCTTGCCATGGGCTATCTG | | SMLEKINPAEVFGNIMVEY | |
| | GAGGCTGCGGAGGCCCAATGCGAACT | | PYVECTDSVVLGLSYFRKY | |
| | CACTCCGTTGCTGGAGGAGCTCCGAGA | | HDYRNEDVDRAISAAIGYII | |
| | CGAAATCTACAAAAAGCCCTACTCGGA | | REQQPDGGFFGSWGVCYC | |
| | GATTGATTTCTCCAAACATTGCAACTC | | YAHMFAMEALETQNLNYN | |
| | CATCTCCGGAGTCGACCTCTACTATCC | | NCSTVQKACDFLAGYQEA | |
| | CCACACCGGCCTTTTGAAGTTTGGCAA | | DGGWAEDFKSCETQMYVR | |
| | CGCGCTTCTCCGACGATACCGCAAGTT | | GPHSLVVPTAMALLSLMSG | |
| | CAGACCGCAGTGGATCAAAGAAAAGG | | RYPQEDKIHAAARFLMSKQ | |
| | TCAAGGAGGAAATTTACAACTTGTGCC | | MSNGEWLKEEMEGVFNHT | |
| | TTCGAGAGGTTTCCAACACACGACACT | | CAIEYPNYRFYFVMKALGL | |
| | TGTGTCTCGCTCCCGTCAACAATGCCA | | YFKGYCQ | |
| | TGACCTCCATTGTCATGTATCTCCATGA | | | |
| | GGGGCCCGATTCGGCGAATTACAAAA | | | |
| | AGATTGCGGCCCGATGGCCCGAATTTC | | | |
| | TGTCTCTGAATCCGTCGGGAATGTTTA | | | |
| | TGAACGGCACCAACGGTCTCCAGGTCT | | | |
| | GGGATACTGCGTTTGCCGTGCAATACG | | | |
| | CGTGTGTTTGTGGCTTTGCCGAACTTCC | | | |
| | CCAGTACCAGAAGACGATCCGAGCGG | | | |
| | CGTTTGATTTTCTCGATCGGTCCCAGAT | | | |
| | CAACGAGCCGACGGAGGAAAATTCCT | | | |
| | ATCGAGACGACCGCGTCGGAGGATGG | | | |
| | CCCTTTAGTACCAAGACCCAGGGGTAT | | | |
| | CCGGTCTCCGACTGTACTGCCGAGGCT | | | |
| | CTCAAGGCCATCATCATGGTCCAGAAT | | | |
| | ACGCCTGGATACGAGGATCTGAAGAA | | | |
| | ACAAGTGTCTGACAAGCGGAAACACA | | | |
| | CTGCCATCGATCTACTTTTGGGAATGC | | | |
| | AGAACGTGGGCTCGTTTGAACCGGGCT | | | |
| | CTTTCGCCTCCTATGAGCCTATCCGGG | | | |
| | CGTCGTCCATGCTGGAGAAGATCAATC | | | |
| | CGGCCGAGGTGTTTGGAAACATCATGG | | | |
| | TGGAGTATCCGTACGTGGAATGCACTG | | | |
| | ATTCTGTTGTTCTGGGTCTCTCCTACTT | | | |
| | TCGAAAGTACCACGATTACCGCAACGA | | | |
| | AGACGTGGACCGAGCCATCTCTGCTGC | | | |
| | CATTGGATACATTATTCGAGAGCAGCA | | | |
| | GCCTGACGGCGGCTTCTTTGGCTCCTG | | | |
| | GGGCGTGTGCTACTGCTACGCTCACAT | | | |
| | GTTTGCCATGGAGGCTCTGGAGACGCA | | | |
| | GAATCTCAACTATAACAACTGTTCCAC | | | |
| | GGTTCAAAAGGCGTGCGACTTTCTGGC | | | |
| | GGGCTACCAGGAAGCAGATGGAGGCT | | | |
| | GGGCCGAGGACTTTAAGTCGTGCGAGA | | | |
| | CCCAGATGTACGTGCGCGGACCCCATT | | | |
| | CGCTGGTCGTGCCTACTGCCATGGCCC | | | |
| | TGTTGAGTTTGATGAGTGGTCGGTATC | | | |
| | CCCAGGAGGACAAGATTCATGCTGCGG | | | |
| | CCCGGTTTCTCATGAGCAAGCAGATGA | | | |
| | GCAACGGTGAGTGGCTCAAGGAGGAG | | | |
| | ATGGAGGGGGTGTTTAACCATACTTGT | | | |
| | GCCATTGAGTATCCCAACTACCGGTTT | | | |
| | TATTTTGTCATGAAGGCTTTGGGGTTGT | | | |
| | ATTTCAAGGGATATTGCCAGTGA | | | |
| 907811 | ATGGGAATCCACGAAAGTGTGTCGAA | 4 | MGIHESVSKQFAKNGHSKY | 3 |
| | ACAGTTTGCGAAAAACGGACATTCCAA | | RSDRYGLPKTDLRRWTFHA | |
| | GTACCGCAGCGACCGATACGGCTTACC | | SDLGAQWWKYDGTTPLEE | |
| | TAAGACGGATCTGCGACGATGGACGTT | | LEKRATDYVRYSLELPGYA | |
| | CCACGCGTCCGATCTGGGGGCGCAATG | | PVTLDSKPVKNAYEAALKS | |
| | GTGGAAGTATGACGGTACCACACCGCT | | WHLFASLQDPDSGAWQSE | |
| | GGAAGAGCTGGAAAAGAGGGCTACCG | | YDGPQFMSIGYVTACYFGG | |
| | ACTACGTCAGATACTCGCTGGAGCTGC | | NEIPTPVKTEMIRYIVNTAH | |
| | CGGGATACGCGCCCGTGACTCTGGACT | | PVDGGWGLHKEDKSTCFG | |
| | CCAAGCCCGTGAAAAATGCCTACGAA | | TSINYVVLRLLGLSRDHPVC | |
| | GCGGCTCTCAAAAGCTGGCATCTGTTT | | VKARKTLLTKFGGAINNPH | |
| | GCGTCGCTGCAAGACCCCGACTCCGGC | | WGKTWLSILNLYKWEGVN | |
| | GCATGGCAGTCGGAATACGACGGACC | | PAPGELWLLPYFVPVHPGR | |
| | GCAGTTCATGTCGATCGGTTATGTGAC | | WWVHTRWIYLAMGYLEA | |
| | GGCGTGCTACTTTGGCGGCAACGAGAT | | AEAQCELTPLLEELRDEIYK | |
| | CCCCACGCCGGTCAAAACCGAAATGAT | | KPYSEIDFSKHCNSISGVDL | |
| | CAGATACATTGTCAACACAGCCCACCC | | YYPHTGLLKFGNALLRRYR | |
| | AGTTGACGGAGGCTGGGGCCTTCACAA | | KFRPQWIKEKVKEEIYNLC | |
| | AGAAGACAAGAGCACCTGTTTCGGTAC | | LREVSNTRHLCLAPVNNAM | |
| | CAGCATCAACTACGTGGTCCTGCGACT | | TSIVMYLHEGPDSANYKKI | |
| | ACTGGGCCTGTCACGGGATCATCCGGT | | AARWPEFLSLNPSGMFMN | |
| | CTGCGTCAAGGCGCGCAAAACGCTGCT | | GTNGLQVWDTAFAVQYAC | |
| | CACCAAGTTTGGCGGCGCCATCAACAA | | VCSFAELPQYQKTIRAAFDF | |
| | CCCCCATTGGGGCAAGACCTGGCTGTC | | LDRSQINEPTEENSYRDDRV | |
| | GATTCTCAATCTCTACAAATGGGAGGG | | GGWPFSTKTQGYPVSDCTA | |
| | TGTGAATCCGGCCCCTGGCGAGCTCTG | | EALKAIIMVQNTPGYEDLK | |
| | GCTGTTGCCCTACTTTGTTCCTGTTCAT | | KQVSDKRKHTAIDLLLGMQ | |
| | CCGGGCCGATGGTGGGTCCATACCCGG | | NVGSFEPGSFASYEPIRASS | |
| | TGGATCTACCTTGCCATGGGCTATCTG | | MLEKINPAEVFGNIMVEYP | |
| | GAGGCTGCGGAGGCCCAATGCGAACT | | YVECTDSVVLGLSYFRKYH | |
| | CACTCCGTTGCTGGAGGAGCTCCGAGA | | DYRNEDVDRAISAAIGYIIR | |
| | CGAAATCTACAAAAAGCCCTACTCGGA | | EQQPDGGFFGSWGVCYCY | |
| | GATTGATTTCTCCAAACATTGCAACTC | | AHMFAMEALVTQNLNYNN | |
| | CATCTCCGGAGTCGACCTCTACTATCC | | CSTVQKACDFLAGYQEAD | |
| | CCACACCGGCCTTTTGAAGTTTGGCAA | | GGWAEDFKSCETQMYVRG | |
| | CGCGCTTCTCCGACGATACCGCAAGTT | | PHSLVVPTAMALLSLMSGR | |
| | CAGACCGCAGTGGATCAAAGAAAAGG | | YPQEDKIHAAARFLMSKQ | |
| | TCAAGGAGGAAATTTACAACTTGTGCC | | MSNGEWLKEEMEGVFNHT | |
| | TTCGAGAGGTTTCCAACACACGACACT | | CAIEYPNYRLYFVMKALGL | |
| | TGTGTCTCGCTCCCGTCAACAATGCCA | | YFKGYCQ | |
| | TGACCTCCATTGTCATGTATCTCCATGA | | | |
| | GGGGCCCGATTCGGCGAATTACAAAA | | | |
| | AGATTGCGGCCCGATGGCCCGAATTTC | | | |
| | TGTCTCTGAATCCGTCGGGAATGTTTA | | | |
| | TGAACGGCACCAACGGTCTGCAGGTCT | | | |
| | GGGATACTGCGTTTGCCGTGCAATACG | | | |
| | CGTGTGTTTGTAGCTTTGCCGAACTTCC | | | |
| | CCAGTACCAGAAGACGATCCGAGCGG | | | |
| | CGTTTGATTTTCTCGATCGGTCCCAGAT | | | |
| | CAACGAGCCGACGGAGGAAAATTCCT | | | |
| | ATCGAGACGACCGCGTCGGAGGATGG | | | |
| | CCCTTTAGTACCAAGACCCAGGGGTAT | | | |
| | CCAGTCTCCGACTGTACTGCCGAGGCT | | | |
| | CTCAAGGCCATCATCATGGTCCAGAAT | | | |
| | ACGCCTGGATACGAGGATCTGAAGAA | | | |
| | ACAAGTGTCTGACAAGCGGAAACACA | | | |
| | CTGCCATCGATCTACTTTTGGGAATGC | | | |
| | AGAACGTGGGCTCGTTTGAACCGGGCT | | | |
| | CTTTCGCCTCCTATGAGCCTATCCGGG | | | |
| | CGTCGTCCATGCTGGAGAAGATCAATC | | | |
| | CGGCCGAGGTGTTTGGAAACATCATGG | | | |
| | TGGAGTATCCGTACGTGGAATGCACTG | | | |
| | ATTCTGTTGTTCTGGGTCTGTCCTACTT | | | |
| | TCGAAAGTACCACGATTACCGCAACGA | | | |
| | AGACGTGGACCGAGCCATCTCTGCTGC | | | |
| | CATCGGATACATTATTCGAGAGCAGCA | | | |
| | GCCTGACGGTGGCTTCTTTGGCTCCTG | | | |
| | GGGCGTGTGCTACTGCTACGCTCACAT | | | |
| | GTTTGCCATGGAGGCTCTGGTGACGCA | | | |
| | GAATCTCAACTATAACAACTGTTCCAC | | | |
| | GGTTCAAAAGGCGTGCGACTTTCTGGC | | | |
| | GGGCTACCAGGAAGCAGATGGAGGCT | | | |
| | GGGCCGAGGACTTTAAGTCGTGCGAGA | | | |
| | CTCAGATGTACGTGCGCGGACCCCATT | | | |
| | CGCTGGTCGTGCCTACTGCCATGGCCC | | | |
| | TGTTGAGTTTGATGAGTGGTCGGTATC | | | |
| | CCCAGGAGGACAAGATTCATGCTGCGG | | | |
| | CCCGGTTTCTCATGAGCAAGCAGATGA | | | |
| | GCAACGGTGAGTGGCTCAAGGAGGAG | | | |
| | ATGGAGGGGGTGTTTAACCATACTTGT | | | |
| | GCCATTGAGTATCCCAACTACCGGTTA | | | |
| | TATTTTGTCATGAAGGCTTTGGGGTTGT | | | |
| | ATTTCAAGGGATATTGCCAGTGA | | | |

TABLE 12
Non-Limiting Examples of CDSs

| | Nucleotide | Protein |
|---|---|---|
| Name | SEQ ID NO | SEQ ID NO |
| A0A0K9RW03_m | 184 | 224 |
| AquAgaCDS1_m | 185 | 225 |
| AquAgaCDS16 | 186 | 226 |
| AquAgaCDS16 | 327 | 226 |
| AquAgaCDS6 | 187 | 227 |
| BenHisCDS2_m | 188 | 228 |
| A0A0D3QY32 | 189 | 229 |
| A0A0D3QXV2 | 190 | 230 |
| CmaCh17G013880.1 | 191 | 231 |
| A0A1S3CBF6 | 192 | 232 |
| CocGraCDS4 | 193 | 233 |
| CocGraCDS6_m | 194 | 234 |
| CSPI06G07180.1 | 195 | 235 |
| CucFoeCDS | 196 | 236 |
| CucMelMakCDS5 | 197 | 237 |
| CucMetCDS | 198 | 238 |
| CucPepOviCDS1_m | 199 | 239 |
| CucPepOviCDS2 | 200 | 240 |
| CucPepOviCDS3 | 201 | 241 |
| CucPepOviCDS3_m | 202 | 242 |
| Cucsa.349060.1 | 203 | 243 |
| F6GYI4 | 204 | 244 |
| GynCarCDS1 | 205 | 245 |
| GynCarCDS4 | 206 | 246 |
| K7NBZ9 | 207 | 247 |
| LagSicCDS2_m | 208 | 248 |
| Lus10014538.g_m | 209 | 249 |
| Lus10032146.g_m | 210 | 250 |
| MomChaCDS2 | 211 | 251 |
| MomChaCDS4 | 212 | 252 |
| O23909_PEA_Y118L | 213 | 253 |
| Q6BE24 | 214 | 254 |
| SecEduCDS | 215 | 255 |
| SgCDS1 | 216 | 256 |
| SgCDS1 | 332 | 256 |
| SgCDS_Scer1 | 217 | 257 |
| TriKirCDS10 | 218 | 258 |
| TriKirCDS4 | 219 | 259 |
| XP_006340479.1 | 220 | 260 |
| XP_008655662.1 | 221 | 261 |
| XP_010541955.1_m | 222 | 262 |
| XP_016688836.1_m | 223 | 263 |

TABLE 13
Non-Limiting Examples of C11 Hydroxylases (P450s),
Cytochrome P450 Reductases, Epoxide Hydrolases
(EPHs), and Squalene Epoxidases

| | Nucleotide | Protein |
|---|---|---|
| Enzyme | SEQ ID NO | SEQ ID NO |
| C11 hydroxylase | 264 | 280 |
| C11 hydroxylase (cucurbitadienol oxidase) | 265 | 281 |
| Cytochrome P450 reductase | 266 | 282 |
| Cytochrome P450 reductase | 267 | 283 |
| Epoxide hydrolase | 268 | 284 |
| Epoxide hydrolase | 269 | 285 |
| Epoxide hydrolase (epoxide hydratase) | 270 | 286 |
| Epoxide hydrolase (epoxide hydratase) | 271 | 287 |
| Epoxide hydrolase (epoxide hydratase) | 272 | 288 |
| Epoxide hydrolase (epoxide hydratase) | 273 | 289 |
| Epoxide hydrolase (epoxide hydratase) | 274 | 290 |
| Epoxide hydrolase (epoxide hydratase) | 275 | 291 |
| Epoxide hydrolase (epoxide hydratase) | 276 | 292 |
| Squalene epoxidase | 277 | 293 |
| Squalene epoxidase | 278 | 294 |
| Squalene epoxidase | 279 | 295 |

TABLE 14
Sequences of Additional Enzymes Associated with the Disclosure

| | Nucleotide | Protein |
|---|---|---|
| Name | SEQ ID NO | SEQ ID NO |
| CYP1798 | 296 | 305 |
| AtCPR1 | 297 | 306 |
| CPR4497 | 298 | 307 |
| sgCDS | 299 | 308 |
| EPH3 | 300 | 309 |
| atEPH2 | 301 | 310 |
| ERG9 | 302 | 311 |
| ERG1 | 303 | 312 |
| ERG7 | 304 | 313 |
| CYP5491 | 314 | 315 |

### EQUIVALENTS

Those skilled in the art will recognize or be able to ascertain using no more than routine experimentation many equivalents to the specific embodiments of the invention described in this application. Such equivalents are intended to be encompassed by the following claims.

All references, including patent documents, disclosed in this application are incorporated by reference in their entirety, particularly for the disclosure referenced in this application.

1. A host cell for producing mogrol, one or more mogrol precursors, and/or one or more mogrosides, wherein the host cell comprises a heterologous polynucleotide encoding a lanosterol synthase with reduced activity as compared to a wild-type lanosterol synthase, wherein the host cell is capable of producing:
(a) one or more mogrol precursors selected from the group consisting of: squalene, 2-3-oxidosqualene, 2,3,22,23-dioxidosqualene, cucurbitadienol, 24, 25-expoxycucurbitadienol, 11-hydroxycucurbitadienol, 11-hydroxy-24,25-epoxycucurbitadienol, 11-hydroxycucurbitadienol, 11-oxo-cucurbitadienol, and 24,25-dihydroxycucurbitadienol;
(b) mogrol; and/or
(c) one or more mogrosides.

2. The host cell of claim 1, wherein the host cell comprises a heterologous polynucleotide encoding a lanosterol synthase, wherein the lanosterol synthase comprises an amino acid substitution or deletion relative to SEQ ID NO: 1 at one or more residues corresponding to position 14, 33, 47, 50, 66, 80, 83, 85, 92, 94, 107, 122, 132, 145, 158, 170, 172, 184, 193, 197, 198, 212, 213, 227, 228, 231, 235, 248, 249, 260, 282, 286, 287, 289, 295, 296, 309, 314, 316, 329, 344, 360, 370, 371, 372, 398, 407, 414, 417, 423, 432, 437, 442, 444, 452, 474, 479, 491, 498, 515, 526, 529, 536, 544, 552, 559, 560, 564, 578, 586, 608, 610, 617, 619, 620, 631, 638, 650, 655, 660, 679, 686, 702, 710, 726, 736, 738, and/or 742 in SEQ ID NO: 1

3. The host cell of claim 1 or 2, wherein the lanosterol synthase comprises 2, 3, 4, 5, 6, 7, 8, 9, 10, 11, or 12 amino acid substitutions and/or deletions relative to SEQ ID NO: 1.

4. The host cell of any one of claims 1-3, wherein the lanosterol synthase comprises:
a) the amino acid Y at the residue corresponding to position 14 in SEQ ID NO:1;
b) the amino acid Q at the residue corresponding to position 33 in SEQ ID NO:1;
c) the amino acid E at the residue corresponding to position 47 in SEQ ID NO:1;
d) the amino acid G at the residue corresponding to position 50 in SEQ ID NO:1;
e) the amino acid R at the residue corresponding to position 66 in SEQ ID NO:1;
f) the amino acid G at the residue corresponding to position 80 in SEQ ID NO: 1;
g) the amino acid L at the residue corresponding to position 83 in SEQ ID NO: 1;
h) the amino acid N at the residue corresponding to position 85 in SEQ ID NO:1;
i) the amino acid I at the residue corresponding to position 92 in SEQ ID NO:1;
j) the amino acid S at the residue corresponding to position 94 in SEQ ID NO:1;
k) the amino acid D at the residue corresponding to position 107 in SEQ ID NO:1;
l) the amino acid C at the residue corresponding to position 122 in SEQ ID NO:1;
m) the amino acid S at the residue corresponding to position 132 in SEQ ID NO:1;
n) the amino acid C at the residue corresponding to position 145 in SEQ ID NO:1;
o) the amino acid S at the residue corresponding to position 158 in SEQ ID NO:1;
p) the amino acid A at the residue corresponding to position 170 in SEQ ID NO: 1;
q) the amino acid N at the residue corresponding to position 172 in SEQ ID NO:1;
r) the amino acid W at the residue corresponding to position 184 in SEQ ID NO:1;
s) the amino acid C or H at the residue corresponding to position 193 in SEQ ID NO:1;
t) the amino acid V at the residue corresponding to position 197 in SEQ ID NO:1;
u) the amino acid I at the residue corresponding to position 198 in SEQ ID NO: 1;
v) the amino acid I at the residue corresponding to position 212 in SEQ ID NO:1;
w) the amino acid L at the residue corresponding to position 213 in SEQ ID NO:1;
x) the amino acid L at the residue corresponding to position 227 in SEQ ID NO:1;
y) the amino acid T at the residue corresponding to position 228 in SEQ ID NO: 1;
z) the amino acid V at the residue corresponding to position 231 in SEQ ID NO:1;
aa) the amino acid M at the residue corresponding to position 235 in SEQ ID NO:1;
bb) the amino acid F at the residue corresponding to position 248 in SEQ ID NO:1;
cc) the amino acid L at the residue corresponding to position 249 in SEQ ID NO:1;
dd) the amino acid R at the residue corresponding to position 260 in SEQ ID NO:1;
cc) the amino acid I at the residue corresponding to position 282 in SEQ ID NO:1;
ff) the amino acid F at the residue corresponding to position 286 in SEQ ID NO: 1;
gg) the amino acid G at the residue corresponding to position 287 in SEQ ID NO:1;
hh) the amino acid G at the residue corresponding to position 289 in SEQ ID NO: 1;
ii) the amino acid I at the residue corresponding to position 295 in SEQ ID NO: 1;
jj) the amino acid T at the residue corresponding to position 296 in SEQ ID NO: 1;
kk) the amino acid F at the residue corresponding to position 309 in SEQ ID NO: 1;
ll) the amino acid S at the residue corresponding to position 314 in SEQ ID NO:1;
mm) the amino acid R at the residue corresponding to position 316 in SEQ ID NO:1;
nn) the amino acid N at the residue corresponding to position 329 in SEQ ID NO:1;
oo) the amino acid A at the residue corresponding to position 344 in SEQ ID NO: 1;
pp) the amino acid S at the residue corresponding to position 360 in SEQ ID NO:1;
qq) the amino acid L at the residue corresponding to position 370 in SEQ ID NO:1;
rr) the amino acid V at the residue corresponding to position 371 in SEQ ID NO:1;
ss) the amino acid P at the residue corresponding to position 372 in SEQ ID NO:1;
tt) the amino acid I at the residue corresponding to position 398 in SEQ ID NO: 1;
uu) the amino acid V at the residue corresponding to position 407 in SEQ ID NO:1;
vv) the amino acid S at the residue corresponding to position 414 in SEQ ID NO:1;
ww) the amino acid S at the residue corresponding to position 417 in SEQ ID NO:1;
xx) the amino acid L at the residue corresponding to position 423 in SEQ ID NO:1;
yy) the amino acid I or S at the residue corresponding to position 432 in SEQ ID NO:1;
zz) the amino acid L at the residue corresponding to position 437 in SEQ ID NO:1;
aaa) the amino acid V at the residue corresponding to position 442 in SEQ ID NO:1;
bbb) the amino acid M or S at the residue corresponding to position 444 in SEQ ID NO:1;
ccc) the amino acid G at the residue corresponding to position 452 in SEQ ID NO:1;
ddd) the amino acid V at the residue corresponding to position 474 in SEQ ID NO:1;
ccc) the amino acid S at the residue corresponding to position 479 in SEQ ID NO:1;
fff) the amino acid Q at the residue corresponding to position 491 in SEQ ID NO:1;
ggg) the amino acid N at the residue corresponding to position 498 in SEQ ID NO: 1;
hhh) the amino acid L at the residue corresponding to position 515 in SEQ ID NO:1;
iii) the amino acid T at the residue corresponding to position 526 in SEQ ID NO:1;
jjj) the amino acid T at the residue corresponding to position 529 in SEQ ID NO:1;
kkk) the amino acid F at the residue corresponding to position 536 in SEQ ID NO:1;
lll) the amino acid Y at the residue corresponding to position 544 in SEQ ID NO:1;
mmm) the amino acid E at the residue corresponding to position 552 in SEQ ID NO:1;
nnn) the amino acid A at the residue corresponding to position 559 in SEQ ID NO:1;
ooo) the amino acid M at the residue corresponding to position 560 in SEQ ID NO:1;
ppp) the amino acid C or N at the residue corresponding to position 564 in SEQ ID NO:1;
qqq) the amino acid P at the residue corresponding to position 578 in SEQ ID NO:1;
rrr) the amino acid F at the residue corresponding to position 586 in SEQ ID NO:1;
sss) the amino acid T at the residue corresponding to position 608 in SEQ ID NO:1;
ttt) the amino acid I at the residue corresponding to position 610 in SEQ ID NO: 1;
uuu) the amino acid V at the residue corresponding to position 617 in SEQ ID NO:1;
vvv) the amino acid L at the residue corresponding to position 619 in SEQ ID NO:1;
www) the amino acid S at the residue corresponding to position 620 in SEQ ID NO:1;
xxx) the amino acid E or R at the residue corresponding to position 631 in SEQ ID NO:1;
yyy) the amino acid D at the residue corresponding to position 638 in SEQ ID NO:1;
zzz) the amino acid L at the residue corresponding to position 650 in SEQ ID NO:1;
aaaa) the amino acid A at the residue corresponding to position 655 in SEQ ID NO:1;
bbbb) the amino acid H at the residue corresponding to position 660 in SEQ ID NO:1;
cccc) the amino acid S at the residue corresponding to position 679 in SEQ ID NO:1;
dddd) the amino acid E at the residue corresponding to position 686 in SEQ ID NO: 1;
eeee) the amino acid D at the residue corresponding to position 702 in SEQ ID NO:1;
ffff) the amino acid Q at the residue corresponding to position 710 in SEQ ID NO:1;
gggg) the amino acid L or V at the residue corresponding to position 726 in SEQ ID NO:1;
hhhh) the amino acid F at the residue corresponding to position 736 in SEQ ID NO:1;
iiii) the amino acid M at the residue corresponding to position 738 in SEQ ID NO:1; and/or
jjjj) a truncation that results in deletion of the residue corresponding to position 742 in SEQ ID NO: 1.

5. The host cell of any one of claims 1-4, wherein the lanosterol synthase comprises the amino acid substitution E617V, G107D, and/or K631E relative to SEQ ID NO: 1.

6. The host cell of any one of claims 1-4, wherein relative to SEQ ID NO: 1, the lanosterol synthase comprises:
a) R33Q, R193C, D289G, N295I, S296T, N620S, and Y736F;
b) R184W, L235M, L260R, and E710Q;
c) K47E, L92I, T360S, S372P, T444M, and R578P;
d) D50G, K66R, N94S, G417S, E617V, and F726L;
e) N14Y, N132S, Y145C, R193H, I286F, L316R, F432I, E442V, T444S, I479S, K631R, and T655A;
f) F432S, D452G, and I536F;
g) E287G, K329N, E617V, and F726V;
h) E231V, A407V, Q423L, A529T, and Y564C;
i) V248F, D371V, and G702D;
j) L197V, K282I, N314S, P370L, A608T, G638D, and F650L;
k) L491Q, Y586F, and R660H;
l) G122C, H249L, and K738M;
m) P227L, E474V, V559A, and Y564N;
n) K85N, G158S, S515L, P526T, Q619L, and a truncation resulting in a deletion of the residue corresponding to Q742 in SEQ ID NO: 1;
o) G107D and K631E;
p) T212I, W213L, N544Y, and V552E;
q) I172N, C414S, L560M, and G679S;
r) R193C, D289G, N295I, S296T, N620S, and Y736F;
s) K85N and G158S;
t) L197V, K282I, N314S, and P370L;
u) I172N, C414S, and L560M;
v) D371V, M610I, and G702D;
w) D371V, K498N, M610I, and G702D;
x) D80G, P83L, T170A, T198I, and A228T;
y) T360S, S372P, T444M, and R578P;
z) D50G, K66R, N94S, G417S, and E617V; or
aa) L309F, V344A, T398I, and K686E.

7. The host cell of any one of claims 1-4, wherein relative to SEQ ID NO: 1, the lanosterol synthase comprises the following amino acid substitutions:
(a) R193C, D289G, N295I, S296T, N620S, and Y736F;
(b) F432S, D452G, and I536F;
(c) K85N and G158S;
(d) L197V, K282I, N314S, and P370L;
(e) I172N, C414S, L560M, and G679S;
(f) I172N, C414S, and L560M;
(g) D371V, M610I, and G702D;
(h) D371V, K498N, M610I, and G702D;
(i) D80G, P83L, T170A, T198I, and A228T;
(j) D50G, K66R, N94S, G417S, E617V, and F726L;
(k) T360S, S372P, T444M, and R578P;
(l) D50G, K66R, N94S, G417S, and E617V; and
(m) L309F, V344A, T398I, and K686E.

8. The host cell of any one of claims 1-4, wherein relative to SEQ ID NO: 1, the lanosterol synthase comprises the following amino acid substitutions:
(a) D50G, K66R, N94S, G417S, E617V, and F726L;
(b) K85N and G158S;
(c) K47E, L92I, T360S, S372P, T444M, and R578P;
(d) F432S, D452G, and I536F;
(e) T360S, S372P, T444M, and R578P;
(f) L491Q, Y586F, and R660H;
(g) K85N, G158S, S515L, P526T, Q619L, and a truncation that results in deletion of the residue corresponding to position 742 in SEQ ID NO: 1; or
(h) I172N, C414S, L560M, and G679S.

9. The host cell of any one of claims 1-4, wherein the lanosterol synthase comprises an amino acid substitution or deletion relative to SEQ ID NO: 1 at one or more residues corresponding to position 14, 33, 47, 50, 66, 85, 92, 94, 122, 132, 145, 158, 193, 231, 248, 249, 286, 287, 289, 295, 296, 316, 329, 360, 371, 372, 407, 417, 423, 432, 442, 444, 479, 515, 526, 529, 564, 578, 617, 619, 620, 631, 655, 702, 726, 736, 738, and/or 742 in SEQ ID NO: 1.

10. The host cell of any one of claims 1-4 and 9, wherein the lanosterol synthase comprises relative to SEQ ID NO: 1:
a) R33Q, R193C, D289G, N295I, S296T, N620S, and Y736F;
b) K47E, L92I, T360S, S372P, T444M, and R578P;
c) D50G, K66R, N94S, G417S, E617V, and F726L;
d) N14Y, N132S, Y145C, R193H, I286F, L316R, F432I, E442V, T444S, I479S, K631R, and T655A;
e) E287G, K329N, E617V, and F726V;
f) E231V, A407V, Q423L, A529T, and Y564C;
g) V248F, D371V, and G702D;
h) G122C, H249L, and K738M; or
i) K85N, G158S, S515L, P526T, and Q619L, and a truncation resulting in a deletion of the residue corresponding to Q742 in SEQ ID NO: 1.

11. The host cell of any one of claims 1-10, wherein the lanosterol synthase comprises a sequence that is at least 90% identical to SEQ ID NO: 3, 83-87, 89-92, 94-95, 99, 118-120, 316-319, 321-326, 329, or 331.

12. The host cell of claim 11, wherein the lanosterol synthase comprises SEQ ID NO: 3, 83-87, 89-92, 94-95, 99, 118-120, 316-319, 321-326, 329, or 331.

13. The host cell of any one of claims 1-12, wherein the heterologous polynucleotide comprises a sequence that is at least 90% identical to SEQ ID NO: 4, 62-66, 68-71, 73-74, 78, 103-109, 111-117, 328, or 330.

14. The host cell of claim 13, wherein the heterologous polynucleotide comprises the sequence of SEQ ID NO: 4, 62-66, 68-71, 73-74, 78, 103-109, 111-117, 328, or 330.

15. A host cell that comprises a heterologous polynucleotide encoding a lanosterol synthase, wherein the lanosterol synthase comprises a sequence that is at least 90% identical to SEQ ID NO: 3, 83-87, 89-92, 94-95, 99, 100-102, 118-120, 316-319, 321-326, 329, or 331.

16. The host cell of claim 15, wherein the lanosterol synthase comprises SEQ ID NO: 3, 83-87, 89-92, 94-95, 99, 100-102, 118-120, 316-319, 321-326, 329, or 331.

17. A host cell that comprises a heterologous polynucleotide encoding a lanosterol synthase, wherein the lanosterol synthase comprises relative to SEQ ID NO: 1:
a) R33Q, R193C, D289G, N295I, S296T, N620S, and Y736F;
b) K47E, L92I, T360S, S372P, T444M, and R578P;
c) D50G, K66R, N94S, G417S, E617V, and F726L;
d) N14Y, N132S, Y145C, R193H, I286F, L316R, F432I, E442V, T444S, I479S, K631R, and T655A;
e) E287G, K329N, E617V, and F726V;
f) E231V, A407V, Q423L, A529T, and Y564C;
g) V248F, D371V, and G702D;
h) G122C, H249L, and K738M; or
i) K85N, G158S, S515L, P526T, and Q619L, and a truncation resulting in a deletion of the residue corresponding to Q742 in SEQ ID NO: 1.

18. A host cell that comprises a heterologous polynucleotide encoding a lanosterol synthase, wherein the heterologous polynucleotide comprises a sequence that is at least 90% identical to SEQ ID NO: 4, 62-66, 68-71, 73-74, 78, 80-82, 103-109, 111-117, 328, or 330.

19. The host cell of claim 18, wherein the heterologous polynucleotide comprises SEQ ID NO: 4, 62-66, 68-71, 73-74, 78, 80-82, 103-109, 111-117, 328, or 330.

20. The host cell of claim 1, wherein the host cell comprises a heterologous polynucleotide encoding a lanosterol synthase, wherein the lanosterol synthase comprises an amino acid substitution or deletion relative to SEQ ID NO: 313 at one or more residues corresponding to position 64, 120, 121, 136, 226, 268, 275, 281, 300, 322, 333, 438, 502, 604, 619, 628, 656, 693, 726, 727, 728, 729, 730, and/or 731.

21. The host cell of claim 20, wherein the lanosterol synthase comprises:
(a) the amino acid G at the residue corresponding to position 64 in SEQ ID NO: 313;
(b) the amino acid V at the residue corresponding to position 120 in SEQ ID NO: 313;
(c) the amino acid S at the residue corresponding to position 121 in SEQ ID NO: 313;
(d) the amino acid V at the residue corresponding to position 136 in SEQ ID NO: 313;
(e) the amino acid I at the residue corresponding to position 226 in SEQ ID NO: 313;
(f) the amino acid S at the residue corresponding to position 268 in SEQ ID NO: 313;
(g) the amino acid I at the residue corresponding to position 275 in SEQ ID NO: 313;
(h) the amino acid A at the residue corresponding to position 281 in SEQ ID NO: 313;
(i) the amino acid G at the residue corresponding to position 300 in SEQ ID NO: 313;
(j) the amino acid G at the residue corresponding to position 322 in SEQ ID NO: 313;
(k) the amino acid A at the residue corresponding to position 333 in SEQ ID NO: 313;
(l) the amino acid E at the residue corresponding to position 438 in SEQ ID NO: 313;
(m) the amino acid L at the residue corresponding to position 502 in SEQ ID NO: 313;
(n) the amino acid N at the residue corresponding to position 604 in SEQ ID NO: 313;
(o) the amino acid S at the residue corresponding to position 619 in SEQ ID NO: 313;
(p) the amino acid E at the residue corresponding to position 628 in SEQ ID NO: 313;
(q) the amino acid T at the residue corresponding to position 656 in SEQ ID NO: 313;
(r) the amino acid G at the residue corresponding to position 693 in SEQ ID NO: 313; and/or
(s) deletion of residues corresponding to positions 726-731 in SEQ ID NO: 313.

22. The host cell of any one of claims 1, 20 and 21, wherein the lanosterol synthase comprises relative to SEQ ID NO: 313:
(a) P121S, A136V, S300G, V322G, K438E, F502L, K628E, and deletion of residues corresponding to positions 726-731 in SEQ ID NO: 313;
(b) K268S, T281A, F502L, T604N, A656T, and E693G; or
(c) C619S, F275I, I120V, M226I, R64G, and T333A.

23. The host cell of any one of claims 1 and 20-22, wherein the lanosterol synthase comprises a sequence that is at least 90% identical to any one of SEQ ID NOs: 100-102.

24. The host cell of claim 23, wherein the lanosterol synthase comprises a sequence selected from SEQ ID NOs: 100-102.

25. The host of any one of claims 1 and 20-24, wherein the heterologous polynucleotide encoding the lanosterol synthase comprises a sequence that is at least 90% identical to a sequence selected from SEQ ID NOs: 80-82.

26. The host cell of claim 25, wherein the heterologous polynucleotide encoding the lanosterol synthase comprises a sequence selected from SEQ ID NOs: 80-82.

27. The host cell of any one of claims 1-26, wherein the host cell is capable of producing mevalonate.

28. The host cell of any one of claims 1-27, wherein the host cell is capable of producing at least 0.2 g/L mevalonate.

29. The host cell of any one of claims 1-28, wherein the host cell is capable of producing at least 0.7 g/L mevalonate.

30. The host cell of any one of claims 1-29, wherein the host cell is capable of producing at least 9 mg/L cucurbitadienol.

31. The host cell of any one of claims 1-30, wherein the host cell is capable of producing at least 1.1 fold more cucurbitadienol than a control host cell comprising SEQ ID NO: 1 and/or a control host cell comprising SEQ ID NO: 313.

32. The host cell of any one of claims 1-31, wherein the host cell is capable of producing at least 3 fold more cucurbitadienol than a control host cell comprising SEQ ID NO: 1 and/or a control host cell comprising SEQ ID NO: 313.

33. The host cell of any one of claims 1-32, wherein the host cell is capable of producing at most 200 mg/L lanosterol.

34. The host cell of any one of claims 1-33, wherein the host cell is capable of producing at least 5 mg/L oxidosqualene.

35. The host cell of any one of claims 1-34, wherein the host cell is capable of producing more mevalonate than a control host cell that does not comprise the heterologous polynucleotide.

36. The host cell of any one of claims 1-35, wherein the host cell further comprises one or more heterologous polynucleotides encoding one or more of: a UDP-glycosyltransferases (UGT) enzyme, a cucurbitadienol synthase (CDS) enzyme, a C11 hydroxylase, an epoxide hydrolase (EPH), and squalene epoxidase (SQE).

37. The host cell of claim 36, wherein the UGT enzyme comprises a sequence that is at least 90% identical to SEQ ID NO: 121.

38. The host cell of claim 36 or 37, wherein the CDS enzyme comprises a sequence that is at least 90% identical to any one of SEQ ID NOs: 226, SEQ ID NO: 235, SEQ ID NO: 232, and SEQ ID NO: 256.

39. The host cell of any one of claims 36-38, wherein the C11 hydroxylase comprises a sequence that is at least 90% identical to any one of SEQ ID NOs: 280-281, 305, and 315.

40. The host cell of any one of claims 36-39, wherein the EPH comprises a sequence that is at least 90% identical to any one of SEQ ID NO: 284-292 and 309-310.

41. The host cell of any one of claims 36-40, wherein the SQE comprises a sequence that is at least 90% identical to any one of SEQ ID NOs: 293-295 and 312.

42. The host cell of any one of claims 1-41, wherein the host cell further comprises a heterologous polynucleotide encoding a cytochrome P450 reductase.

43. The host cell of claim 42, wherein the cytochrome P450 reductase comprises a sequence that is at least 90% identical to any one of SEQ ID NOs: 282-283 and 306-307.

44. The host cell of any one of claims 1-41, wherein the host cell further comprises a heterologous polynucleotide encoding a cytochrome P450 reductase with reduced activity as compared to a control cytochrome P450 reductase or a heterologous polynucleotide that reduces cytochrome P450 activity.

45. The host cell of claim 44, wherein the control cytochrome P450 reductase is a wild-type P450 reductase.

46. The host cell of any one of claims 1-45, wherein the host cell is a yeast cell, a plant cell, or a bacterial cell.

47. The host cell of claim 46, wherein the host cell is a yeast cell.

48. The host cell of claim 47, wherein the yeast cell is a Saccharomyces cerevisiae cell.

49. The host cell of claim 47, wherein the yeast cell is a Yarrowia lipolytica cell.

50. The host cell of claim 46, wherein the host cell is a bacterial cell.

51. The host cell of claim 50, wherein the bacterial cell is an E. coli cell.

52. A method of producing a mogroside comprising culturing the host cell of any one of claims 1-51.

53. A method of producing mogrol comprising culturing the host cell of any one of claims 1-51.

54. The method of claim 52, wherein the mogroside is selected from mogroside I-A1 (MIA1), mogroside IE (MIE), mogroside II-A1 (MIIA1), mogroside II-A2 (MIIA2), mogroside III-A1 (MIIIA1), mogroside II-E (MIIE), mogroside III (MIII), siamenoside I, mogroside IV (MIV), mogroside IVa (MIVA), isomogroside IV, mogroside III-E (MIIIE), mogroside V (MV), mogroside VIA (MVIA), mogroside VIB (MVIB), isomogroside V, mogroside VIa1 (MVIa1), and/or mogroside VI (MVI).

55. The host cell of any one of claims 1-51, wherein the one or more mogrosides is selected from mogroside I-A1 (MIA1), mogroside IE (MIE), mogroside II-A1 (MIIA1), mogroside II-A2 (MIIA2), mogroside III-A1 (MIIIA1), mogroside II-E (MIIE), mogroside III (MIII), siamenoside I, mogroside IV (MIV), mogroside IVa (MIVA), isomogroside IV, mogroside III-E (MIIIE), mogroside V (MV), mogroside VIA (MVIA), mogroside VIB (MVIB), isomogroside V, mogroside VIa1 (MVIa1), and/or mogroside VI (MVI).

56. The host cell of any one of claims 1-51 and 55, further comprising a heterologous polynucleotide encoding an acetoacetyl COA synthase.

57. The host cell of claim 56, wherein the acetoacetyl COA synthase comprises a sequence that is at least 90% identical to SEQ ID NO: 6.

58. The host cell of claim 57, wherein the heterologous polynucleotide encoding the acetoacetyl COA synthase comprises a sequence that is at least 90% identical to SEQ ID NO: 7.

59. A method of producing mogrol, one or more mogrol precursors, and/or one or more mogrosides comprising culturing a host cell that comprises a heterologous polynucleotide encoding a lanosterol synthase, wherein the lanosterol synthase comprises an amino acid substitution or deletion relative to SEQ ID NO: 1 at one or more residues corresponding to position 14, 33, 47, 50, 66, 80, 83, 85, 92, 94, 107, 122, 132, 145, 158, 170, 172, 184, 193, 197, 198, 212, 213, 227, 228, 231, 235, 248, 249, 260, 282, 286, 287, 289, 295, 296, 309, 314, 316, 329, 344, 360, 370, 371, 372, 398, 407, 414, 417, 423, 432, 437, 442, 444, 452, 474, 479, 491, 498, 515, 526, 529, 536, 544, 552, 559, 560, 564, 578, 586, 608, 610, 617, 619, 620, 631, 638, 650, 655, 660, 679, 686, 702, 710, 726, 736, 738, and/or 742 in SEQ ID NO: 1 and wherein the host cell is capable of producing:
(a) one or more mogrol precursors selected from the group consisting of: squalene, 2-3-oxidosqualene, 2,3,22,23-dioxidosqualene, cucurbitadienol, 24, 25-expoxycucurbitadienol, 11-hydroxycucurbitadienol, 11-hydroxy-24,25-epoxycucurbitadienol, 11-hydroxycucurbitadienol, 11-oxo-cucurbitadienol, and 24,25-dihydroxycucurbitadienol;
(b) mogrol; and/or
(c) one or more mogrosides.

60. The method of claim 59, wherein the lanosterol synthase comprises 2, 3, 4, 5, 6, 7, 8, 9, 10, 11, or 12 amino acid substitutions and/or deletions relative to SEQ ID NO: 1.

61. The method of claim 59 or 60, wherein the lanosterol synthase comprises:
a) the amino acid Y at the residue corresponding to position 14 in SEQ ID NO:1;
b) the amino acid Q at the residue corresponding to position 33 in SEQ ID NO:1;
c) the amino acid E at the residue corresponding to position 47 in SEQ ID NO:1;
d) the amino acid G at the residue corresponding to position 50 in SEQ ID NO:1;
e) the amino acid R at the residue corresponding to position 66 in SEQ ID NO:1;
f) the amino acid G at the residue corresponding to position 80 in SEQ ID NO: 1;
g) the amino acid L at the residue corresponding to position 83 in SEQ ID NO: 1;
h) the amino acid N at the residue corresponding to position 85 in SEQ ID NO:1;
i) the amino acid I at the residue corresponding to position 92 in SEQ ID NO:1;
j) the amino acid S at the residue corresponding to position 94 in SEQ ID NO:1;
k) the amino acid D at the residue corresponding to position 107 in SEQ ID NO:1;
l) the amino acid C at the residue corresponding to position 122 in SEQ ID NO:1;
m) the amino acid S at the residue corresponding to position 132 in SEQ ID NO:1;
n) the amino acid C at the residue corresponding to position 145 in SEQ ID NO:1;
o) the amino acid S at the residue corresponding to position 158 in SEQ ID NO:1;
p) the amino acid A at the residue corresponding to position 170 in SEQ ID NO: 1;
q) the amino acid N at the residue corresponding to position 172 in SEQ ID NO:1;
r) the amino acid W at the residue corresponding to position 184 in SEQ ID NO:1;
s) the amino acid C or H at the residue corresponding to position 193 in SEQ ID NO:1;
t) the amino acid V at the residue corresponding to position 197 in SEQ ID NO:1;
u) the amino acid I at the residue corresponding to position 198 in SEQ ID NO: 1;
v) the amino acid I at the residue corresponding to position 212 in SEQ ID NO:1;
w) the amino acid L at the residue corresponding to position 213 in SEQ ID NO:1;
x) the amino acid L at the residue corresponding to position 227 in SEQ ID NO:1;
y) the amino acid T at the residue corresponding to position 228 in SEQ ID NO: 1;
z) the amino acid V at the residue corresponding to position 231 in SEQ ID NO:1;
aa) the amino acid M at the residue corresponding to position 235 in SEQ ID NO:1;
bb) the amino acid F at the residue corresponding to position 248 in SEQ ID NO:1;
cc) the amino acid L at the residue corresponding to position 249 in SEQ ID NO:1;
dd) the amino acid R at the residue corresponding to position 260 in SEQ ID NO:1;
cc) the amino acid I at the residue corresponding to position 282 in SEQ ID NO:1;
ff) the amino acid F at the residue corresponding to position 286 in SEQ ID NO: 1;
gg) the amino acid G at the residue corresponding to position 287 in SEQ ID NO:1;
hh) the amino acid G at the residue corresponding to position 289 in SEQ ID NO: 1;
ii) the amino acid I at the residue corresponding to position 295 in SEQ ID NO: 1;
jj) the amino acid T at the residue corresponding to position 296 in SEQ ID NO: 1;
kk) the amino acid F at the residue corresponding to position 309 in SEQ ID NO: 1;
ll) the amino acid S at the residue corresponding to position 314 in SEQ ID NO:1;
mm) the amino acid R at the residue corresponding to position 316 in SEQ ID NO:1;
nn) the amino acid N at the residue corresponding to position 329 in SEQ ID NO:1;
oo) the amino acid A at the residue corresponding to position 344 in SEQ ID NO: 1;
pp) the amino acid S at the residue corresponding to position 360 in SEQ ID NO:1;
qq) the amino acid L at the residue corresponding to position 370 in SEQ ID NO:1;
rr) the amino acid V at the residue corresponding to position 371 in SEQ ID NO:1;
ss) the amino acid P at the residue corresponding to position 372 in SEQ ID NO:1;
tt) the amino acid I at the residue corresponding to position 398 in SEQ ID NO: 1;
uu) the amino acid V at the residue corresponding to position 407 in SEQ ID NO:1;
vv) the amino acid S at the residue corresponding to position 414 in SEQ ID NO:1;
ww) the amino acid S at the residue corresponding to position 417 in SEQ ID NO:1;
xx) the amino acid L at the residue corresponding to position 423 in SEQ ID NO:1;
yy) the amino acid I or S at the residue corresponding to position 432 in SEQ ID NO:1;
zz) the amino acid L at the residue corresponding to position 437 in SEQ ID NO:1;
aaa) the amino acid V at the residue corresponding to position 442 in SEQ ID NO:1;
bbb) the amino acid M or S at the residue corresponding to position 444 in SEQ ID NO:1;
ccc) the amino acid G at the residue corresponding to position 452 in SEQ ID NO:1;
ddd) the amino acid V at the residue corresponding to position 474 in SEQ ID NO:1;
ccc) the amino acid S at the residue corresponding to position 479 in SEQ ID NO:1;
fff) the amino acid Q at the residue corresponding to position 491 in SEQ ID NO:1;
ggg) the amino acid N at the residue corresponding to position 498 in SEQ ID NO: 1;
hhh) the amino acid L at the residue corresponding to position 515 in SEQ ID NO:1;
iii) the amino acid T at the residue corresponding to position 526 in SEQ ID NO:1;
jjj) the amino acid T at the residue corresponding to position 529 in SEQ ID NO:1;
kkk) the amino acid F at the residue corresponding to position 536 in SEQ ID NO:1;
lll) the amino acid Y at the residue corresponding to position 544 in SEQ ID NO:1;
mmm) the amino acid E at the residue corresponding to position 552 in SEQ ID NO:1;
nnn) the amino acid A at the residue corresponding to position 559 in SEQ ID NO:1;
ooo) the amino acid M at the residue corresponding to position 560 in SEQ ID NO:1;
ppp) the amino acid C or N at the residue corresponding to position 564 in SEQ ID NO:1;
qqq) the amino acid P at the residue corresponding to position 578 in SEQ ID NO:1;
rrr) the amino acid F at the residue corresponding to position 586 in SEQ ID NO:1;
sss) the amino acid T at the residue corresponding to position 608 in SEQ ID NO:1;
ttt) the amino acid I at the residue corresponding to position 610 in SEQ ID NO: 1;
uuu) the amino acid V at the residue corresponding to position 617 in SEQ ID NO:1;
vvv) the amino acid L at the residue corresponding to position 619 in SEQ ID NO:1;
www) the amino acid S at the residue corresponding to position 620 in SEQ ID NO:1;
xxx) the amino acid E or R at the residue corresponding to position 631 in SEQ ID NO:1;
yyy) the amino acid D at the residue corresponding to position 638 in SEQ ID NO:1;
zzz) the amino acid L at the residue corresponding to position 650 in SEQ ID NO:1;
aaaa) the amino acid A at the residue corresponding to position 655 in SEQ ID NO:1;
bbbb) the amino acid H at the residue corresponding to position 660 in SEQ ID NO:1;
cccc) the amino acid S at the residue corresponding to position 679 in SEQ ID NO:1;
dddd) the amino acid E at the residue corresponding to position 686 in SEQ ID NO: 1;
eeee) the amino acid D at the residue corresponding to position 702 in SEQ ID NO:1;
ffff) the amino acid Q at the residue corresponding to position 710 in SEQ ID NO:1;
gggg) the amino acid L or V at the residue corresponding to position 726 in SEQ ID NO:1;
hhhh) the amino acid F at the residue corresponding to position 736 in SEQ ID NO:1;
iiii) the amino acid M at the residue corresponding to position 738 in SEQ ID NO:1; and/or
jjjj) a truncation that results in deletion of the residue corresponding to position 742 in SEQ ID NO: 1.

62. The method of any one of claims 59-61, wherein the lanosterol synthase comprises the amino acid substitution E617V, G107D, and/or K631E relative to SEQ ID NO: 1.

63. The method of any one of claims 59-61, wherein relative to SEQ ID NO: 1, the lanosterol synthase comprises:
a) R33Q, R193C, D289G, N295I, S296T, N620S, and Y736F;
b) R184W, L235M, L260R, and E710Q;
c) K47E, L92I, T360S, S372P, T444M, and R578P;
d) D50G, K66R, N94S, G417S, E617V, and F726L;
e) N14Y, N132S, Y145C, R193H, I286F, L316R, F432I, E442V, T444S, I479S, K631R, and T655A;
f) F432S, D452G, and I536F;
g) E287G, K329N, E617V, and F726V;
h) E231V, A407V, Q423L, A529T, and Y564C;
i) V248F, D371V, and G702D;
j) L197V, K282I, N314S, P370L, A608T, G638D, and F650L;
k) L491Q, Y586F, and R660H;
l) G122C, H249L, and K738M;
m) P227L, E474V, V559A, and Y564N;
n) K85N, G158S, S515L, P526T, Q619L, and a truncation resulting in a deletion of the residue corresponding to Q742 in SEQ ID NO: 1;
o) G107D and K631E;
p) T212I, W213L, N544Y, and V552E;
q) I172N, C414S, L560M, and G679S;
r) R193C, D289G, N295I, S296T, N620S, and Y736F;
s) K85N and G158S;
t) L197V, K282I, N314S, and P370L;
u) I172N, C414S, and L560M;
v) D371V, M610I, and G702D;
w) D371V, K498N, M610I, and G702D;
x) D80G, P83L, T170A, T198I, and A228T;
y) T360S, S372P, T444M, and R578P;
z) D50G, K66R, N94S, G417S, and E617V; or
aa) L309F, V344A, T398I, and K686E.

64. The method of any one of claims 59-61 and 63, wherein relative to SEQ ID NO: 1, the lanosterol synthase comprises the following amino acid substitutions:
a) R193C, D289G, N295I, S296T, N620S, and Y736F;
b) F432S, D452G, and I536F;
c) K85N and G158S;
d) L197V, K282I, N314S, and P370L;
e) I172N, C414S, L560M, and G679S;
f) I172N, C414S, and L560M;
g) D371V, M610I, and G702D;
h) D371V, K498N, M610I, and G702D;
i) D80G, P83L, T170A, T198I, and A228T;
j) D50G, K66R, N94S, G417S, E617V, and F726L;
k) T360S, S372P, T444M, and R578P;
l) D50G, K66R, N94S, G417S, and E617V; and
m) L309F, V344A, T398I, and K686E.

65. The method of any one of claims 59-61 and 63, wherein relative to SEQ ID NO: 1, the lanosterol synthase comprises the following amino acid substitutions:
a) D50G, K66R, N94S, G417S, E617V, and F726L;
b) K85N and G158S;
c) K47E, L92I, T360S, S372P, T444M, and R578P;
d) F432S, D452G, and I536F;
e) T360S, S372P, T444M, and R578P;
f) L491Q, Y586F, and R660H;
g) K85N, G158S, S515L, P526T, Q619L, and a truncation that results in deletion of the residue corresponding to position 742 in SEQ ID NO: 1; or
h) I172N, C414S, L560M, and G679S.

66. The method of any one of claims 59-61, wherein the lanosterol synthase comprises an amino acid substitution or deletion relative to SEQ ID NO: 1 at one or more residues corresponding to position 14, 33, 47, 50, 66, 85, 92, 94, 122, 132, 145, 158, 193, 231, 248, 249, 286, 287, 289, 295, 296, 316, 329, 360, 371, 372, 407, 417, 423, 432, 442, 444, 479, 515, 526, 529, 564, 578, 617, 619, 620, 631, 655, 702, 726, 736, 738, and/or 742 in SEQ ID NO: 1.

67. The method of any one of claims 59-61 and 66, wherein the lanosterol synthase comprises relative to SEQ ID NO: 1:
a) R33Q, R193C, D289G, N295I, S296T, N620S, and Y736F;
b) K47E, L92I, T360S, S372P, T444M, and R578P;
c) D50G, K66R, N94S, G417S, E617V, and F726L;
d) N14Y, N132S, Y145C, R193H, I286F, L316R, F432I, E442V, T444S, I479S, K631R, and T655A;
e) E287G, K329N, E617V, and F726V;
f) E231V, A407V, Q423L, A529T, and Y564C;
g) V248F, D371V, and G702D;
h) G122C, H249L, and K738M; or
i) K85N, G158S, S515L, P526T, and Q619L, and a truncation resulting in a deletion of the residue corresponding to Q742 in SEQ ID NO: 1.

68. The method of any one of claims 59-65, wherein the lanosterol synthase comprises a sequence that is at least 90% identical to SEQ ID NO: 3, 83-87, 89-92, 94-95, 99, 118-120, 316-319, 321-326, 329, or 331.

69. The method of claim 68, wherein the lanosterol synthase comprises SEQ ID NO: 3, 83-87, 89-92, 94-95, 99, 118-120, 316-319, 321-326, 329, or 331.

70. The method of any one of claims 59-69, wherein the heterologous polynucleotide comprises a sequence that is at least 90% identical to SEQ ID NO: 4, 62-66, 68-71, 73-74, 78, 103-109, 111-117, 328, or 330.

71. The method of claim 70, wherein the heterologous polynucleotide comprises the sequence of SEQ ID NO: 4, 62-66, 68-71, 73-74, 78, 103-109, 111-117, 328, or 330.

72. A method of producing mogrol, one or more mogrol precursors, and/or one or more mogrosides comprising culturing a host cell that comprises a heterologous polynucleotide encoding a lanosterol synthase, wherein the lanosterol synthase comprises an amino acid substitution or deletion relative to SEQ ID NO: 313 at one or more residues corresponding to position 64, 120, 121, 136, 226, 268, 275, 281, 300, 322, 333, 438, 502, 604, 619, 628, 656, 693, 726, 727, 728, 729, 730, and/or 731.

73. The method of claim 72, wherein the lanosterol synthase comprises:
(a) the amino acid G at the residue corresponding to position 64 in SEQ ID NO: 313;
(b) the amino acid V at the residue corresponding to position 120 in SEQ ID NO: 313;
(c) the amino acid S at the residue corresponding to position 121 in SEQ ID NO: 313;
(d) the amino acid V at the residue corresponding to position 136 in SEQ ID NO: 313;
(e) the amino acid I at the residue corresponding to position 226 in SEQ ID NO: 313;
(f) the amino acid S at the residue corresponding to position 268 in SEQ ID NO: 313;
(g) the amino acid I at the residue corresponding to position 275 in SEQ ID NO: 313;
(h) the amino acid A at the residue corresponding to position 281 in SEQ ID NO: 313;
(i) the amino acid G at the residue corresponding to position 300 in SEQ ID NO: 313;
(j) the amino acid G at the residue corresponding to position 322 in SEQ ID NO: 313;
(k) the amino acid A at the residue corresponding to position 333 in SEQ ID NO: 313;
(l) the amino acid E at the residue corresponding to position 438 in SEQ ID NO: 313;
(m) the amino acid L at the residue corresponding to position 502 in SEQ ID NO: 313;
(n) the amino acid N at the residue corresponding to position 604 in SEQ ID NO: 313;
(o) the amino acid S at the residue corresponding to position 619 in SEQ ID NO: 313;
(p) the amino acid E at the residue corresponding to position 628 in SEQ ID NO: 313;
(q) the amino acid T at the residue corresponding to position 656 in SEQ ID NO: 313;
(r) the amino acid G at the residue corresponding to position 693 in SEQ ID NO: 313; and/or
(s) deletion of residues corresponding to positions 726-731 in SEQ ID NO: 313.

74. The method of claim 72 or 73, wherein the lanosterol synthase comprises relative to SEQ ID NO: 313:
(a) P121S, A136V, S300G, V322G, K438E, F502L, K628E, and deletion of residues corresponding to positions 726-731 in SEQ ID NO: 313;
(b) K268S, T281A, F502L, T604N, A656T, and E693G; or
(c) C619S, F275I, I120V, M226I, R64G, and T333A.

75. The method of any one of claims 72-74, wherein the lanosterol synthase comprises a sequence that is at least 90% identical to any one of SEQ ID NOs: 100-102.

76. The method of claim 75, wherein the lanosterol synthase comprises a sequence selected from SEQ ID NOs: 100-102.

77. The method of any one of claims 72-76, wherein the heterologous polynucleotide encoding the lanosterol synthase comprises a sequence that is at least 90% identical to a sequence selected from SEQ ID NOs: 80-82.

78. The method of claim 77, wherein the heterologous polynucleotide encoding the lanosterol synthase comprises a sequence selected from SEQ ID NOs: 80-82.

79. The method of any one of claims 59-78, wherein the host cell is capable of producing mevalonate.

80. The method of any one of claims 59-79, wherein the host cell is capable of producing at least 0.2 g/L mevalonate.

81. The method of any one of claims 59-80, wherein the host cell is capable of producing at least 0.7 g/L mevalonate.

82. The method of any one of claims 59-81, wherein the host cell is capable of producing at least 9 mg/L cucurbitadienol.

83. The method of any one of claims 59-82, wherein the host cell is capable of producing at least 1.1 fold more cucurbitadienol than a control host cell comprising SEQ ID NO: 1 and/or a control host cell comprising SEQ ID NO: 313.

84. The method of any one of claims 59-83, wherein the host cell is capable of producing at least 3 fold more cucurbitadienol than a control host cell comprising SEQ ID NO: 1 and/or a control host cell comprising SEQ ID NO: 313.

85. The method of any one of claims 59-84, wherein the host cell is capable of producing at most 200 mg/L lanosterol.

86. The method of any one of claims 59-85, wherein the host cell is capable of producing at least 5 mg/L oxidosqualene.

87. The method of any one of claims 59-86, wherein the host cell is capable of producing more mevalonate than a control host cell that does not comprise the heterologous polynucleotide.

88. The method of any one of claims 59-87, wherein the host cell further comprises one or more heterologous polynucleotides encoding one or more of: a UDP-glycosyltransferases (UGT) enzyme, a cucurbitadienol synthase (CDS) enzyme, a C11 hydroxylase, an epoxide hydrolase (EPH), and squalene epoxidase (SQE).

89. The method of claim 88, wherein the UGT enzyme comprises a sequence that is at least 90% identical to SEQ ID NO: 121.

90. The method of claim 88 or 89, wherein the CDS enzyme comprises a sequence that is at least 90% identical to any one of SEQ ID NOs: 226, SEQ ID NO: 235, SEQ ID NO: 232, and SEQ ID NO: 256.

91. The method of any one of claims 88-90, wherein the C11 hydroxylase comprises a sequence that is at least 90% identical to any one of SEQ ID NOs: 280-281, 305, and 315.

92. The method of any one of claims 88-91, wherein the EPH comprises a sequence that is at least 90% identical to any one of SEQ ID NO: 284-292 and 309-310.

93. The method of any one of claims 88-92, wherein the SQE comprises a sequence that is at least 90% identical to any one of SEQ ID NOs: 293-295 and 312.

94. The method of any one of claims 59-93, wherein the host cell further comprises a heterologous polynucleotide encoding a cytochrome P450 reductase.

95. The method of claim 94, wherein the cytochrome P450 reductase comprises a sequence that is at least 90% identical to any one of SEQ ID NOs: 282-283 and 306-307.

96. The method of any one of claims 59-93, wherein the host cell further comprises a heterologous polynucleotide encoding a cytochrome P450 reductase with reduced activity as compared to a control cytochrome P450 reductase or a heterologous polynucleotide that reduces cytochrome P450 activity.

97. The method of claim 96, wherein the control cytochrome P450 reductase is a wild-type P450 reductase.

98. The method of any one of claims 59-97, wherein the host cell is a yeast cell, a plant cell, or a bacterial cell.

99. The method of claim 98, wherein the host cell is a yeast cell.

100. The method of claim 99, wherein the yeast cell is a Saccharomyces cerevisiae cell.

101. The method of claim 99, wherein the yeast cell is a Yarrowia lipolytica cell.

102. The method of claim 98, wherein the host cell is a bacterial cell.

103. The method of claim 102, wherein the bacterial cell is an E. coli cell.

104. The method of any one of claims 59-103, wherein the host cell further comprises a heterologous polynucleotide encoding an acetoacetyl COA synthase.

105. The method of claim 104, wherein the acetoacetyl COA synthase comprises a sequence that is at least 90% identical to SEQ ID NO: 6.

106. The method of claim 105, wherein the heterologous polynucleotide encoding the acetoacetyl COA synthase comprises a sequence that is at least 90% identical to SEQ ID NO: 7.

107. The method of any one of claims 59-106, wherein the mogroside is selected from mogroside I-A1 (MIA1), mogroside IE (MIE), mogroside II-A1 (MIIA1), mogroside II-A2 (MIIA2), mogroside III-A1 (MIIIA1), mogroside II-E (MIIE), mogroside III (MIII), siamenoside I, mogroside IV (MIV), mogroside IVa (MIVA), isomogroside IV, mogroside III-E (MIIIE), mogroside V (MV), mogroside VIA (MVIA), mogroside VIB (MVIB), isomogroside V, mogroside VIa1 (MVIa1), and/or mogroside VI (MVI).
